(19) United States
(12) Patent Application Publication
(10) Pub. No.: US 2024/0415939 A1
(43) Pub. Date: Dec. 19, 2024

(54) COMPOSITIONS FOR GENOME EDITING AND METHODS OF USE THEREOF

(71) Applicant: ARIZONA BOARD OF REGENTS ON BEHALF OF THE UNIVERSITY OF ARIZONA, Tucson, AZ (US)

(72) Inventors: Arun Dhar, Tucson, AZ (US); F. C. Thomas Allnutt, Glenelg, MD (US); Roberto Cruz Flores, Tucson, AZ (US)

(21) Appl. No.: 18/002,767

(22) PCT Filed: Jun. 30, 2021
(86) PCT No.: PCT/US21/39954

§ 371 Date: Dec. 21, 2022

Related U.S. Application Data
(60) Provisional application No. 63/046,493, filed on Jun. 30, 2020.

(51) Int. Cl. A61K 38/46; A61K 48/00; C12N 9/22; C12N 15/11; C12N 15/85

(52) U.S. Cl. CPC A61K 38/465; A61K 48/0075; C12N 9/22; C12N 15/11; C12N 15/85; C12N 2310/20; C12N 2800/80

(57) ABSTRACT

The present disclosure concerns methods and compositions for inhibiting replication of viruses within an organism in need thereof. In some cases, the methods and compositions can be applicable to aquacultured organisms. The aquacultured organisms can include, but are not limited to, crustacean organisms such as shrimp.

### CROSS-REFERENCE

This application claims the benefit of U.S. Provisional Application No. 63/046,493, entitled “COMPOSITIONS FOR GENOME EDITING AND METHODS OF USE THEREOF”, filed on Jun. 30, 2020, which is incorporated by reference herein in its entirety.

### STATEMENT REGARDING FEDERALLY SPONSORED RESEARCH OR DEVELOPMENT

This invention was made with government support under Grant No. 2016-33610-25458 awarded by USDA/NIFA. The government has certain rights in the invention.

### REFERENCE TO A SEQUENCE LISTING

Applicant asserts that the information recorded in the form of an Annex C/ST.25 text file submitted under Rule 13ter.1 (a), entitled UNIA_20_09_PCT_Sequence_Listing_ST25, is identical to that forming part of the international application as filed. The content of the sequence listing is incorporated herein by reference in its entirety.

### BACKGROUND OF THE INVENTION

Viral diseases are a major problem in animal husbandry, such as in aquaculture of crustaceans such as shrimp and prawns. Periodic outbreaks of several major viral disease have caused catastrophic losses to shrimp farmers around the globe; examples include an outbreak of white spot disease caused by white spot syndrome virus (WSSV) in the US in 2017, Australia in 2016, Mozambique and Madagascar in 2010-2011, and Saudi Arabia in 2012 among many others which nearly collapsed the shrimp farming industry in those countries. As crustacean viruses, such as WSSV, are now believed to be present worldwide, they continue to threaten the long-term sustainability of crustacean aquaculture.

### SUMMARY OF THE INVENTION

Provided herein are methods, compositions, and systems for inhibiting replication of viruses. In some embodiments, the present disclosure provides for a method for inhibiting infection of or reducing replication of a virus in an animal in need thereof. In some embodiments, the present disclosure provides for a method for inhibiting infection of or reducing replication of a virus in an animal in need thereof, comprising introducing to a cell of the animal a nuclease comprising a gene-binding moiety, wherein the gene binding moiety is configured to bind to at least one gene of the virus.

In some aspects, the present disclosure provides for a method for inhibiting infection of or reducing replication of a virus in an animal in need thereof, comprising introducing to a cell of the animal a nuclease comprising a gene-binding moiety, wherein the gene binding moiety is configured to bind at least one gene of the virus, wherein the one or more genes of the virus encode a gene essential for replication of the virus.

In some aspects, the present disclosure provides for a method for inhibiting infection of or reducing replication of a virus in an animal in need thereof, comprising introducing to a cell of the animal a nuclease comprising a gene-binding moiety, wherein the gene binding moiety is configured to bind at least one gene of the virus, wherein the one or more genes of the virus encode one or more genes essential to replication of the virus, wherein the virus belongs to the family Nimaviridae.

In some aspects, the present disclosure provides for a method for inhibiting infection of or reducing replication of a virus in an animal in need thereof, comprising introducing to a cell of the animal a nuclease comprising a gene-binding moiety, wherein the gene binding moiety is configured to bind at least one gene of the virus, wherein the one or more genes of the virus encode ICP11 or a fragment thereof, VP19 or a fragment thereof, VP26 or a fragment thereof, collagen-like protein (WSSV-CLP) or a fragment thereof, or any combination thereof, wherein the virus belongs to the family Nimaviridae. In some embodiments, the animal is a crustacean. In some embodiments, the crustacean is a decapod, shrimp, a prawn, a crab, or a crayfish. In some embodiments, the method comprises inhibiting infection in a shrimp, wherein the shrimp is Litopenaeus vannamei. In some embodiments, the virus belongs to the genus Whispovirus. In some embodiments, the virus is White spot syndrome virus (WSSV). In some embodiments, the gene-binding moiety is configured to bind a plurality of different portions of the one or more genes of the virus. In some embodiments, the gene-binding moiety is configured to bind a plurality of different portions of the one or more genes of the virus that are important in replication of the virus. In some embodiments, the gene-binding moiety is configured to bind a plurality of different portions of the one or more genes of the virus wherein the gene is essential to the virus. In some embodiments, the gene-binding moiety is configured to bind a combination of at least one of ICP11, VP19, VP26, collagen-like protein (WSSV-CLP), or any combination thereof. In some embodiments, the gene-binding moiety is configured to bind a combination of at least two, at least three, or all four of ICP11, VP19, VP26, collagen-like protein (WSSV-CLP), or any combination thereof. In some embodiments, the gene binding moiety is configured to further bind at least one additional gene of the virus comprising DNA polymerase, ribonucleotide reductase subunit 1 (RR1), VP28, or any combination thereof. In some embodiments, the nuclease is a programmable nuclease comprising at least one of a CRISPR-associated (Cas) polypeptide, a zinc finger nuclease (ZFN), a transcription activator-like effector nuclease (TALEN), or a combination thereof. In some embodiments, the nuclease is configured to bind at least 5, or at least 18-24 consecutive nucleotides at least one sequence selected from SEQ ID NOs: 22-82 or a variant having at least 80%, 90%, 95%, or 99% identity thereto. In some embodiments, the nuclease is further configured to bind at least 5, or at least 18-24 consecutive nucleotides of at least one, at least two, or at least three sequences selected from SEQ ID NOs: 1-9 or a variant having at least 80%, 90%, 95%, or 99% identity thereto. In some embodiments, the nuclease is a programmable nuclease comprising a CRISPR-associated (Cas) polypeptide, wherein the Cas polypeptide is a type I CRISPR-associated (Cas) polypeptide, a type II CRISPR-associated (Cas) polypeptide, a type III CRISPR-associated (Cas) polypeptide, a type IV CRISPR-associated (Cas) polypeptide, a type V CRISPR-associated (Cas) polypeptide, a type VI CRISPR-associated (Cas) polypeptide. In some embodiments, the gene-binding moiety of the nuclease comprises a heterologous RNA polynucleotide configured to hybridize to the one or more genes of the virus. In some embodiments, the heterologous RNA polynucleotide comprises at least one, at least two, or at least three targeting sequences, wherein the targeting sequence comprises at least 17 consecutive nucleotides of at least one sequence selected from SEQ ID NOs: 83-143 (or a complement thereof) or a variant having at least 80%, 90%, 95%, or 99% identity thereto. In some embodiments, the heterologous RNA polynucleotide further comprises at least one, at least two, or at least three targeting sequences, wherein the targeting sequence comprises at least 17 consecutive nucleotides of at least one sequence selected from SEQ ID NOs: 10-18 or a variant having at least 80%, 90%, 95%, or 99% identity thereto. In some embodiments, introducing a nuclease comprising a gene-binding moiety to the cell of the animal comprises contacting the cell with the nuclease. In some embodiments, the nuclease comprises a ribonucleoprotein complex comprising a Cas polypeptide and at least one, at least two, or at least three heterologous RNA polynucleotides configured to hybridize to the one or more genes of the virus. In some embodiments, introducing a nuclease comprising a gene-binding moiety to the cell of the animal comprises contacting the cell with a capped mRNA comprising a sequence encoding the nuclease. In some embodiments, the nuclease comprises a Cas polypeptide, wherein introducing a nuclease comprising a gene-binding moiety to the cell of the animal further comprises contacting the cell with at least one, at least two, or at least three heterologous RNA polynucleotides configured to hybridize to the one or more genes of the virus. In some embodiments, the capped mRNA and the heterologous RNA polynucleotide are separate RNAs. In some embodiments, introducing a nuclease comprising a gene-binding moiety to the cell of the animal comprises contacting the cell with a vector comprising a sequence encoding the nuclease. In some embodiments, the nuclease comprises a Cas polypeptide, wherein the vector further encodes at least one, at least two, or at least three heterologous RNA polynucleotides configured to hybridize to the one or more genes of the virus. In some embodiments, the vector is a plasmid, a minicircle, or a viral vector. In some embodiments, the vector is a viral vector, wherein the viral vector is a baculoviral vector. In some embodiments, the sequence encoding the nuclease is codon-optimized for expression in the crustacean. In some embodiments, the introducing occurs in vivo, ex vivo, or in vitro. In some embodiments, the nuclease cleaves viral genomic DNA encoding the one or more genes of the virus within the cell of the animal. In some embodiments, the method results in delay of mortality of the animal upon infection with the virus belonging to the family Nimaviridae. In some embodiments, the method results in reduced mortality of the animal upon infection with the virus belonging to the family Nimaviridae. In some embodiments, introducing to a cell of the animal the nuclease comprises injecting the animal with the nuclease or a vector encoding the nuclease. In some embodiments, introducing to a cell of the animal the nuclease comprises administering orally to the animal the nuclease or a vector encoding the nuclease.

In some aspects, the present disclosure provides for a vector comprising a sequence encoding at least one programmable nuclease configured to bind at least one viral gene of a virus from the family Nimaviridae, wherein the at least one viral gene comprises ICP11 or a fragment thereof, VP19 or a fragment thereof, VP26 or a fragment thereof, collagen like protein (WSSV-CLP) or a fragment thereof, or any combination thereof. In some embodiments, the vector is a plasmid, a minicircle, or a viral vector. In some embodiments, the viral vector is a baculoviral vector. In some embodiments, the nuclease is a programmable nuclease comprising at least one of a CRISPR-associated (Cas) polypeptide, a zinc finger nuclease (ZFN), a transcription activator-like effector nuclease (TALEN), or a combination thereof. In some embodiments, the programmable nuclease is configured to bind a plurality of different portions of the one or more genes of the virus. In some embodiments, the programmable nuclease is configured to bind a combination of at least two, at least three, or all four of ICP11, VP19, VP26, or collagen-like protein. In some embodiments, the programmable nuclease is configured to further bind at least additional gene of the virus comprising DNA polymerase, RR1, VP28, or any combination thereof. In some embodiments, the nuclease is configured to bind at least 5, or at least 18-24 consecutive nucleotides at least one sequence selected from SEQ ID NOs: 22-70 or a variant having at least 80%, 90%, 95%, or 99% identity thereto. In some embodiments, the nuclease is further configured to bind at least 5, or at least 18-24 consecutive nucleotides of at least one, at least two, or at least three sequences selected from SEQ ID NOs: 1-9 or a variant having at least 80%, 90%, 95%, or 99% identity thereto. In some embodiments, the programmable nuclease comprises a CRISPR-associated (Cas) polypeptide, wherein the Cas polypeptide is a type I CRISPR-associated (Cas) polypeptide, a type II CRISPR-associated (Cas) polypeptide, a type III CRISPR-associated (Cas) polypeptide, a type IV CRISPR-associated (Cas) polypeptide, a type V CRISPR-associated (Cas) polypeptide, a type VI CRISPR-associated (Cas) polypeptide. In some embodiments, the vector further comprises a second sequence encoding at least one, at least two, or at least three heterologous RNA polynucleotides configured to hybridize to the one or more genes of the virus. In some embodiments, the heterologous RNA polynucleotide comprises at least one, at least two, or at least three targeting sequences, wherein the targeting sequence comprises at least 17 consecutive nucleotides of at least one sequence selected from SEQ ID NOs: 83-143 (or a complement thereof), a variant having at least 80%, 90%, 95%, or 99% identity thereto, or a variant substantially identical thereto. In some embodiments, the heterologous RNA polynucleotide comprises at least one, at least two, or at least three targeting sequences, wherein the targeting sequence comprises at least 17 consecutive nucleotides of at least one sequence selected from SEQ ID NOs: 10-18. In some embodiments, the sequence encoding the heterologous RNA polynucleotide is operably linked to a sequence comprising an ie1 promoter from the virus from the family Nimaviridae. In some embodiments, the sequence encoding the heterologous RNA polynucleotide is operably linked to a sequence comprising an ie1 promoter from white spot syndrome virus (WSSV). In some embodiments, the sequence encoding the heterologous RNA polynucleotide is operably linked to a sequence comprising at least 100 consecutive nucleotides of SEQ ID NO:21, a variant having at least 80%, at least 90%, at least 95%, at least 99% identity thereto, or a variant substantially identical thereto. In some embodiments, the programmable nuclease is operably linked to a sequence comprising a P2 promoter from infectious hypodermal and hematopoietic necrosis virus (IHHNV) of shrimp. In some embodiments, the programmable nuclease is operably linked to a sequence comprising at least 100 consecutive nucleotides of any one of SEQ ID NOs: 20-162, a variant having at least 80%, at least 90%, at least 95%, at least 99% identity thereto, or a variant substantially identical thereto. In some embodiments, the sequence encoding the programmable nuclease is codon-optimized for expression in a crustacean species. In some embodiments, the crustacean species is a shrimp, a prawn, a crab, or a crayfish. In some embodiments, the shrimp is Litopenaeus vanmamei.

In some aspects, the present disclosure provides for a pharmaceutically-acceptable composition, comprising any of the vectors described herein and a pharmaceutically-acceptable excipient.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows a schematic depicting design of the p14 (RR1-targeted) multiplex CRISPR/Cas vector for protection of shrimp from Nimaviridae (e.g., WSSV) infection; this design was used for analogous VP28 targeting plasmid (p12) and DNApol targeting plasmid (p13).

FIG. 2 demonstrates that oral delivery of select targeting sequences for DNApol results in indel formation by select targeting sequences using the method of Example 5.

FIG. 3 shows that intramuscular delivery of select targeting sequences for RR1 results in indel formation by select targeting sequences using the method of Example 5.

FIG. 4 depicts a mortality plot depicting mortality of shrimp post oral Nimaviridae challenge during oral challenge run 1 in Example 5, demonstrating that gene-targeting vectors cause delay of mortality from Nimaviridae challenge.

FIG. 5 depicts a mortality plot depicting mortality of older shrimp post oral Nimaviridae challenge during oral challenge run 2 in Example 5, demonstrating that gene-targeting vectors cause delay of mortality from Nimaviridae challenge in older shrimp.

FIG. 6 depicts a schematic showing a plasmid diagram of the p23 plasmid targeting VP26, containing P2-Cas9 (shrimp optimized), followed by ie1 promoter−tRNA−sgRNA1 (target+scaffold)−tRNA−sgRNA2 (target+scaffold)−tRNA−sgRNA3 (target+scaffold), followed by UUUUUU, followed by ampicillin+ bacterial ori. The three sgRNA targeting sequences were: TTTGCTGCACACGTCAATGA (VP26_1), TGCGAGTCCCAATTCAAAGA (VP26_2), and CAATACTCCTCTTGGAAAGG (VP26_2).

FIG. 7 depicts a schematic showing a plasmid diagram of the p24 plasmid targeting ICP11, containing P2-Cas9 (shrimp optimized), followed by ie1 promoter−IRNA−sgRNA1 (target+scaffold)−tRNA−sgRNA2 (target+scaffold)−tRNA−sgRNA3 (target+scaffold), followed by UUUUUU, followed by ampicillin+ bacterial ori. The three sgRNA targeting sequences were: TCTGTTGTTGGCACAATCAT (ICP11_1), TTCTGTTGTTGGCACAATCA (ICP11_2), and TTTGACTGGAGATTCAACGC (ICP11_3).

FIG. 8 depicts a schematic showing a plasmid diagram of the p25 plasmid targeting VP19, containing P2-Cas9 (shrimp optimized), followed by ie1 promoter-tRNA−sgRNA1 (target+scaffold)−tRNA−sgRNA2 (target+scaffold)−tRNA−sgRNA3 (target+scaffold), followed by UUUUUU, followed by ampicillin+ bacterial ori. The three sgRNA targeting sequences are: TGTATGGATGGGACCAAAGA (VP19_1), TTATGTCTTACCCCAAGAGG (VP19_2), and TACACCATGGAAGATCTTGA (VP19_3).

FIG. 9 depicts a schematic showing a plasmid diagram of the p26 plasmid targeting collagenase-like gene, containing P2-Cas9 (shrimp optimized), followed by ie1 promoter-tRNA-sgRNA1 (target+scaffold)−tRNA−sgRNA2 (target+scaffold)−tRNA−sgRNA3 (target+scaffold), followed by UUUUUU, followed by ampicillin+ bacterial ori. The three sgRNA targeting sequences were: TTGTTTGGAGAGCCTATTAG (collagenase-like gene_1), TGTTTGGAGAGCCTATTAGA (collagenase-like gene_2), and TACATTGACCAAGGGGCGTT (collagenase-like gene_3).

FIGS. 10, 11, 12, 13, 14, 15, 16, 17, and 18 depict schematics showing plasmid diagrams of vector plasmids described herein (e.g., including SEQ ID NOs: 145, 146, 147, 148, and 149).

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

### Definitions

The practice of some methods disclosed herein employ, unless otherwise indicated, techniques of immunology, biochemistry, chemistry, molecular biology, microbiology, cell biology, genomics and recombinant DNA. See for example Sambrook and Green, Molecular Cloning: A Laboratory Manual, 4th Edition (2012); the series Current Protocols in Molecular Biology (F. M. Ausubel, et al. eds.); the series Methods In Enzymology (Academic Press, Inc.), PCR 2: A Practical Approach (M.J. MacPherson, B. D. Hames and G. R. Taylor eds. (1995), Harlow and Lane, eds. (1988) Antibodies, A Laboratory Manual, and Culture of Animal Cells: A Manual of Basic Technique and Specialized Applications, 6th Edition (R.I. Freshney, ed. (2010)) (which is entirely incorporated by reference herein)

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

As used herein, a “cell” generally refers to a biological cell. A cell may be the basic structural, functional and/or biological unit of a living organism. A cell may originate from any organism having one or more cells. Some non-limiting examples include: a prokaryotic cell, eukaryotic cell, a bacterial cell, an archaeal cell, a cell of a single-cell eukaryotic organism, a protozoa cell, a cell from a plant (e.g., cells from plant crops, fruits, vegetables, grains, soy bean, corn, maize, wheat, seeds, tomatoes, rice, cassava, sugarcane, pumpkin, hay, potatoes, cotton, cannabis, tobacco, flowering plants, conifers, gymnosperms, ferns, clubmosses, hornworts, liverworts, mosses), an algal cell, (e.g., Botryococcus braunii, Chlamydomonas reinhardtii, Namnochloropsis gaditana, Chlorella pyrenoidosa, Sargassum patens, C. agardh, and the like), seaweeds (e.g., kelp), a fungal cell (e.g., a yeast cell, a cell from a mushroom), an animal cell, a cell from an invertebrate animal (e.g., fruit fly, cnidarian, echinoderm, nematode, crustacean, etc.), a cell from a vertebrate animal (e.g., fish, amphibian, reptile, bird, mammal), a cell from a mammal (e.g., a pig, a cow, a goat, a sheep, a rodent, a rat, a mouse, a non-human primate, a buman, etc.), and etcetera. Sometimes a cell is not originating from a natural organism (e.g., a cell can be a synthetically made, sometimes termed an artificial cell).

The term “nucleotide,” as used herein, generally refers to a base-sugar-phosphate combination. A nucleotide may comprise a synthetic nucleotide. A nucleotide may comprise a synthetic nucleotide analog. Nucleotides may be monomeric units of a nucleic acid sequence (e.g., deoxyribonucleic acid (DNA) and ribonucleic acid (RNA)). The term nucleotide may include ribonucleoside triphosphates adenosine triphosphate (ATP), uridine triphosphate (UTP), cytosine triphosphate (CTP), guanosine triphosphate (GTP) and deoxyribonucleoside triphosphates such as dATP, dCTP, dITP, dUTP, dGTP, dTTP, or derivatives thereof.

The terms “polynucleotide,” “oligonucleotide,” and “nucleic acid” are used interchangeably to generally refer to a polymeric form of nucleotides of any length, either deoxyribonucleotides or ribonucleotides, or analogs thereof, either in single-, double-, or multi-stranded form. A polynucleotide may be exogenous or endogenous to a cell. A polynucleotide may exist in a cell-free environment. A polynucleotide may be a gene or fragment thereof. A polynucleotide may be DNA. A polynucleotide may be RNA. A polynucleotide may have any three-dimensional structure and may perform any function. A polynucleotide may comprise one or more analogs (e.g., altered backbone, sugar, or nucleobase).

The terms “peptide,” “polypeptide,” and “protein” are used interchangeably herein to generally refer to a polymer of at least two amino acid residues joined by peptide bond(s). This term does not connote a specific length of polymer, nor is it intended to imply or distinguish whether the peptide is produced using recombinant techniques, chemical or enzymatic synthesis, or is naturally occurring. The terms apply to naturally occurring amino acid polymers as well as amino acid polymers comprising at least one modified amino acid. In some cases, the polymer may be interrupted by non-amino acids. The terms include amino acid chains of any length, including full length proteins, and proteins with or without secondary and/or tertiary structure (e.g., domains). The terms also encompass an amino acid polymer that has been modified, for example, by disulfide bond formation, glycosylation, lipidation, acetylation, phosphorylation, oxidation, and any other manipulation such as conjugation with a labeling component. The terms “amino acid” and “amino acids,” as used herein, generally refer to natural and non-natural amino acids, including, but not limited to, modified amino acids and amino acid analogues. Modified amino acids may include natural amino acids and non-natural amino acids, which have been chemically modified to include a group or a chemical moiety not naturally present on the amino acid. Amino acid analogues may refer to amino acid derivatives. The term “amino acid” includes both D-amino acids and L-amino acids.

The term “promoter”, as used herein, generally refers to the regulatory DNA region which controls transcription or expression of a gene and which may be located adjacent to or overlapping a nucleotide or region of nucleotides at which RNA transcription is initiated. A promoter may contain specific DNA sequences which bind protein factors, often referred to as transcription factors, which facilitate binding of RNA polymerase to the DNA leading to gene transcription. A ‘basal promoter’, also referred to as a ‘core promoter’, may generally refer to a promoter that contains all the basic necessary elements to promote transcriptional expression of an operably linked polynucleotide. Eukaryotic basal promoters typically, though not necessarily, contain a TATA-box and/or a CAAT box.

The term “expression”, as used herein, generally refers to the process by which a nucleic acid sequence or a polynucleotide is transcribed from a DNA template (such as into mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

As used herein, “operably linked”, “operable linkage”, “operatively linked”, or grammatical equivalents thereof generally refers to juxtaposition of genetic elements, e.g., a promoter, an enhancer, a polyadenylation sequence, etc., wherein the elements are in a relationship permitting them to operate in the expected manner. For instance, a regulatory element, which may comprise promoter and/or enhancer sequences, is operatively linked to a coding region if the regulatory element helps initiate transcription of the coding sequence. There may be intervening residues between the regulatory element and coding region so long as this functional relationship is maintained.

A “vector” as used herein, generally refers to a macromolecule or association of macromolecules that comprises or associates with a polynucleotide and which may be used to mediate delivery of the polynucleotide to a cell. Examples of vectors include plasmids, viral vectors (including baculoviral vectors), liposomes, and other gene delivery vehicles. The vector generally comprises genetic elements, e.g., regulatory elements, operatively linked to a gene to facilitate expression of the gene in a target.

As used herein, a “guide nucleic acid” can generally refer to a nucleic acid that may hybridize to another nucleic acid. A guide nucleic acid may be RNA. A guide nucleic acid may be DNA. The guide nucleic acid may be programmed to bind to a sequence of nucleic acid site-specifically. The nucleic acid to be targeted, or the target nucleic acid, may comprise nucleotides. The guide nucleic acid may comprise nucleotides. A portion of the target nucleic acid may be complementary to a portion of the guide nucleic acid. The strand of a double-stranded target polynucleotide that is complementary to and hybridizes with the guide nucleic acid may be called the complementary strand. The strand of the double-stranded target polynucleotide that is complementary to the complementary strand, and therefore may not be complementary to the guide nucleic acid may be called noncomplementary strand. A guide nucleic acid may comprise a polynucleotide chain and can be called a “single guide nucleic acid” A guide nucleic acid may comprise two polynucleotide chains and may be called a “double guide nucleic acid.” If not otherwise specified, the term “guide nucleic acid” may be inclusive, referring to both single guide nucleic acids and double guide nucleic acids. A guide nucleic acid may comprise a segment that can be referred to as a “nucleic acid-targeting segment” or a “nucleic acid-targeting sequence.” A nucleic acid-targeting segment may comprise a sub-segment that may be referred to as a “protein binding segment” or “protein binding sequence” or “Cas protein binding segment”.

The terms “complement,” “complements,” “complementary,” and “complementarity,” as used herein, generally refer to a sequence that is fully complementary to and hybridizable to the given sequence. In some cases, a sequence hybridized with a given nucleic acid is referred to as the “complement” or “reverse-complement” of the given molecule if its sequence of bases over a given region is capable of complementarily binding those of its binding partner, such that, for example, A-T, A-U, G-C, and G-U base pairs are formed. In general, a first sequence that is hybridizable to a second sequence is specifically or selectively hybridizable to the second sequence, such that hybridization to the second sequence or set of second sequences is preferred (e.g., thermodynamically more stable under a given set of conditions, such as stringent conditions commonly used in the art) to hybridization with non-target sequences during a hybridization reaction. Typically, hybridizable sequences share a degree of sequence complementarity over all or a portion of their respective lengths, such as between 25%-100% complementarity, including at least 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, and 100% sequence complementarity. Sequence identity, such as for the purpose of assessing percent complementarity, can be measured by any suitable alignment algorithm, including but not limited to the Needleman-Wunsch algorithm (see e.g., the EMBOSS Needle aligner available at www.ebi.ac.uk/Tools/psa/emboss_needle/nucleotide.html, optionally with default settings), the BLAST algorithm (see e.g., the BLAST alignment tool available at blast.ncbi nlm.nih.gov/Blast.cgi, optionally with default settings), or the Smith-Waterman algorithm (see e.g., the EMBOSS Water aligner available at www.ebi.ac.uk/Tools/psa/emboss_water/nucleotide.html, optionally with default settings). Optimal alignment can be assessed using any suitable parameters of a chosen algorithm, including default parameters.

The term “percent (%) identity,” as used herein, generally refers to the percentage of amino acid (or nucleic acid) residues of a candidate sequence that are identical to the amino acid (or nucleic acid) residues of a reference sequence after aligning the sequences and introducing gaps, if necessary, to achieve the maximum percent identity (i.e., gaps can be introduced in one or both of the candidate and reference sequences for optimal alignment and non-homologous sequences can be disregarded for comparison purposes). Alignment, for purposes of determining percent identity, can be achieved in various ways that are within the skill in the art, for instance, using publicly available computer software such as BLAST, ALIGN, or Megalign (DNASTAR) software. Percent identity of two sequences can be calculated by aligning a test sequence with a comparison sequence using BLAST, determining the number of amino acids or nucleotides in the aligned test sequence that are identical to amino acids or nucleotides in the same position of the comparison sequence, and dividing the number of identical amino acids or nucleotides by the number of amino acids or nucleotides in the comparison sequence.

As used herein, the term “in vivo” can be used to describe an event that takes place in a subject's body.

As used herein, the term “ex vivo” can be used to describe an event that takes place outside of a subject's body. An “ex vivo” assay cannot be performed on a subject. Rather, it can be performed upon a sample separate from a subject. Ex vivo can be used to describe an event occurring in an intact cell outside a subject's body.

As used herein, the term “in vitro” can be used to describe an event that takes places contained in a container for holding laboratory reagent such that it is separated from the living biological source organism from which the material is obtained. In vitro assays can encompass cell-based assays in which cells alive or dead are employed. In vitro assays can also encompass a cell-free assay in which no intact cells are employed.

“Treating” or “treatment” can refer to both therapeutic treatment and prophylactic or preventative measures, wherein the object is to prevent or slow down (lessen) a targeted pathologic condition or disorder

The term “pharmaceutically acceptable carrier,” “pharmaceutically acceptable excipient,” “physiologically acceptable carrier,” or “physiologically acceptable excipient” refers to a pharmaceutically-acceptable material, composition, or vehicle, such as a liquid or solid filler, diluent, excipient, solvent, or encapsulating material. A component can be “pharmaceutically acceptable” in the sense of being compatible with the other ingredients of a pharmaceutical formulation. It can also be suitable for use in contact with the tissue or organ of humans and animals without excessive toxicity, irritation, allergic response, immunogenicity, or other problems or complications, commensurate with a reasonable benefit/risk ratio. See, Remington. The Science and Practice of Pharmacy, 21st Edition; Lippincott Williams & Wilkins: Philadelphia, PA, 2005; Handbook of Pharmaceutical Excipients, 5th Edition”; Rowe et al., Eds., The Pharmaceutical Press and the American Pharmaceutical Association: 2005; and Handbook of Pharmaceutical Additives, 3rd Edition; Ash and Ash Eds., Gower Publishing Company: 2007; Pharmaceutical Preformulation and Formulation, Gibson Ed., CRC Press LLC: Boca Raton, FL, 2004).

The term “pharmaceutical composition” refers to a mixture of a compound disclosed herein with other chemical components, such as diluents or carriers. The pharmaceutical composition can facilitate administration of the compound to an organism. Multiple techniques of administering a compound exist in the art including, but not limited to, oral, injection, aerosol, parenteral, and topical administration.

### Overview

There is a need for improved methods and compositions for control of DNA viruses such as Nimaviridae (such as White spot syndrome virus), e.g., in farmed crustaceans. Accordingly, provided herein are methods for nuclease-based targeting of DNA viruses such as Nimaviridae and compositions for performing such methods.

### Antiviral Methods

In one aspect, the present disclosure provides for a method for inhibiting infection of or reducing replication of a virus in an animal in need thereof, comprising introducing to a cell of said animal a nuclease (e.g., a Cas protein) and a gene-binding moiety (e.g., a guide RNA). In some embodiments, the nuclease and the gene binding moiety are complexed (e.g., a programmable nuclease)

In some cases, the gene binding moiety is configured to bind at least one gene of said virus. In some embodiments, the virus is a DNA virus. In some embodiments, the virus belongs to the family Nimaviridae. In some embodiments, the virus is white spot syndrome virus (WSSV). The at least one gene can include any of the genes described in Table B, or any combination thereof. The at least one gene can include at least two, at least three, at least four, at least five, at least six, at least 7, at least 8, at least 9, at least 10, at least 11, at least 12, at least 13, at least 14, at least 15, at least 16, or all 17 genes described in Table B, or any combination thereof. The at least one gene can include ICP11 or a fragment thereof, VP19 or a fragment thereof, VP26 or a fragment thereof, collagen-like protein (WSSV-CLP) or a fragment thereof, DNApol or a fragment thereof, VP28 or a fragment thereof, or RR1 or a fragment thereof, or any combination thereof (e.g., any two of the preceding, any three of the preceding, any four of the preceding). The at least one gene can further include DNA polymerase (DNApol), ribonucleotide reductase subunit 1 (RR1), VP28, or any combination thereof (e.g., any two of the preceding, any three of the preceding). A fragment that is bound by the gene-binding moiety can include a sequence of a length sufficient to drive binding of the nuclease. Such sequence lengths can generally include from at least about 9 nucleotides to about 20 nucleotides, including at least 10, at least 11, at least 12, at least 13, at least 14, at least 15, at least 16, at least 17, at least 18, at least 19, at least 20, at most 20, at most 19, at most 18, at most 17, at most 16, at most 15, at most 14, at most 13, at most 12 nucleotides, at most 11 nucleotides, at most 10 nucleotides, or at most 9 nucleotides. The gene-binding moiety can be configured to bind a plurality of different (e.g., non-contiguous) portions of said one or more genes of said virus, such as at least 1 portion, at least 2 portions, at least 3 portions, at least 4 portions, at least 5 portions, or more. The gene binding moiety can be configured to bind at least one, at least two, at least three, or all four of ICP11, VP19, VP26, or collagen-like protein (WSSV-CLP), or any combination thereof. The gene binding moiety can be configured to bind a combination of at least two, at least three, or all four of ICP11, VP19, VP26, or collagen-like protein (WSSV-CLP), or any combination thereof.

In some cases, the gene-binding moiety is configured to bind a specific sequence within the viral gene targeted. The programmable nuclease can be configured to bind at least 5, or at least 18-24 consecutive nucleotides of at least one sequence selected from SEQ ID NOs: 22-82. The programmable nuclease can be configured to bind at least 6, at least 7, at least 8, at least 9, at least 10, at least 11, at least 12, at least 13, at least 14, at least 15, at least 16, at least 17, at least 18, at least 19, or at least 20 nucleotides of at least one sequence selected from SEQ ID NOs: 22-82. The programmable nuclease can be configured to bind at least 6, at least 7, at least 8, at least 9, at least 10, at least 11, at least 12, at least 13, at least 14, at least 15, at least 16, at least 17, at least 18, at least 19, or at least 20 nucleotides of a variant having at least 80%, at least 85%, at least 90%, at least 91%, at least 92%, at least 93%, at least 94%, at least 95%, at least 96%, at least 97%, at least 98%, at least 99% identity to any one of SEQ ID NOs: 22-82, or a variant being substantially identical to any one of SEQ ID NOs: 22-82. The programmable nuclease can be further configured to bind at least 5, or at least 18-24 consecutive nucleotides of at least one (e.g., at least two, or at least three) sequences selected from SEQ ID NOs. 1-9. The programmable nuclease can be configured to bind at least 6, at least 7, at least 8, at least 9, at least 10, at least 11, at least 12, at least 13, at least 14, at least 15, at least 16, at least 17, at least 18, at least 19, or at least 20 nucleotides of at least one sequence selected from SEQ ID NOs: 1-9. The programmable nuclease can be configured to bind at least 6, at least 7, at least 8, at least 9, at least 10, at least 11, at least 12, at least 13, at least 14, at least 15, at least 16, at least 17, at least 18, at least 19, or at least 20 nucleotides of a variant having at least 80%, at least 85%, at least 90%, at least 91%, at least 92%, at least 93%, at least 94%, at least 95%, at least 96%, at least 97%, at least 98%, at least 99% identity to any one of SEQ ID NOs: 1-9, or a variant substantially identical to any one of SEQ ID NOs: 1-9.

In some cases, the nuclease comprising a gene-binding moiety can comprise a programmable nuclease. Programmable nucleases include at least one of a CRISPR-associated (Cas) polypeptide, a zinc finger nuclease (ZFN), a transcription activator-like effector nuclease (TALEN), or a combination thereof.

Cas polypeptides can include Class 1 CRISPR-associated (Cas) polypeptides, Class 2 Cas polypeptides, type I Cas polypeptides, type II Cas polypeptides, type III Cas polypeptides, type IV Cas polypeptides, type V Cas polypeptides, and type VI, CRISPR-associated RNA binding proteins, or functional fragments thereof. Cas polypeptides suitable for use with the present disclosure can include Cas9, Cas12, Cas13, Cpf1 (or Cas12a), C2C1, C2C2 (or Cas13a), Cas13b, Cas13c, Cas13d, C2C3, Cas1, Cas1B, Cas2, Cas3, Cas4, Cas5, Cas5e (CasD), Caso, Cas6e, Casof, Cas7, Cas8a, CasSal, Cas8a2, Cas8b, Cas8c, Csn1, Csx12, Cas10, Cas10d, Cas10, Cas10d, CasF, CasG, CasH, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, or Cul966. Cas13 can include Cas13a, Cas13b, Cas13c, and Cas 13d (e.g., CasRx). Cas can be DNA (e.g., Cpf1, Cas9) and/or RNA cleaving (e.g., Cas13).

In some embodiments, the nuclease disclosed herein can be a protein that lacks nucleic acid cleavage activity. In some cases, the Cas protein is a dead Cas protein. A dead Cas protein can be a protein that lacks nucleic acid cleavage activity, which can comprise a modified (e.g., mutated) form of a wild type Cas protein. The modified form of the wild type Cas protein can comprise an amino acid change (e.g., deletion, insertion, or substitution) that reduces the nucleic acid-cleaving activity of the Cas protein. When a Cas protein is a modified form that has no substantial nucleic acid-cleaving activity, it can be referred to as enzymatically inactive and/or “dead” (abbreviated by “d”). A dead Cas protein (e.g., dCas, dCas9) can bind to a target polynucleotide but may not cleave the target polynucleotide. In some aspects, a dead Cas protein is a dead Cas9 protein.

In some embodiments, a dCas (e.g., dCas9) polypeptide can associate with a single guide RNA (sgRNA) to repress transcription of target DNA (e.g., when the nuclease further comprises a protein acting as a genetic repressor).

In some cases, the gene binding moiety of the nuclease can comprise a heterologous RNA polynucleotide configured to hybridize to said one or more genes of said virus (e.g., when the nuclease is a Cas polypeptide). The heterologous RNA can be a guide RNA, comprising both a targeting sequence directed against a particular gene sequence, and a scaffold sequence binding to a Cas polypeptide.

The heterologous RNA polynucleotide can comprise at least one heterologous RNA polynucleotide targeting at least one (e.g., at least two, at least three) sequences. The heterologous RNA polynucleotide can comprise at least one heterologous RNA polynucleotide targeting at least four, at least five, at least six, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, or more sequences. The targeting sequences can comprise at least 17 (e.g., at least 18, at least 19, at least 20, at least 21, at least 22, at most 22, at most 20, at most 19, at most 18, or at most 17) consecutive nucleotides of at least one sequence selected from SEQ ID NOs: 83-143, or a complement thereof. The targeting sequences can comprise at least 17 (e.g., at least 18, at least 19, at least 20, at least 21, at least 22, at most 22, at most 20, at most 19, at most 18, or at most 17) consecutive nucleotides of a sequence variant having at least 80%, at least 85%, at least 90%, at least 91%, at least 92%, at least 93%, at least 94%, at least 95%, at least 96%, at least 97%, at least 98%, at least 99% identity to any one of SEQ ID NOs: 83-143 (or a complement thereof), or a sequence variant substantially identical to any one of SEQ ID NOs. 83-143 (or a complement thereof). The targeting sequences can further comprise at least one (e.g., at least two, at least three) targeting sequences comprising at least 17 (e.g., at least 18, at least 19, at least 20, at least 21, at least 22, at most 22, at most 20, at most 19, at most 18, or at most 17) consecutive nucleotides of at least one sequence selected from SEQ ID NOs: 10-18. The targeting sequences can further comprise at least one (e.g., at least two, at least three) targeting sequences comprising at least 17 (e.g., at least 18, at least 19, at least 20, at least 21, at least 22, at most 22, at most 20, at most 19, at most 18, or at most 17) consecutive nucleotides of a sequence variant having at least 80%, at least 85%, at least 90%, at least 91%, at least 92%, at least 93%, at least 94%, at least 95%, at least 96%, at least 97%, at least 98%, at least 99% identity to any one of SEQ ID NOs: 10-18, or a sequence variant substantially identical to any one of SEQ ID NOs: 10-18.

In some cases, introducing a nuclease comprising a gene-binding moiety to said cell of said animal comprises contacting said cell with the nuclease. The nuclease can be a polypeptide alone (e.g., a zinc-finger or TALEN nuclease) or a ribonucleoprotein complex with a heterologous RNA (e.g., when the nuclease comprises a Cas protein). The nuclease can be contacted to the cell in the presence of a transfection agent (e.g., various lipid-based systems including oil-in-water emulsions, micelles, mixed micelles, and liposomes) and/or with the aid of a physical stimulus promoting entry of macromolecules into cells (e.g., electroporation, heat). The ribonucleoprotein complex can comprise a Cas enzyme together with multiple (e.g., at least one, two, three, or more heterologous RNA polynucleotides targeted against different regions of a same viral gene or different genes.

In some cases, introducing a nuclease comprising a gene-binding moiety to the cell of the animal comprises contacting said cell with a capped mRNA comprising a sequence encoding the nuclease. Such capped mRNAs can be chemically synthesized or in-vitro transcribed by a variety of suitable methods. The capped mRNA can be contacted to the cell in the presence of a transfection agent (e.g., various lipid-based systems including oil-in-water emulsions, micelles, mixed micelles, and liposomes) and/or with the aid of a physical stimulus promoting entry of macromolecules into cells (e.g., electroporation, heat). The capped mRNA can also be contacted to the cell in the presence of at least one (e.g., at least two, at least three) heterologous RNA polynucleotides directed against one or more regions of a viral gene, or one or more viral genes.

In some embodiments, a nuclease comprising a gene-binding moiety (or a polynucleotide encoding the gene binding moiety) is provided to said cell of said animal provided in the feed of said animal, or is provided orally to the animal. In some embodiments, a nuclease comprising a gene-binding moiety (or a polynucleotide encoding the gene binding moiety) is provided to the cell of the animal is provided in the water in which the animal is housed. In some embodiments, a nuclease comprising a gene-binding moiety (or a polynucleotide encoding the gene binding moiety) is provided to the cell of the animal is provided in the environment in which the animal is housed. In some embodiments, a nuclease comprising a gene binding moiety (or a polynucleotide encoding the gene binding moiety) is contacted to a cell of the organism. In some embodiments, a nuclease comprising a gene binding moiety (or a polynucleotide encoding the gene binding moiety) is transfected into a cell of the organism. In some embodiments, a nuclease comprising a gene binding moiety (or a polynucleotide encoding the gene binding moiety) is injected into the organism.

### Vectors

In some cases, introducing a nuclease comprising a gene-binding moiety to a cell of the animal comprises contacting the cell with a vector comprising a sequence encoding the nuclease and/or sequence encoding heterologous RNA polynucleotides targeting at least one viral gene.

The vector can be a plasmid, a minicircle (see e.g., U.S. Pat. No. 10,612,030B2, which describes methods of producing minicircles), or a viral vector. Exemplary viral vectors include retroviral vectors, adenoviral vectors, adeno-associated viral vectors (AAVs), pox vectors, parvoviral vectors, baculovirus vectors, measles viral vectors, herpes simplex virus vectors (HSVs), Infectious Hypodermal and Haematopoietic Necrosis Virus (IHHNV vectors), or Taura syndrome virus (TSV).

The nuclease can comprise any of the nucleases comprising gene-binding moieties described herein, including a CRISPR-associated (Cas) polypeptide, a zinc finger nuclease (ZFN), a transcription activator-like effector nuclease (TALEN). Cas polypeptides can include Class 1 CRISPR-associated (Cas) polypeptides, Class 2 Cas polypeptides, type I Cas polypeptides, type II Cas polypeptides, type III Cas polypeptides, type IV Cas polypeptides, type V Cas polypeptides, and type VI, CRISPR-associated RNA binding proteins, or functional fragments thereof. Cas polypeptides suitable for use with the present disclosure can include Cas9, Cas12, Cas13, Cpf1 (or Cas12a), C2C1, C2C2 (or Cas13a), Cas13b, Cas13c, Cas13d, C2C3, Cas1, Cas1B, Cas2, Cas3, Cas4, Cas5, Cas5e (CasD), Cas6, Case, Cas6f, Cas7, Cas8a, Cas8al, Cas8a2, Cas8b, Cas8c, Csn1, Csx12, Cas10, Cas10d, Cas10, Caslod, CasF, CasG, CasH, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, or Cul966. Cas13 can include Cas13a, Cas13b, Cas13c, and Cas 13d (e.g., CasRx). Cas can be DNA (e.g., Cpf1, Cas9) and/or RNA cleaving (e.g., Cas13). Explicitly included in the disclosure is the use of any RNA targeting Cas enzymes to target e.g. mRNA sequences transcribed by any of the genes described herein.

The vector can comprise a sequence encoding the nuclease (e.g., a programmable nuclease, a Cas polypeptide, or any of the other nucleases comprising gene-binding moieties described herein). In some embodiments, the gene binding moieties are under the control of or operably linked to a promoter sequence suitable for the animal into which the vector is being introduced. In the case of crustaceans, an exemplary promoter sequence is the P2 promoter from infectious bypodermal and hematopoietic necrosis virus (IHHNV) of shrimp or a functional fragment thereof. Such a functional P2 promoter can comprise at least 100 consecutive nucleotides (e.g., at least 150, at least 200, at least 250, at least 300, at least 400, at least 500, at least 750, at least 1000, at most 1000, at most 750, at most 500, at most 400, at most 300, at most 250, at most 200, at most 150, or at most 100) of any one of SEQ ID NOs: 20-162, or at least 100 consecutive nucleotides (e.g., at least 150, at least 200, at least 250, at least 300, at least 400, at least 500, at least 750, at least 1000, at most 1000, at most 750, at most 500, at most 400, at most 300, at most 250, at most 200, at most 150, or at most 100) of a sequence variant having at least 80%, at least 85%, at least 90%, at least 91%, at least 92%, at least 93%, at least 94%, at least 95%, at least 96%, at least 97%, at least 98%, at least 99% identity to any one of SEQ ID NOs: 20-162, or a sequence variant substantially identical to any one of SEQ ID NOs: 20-162.

In some cases, the programmable nuclease is configured to bind at least one gene of said virus. The at least one gene can include any of the genes described herein, including any of the genes described in Table B, or a combination thereof. The one or more genes can include ICP11 or a fragment thereof, VP19 or a fragment thereof, VP26 or a fragment thereof, collagen-like protein (WSSV-CLP) or a fragment thereof, or any combination thereof (e.g., any two of the preceding, any three of the preceding, any four of the preceding). The genes can further include DNA polymerase, RR1, VP28, or any combination thereof (e.g., any two of the preceding, any three of the preceding). A fragment that is bound by the gene-binding moiety can include a sequence of a length sufficient to drive binding of the nuclease. Such sequence lengths can generally include from at least about 9 nucleotides to about 20 nucleotides, including at least 10, at least 11, at least 12, at least 13, at least 14, at least 15, at least 16, at least 17, at least 18, at least 19, at least 20, at most 20, at most 19, at most 18, at most 17, at most 16, at most 15, at most 14, at most 13, at most 12 nucleotides, at most 11 nucleotides, at most 10 nucleotides, or at most 9 nucleotides. The gene-binding moiety can be configured to bind a plurality of different (e.g., non-contiguous) portions of said one or more genes of said virus, such as at least 1 portion, at least 2 portions, at least 3 portions, at least 4 portions, at least 5 portions, or more. The gene binding moiety can be configured to bind a combination of at least two, at least three, or all four of ICP11, VP19, VP26, or collagen-like protein (WSSV-CLP), or any combination thereof.

In some cases, the programmable nuclease is directed against a specific sequence within the viral gene targeted. The programmable nuclease can be configured to bind at least 5, or at least 18-24 consecutive nucleotides of at least one sequence selected from SEQ ID NOs: 22-82. The programmable nuclease can be configured to bind at least 6, at least 7, at least 8, at least 9, at least 10, at least 11, at least 12, at least 13, at least 14, at least 15, at least 16, at least 17, at least 18, at least 19, or at least 20 nucleotides of at least one sequence selected from SEQ ID NOs: 22-82. The programmable nuclease can be configured to bind at least 6, at least 7, at least 8, at least 9, at least 10, at least 11, at least 12, at least 13, at least 14, at least 15, at least 16, at least 17, at least 18, at least 19, or at least 20 nucleotides of a variant having at least 80%, at least 85%, at least 90%, at least 91%, at least 92%, at least 93%, at least 94%, at least 95%, at least 96%, at least 97%, at least 98%, at least 99% identity to any one of SEQ ID NOs: 22-82, or a variant being substantially identical to any one of SEQ ID NOs: 22-82. The programmable nuclease can be further configured to bind at least 5, or at least 18-24 consecutive nucleotides of at least one (e.g., at least two, or at least three) sequences selected from SEQ ID NOs: 1-9. The programmable nuclease can be configured to bind at least 6, at least 7, at least 8, at least 9, at least 10, at least 11, at least 12, at least 13, at least 14, at least 15, at least 16, at least 17, at least 18, at least 19, or at least 20 nucleotides of at least one sequence selected from SEQ ID NOs: 1-9. The programmable nuclease can be configured to bind at least 6, at least 7, at least 8, at least 9, at least 10, at least 11, at least 12, at least 13, at least 14, at least 15, at least 16, at least 17, at least 18, at least 19, or at least 20 nucleotides of a variant having at least 80%, at least 85%, at least 90%, at least 91%, at least 92%, at least 93%, at least 94%, at least 95%, at least 96%, at least 97%, at least 98%, at least 99% identity to any one of SEQ ID NOs: 1-9, or a variant substantially identical to any one of SEQ ID NOs: 1-9.

In some cases (e.g., when the nuclease is a Cas polypeptide) the vector can comprise at least one (e.g., at least two, at least three), sequences encoding heterologous RNA polynucleotides comprising targeting sequences against at least one viral gene. The targeting sequences can comprise at least 17 (e.g., at least 18, at least 19, at least 20, at least 21, at least 22, at most 22, at most 20, at most 19, at most 18, or at most 17) consecutive nucleotides of at least one sequence selected from SEQ ID NOs: 83-143 or a complement thereof. The targeting sequences can comprise at least 17 (e.g., at least 18, at least 19, at least 20, at least 21, at least 22, at most 22, at most 20, at most 19, at most 18, or at most 17) consecutive nucleotides of a sequence variant having at least 80%, at least 85%, at least 90%, at least 91%, at least 92%, at least 93%, at least 94%, at least 95%, at least 96%, at least 97%, at least 98%, at least 99% identity to any one of SEQ ID NOs. 83-143 or a complement thereof, or a sequence variant substantially identical to any one of SEQ ID NOs: 83-143 or a complement thereof. The targeting sequences can further comprise at least one (e.g., at least two, at least three) targeting sequences comprising at least 17 (e.g., at least 18, at least 19, at least 20, at least 21, at least 22, at most 22, at most 20, at most 19, at most 18, or at most 17) consecutive nucleotides of at least one sequence selected from SEQ ID NOs: 10-18. The targeting sequences can further comprise at least one (e.g., at least two, at least three) targeting sequences comprising at least 17 (e.g., at least 18, at least 19, at least 20, at least 21, at least 22, at most 22, at most 20, at most 19, at most 18, or at most 17) consecutive nucleotides of a sequence variant having at least 80%, at least 85%, at least 90%, at least 91%, at least 92%, at least 93%, at least 94%, at least 95%, at least 96%, at least 97%, at least 98%, at least 99% identity to any one of SEQ ID NOs: 10-18, or a sequence variant substantially identical to any one of SEQ ID NOs: 10-18. The sequences encoding heterologous RNA polynucleotides can further comprise a 3′ protein binding segment (or scaffold) capable of binding the nuclease (e.g., when the programmable nuclease is a Cas polypeptide).

In some cases, the at least one (e.g., at least two, at least three), sequences encoding heterologous RNA polynucleotides can be under the control of or operably linked to a viral promoter sequence. An exemplary viral promoter is the ie1 (intermediate early 1) promoter of WSSV, or a functional fragment thereof. Such a promoter sequence can comprise at least 100 (e.g., at least 150, at least 200, at least 250, at least 300, at least 400, at least 500, at least 750, at least 1000, at most 1000, at most 750, at most 500, at most 400, at most 300, at most 250, at most 200, at most 150, or at most 100) consecutive nucleotides of SEQ ID NO:21, or at least 100 consecutive nucleotides (e.g., at least 150, at least 200, at least 250, at least 300, at least 400, at least 500, at least 750, at least 1000, at most 1000, at most 750, at most 500, at most 400, at most 300, at most 250, at most 200, at most 150, or at most 100) of a sequence variant having at least 80%, at least 85%, at least 90%, at least 91%, at least 92%, at least 93%, at least 94%, at least 95%, at least 96%, at least 97%, at least 98%, at least 99% identity to any one of SEQ ID NOs: 20-162, or a sequence variant substantially identical to any one of SEQ ID NOs: 20-162.

TABLE A
Sequences of vectors and nucleic acid elements useful in embodiments according to the
disclosure

| SEQ | | |
|---|---|---|
| ID | | |
| NO: | Description | Sequence (nucleotide or polypeptide) |
| 144 | P23 | ttactcatatatactttagattgatttaaaacttcatttttaatttaaaaggatctaggtgaagatcctttttgat |
| | Vector | aatctcatgaccaaaatcccttaacgtgagttttcgttccactgagcgtcagaccccgtagaaaagatcaaaggat |
| | targeting | cttcttgagatcctttttttctgcgcgtaatctgctgcttgcaaacaaaaaaaccaccgctaccagcggtggtttg |
| | p23 gene | tttgccggatcaagagctaccaactctttttccgaaggtaactggcttcagcagagcgcagataccaaatactgtt |
| | of WSSV | cttctagtgtagccgtagttaggccaccacttcaagaactctgtagcaccgcctacatacctcgctctgctaatcc |
| | including | tgttaccagtggctgctgccagtggcgataagtcgtgtcttaccgggttggactcaagacgatagttaccggataa |
| | 3 targeting | ggcgcagcggtcgggctgaacggggggttcgtgcacacagcccagcttggagcgaacgacctacaccgaa |
| | sgRNAs | ctgagatacctacagcgtgagctatgagaaagcgccacgcttcccgaagggagaaaggcggacaggtatcc |
| | and | ggtaagcggcagggtcggaacaggagagcgcacgagggagcttccagggggaaacgcctggtatctttat |
| | NLS-Cas9, | agtcctgtcgggtttcgccacctctgacttgagcgtcgatttttgtgatgctcgtcaggggggcggagcctatgg |
| | driven by | aaaaacgccagcaacgcggcctttttacggttcctggccttttgctggccttttgctcacatgtgctagcctgcga |
| | P2 promoter | gcgcttcgcagaaaccgttacaacctatgacgtcataggtcctatataagagatgacggactcaccggtctccc |
| | of IHHNV | agtttctaactgacgagtgaagaggctattccaagtggtaccgccaccatggacaagaagtactccatcggcc |
| | | tggacatcggcaccaactccgtgggctgggccgtgatcaccgacgagtacaaggtgccctccaagaagttc |
| | | aaggtgctgggcaacaccgaccgccactccatcaagaagaacctgatcggcgccctgctgttcgactccgg |
| | | cgagaccgccgaggccacccgcctgaagcgcaccgcccgccgccgctacacccgccgcaagaaccgca |
| | | tctgctacctgcaggagatcttctccaacgagatggccaaggtggacgactccttcttccaccgcctggagga |
| | | gtccttcctggtggaggaggacaagaagcacgagcgccaccccatcttcggcaacatcgtggacgaggtgg |
| | | cctaccacgagaagtaccccaccatctaccacctgcgcaagaagctggtggactccaccgacaaggccgac |
| | | ctgcgcctgatctacctggccctggcccacatgatcaagttccgcggccacttcctgatcgagggcgacctga |
| | | accccgacaactccgacgtggacaagctgttcatccagctggtgcagacctacaaccagctgttcgaggaga |
| | | accccatcaacgcctccggcgtggacgccaaggccatcctgtccgcccgcctgtccaagtcccgccgcctg |
| | | gagaacctgatcgcccagctgcccggcgagaagaagaacggcctgttcggcaacctgatcgccctgtccct |
| | | gggcctgacccccaacttcaagtccaacttcgacctggccgaggacgccaagctgcagctgtccaaggaca |
| | | cctacgacgacgacctggacaacctgctggcccagatcggcgaccagtacgccgacctgttcctggccgcc |
| | | aagaacctgtccgacgccatcctgctgtccgacatcctgcgcgtgaacaccgagatcaccaaggcccccctg |
| | | tccgcctccatgatcaagcgctacgacgagcaccaccaggacctgaccctgctgaaggccctggtgcgcca |
| | | gcagctgcccgagaagtacaaggagatcttcttcgaccagtccaagaacggctacgccggctacatcgacg |
| | | gcggcgcctcccaggaggagttctacaagttcatcaagcccatcctggagaagatggacggcaccgagga |
| | | gctgctggtgaagctgaaccgcgaggacctgctgcgcaagcagcgcaccttcgacaacggctccatccccc |
| | | accagatccacctgggcgagctgcacgccatcctgcgccgccaggaggacttctaccccttcctgaaggac |
| | | aaccgcgagaagatcgagaagatcctgaccttccgcatcccctactacgtgggccccctggcccgcggcaa |
| | | ctcccgcttcgcctggatgacccgcaagtccgaggagaccatcaccccctggaacttcgaggaggtggtgg |
| | | acaagggcgcctccgcccagtccttcatcgagcgcatgaccaacttcgacaagaacctgcccaacgagaag |
| | | gtgctgcccaagcactccctgctgtacgagtacttcaccgtgtacaacgagctgaccaaggtgaagtacgtga |
| | | ccgagggcatgcgcaagcccgccttcctgtccggcgagcagaagaaggccatcgtggacctgctgttcaag |
| | | accaaccgcaaggtgaccgtgaagcagctgaaggaggactacttcaagaagatcgagtgcttcgactccgt |
| | | ggagatctccggcgtggaggaccgcttcaacgcctccctgggcacctaccacgacctgctgaagatcatcaa |
| | | ggacaaggacttcctggacaacgaggagaacgaggacatcctggaggacatcgtgctgaccctgaccctgt |
| | | tcgaggaccgcgagatgatcgaggagcgcctgaagacctacgcccacctgttcgacgacaaggtgatgaa |
| | | gcagctgaagcgccgccgctacaccggctggggccgcctgtcccgcaagctgatcaacggcatccgcgac |
| | | aagcagtccggcaagaccatcctggacttcctgaagtccgacggcttcgccaaccgcaacttcatgcagctg |
| | | atccacgacgactccctgaccttcaaggaggacatccagaaggcccaggtgtccggccagggcgactccct |
| | | gcacgagcacatcgccaacctggccggctcccccgccatcaagaagggcatcctgcagaccgtgaaggtg |
| | | gtggacgagctggtgaaggtgatgggccgccacaagcccgagaacatcgtgatcgagatggcccgcgaga |
| | | accagaccacccagaagggccagaagaactcccgcgagcgcatgaagcgcatcgaggagggcatcaag |
| | | gagctgggctcccagatcctgaaggagcaccccgtggagaacacccagctgcagaacgagaagctgtacc |
| | | tgtactacctgcagaacggccgcgacatgtacgtggaccaggagctggacatcaaccgcctgtccgactacg |
| | | acgtggaccacatcgtgccccagtccttcctgaaggacgactccatcgacaacaaggtgctgacccgctccg |
| | | acaagaaccgcggcaagtccgacaacgtgccctccgaggaggtggtgaagaagatgaagaactactggcg |
| | | ccagctgctgaacgccaagctgatcacccagcgcaagttcgacaacctgaccaaggccgagcgcggcggc |
| | | ctgtccgagctggacaaggccggcttcatcaagcgccagctggtggagacccgccagatcaccaagcacgt |
| | | ggcccagatcctggactcccgcatgaacaccaagtacgacgagaacgacaagctgatccgcgaggtgaag |
| | | gtgatcaccctgaagtccaagctggtgtccgacttccgcaaggacttccagttctacaaggtgcgcgagatca |
| | | acaactaccaccacgcccacgacgcctacctgaacgccgtggtgggcaccgccctgatcaagaagtacccc |
| | | aagctggagtccgagttcgtgtacggcgactacaaggtgtacgacgtgcgcaagatgatcgccaagtccga |
| | | gcaggagatcggcaaggccaccgccaagtacttcttctactccaacatcatgaacttcttcaagaccgagatc |
| | | accctggccaacggcgagatccgcaagcgccccctgatcgagaccaacggcgagaccggcgagatcgtg |
| | | tgggacaagggccgcgacttcgccaccgtgcgcaaggtgctgtccatgccccaggtgaacatcgtgaagaa |
| | | gaccgaggtgcagaccggcggcttctccaaggagtccatcctgcccaagcgcaactccgacaagctgatcg |
| | | cccgcaagaaggactgggaccccaagaagtacggcggcttcgactcccccaccgtggcctactccgtgctg |
| | | gtggtggccaaggtggagaagggcaagtccaagaagctgaagtccgtgaaggagctgctgggcatcacca |
| | | tcatggagcgctcctccttcgagaagaaccccatcgacttcctggaggccaagggctacaaggaggtgaag |
| | | aaggacctgatcatcaagctgcccaagtactccctgttcgagctggagaacggccgcaagcgcatgctggcc |
| | | tccgccggcgagctgcagaagggcaacgagctggccctgccctccaagtacgtgaacttcctgtacctggc |
| | | ctcccactacgagaagctgaagggctcccccgaggacaacgagcagaagcagctgttcgtggagcagcac |
| | | aagcactacctggacgagatcatcgagcagatctccgagttctccaagcgcgtgatcctggccgacgccaac |
| | | ctggacaaggtgctgtccgcctacaacaagcaccgcgacaagcccatccgcgagcaggccgagaacatca |
| | | tccacctgttcaccctgaccaacctgggcgcccccgccgccttcaagtacttcgacaccaccatcgaccgcaa |
| | | gcgctacacctccaccaaggaggtgctggacgccaccctgatccaccagtccatcaccggcctgtacgaga |
| | | cccgcatcgacctgtcccagctgggcggcgacggctcccccaagaagaagcgcaaggtgtcctccggcgg |
| | | cgccgccggctgactcgagtaatgaatttccttgttactcatttattcctagaaatggtgtaatcgctgttgtggg |
| | | cggagcatatttgtgtatataagagcccgtgttagctcctcgattcagtcacaagagcgcacacacacgcttataa |
| | | ctagctctctctctccactcaagatggcctttaattttgaagactctacaaatctctttgccaaacaaagcaccag |
| | | tggtctagtggtagaatagtaccctgccacggtacagacccgggttcgattcccggctggtgcatttgctgcaca |
| | | cgtcaatgagttttagagctagaaatagcaagttaaaataaggctagtccgttatcaacttgaaaaagtggcacc |
| | | gagtcggtgcaacaaagcaccagtggtctagtggtagaatagtaccctgccacggtacagacccgggttcga |
| | | ttcccggctggtgcatgcgagtcccaattcaaagagttttagagctagaaatagcaagttaaaataaggctagt |
| | | ccgttatcaacttgaaaaagtggcaccgagtcggtgcaacaaagcaccagtggtctagtggtagaatagtacc |
| | | ctgccacggtacagacccgggttcgattcccggctggtgcacaatactcctcttggaaagggttttagagctag |
| | | aaatagcaagttaaaataaggctagtccgttatcaacttgaaaaagtggcaccgagtcggtgctttttttctagac |
| | | ttgtcgactttcaattgaaagggcctcgtgatacgcctatttttataggttaatgtcatgataataatggtttctt |
| | | agacgtcaggtggcacttttcggggaaatgtgcgcggaacccctatttgtttatttttctaaatacattcaaatat |
| | | gtatccgctcatgagacaataaccctgataaatgcttcaataatattgaaaaaggaagagtatgagtattcaacat |
| | | ttccgtgtcgcccttattcccttttttgcggcattttgccttcctgtttttgctcacccagaaacgctggtgaaag |
| | | taaaagatgctgaagatcagttgggtgcacgagtgggttacatcgaactggatctcaacagcggtaagatccttga |
| | | gagttttcgccccgaagaacgttttccaatgatgagcacttttaaagttctgctatgtggcgcggtattatcccgt |
| | | attgacgccgggcaagagcaactcggtcgccgcatacactattctcagaatgacttggttgagtactcaccagtca |
| | | cagaaaagcatcttacggatggcatgacagtaagagaattatgcagtgctgccataaccatgagtgataacactg |
| | | cggccaacttacttctgacaacgatcggaggaccgaaggagctaaccgcttttttgcacaacatgggggatca |
| | | tgtaactcgccttgatcgttgggaaccggagctgaatgaagccataccaaacgacgagcgtgacaccacgat |
| | | gcctgtagcaatggcaacaacgttgcgcaaactattaactggcgaactacttactctagcttcccggcaacaatt |
| | | aatagactggatggaggcggataaagttgcaggaccacttctgcgctcggcccttccggctggctggtttattg |
| | | ctgataaatctggagccggtgagcgtgggagtcgcggtatcattgcagcactggggccagatggtaagccct |
| | | cccgtatcgtagttatctacacgacggggagtcaggcaactatggatgaacgaaatagacagatcgctgagat |
| | | aggtgcctcactgattaagcattggtaactgtcagaccaagt |
| 145 | P32 | ttactcatatatactttagattgatttaaaacttcatttttaatttaaaaggatctaggtgaagatcctttttgat |
| | Vector | aatctcatgaccaaaatcccttaacgtgagttttcgttccactgagcgtcagaccccgtagaaaagatcaaaggat |
| | targeting | cttcttgagatcctttttttctgcgcgtaatctgctgcttgcaaacaaaaaaaccaccgctaccagcggtggtttg |
| | VP26 | tttgccggatcaagagctaccaactctttttccgaaggtaactggcttcagcagagcgcagataccaaatactgtt |
| | gene of | cttctagtgtagccgtagttaggccaccacttcaagaactctgtagcaccgcctacatacctcgctctgctaatcc |
| | WSSV | tgttaccagtggctgctgccagtggcgataagtcgtgtcttaccgggttggactcaagacgatagttaccggataa |
| | expressing | ggcgcagcggtcgggctgaacggggggttcgtgcacacagcccagcttggagcgaacgacctacaccgaa |
| | 3 different | ctgagatacctacagcgtgagctatgagaaagcgccacgcttcccgaagggagaaaggcggacaggtatcc |
| | sgRNAs | ggtaagcggcagggtcggaacaggagagcgcacgagggagcttccagggggaaacgcctggtatctttat |
| | driven by | agtcctgtcgggtttcgccacctctgacttgagcgtcgatttttgtgatgctcgtcaggggggggagcctatgg |
| | WSSV ie1 | aaaaacgccagcaacgcggcctttttacggttcctggccttttgctggccttttgctcacatgtgctagcGTTA |
| | promoter | TCACAATCGTGCAATATATCAATAAATTATTTGATGTTCATCAGTGTT |
| | and | TCTAAAGAagttatttataccacgggctacggttgcataaagccgggccacgtgatattctcgcagacG |
| | NLS-Cas9 | GTCTCCCGCGTCTGGACCTCGCTGGCTGGCGGCTTTGCCATGAGGT |
| | driven by | CGCTCCCCACGTGgcgaaggaaccgctgtcatgttggcacgcctggtacgccgagctgttctgca |
| | hsp70 | gtgacgcgcgtaaatttcccgctaaataccgtccaatagcatcgaccattccggcattgttgaccaataagaac |
| | promoter | gtttcacttaccacgtcacggttatgaagccctacgattggaaccagaagtgaCGTCATAAACTAG |
| | of | GAACTCTGTGAATGGCCAACGTTCCGAAGAAGGTTCTAGAAGGTT |
| | Penaeus | TACAAAGGGGTCGATGTCGCCGCTCTATGGATTTGCCGAAGTCATgg |
| | monodon | taccgccaccatggacaagaagtactccatcggcctggacatcggcaccaactccgtgggctgggccgtgat |
| | | caccgacgagtacaaggtgccctccaagaagttcaaggtgctgggcaacaccgaccgccactccatcaaga |
| | | agaacctgatcggcgccctgctgttcgactccggcgagaccgccgaggccacccgcctgaagcgcaccgc |
| | | ccgccgccgctacacccgccgcaagaaccgcatctgctacctgcaggagatcttctccaacgagatggcca |
| | | aggtggacgactccttcttccaccgcctggaggagtccttcctggtggaggaggacaagaagcacgagcgc |
| | | caccccatcttcggcaacatcgtggacgaggtggcctaccacgagaagtaccccaccatctaccacctgcgc |
| | | aagaagctggtggactccaccgacaaggccgacctgcgcctgatctacctggccctggcccacatgatcaa |
| | | gttccgcggccacttcctgatcgagggcgacctgaaccccgacaactccgacgtggacaagctgttcatcca |
| | | gctggtgcagacctacaaccagctgttcgaggagaaccccatcaacgcctccggcgtggacgccaaggcc |
| | | atcctgtccgcccgcctgtccaagtcccgccgcctggagaacctgatcgcccagctgcccggcgagaagaa |
| | | gaacggcctgttcggcaacctgatcgccctgtccctgggcctgacccccaacttcaagtccaacttcgacctg |
| | | gccgaggacgccaagctgcagctgtccaaggacacctacgacgacgacctggacaacctgctggcccaga |
| | | tcggcgaccagtacgccgacctgttcctggccgccaagaacctgtccgacgccatcctgctgtccgacatcct |
| | | gcgcgtgaacaccgagatcaccaaggcccccctgtccgcctccatgatcaagcgctacgacgagcaccacc |
| | | aggacctgaccctgctgaaggccctggtgcgccagcagctgcccgagaagtacaaggagatcttcttcgac |
| | | cagtccaagaacggctacgccggctacatcgacggcggcgcctcccaggaggagttctacaagttcatcaa |
| | | gcccatcctggagaagatggacggcaccgaggagctgctggtgaagctgaaccgcgaggacctgctgcgc |
| | | aagcagcgcaccttcgacaacggctccatcccccaccagatccacctggggagctgcacgccatcctgcg |
| | | ccgccaggaggacttctaccccttcctgaaggacaaccgcgagaagatcgagaagatcctgaccttccgcat |
| | | cccctactacgtgggccccctggcccgcggcaactcccgcttcgcctggatgacccgcaagtccgaggaga |
| | | ccatcaccccctggaacttcgaggaggtggtggacaagggcgcctccgcccagtccttcatcgagcgcatg |
| | | accaacttcgacaagaacctgcccaacgagaaggtgctgcccaagcactccctgctgtacgagtacttcacc |
| | | gtgtacaacgagctgaccaaggtgaagtacgtgaccgagggcatgcgcaagcccgccttcctgtccggcga |
| | | gcagaagaaggccatcgtggacctgctgttcaagaccaaccgcaaggtgaccgtgaagcagctgaaggag |
| | | gactacttcaagaagatcgagtgcttcgactccgtggagatctccggcgtggaggaccgcttcaacgcctccc |
| | | gggcacctaccacgacctgctgaagatcatcaaggacaaggacttcctggacaacgaggagaacgagga |
| | | catcctggaggacatcgtgctgaccctgaccctgttcgaggaccgcgagatgatcgaggagcgcctgaaga |
| | | cctacgcccacctgttcgacgacaaggtgatgaagcagctgaagcgccgccgctacaccggctggggccg |
| | | cctgtcccgcaagctgatcaacggcatccgcgacaagcagtccggcaagaccatcctggacttcctgaagtc |
| | | cgacggcttcgccaaccgcaacttcatgcagctgatccacgacgactccctgaccttcaaggaggacatcca |
| | | gaaggcccaggtgtccggccagggcgactccctgcacgagcacatcgccaacctggccggctcccccgcc |
| | | atcaagaagggcatcctgcagaccgtgaaggtggtggacgagctggtgaaggtgatgggccgccacaagc |
| | | ccgagaacatcgtgatcgagatggcccgcgagaaccagaccacccagaagggccagaagaactcccgcg |
| | | agcgcatgaagcgcatcgaggagggcatcaaggagctgggctcccagatcctgaaggagcaccccgtgg |
| | | agaacacccagctgcagaacgagaagctgtacctgtactacctgcagaacggccgcgacatgtacgtggac |
| | | caggagctggacatcaaccgcctgtccgactacgacgtggaccacatcgtgccccagtccttcctgaaggac |
| | | gactccatcgacaacaaggtgctgacccgctccgacaagaaccgcggaagtccgacaacgtgccctccg |
| | | aggaggtggtgaagaagatgaagaactactggcgccagctgctgaacgccaagctgatcacccagcgcaa |
| | | gttcgacaacctgaccaaggccgagcgcggcggcctgtccgagctggacaaggccggcttcatcaagcgc |
| | | cagctggtggagacccgccagatcaccaagcacgtggcccagatcctggactcccgcatgaacaccaagta |
| | | cgacgagaacgacaagctgatccgcgaggtgaaggtgatcaccctgaagtccaagctggtgtccgacttcc |
| | | gcaaggacttccagttctacaaggtgcgcgagatcaacaactaccaccacgcccacgacgcctacctgaac |
| | | gccgtggtgggcaccgccctgatcaagaagtaccccaagctggagtccgagttcgtgtacggcgactacaa |
| | | ggtgtacgacgtgcgcaagatgatcgccaagtccgagcaggagatcggcaaggccaccgccaagtacttct |
| | | tctactccaacatcatgaacttcttcaagaccgagatcaccctggccaacggcgagatccgcaagcgccccct |
| | | gatcgagaccaacggcgagaccggcgagatcgtgtgggacaagggccgcgacttcgccaccgtgcgcaa |
| | | ggtgctgtccatgccccaggtgaacatcgtgaagaagaccgaggtgcagaccggcggcttctccaaggagt |
| | | ccatcctgcccaagcgcaactccgacaagctgatcgcccgcaagaaggactgggaccccaagaagtacgg |
| | | cggcttcgactcccccaccgtggcctactccgtgctggtggtggccaaggtggagaagggcaagtccaaga |
| | | agctgaagtccgtgaaggagctgctgggcatcaccatcatggagcgctcctccttcgagaagaaccccatcg |
| | | acttcctggaggccaagggctacaaggaggtgaagaaggacctgatcatcaagctgcccaagtactccctgt |
| | | tcgagctggagaacggccgcaagcgcatgctggcctccgccggcgagctgcagaagggcaacgagctgg |
| | | ccctgccctccaagtacgtgaacttcctgtacctggcctcccactacgagaagctgaagggctcccccgagg |
| | | acaacgagcagaagcagctgttcgtggagcagcacaagcactacctggacgagatcatcgagcagatctcc |
| | | gagttctccaagcgcgtgatcctggccgacgccaacctggacaaggtgctgtccgcctacaacaagcaccg |
| | | cgacaagcccatccgcgagcaggccgagaacatcatccacctgttcaccctgaccaacctgggcgcccccg |
| | | ccgccttcaagtacttcgacaccaccatcgaccgcaagcgctacacctccaccaaggaggtgctggacgcc |
| | | accctgatccaccagtccatcaccggcctgtacgagacccgcatcgacctgtcccagctgggcggcgacgg |
| | | ctcccccaagaagaagcgcaaggtgtcctccggcggcgccgccggctgactcgagtaatgaatttccttgtta |
| | | ctcatttattcctagaaatggtgtaatcgctgttgtgggcggagcatatttgtgtatataagagcccgtgttagct |
| | | cctcgattcagtcacaagagcgcacacacacgcttataactagctctctctctccactcaagatggcctttaattt |
| | | tgaagactctacaaatctctttgccaaacaaagcaccagtggtctagtggtagaatagtaccctgccacggtaca |
| | | gacccgggttcgattcccggctggtgcatttgctgcacacgtcaatgagttttagagctagaaatagcaagttaa |
| | | aataaggctagtccgttatcaacttgaaaaagtggcaccgagtcggtgcaacaaagcaccagtggtctagtgg |
| | | tagaatagtaccctgccacggtacagacccgggttcgattcccggctggtgcatgcgagtcccaattcaaaga |
| | | gttttagagctagaaatagcaagttaaaataaggctagtccgttatcaacttgaaaaagtggcaccgagtcggt |
| | | gcaacaaagcaccagtggtctagtggtagaatagtaccctgccacggtacagacccgggttcgattcccggc |
| | | tggtgcacaatactcctcttggaaagggttttagagctagaaatagcaagttaaaataaggctagtccgttatca |
| | | acttgaaaaagtggcaccgagtcggtgctttttttctagacttgtcgactttcaattgaaagggcctcgtgatacg |
| | | cctatttttataggttaatgtcatgataataatggtttcttagacgtcaggtggcacttttcggggaaatgtgcgc |
| | | ggaacccctatttgtttatttttctaaatacattcaaatatgtatccgctcatgagacaataaccctgataaatgc |
| | | ttcaataatattgaaaaaggaagagtatgagtattcaacatttccgtgtcgcccttattcccttttttgcggcatt |
| | | ttgccttcctgtttttgctcacccagaaacgctggtgaaagtaaaagatgctgaagatcagttgggtgcacgagtg |
| | | ggttacatcgaactggatctcaacagcggtaagatccttgagagttttcgccccgaagaacgttttccaatgatga |
| | | gcacttttaaagttctgctatgtggcgcggtattatcccgtattgacgccgggcaagagcaactcggtcgcgcata |
| | | cactattctcagaatgacttggttgagtactcaccagtcacagaaaagcatcttacggatggcatgacagtaaga |
| | | gaattatgcagtgctgccataaccatgagtgataacactgcggccaacttacttctgacaacgatcggaggac |
| | | cgaaggagctaaccgcttttttgcacaacatgggggatcatgtaactcgccttgatcgttgggaaccggagctg |
| | | aatgaagccataccaaacgacgagcgtgacaccacgatgcctgtagcaatggcaacaacgttgcgcaaact |
| | | attaactggcgaactacttactctagcttcccggcaacaattaatagactggatggaggggataaagttgcag |
| | | gaccacttctgcgctcggcccttccggctggctggtttattgctgataaatctggagccggtgagcgtgggagt |
| | | cgcggtatcattgcagcactggggccagatggtaagccctcccgtatcgtagttatctacacgacggggagtc |
| | | aggcaactatggatgaacgaaatagacagatcgctgagataggtgcctcactgattaagcattggtaactgtca |
| | | gaccaagt |
| 146 | P12 | GCTCACATGTgctagcctgcgagcgcttcgcagaaaccgttacaacctatgacgtcataggtcctata |
| | Vector | taagagatgacggactcaccggtctcccagtttctaactgacgagtgaagaggctattcctaatacgactcact |
| | targeting | atagggtaccgccaccATGGACAAGAAGTACTCCATCGGCCTGGACATCGG |
| | VP28 | CACCAACTCCGTGGGCTGGGCCGTGATCACCGACGAGTACAAGGT |
| | gene of | GCCCTCCAAGAAGTTCAAGGTGCTGGGCAACACCGACCGCCACTC |
| | WSSV | CATCAAGAAGAACCTGATCGGCGCCCTGCTGTTCGACTCCGGCGA |
| | expressing | GACCGCCGAGGCCACCCGCCTGAAGCGCACCGCCCGCCGCCGCTA |
| | 3 different | CACCCGCCGCAAGAACCGCATCTGCTACCTGCAGGAGATCTTCTCC |
| | sgRNAs | AACGAGATGGCCAAGGTGGACGACTCCTTCTTCCACCGCCTGGAG |
| | driven by | GAGTCCTTCCTGGTGGAGGAGGACAAGAAGCACGAGCGCCACCC |
| | WSSV ie1 | CATCTTCGGCAACATCGTGGACGAGGTGGCCTACCACGAGAAGTA |
| | promoter | CCCCACCATCTACCACCTGCGCAAGAAGCTGGTGGACTCCACCGA |
| | and | CAAGGCCGACCTGCGCCTGATCTACCTGGCCCTGGCCCACATGATC |
| | NLS-Cas9 | AAGTTCCGCGGCCACTTCCTGATCGAGGGCGACCTGAACCCCGAC |
| | driven by | AACTCCGACGTGGACAAGCTGTTCATCCAGCTGGTGCAGACCTAC |
| | P2 | AACCAGCTGTTCGAGGAGAACCCCATCAACGCCTCCGGCGTGGAC |
| | promoter | GCCAAGGCCATCCTGTCCGCCCGCCTGTCCAAGTCCCGCCGCCTGG |
| | of shrimp | AGAACCTGATCGCCCAGCTGCCCGGCGAGAAGAAGAACGGCCTGT |
| | virus | TCGGCAACCTGATCGCCCTGTCCCTGGGCCTGACCCCCAACTTCAA |
| | IHHNV | GTCCAACTTCGACCTGGCCGAGGACGCCAAGCTGCAGCTGTCCAA |
| | | GGACACCTACGACGACGACCTGGACAACCTGCTGGCCCAGATCGG |
| | | CGACCAGTACGCCGACCTGTTCCTGGCCGCCAAGAACCTGTCCGA |
| | | CGCCATCCTGCTGTCCGACATCCTGCGCGTGAACACCGAGATCACC |
| | | AAGGCCCCCCTGTCCGCCTCCATGATCAAGCGCTACGACGAGCAC |
| | | CACCAGGACCTGACCCTGCTGAAGGCCCTGGTGCGCCAGCAGCTG |
| | | CCCGAGAAGTACAAGGAGATCTTCTTCGACCAGTCCAAGAACGGC |
| | | TACGCCGGCTACATCGACGGCGGCGCCTCCCAGGAGGAGTTCTAC |
| | | AAGTTCATCAAGCCCATCCTGGAGAAGATGGACGGCACCGAGGAG |
| | | CTGCTGGTGAAGCTGAACCGCGAGGACCTGCTGCGCAAGCAGCGC |
| | | ACCTTCGACAACGGCTCCATCCCCCACCAGATCCACCTGGGCGAG |
| | | CTGCACGCCATCCTGCGCCGCCAGGAGGACTTCTACCCCTTCCTGA |
| | | AGGACAACCGCGAGAAGATCGAGAAGATCCTGACCTTCCGCATCC |
| | | CCTACTACGTGGGCCCCCTGGCCCGCGGCAACTCCCGCTTCGCCTG |
| | | GATGACCCGCAAGTCCGAGGAGACCATCACCCCCTGGAACTTCGA |
| | | GGAGGTGGTGGACAAGGGCGCCTCCGCCCAGTCCTTCATCGAGCG |
| | | CATGACCAACTTCGACAAGAACCTGCCCAACGAGAAGGTGCTGCC |
| | | CAAGCACTCCCTGCTGTACGAGTACTTCACCGTGTACAACGAGCTG |
| | | ACCAAGGTGAAGTACGTGACCGAGGGCATGCGCAAGCCCGCCTTC |
| | | CTGTCCGGCGAGCAGAAGAAGGCCATCGTGGACCTGCTGTTCAAG |
| | | ACCAACCGCAAGGTGACCGTGAAGCAGCTGAAGGAGGACTACTTC |
| | | AAGAAGATCGAGTGCTTCGACTCCGTGGAGATCTCCGGCGTGGAG |
| | | GACCGCTTCAACGCCTCCCTGGGCACCTACCACGACCTGCTGAAG |
| | | ATCATCAAGGACAAGGACTTCCTGGACAACGAGGAGAACGAGGA |
| | | CATCCTGGAGGACATCGTGCTGACCCTGACCCTGTTCGAGGACCGC |
| | | GAGATGATCGAGGAGCGCCTGAAGACCTACGCCCACCTGTTCGAC |
| | | GACAAGGTGATGAAGCAGCTGAAGCGCCGCCGCTACACCGGCTGG |
| | | GGCCGCCTGTCCCGCAAGCTGATCAACGGCATCCGCGACAAGCAG |
| | | TCCGGCAAGACCATCCTGGACTTCCTGAAGTCCGACGGCTTCGCC |
| | | AACCGCAACTTCATGCAGCTGATCCACGACGACTCCCTGACCTTCA |
| | | AGGAGGACATCCAGAAGGCCCAGGTGTCCGGCCAGGGCGACTCCC |
| | | TGCACGAGCACATCGCCAACCTGGCCGGCTCCCCCGCCATCAAGA |
| | | AGGGCATCCTGCAGACCGTGAAGGTGGTGGACGAGCTGGTGAAG |
| | | GTGATGGGCCGCCACAAGCCCGAGAACATCGTGATCGAGATGGCC |
| | | CGCGAGAACCAGACCACCCAGAAGGGCCAGAAGAACTCCCGCGA |
| | | GCGCATGAAGCGCATCGAGGAGGGCATCAAGGAGCTGGGCTCCCA |
| | | GATCCTGAAGGAGCACCCCGTGGAGAACACCCAGCTGCAGAACG |
| | | AGAAGCTGTACCTGTACTACCTGCAGAACGGCCGCGACATGTACGT |
| | | GGACCAGGAGCTGGACATCAACCGCCTGTCCGACTACGACGTGGA |
| | | CCACATCGTGCCCCAGTCCTTCCTGAAGGACGACTCCATCGACAAC |
| | | AAGGTGCTGACCCGCTCCGACAAGAACCGCGGCAAGTCCGACAA |
| | | CGTGCCCTCCGAGGAGGTGGTGAAGAAGATGAAGAACTACTGGCG |
| | | CCAGCTGCTGAACGCCAAGCTGATCACCCAGCGCAAGTTCGACAA |
| | | CCTGACCAAGGCCGAGCGCGGCGGCCTGTCCGAGCTGGACAAGG |
| | | CCGGCTTCATCAAGCGCCAGCTGGTGGAGACCCGCCAGATCACCA |
| | | AGCACGTGGCCCAGATCCTGGACTCCCGCATGAACACCAAGTACG |
| | | ACGAGAACGACAAGCTGATCCGCGAGGTGAAGGTGATCACCCTGA |
| | | AGTCCAAGCTGGTGTCCGACTTCCGCAAGGACTTCCAGTTCTACA |
| | | AGGTGCGCGAGATCAACAACTACCACCACGCCCACGACGCCTACC |
| | | TGAACGCCGTGGTGGGCACCGCCCTGATCAAGAAGTACCCCAAGC |
| | | TGGAGTCCGAGTTCGTGTACGGCGACTACAAGGTGTACGACGTGC |
| | | GCAAGATGATCGCCAAGTCCGAGCAGGAGATCGGCAAGGCCACCG |
| | | CCAAGTACTTCTTCTACTCCAACATCATGAACTTCTTCAAGACCGA |
| | | GATCACCCTGGCCAACGGCGAGATCCGCAAGCGCCCCCTGATCGA |
| | | GACCAACGGCGAGACCGGCGAGATCGTGTGGGACAAGGGCCGCG |
| | | ACTTCGCCACCGTGCGCAAGGTGCTGTCCATGCCCCAGGTGAACAT |
| | | CGTGAAGAAGACCGAGGTGCAGACCGGCGGCTTCTCCAAGGAGT |
| | | CCATCCTGCCCAAGCGCAACTCCGACAAGCTGATCGCCCGCAAGA |
| | | AGGACTGGGACCCCAAGAAGTACGGCGGCTTCGACTCCCCCACCG |
| | | TGGCCTACTCCGTGCTGGTGGTGGCCAAGGTGGAGAAGGGCAAGT |
| | | CCAAGAAGCTGAAGTCCGTGAAGGAGCTGCTGGGCATCACCATCA |
| | | TGGAGCGCTCCTCCTTCGAGAAGAACCCCATCGACTTCCTGGAGG |
| | | CCAAGGGCTACAAGGAGGTGAAGAAGGACCTGATCATCAAGCTGC |
| | | CCAAGTACTCCCTGTTCGAGCTGGAGAACGGCCGCAAGCGCATGC |
| | | TGGCCTCCGCCGGCGAGCTGCAGAAGGGCAACGAGCTGGCCCTGC |
| | | CCTCCAAGTACGTGAACTTCCTGTACCTGGCCTCCCACTACGAGAA |
| | | GCTGAAGGGCTCCCCCGAGGACAACGAGCAGAAGCAGCTGTTCGT |
| | | GGAGCAGCACAAGCACTACCTGGACGAGATCATCGAGCAGATCTC |
| | | CGAGTTCTCCAAGCGCGTGATCCTGGCCGACGCCAACCTGGACAA |
| | | GGTGCTGTCCGCCTACAACAAGCACCGCGACAAGCCCATCCGCGA |
| | | GCAGGCCGAGAACATCATCCACCTGTTCACCCTGACCAACCTGGG |
| | | CGCCCCCGCCGCCTTCAAGTACTTCGACACCACCATCGACCGCAA |
| | | GCGCTACACCTCCACCAAGGAGGTGCTGGACGCCACCCTGATCCA |
| | | CCAGTCCATCACCGGCCTGTACGAGACCCGCATCGACCTGTCCCAG |
| | | CTGGGCGGCGACGGCTCCCCCAAGAAGAAGCGCAAGGTGTCCTCC |
| | | GGCGGCGCCGCCGGCTGActcgagTAATGAATTTCCTTGTTACTCATTT |
| | | ATTCCTAGAAATGGTGTAATCGCTGTTGTGGGCGGAGCATATTTGTG |
| | | TATATAAGAGCCCGTGTTAGCTCCTCGATTCAGTCACAAGAGCGCA |
| | | CACACACGCTTATAACTAGCTCTCTCTCTCCACTCAAGATGGCCTTT |
| | | AATTTTGAAGACTCTACAAATCTCTTTGCCAtaatacgactcactatagaacaaag |
| | | caccagtggtctagtggtagaatagtaccctgccacggtacagacccgggttcgattcccggctggtgcagg |
| | | atctttctttcactctttgttttagagctagaaatagcaagttaaaataaggctagtccgttatcaacttgaaaaa |
| | | gtggcaccgagtcggtgcaacaaagcaccagtggtctagtggtagaatagtaccctgccacggtacagacccg |
| | | ggttcgattcccggctggtgcacacagcagtgatggcgaggagttttagagctagaaatagcaagttaaaata |
| | | aggctagtccgttatcaacttgaaaaagtggcaccgagtcggtgcaacaaagcaccagtggtctagtggtaga |
| | | atagtaccctgccacggtacagacccgggttcgattcccggctggtgcatgtgtgggtttcgatggtctgtttta |
| | | gagctagaaatagcaagttaaaataaggctagtccgttatcaacttgaaaaagtggcaccgagtcggtgcTT |
| | | TTTTctagacttgtcgactttcaattgatgtttCATAGGTTAATGTCATGATAATAATGG |
| | | TTTCTTAGACGTCAGGTGGCACTTTTCGGGGAAATGTGCGCGGAAC |
| | | CCCTATTTGTTTATTTTTCTAAATACATTCAAATATGTATCCGCTCATG |
| | | AGACAATAACCCTGATAAATGCTTCAATAATATTGAAAAAGGAAGA |
| | | GTATGAGTATTCAACATTTCCGTGTCGCCCTTATTCCCTTTTTTGCGG |
| | | CATTTTGCCTTCCTGTTTTTGCTCACCCAGAAACGCTGGTGAAAGT |
| | | AAAAGATGCTGAAGATCAGTTGGGTGCACGAGTGGGTTACATCGA |
| | | ACTGGATCTCAACAGCGGTAAGATCCTTGAGAGTTTTCGCCCCGAA |
| | | GAACGTTTcCCAATGATGAGCACTTTTAAAGTTCTGCTATGTGGCGC |
| | | GGTATTATCCCGTATTGACGCCGGGCAAGAGCAACTCGGTCGCCGC |
| | | ATACACTATTCTCAGAATGACTTGGTTGAGTACTCACCAGTCACAG |
| | | AAAAGCATCTTACGGATGGCATGACAGTAAGAGAATTATGCAGTGC |
| | | TGCCATAACCATGAGTGATAACACTGCGGCCAACTTACTTCTGACA |
| | | ACGATCGGAGGACCGAAGGAGCTAACCGCTTTTTTGCACAACATG |
| | | GGGGATCATGTAACTCGCCTTGATCGTTGGGAACCGGAGCTGAATG |
| | | AAGCCATACCAAACGACGAGCGTGACACCACGATGCCTGTAGCAA |
| | | TGGCAACAACGTTGCGCAAACTATTAACTGGCGAACTACTTACTCT |
| | | AGCTTCCCGGCAACAATTAATAGACTGGATGGAGGCGGATAAAGTT |
| | | GCAGGACCACTTCTGCGCTCGGCCCTTCCGGCTGGCTGGTTTATTG |
| | | CTGATAAATCTGGAGCCGGTGAGCGTGGCTCTCGCGGTATCATTGC |
| | | AGCACTGGGGCCAGATGGTAAGCCCTCCCGTATCGTAGTTATCTAC |
| | | ACGACGGGGAGTCAGGCAACTATGGATGAACGAAATAGACAGATC |
| | | GCTGAGATAGGTGCCTCACTGATTAAGCATTGGTAACTGTCAGACC |
| | | AAGTTTACTCATATATACTTTAGATTGATTTAAAACTTCATTTTTAATT |
| | | TAAAAGGATCTAGGTGAAGATCCTTTTTGATAATCTCATGACCAAA |
| | | ATCCCTTAACGTGAGTTTTCGTTCCACTGAGCGTCAGACCCCGTAG |
| | | AAAAGATCAAAGGATCTTCTTGAGATCCTTTTTTTCTGCGCGTAATC |
| | | TGCTGCTTGCAAACAAAAAAACCACCGCTACCAGCGGTGGTTTGT |
| | | TTGCCGGATCAAGAGCTACCAACTCTTTTTCCGAAGGTAACTGGCT |
| | | TCAGCAGAGCGCAGATACCAAATACTGTTCTTCTAGTGTAGCCGTA |
| | | GTTAGGCCACCACTTCAAGAACTCTGTAGCACCGCCTACATACCTC |
| | | GCTCTGCTAATCCTGTTACCAGTGGCTGCTGCCAGTGGCGATAAGT |
| | | CGTGTCTTACCGGGTTGGACTCAAGACGATAGTTACCGGATAAGGC |
| | | GCAGCGGTCGGGCTGAACGGGGGGTTCGTGCACACAGCCCAGCTT |
| | | GGAGCGAACGACCTACACCGAACTGAGATACCTACAGCGTGAGCT |
| | | ATGAGAAAGCGCCACGCTTCCCGAAGGGAGAAAGGCGGACAGGT |
| | | ATCCGGTAAGCGGCAGGGTCGGAACAGGAGAGCGCACGAGGGAG |
| | | CTTCCAGGGGGAAACGCCTGGTATCTTTATAGTCCTGTCGGGTTTC |
| | | GCCACCTCTGACTTGAGCGTCGATTTTTGTGATGCTCGTCAGGGGG |
| | | GCGGAGCCTATGGAAAAACGCCAGCAACGCGGCCTTTTTACGGTT |
| | | CCTGGCCTTTTGCTGGCCTTTT |
| 147 | P24 | ttactcatatatactttagattgatttaaaacttcatttttaatttaaaaggatctaggtgaagatcctttttgat |
| | Vector | aatctcatgaccaaaatcccttaacgtgagttttcgttccactgagcgtcagaccccgtagaaaagatcaaaggat |
| | targeting | cttcttgagatcctttttttctgcgcgtaatctgctgcttgcaaacaaaaaaaccaccgctaccagcggtggtttg |
| | ICP11 of | tttgccggatcaagagctaccaactctttttccgaaggtaactggcttcagcagagcgcagataccaaatactgtt |
| | WSSV | cttctagtgtagccgtagttaggccaccacttcaagaactctgtagcaccgcctacatacctcgctctgctaatcc |
| | including | tgttaccagtggctgctgccagtggcgataagtcgtgtcttaccgggttggactcaagacgatagttaccggataa |
| | 3 sgRNAs | ggcgcagcggtcgggctgaacggggggttcgtgcacacagcccagcttggagcgaacgacctacaccgaa |
| | driven by | ctgagatacctacagcgtgagctatgagaaagcgccacgcttcccgaagggagaaaggcggacaggtatcc |
| | WSSV ie1 | ggtaagcggcagggtcggaacaggagagcgcacgagggagcttccagggggaaacgcctggtatctttat |
| | promoter | agtcctgtcgggtttcgccacctctgacttgagcgtcgatttttgtgatgctcgtcaggggggcggagcctatgg |
| | and | aaaaacgccagcaacgcggcctttttacggttcctggccttttgctggccttttgctcacatgtgctagcctgcga |
| | NLS-Cas9 | gcgcttcgcagaaaccgttacaacctatgacgtcataggtcctatataagagatgacggactcaccggtctccc |
| | driven by | agtttctaactgacgagtgaagaggctattccaagtggtaccgccaccatggacaagaagtactccatcggcc |
| | P2 | tggacatcggcaccaactccgtgggctgggccgtgatcaccgacgagtacaaggtgccctccaagaagttc |
| | promoter | aaggtgctgggcaacaccgaccgccactccatcaagaagaacctgatcggcgccctgctgttcgactccgg |
| | of shrimp | cgagaccgccgaggccacccgcctgaagcgcaccgcccgccgccgctacacccgccgcaagaaccgca |
| | virus | tctgctacctgcaggagatcttctccaacgagatggccaaggtggacgactccttcttccaccgcctggagga |
| | IHHNV | gtccttcctggtggaggaggacaagaagcacgagcgccaccccatcttcggcaacatcgtggacgaggtgg |
| | | cctaccacgagaagtaccccaccattaccacctgcgcaagaagctggtggactccaccgacaaggccgac |
| | | ctgcgcctgatctacctggccctggcccacatgatcaagttccgcggccacttcctgatcgagggcgacctga |
| | | accccgacaactccgacgtggacaagctgttcatccagctggtgcagacctacaaccagctgttcgaggaga |
| | | accccatcaacgcctccggcgtggacgccaaggccatcctgtccgcccgcctgtccaagtcccgccgcctg |
| | | gagaacctgatcgcccagctgcccggcgagaagaagaacggcctgttcggcaacctgatcgccctgtccct |
| | | gggcctgacccccaacttcaagtccaacttcgacctggccgaggacgccaagctgcagctgtccaaggaca |
| | | cctacgacgacgacctggacaacctgctggcccagatcggcgaccagtacgccgacctgttcctggccgcc |
| | | aagaacctgtccgacgccatcctgctgtccgacatcctgcgcgtgaacaccgagatcaccaaggcccccctg |
| | | tccgcctccatgatcaagcgctacgacgagcaccaccaggacctgaccctgctgaaggccctggtgcgcca |
| | | gcagctgcccgagaagtacaaggagatcttcttcgaccagtccaagaacggctacgccggctacatcgacg |
| | | gcggcgcctcccaggaggagttctacaagttcatcaagcccatcctggagaagatggacggcaccgagga |
| | | gctgctggtgaagctgaaccgcgaggacctgctgcgcaagcagcgcaccttcgacaacggctccatccccc |
| | | accagatccacctgggcgagctgcacgccatcctgcgccgccaggaggacttctaccccttcctgaaggac |
| | | aaccgcgagaagatcgagaagatcctgaccttccgcatcccctactacgtgggccccctggcccgcggcaa |
| | | ctcccgcttcgcctggatgacccgcaagtccgaggagaccatcaccccctggaacttcgaggaggtggtgg |
| | | acaagggcgcctccgcccagtccttcatcgagcgcatgaccaacttcgacaagaacctgcccaacgagaag |
| | | gtgctgcccaagcactccctgctgtacgagtacttcaccgtgtacaacgagctgaccaaggtgaagtacgtga |
| | | ccgagggcatgcgcaagcccgccttcctgtccggcgagcagaagaaggccatcgtggacctgctgttcaag |
| | | accaaccgcaaggtgaccgtgaagcagctgaaggaggactacttcaagaagatcgagtgcttcgactccgt |
| | | ggagatctccggcgtggaggaccgcttcaacgcctccctgggcacctaccacgacctgctgaagatcatcaa |
| | | ggacaaggacttcctggacaacgaggagaacgaggacatcctggaggacatcgtgctgaccctgaccctgt |
| | | tcgaggaccgcgagatgatcgaggagcgcctgaagacctacgcccacctgttcgacgacaaggtgatgaa |
| | | gcagctgaagcgccgccgctacaccggctggggccgcctgtcccgcaagctgatcaacggcatccgcgac |
| | | aagcagtccggcaagaccatcctggacttcctgaagtccgacggcttcgccaaccgcaacttcatgcagctg |
| | | atccacgacgactccctgaccttcaaggaggacatccagaaggcccaggtgtccggccagggcgactccct |
| | | gcacgagcacatcgccaacctggccggctcccccgccatcaagaagggcatcctgcagaccgtgaaggtg |
| | | gtggacgagctggtgaaggtgatgggccgccacaagcccgagaacatcgtgatcgagatggcccgcgaga |
| | | accagaccacccagaagggccagaagaactcccgcgagcgcatgaagcgcatcgaggagggcatcaag |
| | | gagctgggctcccagatcctgaaggagcaccccgtggagaacacccagctgcagaacgagaagctgtacc |
| | | tgtactacctgcagaacggccgcgacatgtacgtggaccaggagctggacatcaaccgcctgtccgactacg |
| | | acgtggaccacatcgtgccccagtccttcctgaaggacgactccatcgacaacaaggtgctgacccgctccg |
| | | acaagaaccgcggcaagtccgacaacgtgccctccgaggaggtggtgaagaagatgaagaactactggcg |
| | | ccagctgctgaacgccaagctgatcacccagcgcaagttcgacaacctgaccaaggccgagcgcggcggc |
| | | ctgtccgagctggacaaggccggcttcatcaagcgccagctggtggagacccgccagatcaccaagcacgt |
| | | ggcccagatcctggactcccgcatgaacaccaagtacgacgagaacgacaagctgatccgcgaggtgaag |
| | | gtgatcaccctgaagtccaagctggtgtccgacttccgcaaggacttccagttctacaaggtgcgcgagatca |
| | | acaactaccaccacgcccacgacgcctacctgaacgccgtggtgggcaccgccctgatcaagaagtacccc |
| | | aagctggagtccgagttcgtgtacggcgactacaaggtgtacgacgtgcgcaagatgatcgccaagtccga |
| | | gcaggagatcggcaaggccaccgccaagtacttcttctactccaacatcatgaacttcttcaagaccgagatc |
| | | accctggccaacggcgagatccgcaagcgccccctgatcgagaccaacggcgagaccggcgagatcgtg |
| | | tgggacaagggccgcgacttcgccaccgtgcgcaaggtgctgtccatgccccaggtgaacatcgtgaagaa |
| | | gaccgaggtgcagaccggcggcttctccaaggagtccatcctgcccaagcgcaactccgacaagctgatcg |
| | | cccgcaagaaggactgggaccccaagaagtacggcggcttcgactcccccaccgtggcctactccgtgctg |
| | | gtggtggccaaggtggagaagggcaagtccaagaagctgaagtccgtgaaggagctgctgggcatcacca |
| | | tcatggagcgctcctccttcgagaagaaccccatcgacttcctggaggccaagggctacaaggaggtgaag |
| | | aaggacctgatcatcaagctgcccaagtactccctgttcgagctggagaacggccgcaagcgcatgctggcc |
| | | tccgccggcgagctgcagaagggcaacgagctggccctgccctccaagtacgtgaacttcctgtacctggc |
| | | ctcccactacgagaagctgaagggctcccccgaggacaacgagcagaagcagctgttcgtggagcagcac |
| | | aagcactacctggacgagatcatcgagcagatctccgagttctccaagcgcgtgatcctggccgacgccaac |
| | | ctggacaaggtgctgtccgcctacaacaagcaccgcgacaagcccatccgcgagcaggccgagaacatca |
| | | tccacctgttcaccctgaccaacctgggcgcccccgccgccttcaagtacttcgacaccaccatcgaccgcaa |
| | | gcgctacacctccaccaaggaggtgctggacgccaccctgatccaccagtccatcaccggcctgtacgaga |
| | | cccgcatcgacctgtcccagctgggcggcgacggctcccccaagaagaagcgcaaggtgtcctccggcgg |
| | | cgccgccggctgactcgagtaatgaatttccttgttactcatttattcctagaaatggtgtaatcgctgttgtggg |
| | | cggagcatatttgtgtatataagagcccgtgttagctcctcgattcagtcacaagagcgcacacacacgcttataa |
| | | ctagctctctctctccactcaagatggcctttaattttgaagactctacaaatctctttgccaaacaaagcaccag |
| | | tggtctagtggtagaatagtaccctgccacggtacagacccgggttcgattcccggctggtgcatctgttgttgg |
| | | cacaatcatgttttagagctagaaatagcaagttaaaataaggctagtccgttatcaacttgaaaaagtggcacc |
| | | gagtcggtgcaacaaagcaccagtggtctagtggtagaatagtaccctgccacggtacagacccgggttcga |
| | | ttcccggctggtgcattctgttgttggcacaatcagttttagagctagaaatagcaagttaaaataaggctagtcc |
| | | gttatcaacttgaaaaagtggcaccgagtcggtgcaacaaagcaccagtggtctagtggtagaatagtaccct |
| | | gccacggtacagacccgggttcgattcccggctggtgcatttgactggagattcaacgcgttttagagctagaa |
| | | atagcaagttaaaataaggctagtccgttatcaacttgaaaaagtggcaccgagtcggtgctttttttctagactt |
| | | gtcgactttcaattgaaagggcctcgtgatacgcctatttttataggttaatgtcatgataataatggtttcttag |
| | | acgtcaggtggcacttttcggggaaatgtgcgcggaacccctatttgtttatttttctaaatacattcaaatatgt |
| | | atccgctcatgagacaataaccctgataaatgcttcaataatattgaaaaaggaagagtatgagtattcaacattt |
| | | ccgtgtcgcccttattcccttttttgcggcattttgccttcctgtttttgctcacccagaaacgctggtgaaagta |
| | | aaagatgctgaagatcagttgggtgcacgagtgggttacatcgaactggatctcaacagcggtaagatccttgaga |
| | | gttttcgccccgaagaacgttttccaatgatgagcacttttaaagttctgctatgtggcgcggtattatcccgtat |
| | | tgacgccgggcaagagcaactcggtcgccgcatacactattctcagaatgacttggttgagtactcaccagtcaca |
| | | gaaaagcatcttacggatggcatgacagtaagagaattatgcagtgctgccataaccatgagtgataacactgc |
| | | ggccaacttacttctgacaacgatcggaggaccgaaggagctaaccgcttttttgcacaacatgggggatcat |
| | | gtaactcgccttgatcgttgggaaccggagctgaatgaagccataccaaacgacgagcgtgacaccacgatg |
| | | cctgtagcaatggcaacaacgttgcgcaaactattaactggcgaactacttactctagcttcccggcaacaatta |
| | | atagactggatggaggcggataaagttgcaggaccacttctgcgctcggcccttccggctggctggtttattgc |
| | | tgataaatctggagccggtgagcgtgggagtcgcggtatcattgcagcactggggccagatggtaagccctc |
| | | ccgtatcgtagttatctacacgacggggagtcaggcaactatggatgaacgaaatagacagatcgctgagata |
| | | ggtgcctcactgattaagcattggtaactgtcagaccaagt |
| 148 | p25 | ttactcatatatactttagattgatttaaaacttcatttttaatttaaaaggatctaggtgaagatcctttttgat |
| | Vector | aatctcatgaccaaaatcccttaacgtgagttttcgttccactgagcgtcagaccccgtagaaaagatcaaaggat |
| | targeting | cttcttgagatcctttttttctgcgcgtaatctgctgcttgcaaacaaaaaaaccaccgctaccagcggtggtttg |
| | VP19 | tttgccggatcaagagctaccaactctttttccgaaggtaactggcttcagcagagcgcagataccaaatactgtt |
| | gene of | cttctagtgtagccgtagttaggccaccacttcaagaactctgtagcaccgcctacatacctcgctctgctaatcc |
| | WSSV | tgttaccagtggctgctgccagtggcgataagtcgtgtcttaccgggttggactcaagacgatagttaccggataa |
| | including | ggcgcagcggtcgggctgaacggggggttcgtgcacacagcccagcttggagcgaacgacctacaccgaa |
| | 3 sgRNAs | ctgagatacctacagcgtgagctatgagaaagcgccacgcttcccgaagggagaaaggcggacaggtatcc |
| | driven by | ggtaagcggcagggtcggaacaggagagcgcacgagggagcttccagggggaaacgcctggtatctttat |
| | WSSV ie1 | agtcctgtcgggtttcgccacctctgacttgagcgtcgatttttgtgatgctcgtcaggggggcggagcctatgg |
| | promoter | aaaaacgccagcaacgcggcctttttacggttcctggccttttgctggccttttgctcacatgtgctagcctgcga |
| | and | gcgcttcgcagaaaccgttacaacctatgacgtcataggtcctatataagagatgacggactcaccggtctccc |
| | NLS-Cas9 | agtttctaactgacgagtgaagaggctattccaagtggtaccgccaccatggacaagaagtactccatcggcc |
| | driven by | tggacatcggcaccaactccgtgggctgggccgtgatcaccgacgagtacaaggtgccctccaagaagttc |
| | P2 | aaggtgctgggcaacaccgaccgccactccatcaagaagaacctgatcggcgccctgctgttcgactccgg |
| | promoter | cgagaccgccgaggccacccgcctgaagcgcaccgcccgccgccgctacacccgccgcaagaaccgca |
| | of shrimp | tctgctacctgcaggagatcttctccaacgagatggccaaggtggacgactccttcttccaccgcctggagga |
| | virus | gtccttcctggtggaggaggacaagaagcacgagcgccaccccatcttcggcaacatcgtggacgaggtgg |
| | IHHNV | cctaccacgagaagtaccccaccatctaccacctgcgcaagaagctggtggactccaccgacaaggccgac |
| | | ctgcgcctgatctacctggccctggcccacatgatcaagttccgcggccacttcctgatcgagggcgacctga |
| | | accccgacaactccgacgtggacaagctgttcatccagctggtgcagacctacaaccagctgttcgaggaga |
| | | accccatcaacgcctccggcgtggacgccaaggccatcctgtccgcccgcctgtccaagtcccgccgcctg |
| | | gagaacctgatcgcccagctgcccggcgagaagaagaacggcctgttcggcaacctgatcgccctgtccct |
| | | gggcctgacccccaacttcaagtccaacttcgacctggccgaggacgccaagctgcagctgtccaaggaca |
| | | cctacgacgacgacctggacaacctgctggcccagatcggcgaccagtacgccgacctgttcctggccgcc |
| | | aagaacctgtccgacgccatcctgctgtccgacatcctgcgcgtgaacaccgagatcaccaaggcccccctg |
| | | tccgcctccatgatcaagcgctacgacgagcaccaccaggacctgaccctgctgaaggccctggtgcgcca |
| | | gcagctgcccgagaagtacaaggagatcttcttcgaccagtccaagaacggctacgccggctacatcgacg |
| | | gcggcgcctcccaggaggagttctacaagttcatcaagcccatcctggagaagatggacggcaccgagga |
| | | gctgctggtgaagctgaaccgcgaggacctgctgcgcaagcagcgcaccttcgacaacggctccatccccc |
| | | accagatccacctgggcgagctgcacgccatcctgcgccgccaggaggacttctaccccttcctgaaggac |
| | | aaccgcgagaagatcgagaagatcctgaccttccgcatcccctactacgtgggccccctggcccgggcaa |
| | | ctcccgcttcgcctggatgacccgcaagtccgaggagaccatcaccccctggaacttcgaggagtggtgg |
| | | acaagggcgcctccgcccagtccttcatcgagcgcatgaccaacttcgacaagaacctgcccaacgagaag |
| | | gtgctgcccaagcactccctgctgtacgagtacttcaccgtgtacaacgagctgaccaaggtgaagtacgtga |
| | | ccgagggcatgcgcaagcccgccttcctgtccggcgagcagaagaaggccatcgtggacctgctgttcaag |
| | | accaaccgcaaggtgaccgtgaagcagctgaaggaggactacttcaagaagatcgagtgcttcgactccgt |
| | | ggagatctccggcgtggaggaccgcttcaacgcctccctgggcacctaccacgacctgctgaagatcatcaa |
| | | ggacaaggacttcctggacaacgaggagaacgaggacatcctggaggacatcgtgctgaccctgaccctgt |
| | | tcgaggaccgcgagatgatcgaggagcgcctgaagacctacgcccacctgttcgacgacaaggtgatgaa |
| | | gcagctgaagcgccgccgctacaccggctggggccgcctgtcccgcaagctgatcaacggcatccgcgac |
| | | aagcagtccggcaagaccatcctggacttcctgaagtccgacggcttcgccaaccgcaacttcatgcagctg |
| | | atccacgacgactccctgaccttcaaggaggacatccagaaggcccaggtgtccggccagggcgactccct |
| | | gcacgagcacatcgccaacctggccggctcccccgccatcaagaagggcatcctgcagaccgtgaaggtg |
| | | gtggacgagctggtgaaggtgatgggccgccacaagcccgagaacatcgtgatcgagatggcccgcgaga |
| | | accagaccacccagaagggccagaagaactcccgcgagcgcatgaagcgcatcgaggagggcatcaag |
| | | gagctgggctcccagatcctgaaggagcaccccgtggagaacacccagctgcagaacgagaagctgtacc |
| | | tgtactacctgcagaacggccgcgacatgtacgtggaccaggagctggacatcaaccgcctgtccgactacg |
| | | acgtggaccacatcgtgccccagtccttcctgaaggacgactccatcgacaacaaggtgctgacccgctccg |
| | | acaagaaccgcggcaagtccgacaacgtgccctccgaggaggtggtgaagaagatgaagaactactggcg |
| | | ccagctgctgaacgccaagctgatcacccagcgcaagttcgacaacctgaccaaggccgagcgcggcggc |
| | | ctgtccgagctggacaaggccggcttcatcaagcgccagctggtggagacccgccagatcaccaagcacgt |
| | | ggcccagatcctggactcccgcatgaacaccaagtacgacgagaacgacaagctgatccgcgaggtgaag |
| | | gtgatcaccctgaagtccaagctggtgtccgacttccgcaaggacttccagttctacaaggtgcgcgagatca |
| | | acaactaccaccacgcccacgacgcctacctgaacgccgtggtgggcaccgccctgatcaagaagtacccc |
| | | aagctggagtccgagttcgtgtacggcgactacaaggtgtacgacgtgcgcaagatgatcgccaagtccga |
| | | gcaggagatcggcaaggccaccgccaagtacttcttctactccaacatcatgaacttcttcaagaccgagatc |
| | | accctggccaacggcgagatccgcaagcgccccctgatcgagaccaacggcgagaccggcgagatcgtg |
| | | tgggacaagggccgcgacttcgccaccgtgcgcaaggtgctgtccatgccccaggtgaacatcgtgaagaa |
| | | gaccgaggtgcagaccggcggcttctccaaggagtccatcctgcccaagcgcaactccgacaagctgatcg |
| | | cccgcaagaaggactgggaccccaagaagtacggcggcttcgactcccccaccgtggcctactccgtgctg |
| | | gtggtggccaaggtggagaagggcaagtccaagaagctgaagtccgtgaaggagctgctgggcatcacca |
| | | tcatggagcgctcctccttcgagaagaaccccatcgacttcctggaggccaagggctacaaggaggtgaag |
| | | aaggacctgatcatcaagctgcccaagtactccctgttcgagctggagaacggccgcaagcgcatgctggcc |
| | | tccgccggcgagctgcagaagggcaacgagctggccctgccctccaagtacgtgaacttcctgtacctggc |
| | | ctcccactacgagaagctgaagggctcccccgaggacaacgagcagaagcagctgttcgtggagcagcac |
| | | aagcactacctggacgagatcatcgagcagatctccgagttctccaagcgcgtgatcctggccgacgccaac |
| | | ctggacaaggtgctgtccgcctacaacaagcaccgcgacaagcccatccgcgagcaggccgagaacatca |
| | | tccacctgttcaccctgaccaacctgggcgcccccgccgccttcaagtacttcgacaccaccatcgaccgcaa |
| | | gcgctacacctccaccaaggaggtgctggacgccaccctgatccaccagtccatcaccggcctgtacgaga |
| | | cccgcatcgacctgtcccagctgggcggcgacggctcccccaagaagaagcgcaaggtgtcctccggcgg |
| | | cgccgccggctgactcgagtaatgaatttccttgttactcatttattcctagaaatggtgtaatcgctgttgtggg |
| | | cggagcatatttgtgtatataagagcccgtgttagctcctcgattcagtcacaagagcgcacacacacgcttataa |
| | | ctagctctctctctccactcaagatggcctttaattttgaagactctacaaatctctttgccaaacaaagcaccag |
| | | tggtctagtggtagaatagtaccctgccacggtacagacccgggttcgattcccggctggtgcatgtatggatg |
| | | ggaccaaagagttttagagctagaaatagcaagttaaaataaggctagtccgttatcaacttgaaaaagtggca |
| | | ccgagtcggtgcaacaaagcaccagtggtctagtggtagaatagtaccctgccacggtacagacccgggttc |
| | | gattcccggctggtgcattatgtcttaccccaagagggttttagagctagaaatagcaagttaaaataaggctag |
| | | tccgttatcaacttgaaaaagtggcaccgagtcggtgcaacaaagcaccagtggtctagtggtagaatagtac |
| | | cctgccacggtacagacccgggttcgattcccggctggtgcatacaccatggaagatcttgagttttagagcta |
| | | gaaatagcaagttaaaataaggctagtccgttatcaacttgaaaaagtggcaccgagtcggtgctttttttctaga |
| | | cttgtcgactttcaattgaaagggcctcgtgatacgcctatttttataggttaatgtcatgataataatggtttct |
| | | tagacgtcaggtggcacttttcggggaaatgtgcgcggaacccctatttgtttatttttctaaatacattcaaata |
| | | tgtatccgctcatgagacaataaccctgataaatgcttcaataatattgaaaaaggaagagtatgagtattcaaca |
| | | tttccgtgtcgcccttattcccttttttgcggcattttgccttcctgtttttgctcacccagaaacgctggtgaaa |
| | | gtaaaagatgctgaagatcagttgggtgcacgagtgggttacatcgaactggatctcaacagcggtaagatccttg |
| | | agagttttcgccccgaagaacgttttccaatgatgagcacttttaaagttctgctatgtggcgcggtattatcccg |
| | | tattgacgccgggcaagagcaactcggtcgccgcatacactattctcagaatgacttggttgagtactcaccagtc |
| | | acagaaaagcatcttacggatggcatgacagtaagagaattatgcagtgctgccataaccatgagtgataacactg |
| | | cggccaacttacttctgacaacgatcggaggaccgaaggagctaaccgcttttttgcacaacatgggggatca |
| | | tgtaactcgccttgatcgttgggaaccggagctgaatgaagccataccaaacgacgagcgtgacaccacgat |
| | | gcctgtagcaatggcaacaacgttgcgcaaactattaactggcgaactacttactctagcttcccggcaacaatt |
| | | aatagactggatggaggcggataaagttgcaggaccacttctgcgctcggcccttccggctggctggtttattg |
| | | ctgataaatctggagccggtgagcgtgggagtcgcggtatcattgcagcactggggccagatggtaagccct |
| | | cccgtatcgtagttatctacacgacggggagtcaggcaactatggatgaacgaaatagacagatcgctgagat |
| | | aggtgcctcactgattaagcattggtaactgtcagaccaagt |
| 149 | P33 vector | GCTCACATGTgctagcGACATTGATTATTGACTAGTTATTAATAGTAATC |
| | targeting | AATTACGGGGTCATTAGTTCATAGCCCATATATGGAGTTCCGCGTTA |
| | DNA pol | CATAACTTACGGTAAATGGCCCGCCTGGCTGACCGCCCAACGACCC |
| | gene of | CCGCCCATTGACGTCAATAATGACGTATGTTCCCATAGTAACGCCAA |
| | WSSV | TAGGGACTTTCCATTGACGTCAATGGGTGGAGTATTTACGGTAAAC |
| | including | TGCCCACTTGGCAGTACATCAAGTGTATCATATGCCAAGTCCGCCC |
| | 3 sgRNAs | CCTATTGACGTCAATGACGGTAAATGGCCCGCCTGGCATTATGCCC |
| | driven by | AGTACATGACCTTACGGGACTTTCCTACTTGGCAGTACATCTACGTA |
| | WSSV ie1 | TTAGTCATCGCTATTACCATGGTGATGCGGTTTTGGCAGTACACCAA |
| | promoter | TGGGCGTGGATAGCGGTTTGACTCACGGGGATTTCCAAGTCTCCAC |
| | and | CCCATTGACGTCAATGGGAGTTTGTTTTGGCACCAAAATCAACGGG |
| | NLS-Cas9 | ACTTTCCAAAATGTCGTAATAACCCCGCCCCGTTGACGCAAATGGG |
| | driven by | CGGTAGGCGTGTACGGTGGGAGGTCTATATAAGCAGAGCTggtaccgcc |
| | CMV | accATGGACAAGAAGTACTCCATCGGCCTGGACATCGGCACCAACT |
| | promoter | CCGTGGGCTGGGCCGTGATCACCGACGAGTACAAGGTGCCCTCCA |
| | | AGAAGTTCAAGGTGCTGGGCAACACCGACCGCCACTCCATCAAGA |
| | | AGAACCTGATCGGCGCCCTGCTGTTCGACTCCGGCGAGACCGCCG |
| | | AGGCCACCCGCCTGAAGCGCACCGCCCGCCGCCGCTACACCCGCC |
| | | GCAAGAACCGCATCTGCTACCTGCAGGAGATCTTCTCCAACGAGAT |
| | | GGCCAAGGTGGACGACTCCTTCTTCCACCGCCTGGAGGAGTCCTT |
| | | CCTGGTGGAGGAGGACAAGAAGCACGAGCGCCACCCCATCTTCGG |
| | | CAACATCGTGGACGAGGTGGCCTACCACGAGAAGTACCCCACCAT |
| | | CTACCACCTGCGCAAGAAGCTGGTGGACTCCACCGACAAGGCCGA |
| | | CCTGCGCCTGATCTACCTGGCCCTGGCCCACATGATCAAGTTCCGC |
| | | GGCCACTTCCTGATCGAGGGCGACCTGAACCCCGACAACTCCGAC |
| | | GTGGACAAGCTGTTCATCCAGCTGGTGCAGACCTACAACCAGCTG |
| | | TTCGAGGAGAACCCCATCAACGCCTCCGGCGTGGACGCCAAGGCC |
| | | ATCCTGTCCGCCCGCCTGTCCAAGTCCCGCCGCCTGGAGAACCTGA |
| | | TCGCCCAGCTGCCCGGCGAGAAGAAGAACGGCCTGTTCGGCAACC |
| | | TGATCGCCCTGTCCCTGGGCCTGACCCCCAACTTCAAGTCCAACTT |
| | | CGACCTGGCCGAGGACGCCAAGCTGCAGCTGTCCAAGGACACCTA |
| | | CGACGACGACCTGGACAACCTGCTGGCCCAGATCGGCGACCAGTA |
| | | CGCCGACCTGTTCCTGGCCGCCAAGAACCTGTCCGACGCCATCCTG |
| | | CTGTCCGACATCCTGCGCGTGAACACCGAGATCACCAAGGCCCCC |
| | | CTGTCCGCCTCCATGATCAAGCGCTACGACGAGCACCACCAGGAC |
| | | CTGACCCTGCTGAAGGCCCTGGTGCGCCAGCAGCTGCCCGAGAAG |
| | | TACAAGGAGATCTTCTTCGACCAGTCCAAGAACGGCTACGCCGGC |
| | | TACATCGACGGCGGCGCCTCCCAGGAGGAGTTCTACAAGTTCATCA |
| | | AGCCCATCCTGGAGAAGATGGACGGCACCGAGGAGCTGCTGGTGA |
| | | AGCTGAACCGCGAGGACCTGCTGCGCAAGCAGCGCACCTTCGACA |
| | | ACGGCTCCATCCCCCACCAGATCCACCTGGGCGAGCTGCACGCCAT |
| | | CCTGCGCCGCCAGGAGGACTTCTACCCCTTCCTGAAGGACAACCG |
| | | CGAGAAGATCGAGAAGATCCTGACCTTCCGCATCCCCTACTACGTG |
| | | GGCCCCCTGGCCCGCGGCAACTCCCGCTTCGCCTGGATGACCCGC |
| | | AAGTCCGAGGAGACCATCACCCCCTGGAACTTCGAGGAGGTGGTG |
| | | GACAAGGGCGCCTCCGCCCAGTCCTTCATCGAGCGCATGACCAAC |
| | | TTCGACAAGAACCTGCCCAACGAGAAGGTGCTGCCCAAGCACTCC |
| | | CTGCTGTACGAGTACTTCACCGTGTACAACGAGCTGACCAAGGTG |
| | | AAGTACGTGACCGAGGGCATGCGCAAGCCCGCCTTCCTGTCCGGC |
| | | GAGCAGAAGAAGGCCATCGTGGACCTGCTGTTCAAGACCAACCGC |
| | | AAGGTGACCGTGAAGCAGCTGAAGGAGGACTACTTCAAGAAGATC |
| | | GAGTGCTTCGACTCCGTGGAGATCTCCGGCGTGGAGGACCGCTTC |
| | | AACGCCTCCCTGGGCACCTACCACGACCTGCTGAAGATCATCAAG |
| | | GACAAGGACTTCCTGGACAACGAGGAGAACGAGGACATCCTGGA |
| | | GGACATCGTGCTGACCCTGACCCTGTTCGAGGACCGCGAGATGATC |
| | | GAGGAGCGCCTGAAGACCTACGCCCACCTGTTCGACGACAAGGTG |
| | | ATGAAGCAGCTGAAGCGCCGCCGCTACACCGGCTGGGGCCGCCTG |
| | | TCCCGCAAGCTGATCAACGGCATCCGCGACAAGCAGTCCGGCAAG |
| | | ACCATCCTGGACTTCCTGAAGTCCGACGGCTTCGCCAACCGCAACT |
| | | TCATGCAGCTGATCCACGACGACTCCCTGACCTTCAAGGAGGACAT |
| | | CCAGAAGGCCCAGGTGTCCGGCCAGGGCGACTCCCTGCACGAGCA |
| | | CATCGCCAACCTGGCCGGCTCCCCCGCCATCAAGAAGGGCATCCTG |
| | | CAGACCGTGAAGGTGGTGGACGAGCTGGTGAAGGTGATGGGCCG |
| | | CCACAAGCCCGAGAACATCGTGATCGAGATGGCCCGCGAGAACCA |
| | | GACCACCCAGAAGGGCCAGAAGAACTCCCGCGAGCGCATGAAGC |
| | | GCATCGAGGAGGGCATCAAGGAGCTGGGCTCCCAGATCCTGAAGG |
| | | AGCACCCCGTGGAGAACACCCAGCTGCAGAACGAGAAGCTGTAC |
| | | CTGTACTACCTGCAGAACGGCCGCGACATGTACGTGGACCAGGAG |
| | | CTGGACATCAACCGCCTGTCCGACTACGACGTGGACCACATCGTGC |
| | | CCCAGTCCTTCCTGAAGGACGACTCCATCGACAACAAGGTGCTGA |
| | | CCCGCTCCGACAAGAACCGCGGCAAGTCCGACAACGTGCCCTCCG |
| | | AGGAGGTGGTGAAGAAGATGAAGAACTACTGGCGCCAGCTGCTG |
| | | AACGCCAAGCTGATCACCCAGCGCAAGTTCGACAACCTGACCAAG |
| | | GCCGAGCGCGGCGGCCTGTCCGAGCTGGACAAGGCCGGCTTCATC |
| | | AAGCGCCAGCTGGTGGAGACCCGCCAGATCACCAAGCACGTGGCC |
| | | CAGATCCTGGACTCCCGCATGAACACCAAGTACGACGAGAACGAC |
| | | AAGCTGATCCGCGAGGTGAAGGTGATCACCCTGAAGTCCAAGCTG |
| | | GTGTCCGACTTCCGCAAGGACTTCCAGTTCTACAAGGTGCGCGAG |
| | | ATCAACAACTACCACCACGCCCACGACGCCTACCTGAACGCCGTG |
| | | GTGGGCACCGCCCTGATCAAGAAGTACCCCAAGCTGGAGTCCGAG |
| | | TTCGTGTACGGCGACTACAAGGTGTACGACGTGCGCAAGATGATCG |
| | | CCAAGTCCGAGCAGGAGATCGGCAAGGCCACCGCCAAGTACTTCT |
| | | TCTACTCCAACATCATGAACTTCTTCAAGACCGAGATCACCCTGGC |
| | | CAACGGCGAGATCCGCAAGCGCCCCCTGATCGAGACCAACGGCGA |
| | | GACCGGCGAGATCGTGTGGGACAAGGGCCGCGACTTCGCCACCGT |
| | | GCGCAAGGTGCTGTCCATGCCCCAGGTGAACATCGTGAAGAAGAC |
| | | CGAGGTGCAGACCGGCGGCTTCTCCAAGGAGTCCATCCTGCCCAA |
| | | GCGCAACTCCGACAAGCTGATCGCCCGCAAGAAGGACTGGGACCC |
| | | CAAGAAGTACGGCGGCTTCGACTCCCCCACCGTGGCCTACTCCGT |
| | | GCTGGTGGTGGCCAAGGTGGAGAAGGGCAAGTCCAAGAAGCTGA |
| | | AGTCCGTGAAGGAGCTGCTGGGCATCACCATCATGGAGCGCTCCTC |
| | | CTTCGAGAAGAACCCCATCGACTTCCTGGAGGCCAAGGGCTACAA |
| | | GGAGGTGAAGAAGGACCTGATCATCAAGCTGCCCAAGTACTCCCT |
| | | GTTCGAGCTGGAGAACGGCCGCAAGCGCATGCTGGCCTCCGCCGG |
| | | CGAGCTGCAGAAGGGCAACGAGCTGGCCCTGCCCTCCAAGTACGT |
| | | GAACTTCCTGTACCTGGCCTCCCACTACGAGAAGCTGAAGGGCTC |
| | | CCCCGAGGACAACGAGCAGAAGCAGCTGTTCGTGGAGCAGCACA |
| | | AGCACTACCTGGACGAGATCATCGAGCAGATCTCCGAGTTCTCCAA |
| | | GCGCGTGATCCTGGCCGACGCCAACCTGGACAAGGTGCTGTCCGC |
| | | CTACAACAAGCACCGCGACAAGCCCATCCGCGAGCAGGCCGAGA |
| | | ACATCATCCACCTGTTCACCCTGACCAACCTGGGCGCCCCCGCCGC |
| | | CTTCAAGTACTTCGACACCACCATCGACCGCAAGCGCTACACCTCC |
| | | ACCAAGGAGGTGCTGGACGCCACCCTGATCCACCAGTCCATCACC |
| | | GGCCTGTACGAGACCCGCATCGACCTGTCCCAGCTGGGCGGCGAC |
| | | GGCTCCCCCAAGAAGAAGCGCAAGGTGTCCTCCGGCGGCGCCGCC |
| | | GGCTGActcgagTAATGAATTTCCTTGTTACTCATTTATTCCTAGAAATG |
| | | GTGTAATCGCTGTTGTGGGCGGAGCATATTTGTGTATATAAGAGCCC |
| | | GTGTTAGCTCCTCGATTCAGTCACAAGAGCGCACACACACGCTTAT |
| | | AACTAGCTCTCTCTCTCCACTCAAGATGGCCTTTAATTTTGAAGACT |
| | | CTACAAATCTCTTTGCCAtaatacgactcactatagaacaaagcaccagtggtctagtggtag |
| | | aatagtaccctgccacggtacagacccgggttcgattcccggctggtgcacaagtgtgaacccaaaaatcgtt |
| | | ttagagctagaaatagcaagttaaaataaggctagtccgttatcaacttgaaaaagtggcaccgagtcggtgca |
| | | acaaagcaccagtggtctagtggtagaatagtaccctgccacggtacagacccgggttcgattcccggctggt |
| | | gcaaccactattgtaaaccgatggttttagagctagaaatagcaagttaaaataaggctagtccgttatcaacttg |
| | | aaaaagtggcaccgagtcggtgcaacaaagcaccagtggtctagtggtagaatagtaccctgccacggtaca |
| | | gacccgggttcgattcccggctggtgcaggtacaactggatttggggggttttagagctagaaatagcaagtta |
| | | aaataaggctagtccgttatcaacttgaaaaagtggcaccgagtcggtgcTTTTTTctagacttgtcgactt |
| | | tcaattgatgtttCATAGGTTAATGTCATGATAATAATGGTTTCTTAGACGTC |
| | | AGGTGGCACTTTTCGGGGAAATGTGCGCGGAACCCCTATTTGTTTA |
| | | TTTTTCTAAATACATTCAAATATGTATCCGCTCATGAGACAATAACCC |
| | | TGATAAATGCTTCAATAATATTGAAAAAGGAAGAGTATGAGTATTCA |
| | | ACATTTCCGTGTCGCCCTTATTCCCTTTTTTGCGGCATTTTGCCTTCC |
| | | TGTTTTTGCTCACCCAGAAACGCTGGTGAAAGTAAAAGATGCTGA |
| | | AGATCAGTTGGGTGCACGAGTGGGTTACATCGAACTGGATCTCAAC |
| | | AGCGGTAAGATCCTTGAGAGTTTTCGCCCCGAAGAACGTTTcCCAA |
| | | TGATGAGCACTTTTAAAGTTCTGCTATGTGGCGCGGTATTATCCCGT |
| | | ATTGACGCCGGGCAAGAGCAACTCGGTCGCCGCATACACTATTCTC |
| | | AGAATGACTTGGTTGAGTACTCACCAGTCACAGAAAAGCATCTTAC |
| | | GGATGGCATGACAGTAAGAGAATTATGCAGTGCTGCCATAACCATG |
| | | AGTGATAACACTGCGGCCAACTTACTTCTGACAACGATCGGAGGA |
| | | CCGAAGGAGCTAACCGCTTTTTTGCACAACATGGGGGATCATGTAA |
| | | CTCGCCTTGATCGTTGGGAACCGGAGCTGAATGAAGCCATACCAA |
| | | ACGACGAGCGTGACACCACGATGCCTGTAGCAATGGCAACAACGT |
| | | TGCGCAAACTATTAACTGGCGAACTACTTACTCTAGCTTCCCGGCA |
| | | ACAATTAATAGACTGGATGGAGGCGGATAAAGTTGCAGGACCACTT |
| | | CTGCGCTCGGCCCTTCCGGCTGGCTGGTTTATTGCTGATAAATCTGG |
| | | AGCCGGTGAGCGTGGcTCTCGCGGTATCATTGCAGCACTGGGGCCA |
| | | GATGGTAAGCCCTCCCGTATCGTAGTTATCTACACGACGGGGAGTC |
| | | AGGCAACTATGGATGAACGAAATAGACAGATCGCTGAGATAGGTG |
| | | CCTCACTGATTAAGCATTGGTAACTGTCAGACCAAGTTTACTCATAT |
| | | ATACTTTAGATTGATTTAAAACTTCATTTTTAATTTAAAAGGATCTAG |
| | | GTGAAGATCCTTTTTGATAATCTCATGACCAAAATCCCTTAACGTGA |
| | | GTTTTCGTTCCACTGAGCGTCAGACCCCGTAGAAAAGATCAAAGG |
| | | ATCTTCTTGAGATCCTTTTTTTCTGCGCGTAATCTGCTGCTTGCAAA |
| | | CAAAAAAACCACCGCTACCAGCGGTGGTTTGTTTGCCGGATCAAG |
| | | AGCTACCAACTCTTTTTCCGAAGGTAACTGGCTTCAGCAGAGCGCA |
| | | GATACCAAATACTGTTCTTCTAGTGTAGCCGTAGTTAGGCCACCACT |
| | | TCAAGAACTCTGTAGCACCGCCTACATACCTCGCTCTGCTAATCCT |
| | | GTTACCAGTGGCTGCTGCCAGTGGCGATAAGTCGTGTCTTACCGGG |
| | | TTGGACTCAAGACGATAGTTACCGGATAAGGCGCAGCGGTCGGGC |
| | | TGAACGGGGGGTTCGTGCACACAGCCCAGCTTGGAGCGAACGACC |
| | | TACACCGAACTGAGATACCTACAGCGTGAGCTATGAGAAAGCGCC |
| | | ACGCTTCCCGAAGGGAGAAAGGCGGACAGGTATCCGGTAAGCGGC |
| | | AGGGTCGGAACAGGAGAGCGCACGAGGGAGCTTCCAGGGGGAAA |
| | | CGCCTGGTATCTTTATAGTCCTGTCGGGTTTCGCCACCTCTGACTTG |
| | | AGCGTCGATTTTTGTGATGCTCGTCAGGGGGGCGGAGCCTATGGAA |
| | | AAACGCCAGCAACGCGGCCTTTTTACGGTTCCTGGCCTTTTGCTGG |
| | | CCTTTT |
| 150 | P35- | ttactcatatatactttagattgatttaaaacttcatttttaatttaaaaggatctaggtgaagatcctttttgat |
| | WSSV | aatctcatgaccaaaatcccttaacgtgagttttcgttccactgagcgtcagaccccgtagaaaagatcaaaggat |
| | VP19 | cttcttgagatcctttttttctgcgcgtaatctgctgcttgcaaacaaaaaaaccaccgctaccagcggtggtttg |
| | triplex_ | tttgccggatcaagagctaccaactctttttccgaaggtaactggcttcagcagagcgcagataccaaatactgtt |
| | PmAV car | cttctagtgtagccgtagttaggccaccacttcaagaactctgtagcaccgcctacatacctcgctctgctaatcc |
| | pBA | tgttaccagtggctgctgccagtggcgataagtcgtgtcttaccgggttggactcaagacgatagttaccggataa |
| | | ggcgcagcggtcgggctgaacggggggttcgtgcacacagcccagcttggagcgaacgacctacaccgaa |
| | | ctgagatacctacagcgtgagctatgagaaagcgccacgcttcccgaagggagaaaggcggacaggtatcc |
| | | ggtaagcggcagggtcggaacaggagagcgcacgagggagcttccagggggaaacgcctggtatctttat |
| | | agtcctgtcgggtttcgccacctctgacttgagcgtcgatttttgtgatgctcgtcaggggggcggagcctatgg |
| | | aaaaacgccagcaacgcggcctttttacggttcctggccttttgctggccttttgctcacatgtgctagcagtccc |
| | | acactccatcaattatcaaactatttcttaaatttcctctgccagcccttcgtgggaagacagcaagtgttgttga |
| | | tttttccgtaaacacttcatgattcctctgttataaaaggaaatggagaatcgccctgcactctgccccgccgatg |
| | | ccacaggtaccgccaccatggacaagaagtactccatcggcctggacatcggcaccaactccgtgggctggg |
| | | ccgtgatcaccgacgagtacaaggtgccctccaagaagttcaaggtgctgggcaacaccgaccgccactcc |
| | | atcaagaagaacctgatcggcgccctgctgttcgactccggcgagaccgccgaggccacccgcctgaagc |
| | | gcaccgcccgccgccgctacacccgccgcaagaaccgcatctgctacctgcaggagatcttctccaacgag |
| | | atggccaaggtggacgactccttcttccaccgcctggaggagtccttcctggtggaggaggacaagaagcac |
| | | gagcgccaccccatcttcggcaacatcgtggacgaggtggcctaccacgagaagtaccccaccatctacca |
| | | cctgcgcaagaagctggtggactccaccgacaaggccgacctgcgcctgatctacctggccctggcccaca |
| | | tgatcaagttccgcggccacttcctgatcgaggggacctgaaccccgacaactccgacgtggacaagctgt |
| | | tcatccagctggtgcagacctacaaccagctgttcgaggagaaccccatcaacgcctccggcgtggacgcc |
| | | aaggccatcctgtccgcccgcctgtccaagtcccgccgcctggagaacctgatcgcccagctgcccggcga |
| | | gaagaagaacggcctgttcggcaacctgatcgccctgtccctgggcctgacccccaacttcaagtccaacttc |
| | | gacctggccgaggacgccaagctgcagctgtccaaggacacctacgacgacgacctggacaacctgctgg |
| | | cccagatcggcgaccagtacgccgacctgttcctggccgccaagaacctgtccgacgccatcctgctgtccg |
| | | acatcctgcgcgtgaacaccgagatcaccaaggcccccctgtccgcctccatgatcaagcgctacgacgag |
| | | caccaccaggacctgaccctgctgaaggccctggtgcgccagcagctgcccgagaagtacaaggagatctt |
| | | cttcgaccagtccaagaacggctacgccggctacatcgacggcggcgcctcccaggaggagttctacaagtt |
| | | catcaagcccatcctggagaagatggacggcaccgaggagctgctggtgaagctgaaccgcgaggacctg |
| | | ctgcgcaagcagcgcaccttcgacaacggctccatcccccaccagatccacctgggcgagctgcacgccat |
| | | cctgcgccgccaggaggacttctaccccttcctgaaggacaaccgcgagaagatcgagaagatcctgacctt |
| | | ccgcatcccctactacgtgggccccctggcccgcggcaactcccgcttcgcctggatgacccgcaagtccg |
| | | aggagaccatcaccccctggaacttcgaggaggtggtggacaagggcgcctccgcccagtccttcatcgag |
| | | cgcatgaccaacttcgacaagaacctgcccaacgagaaggtgctgcccaagcactccctgctgtacgagtac |
| | | ttcaccgtgtacaacgagctgaccaaggtgaagtacgtgaccgagggcatgcgcaagcccgccttcctgtcc |
| | | ggcgagcagaagaaggccatcgtggacctgctgttcaagaccaaccgcaaggtgaccgtgaagcagctga |
| | | aggaggactacttcaagaagatcgagtgcttcgactccgtggagatctccggcgtggaggaccgcttcaacg |
| | | cctccctgggcacctaccacgacctgctgaagatcatcaaggacaaggacttcctggacaacgaggagaac |
| | | gaggacatcctggaggacatcgtgctgaccctgaccctgttcgaggaccgcgagatgatcgaggagcgcct |
| | | gaagacctacgcccacctgttcgacgacaaggtgatgaagcagctgaagcgccgccgctacaccggctgg |
| | | ggccgcctgtcccgcaagctgatcaacggcatccgcgacaagcagtccggcaagaccatcctggacttcct |
| | | gaagtccgacggcttcgccaaccgcaacttcatgcagctgatccacgacgactccctgaccttcaaggagga |
| | | catccagaaggcccaggtgtccggccagggcgactccctgcacgagcacatcgccaacctggccggctcc |
| | | cccgccatcaagaagggcatcctgcagaccgtgaaggtggtggacgagctggtgaaggtgatgggccgcc |
| | | acaagcccgagaacatcgtgatcgagatggcccgcgagaaccagaccacccagaagggccagaagaact |
| | | cccgcgagcgcatgaagcgcatcgaggagggcatcaaggagctgggctcccagatcctgaaggagcacc |
| | | ccgtggagaacacccagctgcagaacgagaagctgtacctgtactacctgcagaacggccgcgacatgtac |
| | | gtggaccaggagctggacatcaaccgcctgtccgactacgacgtggaccacatcgtgccccagtccttcctg |
| | | aaggacgactccatcgacaacaaggtgctgacccgctccgacaagaaccgcggcaagtccgacaacgtgc |
| | | cctccgaggaggtggtgaagaagatgaagaactactggcgccagctgctgaacgccaagctgatcacccag |
| | | cgcaagttcgacaacctgaccaaggccgagcgcggcggcctgtccgagctggacaaggccggcttcatca |
| | | agcgccagctggtggagacccgccagatcaccaagcacgtggcccagatcctggactcccgcatgaacac |
| | | caagtacgacgagaacgacaagctgatccgcgaggtgaaggtgatcaccctgaagtccaagctggtgtccg |
| | | acttccgcaaggacttccagttctacaaggtgcgcgagatcaacaactaccaccacgcccacgacgcctacc |
| | | tgaacgccgtggtgggcaccgccctgatcaagaagtaccccaagctggagtccgagttcgtgtacggcgact |
| | | acaaggtgtacgacgtgcgcaagatgatcgccaagtccgagcaggagatcggcaaggccaccgccaagta |
| | | cttcttctactccaacatcatgaacttcttcaagaccgagatcaccctggccaacggcgagatccgcaagcgcc |
| | | ccctgatcgagaccaacggcgagaccggcgagatcgtgtgggacaagggccgcgacttcgccaccgtgc |
| | | gcaaggtgctgtccatgccccaggtgaacatcgtgaagaagaccgaggtgcagaccggcggcttctccaag |
| | | gagtccatcctgcccaagcgcaactccgacaagctgatcgcccgcaagaaggactgggaccccaagaagt |
| | | acggcggcttcgactcccccaccgtggcctactccgtgctggtggtggccaaggtggagaagggcaagtcc |
| | | aagaagctgaagtccgtgaaggagctgctgggcatcaccatcatggagcgctcctccttcgagaagaacccc |
| | | atcgacttcctggaggccaagggctacaaggaggtgaagaaggacctgatcatcaagctgcccaagtactcc |
| | | ctgttcgagctggagaacggccgcaagcgcatgctggcctccgccggcgagctgcagaagggcaacgag |
| | | ctggccctgccctccaagtacgtgaacttcctgtacctggcctcccactacgagaagctgaagggctcccccg |
| | | aggacaacgagcagaagcagctgttcgtggagcagcacaagcactacctggacgagatcatcgagcagat |
| | | ctccgagttctccaagcgcgtgatcctggccgacgccaacctggacaaggtgctgtccgcctacaacaagca |
| | | ccgcgacaagcccatccgcgagcaggccgagaacatcatccacctgttcaccctgaccaacctgggcgccc |
| | | ccgccgccttcaagtacttcgacaccaccatcgaccgcaagcgctacacctccaccaaggaggtgctggac |
| | | gccaccctgatccaccagtccatcaccggcctgtacgagacccgcatcgacctgtcccagctgggcggcga |
| | | cggctcccccaagaagaagcgcaaggtgtcctccggcggcgccgccggctgactcgagaagcttttagacc |
| | | ttcttacttttggggattatataagtattttctcaataaatatctcatatcttactgtggtttaactgctgaatct |
| | | aaaattttaatacaaaagtagttatatttgttgtacattgtaaactataacttaacttcagtttcagagaaactca |
| | | tgtgctcaaaatgtaaaaaaagtttcctgttaaatattttgtaaatgtattgaagacaaaataagaaaaaaaaaaa |
| | | tataagccactaaatcacactgtccttggtatcagcaagagattctgacataatcagctgtttttgtttattactg |
| | | ccattgaaggccatgtgcattagtcccaagttacacattaaaaagtcacatgtagcttaccaacatcagtgctgtt |
| | | caagcacagcctcatctactattcaaactgtggcaccatctaaaatatgccagaatttttttatttaatgaatttg |
| | | accctgaaatatgtattaatatcactcctgtgatttttttgtaatcagcttacaattacaggaatgcaagcctgat |
| | | tcattacaagtttcactacactttctctgacaacatcacctactgaactcagaccagctagttgctccttaagtat |
| | | acaatcatgtcagtaatcctcatttcaatgaaaaatacccgtattgtacttggtacttggtagataaccacagagc |
| | | agtattatgccattattgtgaatacaataagaggtaaatgacctacagagctgctgctgctgttgtgttagattgt |
| | | aaacacagcacaggatcaaggaggtgtccatcactatgaccaatactagcactttgcacaggctctttgaaaggct |
| | | gaaaagagccttattggcgttatcacaacaaaatacgcaaatacggaaaacaacgtattgaacttcgcaaacaaaa |
| | | aacagcgattttgatgaaaatcgcttaggccttgctcttcaaacaatccagcttctccttctttcactctcaagtt |
| | | gcaagaagcaagtgtagcaatgtgcacgcgacagccgggtgtgtgacgctggaccaatcagagcgcagagctccga |
| | | aagtttaccttttatggctagagccggcatctgccgtcatataaaagagcgcgcccagcgtctcagcctcactttg |
| | | agctcctccacacgcagctagtgcggaatatcatctgcctgtaacccattctctaaagtcgacaaacccccccaaa |
| | | cctaagaacaaagcaccagtggtctagtggtagaatagtaccctgccacggtacagacccgggttcgattcccggc |
| | | tggtgcatgtatggatgggaccaaagagttttagagctagaaatagcaagttaaaataaggctagtccgttatcaa |
| | | cttgaaaaagtggcaccgagtcggtgcaacaaagcaccagtggtctagtggtagaatagtaccctgccacggtaca |
| | | gacccgggttcgattcccggctggtgcattatgtcttaccccaagagggttttagagctagaaatagcaagttaa |
| | | aataaggctagtccgttatcaacttgaaaaagtggcaccgagtcggtgcaacaaagcaccagtggtctagtgg |
| | | tagaatagtaccctgccacggtacagacccgggttcgattcccggctggtgcatacaccatggaagatcttga |
| | | gttttagagctagaaatagcaagttaaaataaggctagtccgttatcaacttgaaaaagtggcaccgagtcggt |
| | | gctttttttctagacttgtcgactttcaattgaaagggcctcgtgatacgcctatttttataggttaatgtcatga |
| | | taataatggtttcttagacgtcaggtggcacttttcggggaaatgtgcgcggaacccctatttgtttatttttcta |
| | | aatacattcaaatatgtatccgctcatgagacaataaccctgataaatgcttcaataatattgaaaaaggaagagt |
| | | atgagtattcaacatttccgtgtcgcccttattcccttttttgcggcattttgccttcctgtttttgctcacccag |
| | | aaacgctggtgaaagtaaaagatgctgaagatcagttgggtgcacgagtgggttacatcgaactggatctcaacag |
| | | cggtaagatccttgagagttttcgccccgaagaacgttttccaatgatgagcacttttaaagttctgctatgtggc |
| | | gcggtattatcccgtattgacgccgggcaagagcaactcggtcgccgcatacactattctcagaatgacttggttg |
| | | agtactcaccagtcacagaaaagcatcttacggatggcatgacagtaagagaattatgcagtgctgccataaccat |
| | | ggatgataacactgcggccaacttacttctgacaacgatcggaggaccgaaggagctaaccgcttttttgcacaa |
| | | catgggggatcatgtaactcgccttgatcgttgggaaccggagctgaatgaagccataccaaacgacgagcg |
| | | tgacaccacgatgcctgtagcaatggcaacaacgttgcgcaaactattaactggcgaactacttactctagcttc |
| | | ccggcaacaattaatagactggatggaggcggataaagttgcaggaccacttctgcgctcggcccttccggct |
| | | ggctggtttattgctgataaatctggagccggtgagcgtgggagtcgcggtatcattgcagcactggggccag |
| | | atggtaagccctcccgtatcgtagttatctacacgacggggagtcaggcaactatggatgaacgaaatagaca |
| | | gatcgctgagataggtgcctcactgattaagcattggtaactgtcagaccaagt |
| 151 | p34 | ttactcatatatactttagattgatttaaaacttcatttttaatttaaaaggatctaggtgaagatcctttttgat |
| | WSSV | aatctcatgaccaaaatcccttaacgtgagttttcgttccactgagcgtcagaccccgtagaaaagatcaaaggat |
| | VP26_LvBA_ | cttcttgagatcctttttttctgcgcgtaatctgctgcttgcaaacaaaaaaaccaccgctaccagcggtggtttg |
| | TCTP | tttgccggatcaagagctaccaactctttttccgaaggtaactggcttcagcagagcgcagataccaaatactgtt |
| | | cttctagtgtagccgtagttaggccaccacttcaagaactctgtagcaccgcctacatacctcgctctgctaatcc |
| | | tgttaccagtggctgctgccagtggcgataagtcgtgtcttaccgggttggactcaagacgatagttaccggataa |
| | | ggcgcagcggtcgggctgaacggggggttcgtgcacacagcccagcttggagcgaacgacctacaccgaa |
| | | ctgagatacctacagcgtgagctatgagaaagcgccacgcttcccgaagggagaaaggcggacaggtatcc |
| | | ggtaagcggcaggcgggtcggaacaggagagcgcacgagggagcttccagggggaaacgcctggtatctttat |
| | | agtcctgtcgggtttcgccacctctgacttgagcgtcgatttttgtgatgctcgtcaggggggcggagcctatgg |
| | | aaaaacgccagcaacgcggcctttttacggttcctggccttttgctggccttttgctcacatgtgctagccaaatg |
| | | aggcggcggcaatgatttacgggcatatattcggtcgaggaggacgaaatattctgaaatgggacgaaaggg |
| | | gatgacgcggcgcggctctcgtcttcccgcctcgcattcaacgctcggctcgaccaatcagcggccgagtttt |
| | | gcgctatgaccatataaggcgatacgtttgtccgggtggggtgggacgagccattgcggcttatcgcgcggg |
| | | ggagtaccctctcaaaatgcactatgcactgccgtaacactctttcggaaagaatataatacatcagtagatacc |
| | | tcttgaaaattaggatccgatgcataccataaatccccaaattagagagaataaaaggggttaattcgatcgag |
| | | agtaatgacacttggaacgacctcccctctggagaaagtcgacgatccgagaggtggagtaagcgccctact |
| | | cactctctcggtaccgccaccatggacaagaagtactccatcggcctggacatcggcaccaactccgtgggc |
| | | tgggccgtgatcaccgacgagtacaaggtgccctccaagaagttcaaggtgctgggcaacaccgaccgcca |
| | | ctccatcaagaagaacctgatcggcgccctgctgttcgactccggcgagaccgccgaggccacccgcctga |
| | | agcgcaccgcccgccgccgctacacccgccgcaagaaccgcatctgctacctgcaggagatcttctccaac |
| | | gagatggccaaggtggacgactccttcttccaccgcctggaggagtccttcctggtggaggaggacaagaa |
| | | gcacgagcgccaccccatcttcggcaacatcgtggacgaggtggcctaccacgagaagtaccccaccatct |
| | | accacctgcgcaagaagctggtggactccaccgacaaggccgacctgcgcctgatctacctggccctggcc |
| | | cacatgatcaagttccgcggccacttcctgatcgagggcgacctgaaccccgacaactccgacgtggacaa |
| | | gtgttcatccagctggtgcagacctacaaccagctgttcgaggagaaccccatcaacgcctccggcgtgga |
| | | cgccaaggccatcctgtccgcccgcctgtccaagtcccgccgcctggagaacctgatcgcccagctgcccg |
| | | gcgagaagaagaacggcctgttcggcaacctgatcgccctgtccctgggcctgacccccaacttcaagtcca |
| | | acttcgacctggccgaggacgccaagctgcagctgtccaaggacacctacgacgacgacctggacaacctg |
| | | ctggcccagatcggcgaccagtacgccgacctgttcctggccgccaagaacctgtccgacgccatcctgctg |
| | | tccgacatcctgcgcgtgaacaccgagatcaccaaggcccccctgtccgcctccatgatcaagcgctacgac |
| | | gagcaccaccaggacctgaccctgctgaaggccctggtgcgccagcagctgcccgagaagtacaaggag |
| | | atcttcttcgaccagtccaagaacggctacgccggctacatcgacggcggcgcctcccaggaggagttctac |
| | | aagttcatcaagcccatcctggagaagatggacggcaccgaggagctgctggtgaagctgaaccgcgagg |
| | | acctgctgcgcaagcagcgcaccttcgacaacggctccatcccccaccagatccacctgggcgagctgcac |
| | | gccatcctgcgccgccaggaggacttctaccccttcctgaaggacaaccgcgagaagatcgagaagatcct |
| | | gaccttccgcatcccctactacgtgggccccctggcccgcggcaactcccgcttcgcctggatgacccgcaa |
| | | gtccgaggagaccatcaccccctggaacttcgaggaggtggtggacaagggcgcctccgcccagtccttca |
| | | tcgagcgcatgaccaacttcgacaagaacctgcccaacgagaaggtgctgcccaagcactccctgctgtacg |
| | | agtacttcaccgtgtacaacgagctgaccaaggtgaagtacgtgaccgagggcatgcgcaagcccgccttcc |
| | | tgtccggcgagcagaagaaggccatcgtggacctgctgttcaagaccaaccgcaaggtgaccgtgaagca |
| | | gctgaaggaggactacttcaagaagatcgagtgcttcgactccgtggagatctccggcgtggaggaccgctt |
| | | caacgcctccctgggcacctaccacgacctgctgaagatcatcaaggacaaggacttcctggacaacgagg |
| | | agaacgaggacatcctggaggacatcgtgctgaccctgaccctgttcgaggaccgcgagatgatcgaggag |
| | | cgcctgaagacctacgcccacctgttcgacgacaaggtgatgaagcagctgaagcgccgccgctacaccg |
| | | gctggggccgcctgtcccgcaagctgatcaacggcatccgcgacaagcagtccggcaagaccatcctgga |
| | | cttcctgaagtccgacggcttcgccaaccgcaacttcatgcagctgatccacgacgactccctgaccttcaag |
| | | gaggacatccagaaggcccaggtgtccggccagggcgactccctgcacgagcacatcgccaacctggcc |
| | | ggctcccccgccatcaagaagggcatcctgcagaccgtgaaggtggtggacgagctggtgaaggtgatgg |
| | | gccgccacaagcccgagaacatcgtgatcgagatggcccgcgagaaccagaccacccagaagggccaga |
| | | agaactcccgcgagcgcatgaagcgcatcgaggagggcatcaaggagctgggctcccagatcctgaagga |
| | | gcaccccgtggagaacacccagctgcagaacgagaagctgtacctgtactacctgcagaacggccgcgac |
| | | atgtacgtggaccaggagctggacatcaaccgcctgtccgactacgacgtggaccacatcgtgccccagtcc |
| | | ttcctgaaggacgactccatcgacaacaaggtgctgacccgctccgacaagaaccgcggcaagtccgacaa |
| | | cgtgccctccgaggaggtggtgaagaagatgaagaactactggcgccagctgctgaacgccaagctgatca |
| | | cccagcgcaagttcgacaacctgaccaaggccgagcgcggcggcctgtccgagctggacaaggccggctt |
| | | catcaagcgccagctggtggagacccgccagatcaccaagcacgtggcccagatcctggactcccgcatga |
| | | acaccaagtacgacgagaacgacaagctgatccgcgaggtgaaggtgatcaccctgaagtccaagctggtg |
| | | tccgacttccgcaaggacttccagttctacaaggtgcgcgagatcaacaactaccaccacgcccacgacgcc |
| | | tacctgaacgccgtggtgggcaccgccctgatcaagaagtaccccaagctggagtccgagttcgtgtacggc |
| | | gactacaaggtgtacgacgtgcgcaagatgatcgccaagtccgagcaggagatcggcaaggccaccgcca |
| | | agtacttcttctactccaacatcatgaacttcttcaagaccgagatcaccctggccaacggcgagatccgcaag |
| | | cgccccctgatcgagaccaacggcgagaccggcgagatcgtgtgggacaagggccgcgacttcgccacc |
| | | gtgcgcaaggtgctgtccatgccccaggtgaacatcgtgaagaagaccgaggtgcagaccggcggcttctc |
| | | caaggagtccatcctgcccaagcgcaactccgacaagctgatcgcccgcaagaaggactgggaccccaag |
| | | aagtacggcggcttcgactcccccaccgtggcctactccgtgctggtggtggccaaggtggagaagggcaa |
| | | gtccaagaagctgaagtccgtgaaggagctgctgggcatcaccatcatggagcgctcctccttcgagaagaa |
| | | ccccatcgacttcctggaggccaagggctacaaggaggtgaagaaggacctgatcatcaagctgcccaagt |
| | | actccctgttcgagctggagaacggccgcaagcgcatgctggcctccgccggcgagctgcagaagggcaa |
| | | cgagctggccctgccctccaagtacgtgaacttcctgtacctggcctcccactacgagaagctgaagggctcc |
| | | cccgaggacaacgagcagaagcagctgttcgtggagcagcacaagcactacctggacgagatcatcgagc |
| | | agatctccgagttctccaagcgcgtgatcctggccgacgccaacctggacaaggtgctgtccgcctacaaca |
| | | agcaccgcgacaagcccatccgcgagcaggccgagaacatcatccacctgttcaccctgaccaacctgggc |
| | | gcccccgccgccttcaagtacttcgacaccaccatcgaccgcaagcgctacacctccaccaaggaggtgct |
| | | ggacgccaccctgatccaccagtccatcaccggcctgtacgagacccgcatcgacctgtcccagctgggcg |
| | | gcgacggctcccccaagaagaagcgcaaggtgtcctccggcggcgccgccggctgactcgagGATAT |
| | | GCATATACTTACTCATTTACTGTAAAGAGATAGTAAAGCAGAGATCA |
| | | TTAATTCGGTTGCTCATCCTCGTTTCCTTCCAATGTACGAAGGAGTG |
| | | GCAACAGCAAGGTAATTGAAGTTCGTTAGTAGAGGGAAAAGGAAG |
| | | CGTGAAACTTAACCGGCGATTGGTGCCTTCGTATCTAGGTCATATTA |
| | | AAAGCCACTGTCTCTTCACGTGATTAATTTACTTCGCTTATGTTGGT |
| | | TCACTGATTAACTTGTAACATTGTATTGATATATTTATATTTCAATGCC |
| | | AGTGAGGTGAATGATCGTCACTGACATTGAAGAAGAATATATATAA |
| | | ATTCAAAAACGCTAAGTAAAGAGTTCATTACTCGTAAATTGACTCC |
| | | CCTTATAGGAATGTACATCGCCATTACCTATGAGAATCTGAAAGAAA |
| | | TGACCAAACAATAAAAAAAGAAAGATTGATAAGAATATCTGAATGG |
| | | AAAGTGGCTGCGCTTGGCGGCCGTCACTTGTTTGTTTACAAGAAC |
| | | GGGCGAGGCGAATCCGGGTCGGTCCTTCCGTGCTCTTCCACGGTG |
| | | AACGAGACCAGCTCCCACACCGGTTGAGAGTTTAGCGAaacaaagcacc |
| | | agtggtctagtggtagaatagtaccctgccacggtacagacccgggttcgattcccggctggtgcatttgctgc |
| | | acacgtcaatgagttttagagctagaaatagcaagttaaaataaggctagtccgttatcaacttgaaaaagtggc |
| | | accgagtcggtgcaacaaagcaccagtggtctagtggtagaatagtaccctgccacggtacagacccgggtt |
| | | cgattcccggctggtgcatgcgagtcccaattcaaagagttttagagctagaaatagcaagttaaaataaggct |
| | | agtccgttatcaacttgaaaaagtggcaccgagtcggtgcaacaaagcaccagtggtctagtggtagaatagt |
| | | accctgccacggtacagacccgggttcgattcccggctggtgcacaatactcctcttggaaagggttttagag |
| | | ctagaaatagcaagttaaaataaggctagtccgttatcaacttgaaaaagtggcaccgagtcggtgctttttttct |
| | | agacttgtcgactttcaattgaaagggcctcgtgatacgcctatttttataggttaatgtcatgataataatggtt |
| | | tcttagacgtcaggtggcacttttcggggaaatgtgcgcggaacccctatttgtttatttttctaaatacattcaa |
| | | atatgtatccgctcatgagacaataaccctgataaatgcttcaataatattgaaaaaggaagagtatgagtattca |
| | | acatttccgtgtcgcccttattcccttttttgcggcattttgccttcctgtttttgctcacccagaaacgctggtg |
| | | aaagtaaaagatgctgaagatcagttgggtgcacgagtgggttacatcgaactggatctcaacagcggtaagatcc |
| | | ttgagagttttcgccccgaagaacgttttccaatgatgagcacttttaaagttctgctatgtggcgcggtattatc |
| | | ccgtattgacgccgggcaagagcaactcggtcgccgcatacactattctcagaatgacttggttgagtactcacca |
| | | gtcacagaaaagcatcttacggatggcatgacagtaagagaattatgcagtgctgccataaccatgagtgataaca |
| | | ctgcggccaacttacttctgacaacgatcggaggaccgaaggagctaaccgcttttttgcacaacatgggggat |
| | | catgtaactcgccttgatcgttgggaaccggagctgaatgaagccataccaaacgacgagcgtgacaccacg |
| | | atgcctgtagcaatggcaacaacgttgcgcaaactattaactggcgaactacttactctagcttcccggcaaca |
| | | attaatagactggatggaggggataaagttgcaggaccacttctgcgctcggcccttccggctggctggttta |
| | | ttgctgataaatctggagccggtgagcgtgggagtcgcggtatcattgcagcactggggccagatggtaagc |
| | | cctcccgtatcgtagttatctacacgacggggagtcaggcaactatggatgaacgaaatagacagatcgctga |
| | | gataggtgcctcactgattaagcattggtaactgtcagaccaagt |

In some embodiments, the methods, compositions, or systems according to the disclosure involve the targeting of any one of the following genes to prevent viral replication (e.g., in a DNA virus, a virus of the family Nimaviridae, or white spot syndrome virus).

TABLE B
Additional Genes That Can Be Targeted According
To The Disclosure To Disrupt Viral Replication

| Gene | Accession Number of gene/protein sequence (if relevant) |
|---|---|
| ie1 (wssv 126) | ATGGCCTTTAATTTTGAAGACTCTACAAATCTCTTTGCCAATAT |
| (KY827813.1: | GGACTTGACGGCTGGCACAACAACAGACCCTACCCGCCCCAA |
| 34508-35182) | TATCATATTCTTTGAAAGTCTACTCCCCAACTCTGGTATTGAGG |
| | TGATGAAGAGGCGTCTCGTACGGCAAGGAAAGTGTGGGAATT |
| | TTGAAGCAAGTGGAGGTGCTATGTCGTATTTCTGGCTCGAAGA |
| | TAATGCAGAAGATATGGAGAATCTCAACAGTGGTTCCCATGTC |
| | AAGACAAACTGCTTGGCATTATTCCTTCAAGAGTTTATCAGCA |
| | ACTGGATTGAAGAGACTGATCGACATGGACAGTACTGTACTTT |
| | TCCCCAATACATGGACGGTGGGGATGGTTCACGTGGGGGATAT |
| | TTTACTTCGCTAGCCATGAAATGGATGGCTAGGGATGTGACTTT |
| | CTTTGTGTTTGTTGATAGGAATAATACTGTAGAAAATGCGGCAT |
| | CCATATGGATGTACCAAAAACTACTAGCAATTGGTGCAAAGGT |
| | AGTAAAGGTGATTGTTGACAATGCATCAAACCCAATGTTTTCT |
| | GTATGTAATGCGTGTAGGTGCAAGTACCCAGGCCCAGTGTCAT |
| | ACGTTATTGAAGGCCATGGAGTGGGTCATTCTGATTTGACATGT |
| | GATGAGATTTCTGGATTCTTTGTATAA |
| ie2 (wssv 418) | ATGTTGTTCTCGTCTCCAGAACCGCCGCCAGATTTCCCTCCAG |
| (AF440570.1: | ATCCTCCTTCCTCATTATCTTCTTCCTCGTCGTCTCCATCTCCAG |
| 242850-243032) | AATCGTCCAATCCTTTACACGCCATCTTCTCCTTTAATAGGGTA |
| | GCATTTGTCTTGGTCAATAACAAGCCACCTCCTTCTGCCAGAA |
| | ACTTGTAA |
| ie3 (wssv 242) | ATGGGGGGGGATACAAAACGGTTGGGGTTTTCTCTTGTAAGTA |
| (AF440570.1: | AGTGTTTAGACGTATCTACTTGCATAATGAGTTTTTTGGTGAAT |
| c131349-131023) | TTGTCAAACACTTCCTGGGCAATCCTCTTTTCCTCCTCGGTTTT |
| | ATATTCTGACTTGATTTCTTCAGCCACTTGGTTGGCTTGCTCCA |
| | TATTCAGACCAGCAACGGTGAACAGTTTGATTGACTCCATTTC |
| | TGGGGTTGTTGGTATTATTGGAGTAATAATGGAAGTTGACGGTA |
| | GGACCGATGACGAGGGAATGATGCTGTGGTGTGAGAAGTGGC |
| | CATATTTATACTCATGCGGCTGA |
| Thymidylate kinase | ATGCAACTCATTCTTTCTCATCATCTAACCATGGCTGGTCGTGT |
| (TK-TMK); | AGAGCTCGTCACTGGACCCATGTTTGCGGGCAAGTCTACCTAC |
| nucleotide sequence | CTGAAAAACATATACCAACAAGAAAATGGAGGCAATAAACATT |
| (AF332093.3: | GCCTGTTTGTCAAACACTCCCTAGAAACTAGGTACGGTTGTGG |
| 236231-237427) | AACTGGAACAATAGTCACTCATGCCGGAGAAGTGATTGAAGG |
| | TTGTACTACAGTTTCTTCTATCAAGGAACTAATCAGTGTGTTAC |
| | CAGAAGTTGTGGATGTGATTCTCATTGACGAAGGGCAATTCTT |
| | CACGGATTTGGTGCTAGTCAATAGACTGGCTGACAAGGGGAA |
| | AAGGATTGTGATTGCAGCACTTGATGGAACTTCTGACCAGCAA |
| | ATGTTCAGTCCTATTCATAAGCTATTGCCTTATACAAATTCCATT |
| | GTTAAGCTAGCATCTAAATGTATGATTTGTAAAATTGATACCAA |
| | AGAAGCTCCTTTTACTGTAAGGTTTGGTAATGACAATGATAATA |
| | ATGTTATATGTGTAGGAGGAGCTGAAATGTACGCTGCTGCCTGC |
| | CGGGACTGTTACAAAAAAATTAACAAGAAAAAGAACAAGGG |
| | GAAACTTGTTGTACTTGAAGGAGGTGACAGGTGCGGTAAGAG |
| | TACCCAAGCCAAACTCTTGTTGACCAATAAAAACTCGCCTCTT |
| | TATGGAGGAGAATATATGTGCTTTCCCGACAGGAGCAGCCATA |
| | CGGGTAAACTCATCAATGATTATTTAACTAAGAAAATTGAACTA |
| | GATGATCATGCAGCTCACTTGTTATTTTCTGCAAATAGATGGGA |
| | AGTTTGTAGTAAAATTAAGCAGTTGTTAGACGATGGAATCCAT |
| | GTTGTGATGGATAGATATTACTACTCGGGGATTGTTTTCTCTTTA |
| | GCTAGAGGAGTGGATACCGTTGAGTGGTGCTCTGCTAGCGATG |
| | AGGGACTTCCTCAGCCCGATCTTGTATTGTTGATGCTTTTAGAT |
| | GTTGAAAAGTGTTCAAATAGGGATACTTTTGGTGTCGAAAGAT |
| | TTGAGACAAATTCCATTCAAGAACGTGCTAGAGCCCTATTCCT |
| | AGACCTCGCAAATAAGGACGAAAAGAATGTATGGATTAAGGTA |
| | GACGCTCGCGGCACCATTGAGGAGGTGCAAACTAAAATTATAA |
| | ATATTGTATATAATATTGTTGAAGAATAA |
| Thymidylate kinase | MQLILSHHLTMAGRVELVTGPMFAGKSTYLKNIYQQENGGNKH |
| (TK-TMK); amino | CLFVKHSLETRYGCGTGTIVTHAGEVIEGCTTVSSIKELISVLPEV |
| acid sequence | VDVILIDEGQFFTDLVLVNRLADKGKRIVIAALDGTSDQQMFSPI |
| (AF332093.3: | HKLLPYTNSIVKLASKCMICKIDTKEAPFTVRFGNDNDNNVICVG |
| 236231-237427) | GAEMYAAACRDCYKKINKKKNKGKLVVLEGGDRCGKSTQAKL |
| | LLTNKNSPLYGGEYMCFPDRSSHTGKLINDYLTKKIELDDHAAHL |
| | LFSANRWEVCSKIKQLLDDGIHVVMDRYYYSGIVFSLARGVDTV |
| | EWCSASDEGLPQPDLVLLMLLDVEKCSNRDTFGVERFETNSIQE |
| | RARALFLDLANKDEKNVWIKVDARGTIEEVQTKIINIVYNIVEE |
| Thymidylate kinase | ATGGCTGGTCGTGTAGAGCTCGTCACTGGACCCATGTTTGCGG |
| (TK-TMK); | GCAAGTCTACCTACCTGAAAAACATATACCAACAAGAAAATGG |
| nucleotide sequence | AGGCAATAAACATTGCCTGTTTGTCAAACACTCCCTAGAAACT |
| (AF440570.1: | AGGTACGGTTGTGGAACTGGAACAATAGTCACTCATGCCGGA |
| 266579-267745) | GAAGTGATTGAAGGTTGTACTACAGTTTCTTCTATCAAGGAAC |
| | TAATCAGTGTGTTACCAGAAGTTGTGGATGTGATTCTCATTGAC |
| | GAAGGGCAATTCTTCACGGATTTGGTGCTAGTCAATAGACTGG |
| | CTGACAAGGGGAAAAGGATTGTGATTGCAGCACTTGATGGAA |
| | CTTCTGACCAGCAAATGTTCAGTCCTATTCATAAGCTATTGCCT |
| | TATACAAATTCCATTGTTAAGCTAGCATCTAAATGTATGATTTGT |
| | AAAATTGATACCAAAGAAGCTCCTTTTACTGTAAGGTTTGGTA |
| | ATGACAATGATAATAATGTTATATGTGTAGGAGGAGCTGAAATG |
| | TACGCTGCTGCCTGCCGGGACTGTTACAAAAAAATTAACAAGA |
| | AAAAGAACAAGGGGAAACTTGTTGTACTTGAAGGAGGTGACA |
| | GGTGCGGTAAGAGTACCCAAGCCAAACTCTTGTTGACCAATAA |
| | AAACTCGCCTCTTTATGGAGGAGAATACATGTGCTTTCCCGAC |
| | AGGAGCAGCCATACGGGTAAACTCATCAATGATTATTTAACTAA |
| | GAAAATTGAACTAGATGATCATGCAGCTCACTTGTTATTTTCTG |
| | CAAATAGATGGGAAGTTTGTAGTAAAATTAAGCAGTTGTTAGA |
| | CGATGGAATCCATGTTGTGATGGATAGATATTACTACTCGGGGA |
| | TTGTTTTCTCTTTAGCTAGAGGAGTGGATACCGTTGAGTGGTG |
| | CTCTGCTAGCGATGAGGGACTTCCTCAGCCCGATCTTGTATTGT |
| | TGATGCTTTTAGATGTTGAAAAGTGTTCAAATAGGGATACTTTT |
| | GGTGTCGAAAGATTTGAGACAAATTCCATTCAAGAACGTGCTA |
| | GAGCCCTATTCCTAGACCTCGCAAATAAGGACGAAAAGAATGT |
| | ATGGATTAAGGTAGACGCTCGCGGCACCATTGAGGAGGTGCA |
| | AACTAAAATTATAAATATTGTATATAATATTGTTGAAGAATAA |
| Ribonucleotide | TATAAATATGGCCACTTCTCACACCACAGCATCATTCCCTCGTC |
| reductase subunit 2 | ATCGGTCCTACCGTCACTTCCATTATTACTCCAATAATACCAAC |
| (RR2); nucleotide | AACCCCAGAAATGGAGTCAATCAAACTGTTCACCGTTGCTGGT |
| sequence | CTGAATATGGAGCAAGCCAACCAAGTGGCTGAAGAAATCAAG |
| (AF440570.1: | TCAGAATATAAAACCGAGGAGGAAAAGAGGATTGCCCAGGAA |
| 131036-132454) | GTGTTTGACAAATTCACCAAAAAACTCATTATGCAAGTAGATA |
| | CGTCTAAACACTTACTTACAAGAGAAAACCCCAACCGTTTTGT |
| | ATCCCGCCCCATTGTCCATGAAGATCTCTGGGAAATGTACAAA |
| | AAAGAGGTTGCCTGTTTTTGGACATTGGAAGAGATTGATTTCG |
| | AAAGGGATCCTAAAGATTGGGAGAAACTCACTCAAGATGAGA |
| | AGGATTTCATTCTCCAGATTCTGGCGTTCTTTGCATCCTCTGAC |
| | GGAATTGTAATTGAAAATCTTACAACACGTCTTCGTCAAGTGG |
| | CGCAGATTCCAGAAGCGAGGAGTTTCTTTGACTTCCAAGTTGG |
| | AATGGAGAGTATTCATGGCAACGTCTACGGAGAACTGATTGAT |
| | AGACTGGTGCCCGACGAAAAAGACAAGGCTATCTTGTTTAAC |
| | GCTGCACAACACTTCCCCGCCATCAAGAAGAAGGAGCAGTGG |
| | GCTATTAATTGGATGCAAAGCAATAACGATTTGGCGGAACTAAT |
| | TGTTGCCTTTGCTGCAGTTGAAGGAATCTTCTTTAGTGGTGCAT |
| | TCGCATCCATTTTCTGGATCAAGAACAGGGGTATTTTGCCTGGT |
| | CTCACCTCCTCCAATGAGTTCATTTCTAGGGACGAAGGTCTTC |
| | ATCGCGACTTTGCATGCATGCTGTTGAAAAAGGGTTTTTTGATA |
| | CCCCATCAAGAGAAAGGATTCTTGAAATTGTCACTGAAGCCGT |
| | CCGAATTGAACAAGAATTTCTCACAGTTTCCCTGCCTGTTAAA |
| | TTAGTGGGAATGAACTGCAAGTTGATGAGCCAGTACATTGAAT |
| | TTGTGGCAGATAAACTATTGGTTGAAATGGGACTAGAAAAGCA |
| | CTATAATGTTACCAACCCCTTCCCATTCATGGACAATATTTCCCT |
| | CGAGAATAAGACCAACTTTTTTGAAAAGAGAGTCGCCGAGTAT |
| | CAACGTGCCCAGGTCATGGCTTCTATCAATAAGATCAAGAAGG |
| | ACCAACAAACCCAAGAAACTGGTTCTCCTCTCCCAATTCTGAC |
| | TGCACCTCCTCCAGTCTCTTCCTCATCATCCGAACAAGAAGAT |
| | GTTGAAGACGGCGTCGGGGACTACATCAGTTATGACGATTTTT |
| | AGTTCCACTATTGTGTCAATAGGTTGTGTATTGTATTATTATTGT |
| | TATAATATTTTTAAAAAATAAATGTTCTATAAGAC |
| Ribonucleotide | MESIKLFTVAGLNMEQANQVAEEIKSEYKTEEEKRIAQEVFDKFT |
| reductase subunit 2 | KKLIMQVDTSKHLLTRENPNRFVSRPIVHEDLWEMYKKEVACFW |
| (RR2), amino acid | TLEEIDFERDPKDWEKLTQDEKDFILQILAFFASSDGIVIENLTTRL |
| sequence | RQVAQIPEARSFFDFQVGMESIHGNVYGELIDRLVPDEKDKAILF |
| (AF440570.1: | NAAQHFPAIKKKEQWAINWMQSNNDLAELIVAFAAVEGIFFSGAF |
| 131036-132454) | ASIFWIKNRGILPGLTSSNEFISRDEGLHRDFACMLLKKGFVDTPS |
| | RERILEIVTEAVRIEQEFLTVSLPVKLVGMNCKLMSQYIEFVADKL |
| | LVEMGLEKHYNVTNPFPFMDNISLENKTNFFEKRVAEYQRAQV |
| | MASINKIKKDQQTQETGSPLPILTAPPPVSSSSSEQEDVEDGVGDY |
| | ISYDDF |
| Ribonucleotide | ATGGAGTCAATCAAACTGTTCACCGTTGCTGGTCTGAATATGG |
| reductase subunit 2 | AGCAAGCCAACCAAGTGGCTGAAGAAATCAAGTCAGAATATA |
| (RR2); nucleotide | AAACCGAGGAGGAAAAGAGGATTGCCCAGGAAGTGTTTGAC |
| sequence | AAATTCACCAAAAAACTCATTATGCAAGTAGATACGTCTAAAC |
| (AF440570.1: | ACTTACTTACAAGAGAAAACCCCAACCGTTTTGTATCCCGCCC |
| 131135-132376) | CATTGTCCATGAAGATCTCTGGGAAATGTACAAAAAAGAGGTT |
| | GCCTGTTTTTGGACATTGGAAGAGATTGATTTCGAAAGGGATC |
| | CTAAAGATTGGGAGAAACTCACTCAAGATGAGAAGGATTTCAT |
| | TCTCCAGATTCTGGCGTTCTTTGCATCCTCTGACGGAATTGTAA |
| | TTGAAAATCTTACAACACGTCTTCGTCAAGTGGCGCAGATTCC |
| | AGAAGCGAGGAGTTTCTTTGACTTCCAAGTTGGAATGGAGAG |
| | TATTCATGGCAACGTCTACGGAGAACTGATTGATAGACTGGTG |
| | CCCGACGAAAAAGACAAGGCTATCTTGTTTAACGCTGCACAA |
| | CACTTCCCCGCCATCAAGAAGAAGGAGCAGTGGGCTATTAATT |
| | GGATGCAAAGCAATAACGATTTGGCGGAACTAATTGTTGCCTT |
| | TGCTGCAGTTGAAGGAATCTTCTTTAGTGGTGCATTCGCATCC |
| | ATTTTCTGGATCAAGAACAGGGGTATTTTGCCTGGTCTCACCTC |
| | CTCCAATGAGTTCATTTCTAGGGACGAAGGTCTTCATCGCGAC |
| | TTTGCATGCATGCTGTTGAAAAAGGGTTTTGTTGATACCCCATC |
| | AAGAGAAAGGATTCTTGAAATTGTCACTGAAGCCGTCCGAATT |
| | GAACAAGAATTTCTCACAGTTTCCCTGCCTGTTAAATTAGTGG |
| | GAATGAACTGCAAGTTGATGAGCCAGTACATTGAATTTGTGGC |
| | AGATAAACTATTGGTTGAAATGGGACTAGAAAAGCACTATAAT |
| | GTTACCAACCCCTTCCCATTCATGGACAATATTTCCCTCGAGAA |
| | TAAGACCAACTTTTTTGAAAAGAGAGTCGCCGAGTATCAACGT |
| | GCCCAGGTCATGGCTTCTATCAATAAGATCAAGAAGGACCAAC |
| | AAACCCAAGAAACTGGTTCTCCTCTCCCAATTCTGACTGCACC |
| | TCCTCCAGTCTCTTCCTCATCATCCGAACAAGAAGATGTTGAA |
| | GACGGCGTCGGGGACTACATCAGTTATGACGATTTTTAG |
| VP19 (nucleotide | ATGGCCACCACGACTAACACTCTTCCTTTCGGCAGGACCGGAG |
| sequence; | CCCAGGCCGCTGGCCCTTCTTACACCATGGAAGATCTTGAAGG |
| AF332093.3: | CTCCATGTCTATGGCTCGCATGGGTCTCTTTTTGATCGTTGCTAT |
| c246265-245900) | CTCAATTGGTATCCTCGTCCTGGCCGTCATGAATGTATGGATGG |
| | GACCAAAGAAGGACAGCGATTCTGACACTGATAAGGACACCG |
| | ATGATGATGACGACACTGCCAACGATAACGATGATGAGGACAA |
| | ATATAAGAACAGGACCAGGGATATGATGCTTCTGGCTGGGTCC |
| | GCTCTTCTGTTCCTCGTTTCCGCCGCCACCGTTTTTATGTCTTA |
| | CCCCAAGAGGAGGCAGTAA |
| VP19 (amino acid | MATTTNTLPFGRTGAQAAGPSYTMEDLEGSMSMARMGLFLIVAI |
| sequence; | SIGILVLAVMNVWMGPKKDSDSDTDKDTDDDDDTANDNDDED |
| AF332093.3: | KYKNRTRDMMLLAGSALLFLVSAATVFMSYPKRRQ |
| c246265-245900) | |
| VP19 (nucleotide | ATGGCCACCACGACTAACACTCTTCCTTTCGGCAGGACCGGAG |
| sequence; Gen-Bank | CCCAGGCCGCTGGTCCTTCTTACACCATGGAAGATCTTGAAGG |
| Accession No. | CTCCATGTCTATGGCTCGCATGGGTCTCTTTTTGATCGTTGCTAT |
| AY316119.1) | CTCAATTGGTATCCTCGTCCTGGCCGTCATGAATGTATGGATGG |
| | GACCAAAGAAGGACAGCGATTCTGACACTGATAAGGACACCG |
| | ATGATGATGACGACACTGCCAACGATAACGATGATGAGGACAA |
| | ATATAAGAACAGGACCAGGGATATGATGCTTCTGGCTGGGTCC |
| | GCTCTTCTGTTCCTCGTTTCCGCCGCCACCGTTTTTATGTCTTA |
| | CCCCAAGAGGAGGCAGTAA |
| VP24 (nucleotide | ATGCACATGTGGGGGGTTTACGCCGCTATACTGGCGGGTTTGA |
| sequence; | CATTGATACTCGTGGTTATATCTATAGTTGTAACCAACATAGAAC |
| AF332093.3: | TTAACAAGAAATTGGACAAGAAGGATAAAGACGCCTACCCTG |
| c5725-5099) | TTGAATCTGAAATAATAAACTTGACCATTAACGGTGTTGCTAGA |
| | GGAAACCACTTTAACTTTGTAAACGGCACATTACAAACCAGGA |
| | ACTATGGAAAGGTATATGTAGCTGGCCAAGGAACGTCCGATTC |
| | TGAACTGGTAAAAAAGAAAGGAGACATAATCCTCACATCTTTA |
| | CTTGGAGACGGAGACCACACACTAAATGTAAACAAAGCCGAA |
| | TCTAAAGAATTAGAATTGTATGCAAGAGTATACAATAATACAAA |
| | GAGGGATATAACAGTGGACTCTGTTTCACTGTCTCCAGGTCTA |
| | AATGCTACAGGAAGGGAATTTTCAGCTAACAAATTTGTATTATA |
| | TTTCAAACCAACAGTTTTGAAGAAAAATAGGATCAACACACTT |
| | GTGTTTGGAGCAACGTTTGACGAAGACATCGATGATACAAATA |
| | GGCATTATCTGTTAAGTATGCGATTTTCTCCTGGCAATGATCTGT |
| | TTAAGGTTGGGGAAAAATAA |
| VP24 (amino acid | MHMWGVYAAILAGLTLILVVISIVVTNIELNKKLDKKDKDAYPV |
| sequence; | ESEIINLTINGVARGNHFNFVNGTLQTRNYGKVYVAGQGTSDSEL |
| AF332093.3: | VKKKGDIILTSLLGDGDHTLNVNKAESKELELYARVYNNTKRDIT |
| c5725-5099) | VDSVSLSPGLNATGREFSANKFVLYFKPTVLKKNRINTLVFGATF |
| | DEDIDDTNRHYLLSMRFSPGNDLFKVGEK |
| VP35 (nucleotide | ATGGTCTCTTCTAGAACATCAACAACATCTTCATCTGCAGTAGC |
| sequence; | AGCCACCTCTACTCTTCTCCCCACCAAGAGGAAGAGGGAGCC |
| AY325896.1) | AGAAGAAGTAAAGGTGAAAGTGGAAGTAAAAATGGAACAAG |
| | AAGAACTGGTAGAGGACTCGTCAAGTAACAAGCGCCCCAGAA |
| | TTAAGGAAGAGAAGGAGGAAGAACACAAAGAAACACATCAC |
| | CTCTCCCTCCCATGTAAAGAAGAAGAAGACGATGGTGAAGAA |
| | GAGGAATATGAGGAAGAGGAGGATGAGGAAGAATATGAAGAC |
| | AGAGTGGACGACGACACTGCAGAGAAAATGGAAAATCTTTTG |
| | GTGCAACTGGACAATACTACCAAAAACATCAAACTGAAAAAC |
| | CCCCTAAGGGAACATGACATGGCAGTTTCACACTATGAGCATG |
| | AATTTGAGGTACAAAATACTGTCAATTTTAGTTTTGGAGTACTA |
| | TCTGATATTGGGTTCCTGATCAACCGTGAAGCCGTTTCTAGGTG |
| | GGGTAATACACCCCCACCAAAAGAGTTTGGCGACATGGAGATT |
| | GGATCTCTTACAGTTAACCAGTTGCTCCACAAGTGTGATAATTT |
| | TGTACAGGCTGTAGTACAGAAAGTGAAGGAAGATATAACCCCT |
| | TCTATTGAAGTTACAATAGATAGTTTGATTGATGATCCTTGTTGG |
| | TAA |
| VP35 (amino acid | MVSSRTSTTSSSAVAATSTLLPTKRKREPEEVKVKVEVKMEQEEL |
| sequence; | VEDSSSNKRPRIKEEKEEEHKETHHLSLPCKEEEDDGEEEEYEEE |
| AY325896.1) | EDEEEYEDRVDDDTAEKMENLLVQLDNTTKNIKLKNPLREHDM |
| | AVSHYEHEFEVQNTVNFSFGVLSDIGFLINREAVSRWGNTPPPKE |
| | FGDMEIGSLTVNQLLHKCDNFVQAVVQKVKEDITPSIEVTIDSLID |
| | DPCW |
| VP35 (nucleotide | ATGGTCTCTTCTAGAACATCAACAACATCTTCATCTGCAGTAGC |
| sequence; | AGCCACCTCTACTCTTCTCCCCACCAGAGGAAGAGGGAGCCA |
| AF440570.1: | GAAGAAGTAAAGGTGAAAGTGGAAGTAAAAATGGAACAAGA |
| c10682-9996) | AGAACTGGTAGAGGACTCGTCAAGTAACAAGCGCCCCAGAAT |
| | TAAGGAAGAGAAGGAGGAAGAACACAAAGAAACACATCACC |
| | TCTCCCTCCCATGTAAAGAAGAAGAAGACGATGGTGAAGAAG |
| | AGGAATATGAGGAAGAGGAGGATGAGGAAGAATATGAAGACA |
| | GAGTGGACGACGACACTGCAGAGAAAATGGAAAATCTTTTGG |
| | TGCAACTGGACAATACTACCAAAAACATCAAACTGAAAAACC |
| | CCCTAAGGGAACATGACATGGCAGTTTCACACTATGAGCATGA |
| | ATTTGAGGTACAAAATACTGTCAATTTTAGTTTTGGAGTACTAT |
| | CTGATATTGGGTTCCTGATCAACCGTGAAGCCGTTTCTAGGTG |
| | GGGTAATACACCCCCACCAAAAGAGTTTGGCGACATGGAGATT |
| | GGATCTCTTACAGTTAACCAGTTGCTCCACAAGTGTGATAATTT |
| | TGTACAGGCTGTAGTACAGAAAGTGAAGGAAGATATAACCCCT |
| | TCTATTGAAGTTACAATAGATAGTTTGATTGATGATCCTTGTTGG |
| | TAA |
| VP39; (nucleotide | ATGTCGTCTAACGGAGATGAGCCTGCTGTGACTGAAGCTGAA |
| sequence; | ATCGCTTCAGTGGAGGCTCAATTGGGAGCTGCTCACCATGACA |
| AF332093.3: | ATTCTTGGATCACAAGAAAGAGTGACCAATTAAAGTATCGCTT |
| c200131-199280) | AGGTGCAATTGCCTATTCGGTAGCAAAAAATGCCTCTATAAAAT |
| | ATATAGAGGATCAAGTAAGGCAAGAAATCAATAGCCATTTAAC |
| | TAATGTAATGACTTTTGAACATCTTTACGAAGACGCTTTCAATC |
| | CTGTTATCTGTGAAGCAATTTTTGAGAAAGGAATACCAGTGGT |
| | TATGGAAAAAGTATACGATGTGAATAGACGGATCATGGAACCC |
| | AGGGAAGATTTCATAACTGAAATTTTAAAAGAGGAGCGGTGG |
| | AGAAGATATATACCTGGTTTTTATCATACATCATTTTCTTTCAAG |
| | TACAATACTATTGCCTTTACCGACTCTTCAACTTCATTTAGTGTA |
| | CCAATAAACGATAAACACATGTTATCAATCACTCCCCCTGGAG |
| | CTGCTCAAGGGGATTTAATTGATTTAAGTTTATCGTTCAAAATA |
| | GATTCTTCAGCCAAAACTCTCACGTTAGAATTTAACCGCAAAT |
| | CCACGTTCGCTGGTATTGTAAACAGACCAAAAAGTGTAGTGAT |
| | ATTATCAAATCTAAGAAATAGTGATTCTTCTGATAACATAGGTG |
| | ATTATCTAAAGAGAAATGATCCTATATATATTAGTCATGATACAA |
| | ATGGCATAATCAACCCATCCGAGGATTCGGCCTCTCTCATTACA |
| | ATTCACATGCCTGAAATCGAAAACGCGAGTGATGATTTATACAT |
| | AGATTTCAATCTGTTTGTTTTTTAG |
| VP39; (amino acid | MSSNGDEPAVTEAEIASVEAQLGAAHHDNSWITRKSDQLKYRLG |
| sequence; | AIAYSVAKNASIKYIEDQVRQEINSHLTNVMTFEHLYEDAFNPVIC |
| AF332093.3: | EAIFEKGIPVVMEKVYDVNRRIMEPREDFITEILKEERWRRYIPGF |
| c200131-199280) | YHTSFSFKYNTIAFTDSSTSFSVPINDKHMLSITPPGAAQGDLIDLS |
| | LSFKIDSSAKTLTLEFNRKSTFAGIVNRPKSVVILSNLRNSDSSDNI |
| | GDYLKRNDPIYISHDTNGIINPSEDSASLITIHMPEIENASDDLYIDF |
| | NLFVF |
| VP39; (nucleotide | ATGTCGTCTAACGGAGATGAGCCTGCTGTGACTGAAGCTGAA |
| sequence; | ATCGCTTCAGTGGAGGCTCAATTGGGAGCTGCTCACCATGACA |
| AY884234.1:99-950) | ATTCTTGGATCACAAGAAAGAGTGACCAATTAAAGTATCGCTT |
| | AGGTGCAATTGCCTATTCGGTAGCAAAAAATGCCTCTATAAAAT |
| | ATATAGAGGATCAAGTAAGGCAAGAAATCAATAGCCATTTAAC |
| | TAATGTAATGACTTTTGAACATCTTTACGAAGACGCTTTCAATC |
| | CTGTTATCTGTGAAGCAATTTTTGAGAAAGGAATACCAGTGGT |
| | TATGGAAAAAGTATACGATGTGAATAGACGGATCATGGAACCC |
| | AGGGAAGATTTCATAACTGAAATTTTAAAAGAGGAGCGGTGG |
| | AGAAGATATATACCTGGTTTTTATCATACATCATTTTCTTTCAAG |
| | TACAATACTATTGCCTTTACCGACTCTTCAACTTCATTTAGTGTA |
| | CCAATAAACGATAAACACATGTTATCAATCACTCCCCCTGGAG |
| | CTGCTCAAGGGGATTTAATTGATTTAAGTTTATCGTTCAAAATA |
| | GATTCTTCAGCCAAAACTCTCACGTTAGAATTTAACCGCAAAT |
| | CCACGTTCGCTGGTATTGTAAACAGACCAAAAAGTGTAGTGAT |
| | ATTATCAAATCTAAGAAATAGTGATTCTTCTGATAACATAGGTG |
| | ATTATCTAAAGAGAAATGATCCTATATATATTAGTCATGATACAA |
| | ATGGCATAATCAACCCATCCGAGGATTCGGCCTCTCTCATTACA |
| | ATTCACATGCCTGAAATCGAAAACGCGAGTGATGATTTATACAT |
| | AGATTTCAATCTGTTTGTTTTTTAG |
| Wsv477 (amino acid | MYIFVEGSPLTGKSSWMSKLIDTGSCGMSFLNFLRMNTSDYYN |
| sequence; | WPAEIGTEHLQLGFRETRVVDGMFEPVLKTFVDSWKKEQGKESL |
| AF332093.3: | KEYLDYNGQVMEIYIAEWLRQRPLAFHVFTYTDEAVKSGFLNEE |
| 279149-279775) | DLDMDTATKWMAEIIREKRGNIQEIKVTPRVVFNGNVCSACFSN |
| | TKRNLYNFGTNYNNVVHCDLLCPFARHRIVHFL |
| Wsv477 (nucleotide | ATGTATATCTTCGTCGAAGGTTCCCCCCTCACAGGGAAGAGTT |
| sequence; | CATGGATGTCCAAGTTGATAGATACAGGATCATGTGGAATGTCT |
| AF332093.3: | TTCCTCAATTTTCTTCGTATGAACACTTCTGACTACTACAACTG |
| 279149-279775) | GCCTGCCGAAATCGGGACAGAACATCTCCAGTTAGGTTTCAGA |
| | GAAACCAGAGTGGTGGATGGAATGTTTGAACCTGTCCTAAAG |
| | ACCTTTGTCGACTCGTGGAAGAAAGAGCAAGGAAAAGAGAG |
| | TTTGAAGGAATATCTGGACTACAACGGCCAAGTCATGGAGATC |
| | TACATCGCAGAATGGTTGAGACAAAGGCCACTAGCCTTCCACG |
| | TGTTTACCTATACAGATGAAGCTGTCAAGAGTGGATTCTTGAA |
| | CGAGGAGGATCTAGATATGGATACTGCAACCAAGTGGATGGCT |
| | GAAATTATTAGAGAGAAGAGGGGCAATATTCAAGAAATAAAAG |
| | TGACCCCTAGAGTAGTCTTCAATGGCAATGTTTGTAGTGCATGT |
| | TTCTCTAACACTAAGAGAAACTTGTATAACTTTGGAACAAACT |
| | ATAACAATGTTGTACATTGTGATTTGTTGTGCCCTTTTGCAAGG |
| | CATAGGATTGTACATTTCTTATAA |
| Orf89 (nucleotide | CTGGTTGAATATATGCAGATGATGCATCAAAATCTACTTCTTCG |
| sequence; | CCGCTCATGCCTGTTTCTAGTCTTGAAAGGAGTGTGTCCAAAT |
| MF768985.1: | CTTCCTCTTCTTCGTCTTCTTCCATTTCTTCTCCACTATCTAAAA |
| 54197-54436) | ATTCGTCTATATCATTATCATCAAAGTCTATGTCTATATCATCATC |
| | ATCATCTTCCCCAAGAGATGAAGAAGGAGATGGGATTCGTGTC |
| | GGGGGAGGTGGAGGAGTAG |
| Orf89 (amino acid | MVEYMQMMHQNLLLRRSCLFLVLKGVCPNLPLLRLLPFLLHYL |
| sequence; | KIRLYHYHQSLCLYHHHHLPQEMKKEMGFVSGEVEE |
| MF768985.1: | |
| 54197-54436) | |
| Protein kinase 1 (PK1) | ATGGGGGGACCCACTGTAATTACTACTACCATCAATACTGGTGG |
| (nucleotide sequence; | AGACCACCACCACCAGCAGTATGTTTACCATCAGGGGAATAAA |
| AF332093.3: | AAACGGCCTGTGGAAGAATATAACAACAACAACTACGCGTCT |
| c45326-43581) | GGTTCAACCTCCGAAGCCACAACTGTTCCCGCTTACAACAAC |
| | AACAACAACAACATCACTATCAAGACTTGGGATGACGTCATCA |
| | ACCTTAGCATCACGCCCCCTCCCCCTAAACGTTTCAAGAAGTC |
| | TGAAGTTGCTCCCTCTCCTCCCACTACTCGCACCTTTTCAAAC |
| | GTGTGTGCGTCCAAGGTGATTAGGCAGTGTAAGAGGCAGTATA |
| | ATGAGTGGATTGAACGTGATTCCCCTTACTACTTTAAAGGCATT |
| | GAGAAGAGTTGTAGTCTTGAGGACAATTATGATACCTGTCAAC |
| | AGTTGAGAATTGGCCATAGGTCAATTGTTAAGTCTAGCAAGTAT |
| | GTCCATGATACCTGTTTCTATGGAAAGGACCCTAAAGTTGGCTT |
| | CTATTGGCCCACCTCTTCTTGCGATGAAGAGATGAGATTTTTTG |
| | ACACTAGACACATTCTTAAGGAGTTGTCTAGTCGTAATATTCCG |
| | TCCTCCCAGATTATGGACATAATGTATATGGCTGTAGAGGTGTT |
| | CCAATTGCCTTCAAGTGCCTGTGAGCGAATTAGACAAAAGACT |
| | AGCACGCTAATTAAGGAAGTTTCTGACCAGTGTGAGAACTGG |
| | GAAAACTTCCGTAAGACTGCTCGTGGTTGTTTGTCTGATTTGG |
| | TCGAAGTGCCTGAAGATGTGAAGGACTTTAACACTTTCATCTG |
| | TCCCTGGGAGACCTTTTTTGAGATTAAATATGGGGTCTATTACA |
| | TTGTGAATAGGGGGACTGTTGTCAAGTTTATGAAGGATATGAA |
| | CTATGAAGAGTTTGTTTTTGAGTGTGTTAATGGCCTTTCTGTAT |
| | ACAGAAAGAATATTAAGGGGGTAGTTGGGGTGACTGGTGTGT |
| | GTCCTCAGGGGTTATGTTTAGAGATGCCATTTGCAGGTATCAGT |
| | ATTGATGATGTCATTAGGTGTGTCAAGGATAGTTTAGATGGTGG |
| | GGAGTATTATGAGTCAAGGGACGCACGCTTGTTGTATGGGGTT |
| | GTCATGCTTCAAAGGATGGGACGTTTACCAGAGGTAAAGGGG |
| | GTTGATACAGTCGCACCAATAACAGACTCTTTCATTGCCCGAA |
| | AGGTTGTAAGAAGTATGTTTGAAAAACTAAAGGTGAACATGCC |
| | TTTTGTTTTGGCTGAGACTTGTAATGTAATTACAAGAGTTGCAA |
| | ATGAGGGAATTATTAATGTCGATATAAAGGCTGATAACTTTGTT |
| | ATAGATAGCATATCTGGCCAACCTAAAATGATTGACTTGGGACT |
| | CTCATACCCTCTAGGTTATTGTTACAACGATGAATATTTTAGGA |
| | ACACGGAAGAACTAATCAGGCAGTACATTCACACACCTCCCG |
| | AGTTCTTTAGGGGACACTGTCTAGGTGCCTATTCAATGACGTAC |
| | AGTTTCAGTGTAATGGCTTCCAGTATACTGGAAGATGTTGTTGC |
| | TTGTTCTAACATGGAAGGCCCTGCCTTTAATTTGATGTCAAACA |
| | TGCACTTTTTGATGTTGTTGCAAAGCGGAACAGACACTGATTT |
| | CTATCAAAATCGCCCTTCAATCACAGAATATGCCCTTGCCATGA |
| | AGCACATATTCCCTTTTAAGGGGACTGTAATGAACCTGTTTAAA |
| | GTAAAGAAATGA |
| Protein kinase 1 (PK1) | MGGPTVITTTINTGGDHHHQQYVYHQGNKKRPVEEYNNNNYAS |
| (amino acid sequence; | GSTSEATTVPAYNNNNNNITIKTWDDVINLSITPPPPKRFKKSEVA |
| AF332093.3: | PSPPTTRTFSNVCASKVIRQCKRQYNEWIERDSPYYFKGIEKSCSL |
| c45326-43581) | EDNYDTCQQLRIGHRSIVKSSKYVHDTCFYGKDPKVGFYWPTSS |
| | CDEEMRFFDTRHILKELSSRNIPSSQIMDIMYMAVEVFQLPSSACE |
| | RIRQKTSTLIKEVSDQCENWENFRKTARGCLSDLVEVPEDVKDF |
| | NTFICPWETFFEIKYGVYYIVNRGTVVKFMKDMNYEEFVFECVN |
| | GLSVYRKNIKGVVGVTGVCPQGLCLEMPFAGISIDDVIRCVKDSL |
| | DGGEYYESRDARLLYGVVMLQRMGRLPEVKGVDTVAPITDSFIA |
| | RKVVRSMFEKLKVNMPFVLAETCNVITRVANEGIINVDIKADNF |
| | VIDSISGQPKMIDLGLSYPLGYCYNDEYFRNTEELIRQYIHTPPEF |
| | FRGHCLGAYSMTYSFSVMASSILEDVVACSNMEGPAFNLMSNM |
| | HFLMLLQSGTDTDFYQNRPSITEYALAMKHIFPFKGTVMNLFKV |
| | KK |
| Protein kinase 1 (PK1) | TATAAAAAACGTCGTTAAAATGGAGGGTGGGGACCAACGGAC |
| (nucleotide sequence, | AAAACTTACGCCAGCAACCGTGATGGGACTTTACCAATCGAAA |
| AF440570.1: | ACGCCAGGAGAAGGAGAAGGAGGAGAAGGAGGAGGGCAATT |
| c281785-279549) | CAAGATACCTTCAGCCATAGCTGTGAAATCTTGTTGCTCTAAA |
| | AACGCTACTCGCCGATCCCCTCCCTCAGATTCTCCTTATTCTCT |
| | TAGGCCCATGAAGAGACTAAAGAAGAATAATGGAGAGGTGGG |
| | AGGAAAAGCACCGCCTCCTGTGACTTTGAGGCTCCGCGAGGA |
| | CTACGAGAGCACACCTTACAACTTTAATAGAAATAAGAAGAAG |
| | AGGCCTATTACTATTGATGAAAATCAATTTGCAACATTAAATCC |
| | AACGTATGCGACAGACATTATCAAGAAGCAGCAATTGCCTTCT |
| | GTTAGTGCCGCGTCTGTGTTGAGGAAGCACCGCGCCAATGCC |
| | GACACCCAGTACAGAAAAAGATTCTCTCATCCAAATTGTGCAA |
| | AATTCTCTACTGTCAATTTGAAGGCTAGAGACTATACTCCACTG |
| | TCTGTCCTCCGTTCCCATGTCAAGGGGCCAAAACACTTGAAAT |
| | CTTCTTGTGATACCGTGACTGAAACAAATGTAGTAAAGAGGAA |
| | CTTTTCTTCCATTGACAAGTGGGTCAAGCTAGAAAAACCCCCG |
| | TGTTACTTTGCAGTGGCAGAGGCTGATACCAATATTGCAGCCG |
| | GTCTAGAATCTCCGTTCCATTTGATTAGACAGGCCGCAAAATTA |
| | GGCCTCATTTCTGACGTGCAAGATGTGTCGTCCAACTACGAGA |
| | CCATAAAACAGAGCTGTATTGACGCAAAGGAAAAAGCGTCCA |
| | AGTTTTTGTGGTCTAACAACCGTACTAAACAACCCCCTTCATCT |
| | TGGTGGCCTGTTGGGTTTGGTAGTAAAAACCTATCCGTTTTAG |
| | ACACTAGCCCTCTCTTGAACTGGAACAGGTTATGCAAGAATAA |
| | TGGTAAAGGGTGGATAAAAACCATGAGCATCGATCACATGGCA |
| | AAGAATGTTTTTAAGCTTTCCCCTGGAGCATGTGAATCTATATT |
| | GGAGAAGAAAACTACACTCTTGGGGGAGGTCACTGCCCAATG |
| | TAAGAAATGGGAAAGTTACCGCAGAAATATTCCTGTACCAGCA |
| | CACGTCCAACCAGAATATGCTTCTCAAGTCGTAATGATTGGAC |
| | CATCTGAATTATATCTCGAAGTTAAAGTCGGGGTATATTACATGC |
| | TTGAAACTGGAAAAGTTATCAAGTTTATGACGGACAAGGAAAT |
| | GTACTGTGAATTTGTATTTGAAACTGTTTTTAGTCACGCTCTTG |
| | AGGGAAGAATGAAAGGCGCAGTAGGTGTGAGAAAGATGTGTG |
| | TTGAAGGTTTTTGTGTCGAGATGGATTTTGCAGGCATTTCTGTG |
| | ATTGATGTATTAAATGGAGACCTGAAATGTAAAATGGACGAGA |
| | ATGTTGTACAGCAACCTAACCCCTCGACTACTTCCTCCAAGCC |
| | AGCCGCTGAGCTCATGCAAGATCATGGCAGCTTGTGTAGGATG |
| | AGGGATACTCTGTACGGTGTTAGGATGCTTCAAGCTACTGGCC |
| | GCCTGCCTGAAGGTCTACAATCTAAATGCAAGAAACCCATTAC |
| | GGATTCAATTTCAGCCATAGCTATCGTTGGAAAAATGAGGGAG |
| | AGAATGTTAAACCAATTGCCCTTTGTTTTGGTAGAAATTGTAAA |
| | TATTGTCACTCGGTTGTCTCAACAAGGATTAGTGAATCCGGAC |
| | ATAAAAAGTGACAATATAGTAATTGATGGAATAACTGGTCAAC |
| | CTAAGATGATTGATTTTGGTTTAATTGTACCATGTAAAAAGTAC |
| | TACAATTTTAAATGTTGGGGAACTGATGAGAGGTTCTTTAGTAA |
| | CCATCCTCATACAGCTCCTGAATTTATTAACAGTGAGTTGTGTT |
| | CAGAAACTGCCATGACTTTTGGGTTGGCTTATTTGTTAATAGAC |
| | ATGTTGTCCATTTTGATTAAGAGAACTGCAGATTTGTCTGCCAA |
| | TTCTATCTATACAAACATTCCATTTTTGTCTATTGTATCTAAAATG |
| | TATGACCAGGAAAAGACCAATAGGCCGAGAGCGTATGAAATT |
| | GCGCCTGTAATTGGTGCATGTTTCCCGTTCAAGGATAATATTGC |
| | TAAACTTTTCCAGTCACCTAAACATTCATTGTATAGCAAGAAGG |
| | TTAAGTAGGTCAATAAAATATGGTATAAATTTC |
| VP12 (nucleotide | ATGGACCAGCTTACAGAAAATCCTTCTCTTTTAGCAGAGAGGC |
| sequence; | CTGTCTTTAGGCAGAAGGTTATAGATTTGTATAGGGAGGAAATA |
| AF332093.3: | CTACGTGATCTTATTAAAAAAATGACAGCTGACATGACTTCTGC |
| c7279-6992) | CATTGAAGAAGAATGCACGGCCGCTGTTGCGGATGGATCTTTT |
| | TGGCGAGATATGCGATATGAAATGGTAGCTCATCAACATGACGT |
| | GACCTTTGCAGCAAAGAATATGGAAGGGTATGAAGAATTCGCG |
| | GCAGAGGTCAGGCGTGTGGAGGGGTAA |
| VP12 (amino acid | MDQLTENPSLLAERPVFRQKVIDLYREEILRDLIKKMTADMTSAI |
| sequence; | EEECTAAVADGSFWRDMRYEMVAHQHDVTFAAKNMEGYEEFA |
| AF332093.3: | AEVRRVEG |
| c7279-6992) | |
| VP190 (nucleotide | ATGTCAACAACGCAAACGCAAACAATAGAACGGCCCCTGCCT |
| sequence; | GGTAAGAACAACGAAGACAATAGCCGTTTAGCTTGCCTATTGG |
| AF332093.3: | CTGAAGGGCTACAGCAACAACAACAACAACAAGATGGCGATA |
| c174441-169744) | GCGAAATTTCTCTACCGCTTGTTAATGCGGGAACTTTTGCGTGT |
| | TATGATTCTACCCTTGCAAACCTCACCGAGGGGCGTTTAGGAA |
| | GTGAAACAGAAAATGCAAAAATAAGGGTAAAAATACACCCGT |
| | AATATCTACAAAATCTCTAAATGCATTAGTGGAAAAAAGAGCA |
| | CTGTGTTTATTATAGAGACGAATAAAGAGATGACTATTGAAGA |
| | CGAGAAGCACGACGATTTTCATCTCTGACAGAACAAAAATTTC |
| | CTCGAGGAGGAGGAGGATGTTATTCTCGTAAAAATGAGAGGTT |
| | TATCGAAGGCGAAATTAACAACATCAAGCTGAACATGGAAGA |
| | AACTGCTTCGTCTTTAGAGAGATTGGCAGGGCTTTTGCCTGTT |
| | GTCATTAATATTAAGGACTGGACAATGCATGATGAGAAAGAAA |
| | TACGGCTAGATCTTAAAGGAAACGACGGCATGGAAGAACTTG |
| | TCAATATATCCCATCTGAATCAAGAAGAATGGGAAATGGAAAG |
| | ACTTTCTTCCTCAATAGTATTGAAAGACGCCTATGGTGTGTTTT |
| | ATGCCCATCACGGCATACTTGATATTGTTCTTACGACCTCTAGAT |
| | TCACAGGGAAATTATTACAACACCCTGTGATATTTCGTCTAATG |
| | GATGTGAAGGTGTGGATAAACACCCCCCTCCAGATAGCATTTC |
| | CTGACACCAGTAAGAACCCAAATGCAAAGAAAATTCTCTATCA |
| | ACATCCCTCTCTTACAAGGCTACGTGATTTAAATGACATGGCTT |
| | CAAACTCCAAGTCAGTTTCTTCCATTATCATACCAGAGTTGTCA |
| | AAATTTAACTCGACTGAATTTGGTATGCACTACTTTACGGCCCA |
| | GTGCTTTTTTGGAAAAAATACCAATTCTTTAAAGGATTTGGTTA |
| | CACGGTATTATCAACTATCATTTAAAAATAAACCTCAACCCAAA |
| | CTTTATGAACCAACAGCAACAGCAACAGCAGCCTCCTCCTCCT |
| | CCTCAACCGCCTCATTGACGACCGAACAAAAGGAAAAAATCG |
| | CACAATCGATTTTATCGTCTAAAGGAAAATCTTTAGGAGATGTA |
| | TCTTCCACCCTTTCTAAGGAATACGATGAAAATAGAAAACGTA |
| | CAAAGAGGCAAAAAACTTCTACCGATACCAACATTGTCCCTTC |
| | AGGGGCACCTACTTCCATTTCCATGAAAAACCCAGTTACTTGC |
| | TTCTTTGGGCCTCAATACACTTCAATCATGGACTGTATATCGGA |
| | GAAAACAGACTGGATAGAAATGCACCTTTTTTTAACCTCTCTT |
| | AACGACGCTGAACATAATAAAACACTCGTTGTCGACAGAAAA |
| | AGTAACGTGTCGGAAATACACGATTCTGGAAGGTTTCTTACAT |
| | TCGGTCAAAATAATACTACAGCCTTTATACCAGATGTTGATATTC |
| | CTACGTTAAAATTAATTCTGAGAGACGATTCTGGGGAATCTTCT |
| | GCTGCTATAATAGCATCTCTGATATATTATAATAATGTAAATCTAG |
| | AAGGGAGGGAATTCTCAAATGTCTCTGATGCTGTTGTGGGACT |
| | TTTCTCGGGCGGATCTGCTATTACGGTAGGAGATATTGCTCGAG |
| | AGATTGCATCCATATACAATATTGGAAGAGAATCTAACTGCGAT |
| | TCCATACTATTCCCAGGGGAGCCTATACTGGCCGGAAGAAGAA |
| | GTTATGGACGGCAGTACAGATGGTACGATCCCATTAACTGCGT |
| | GGTGGGTCTTTATCGGTCGTGTTTAGAAACAATGACAAGAAAT |
| | ATTATGAGAGGACAGCCAGTGAAAGTGGATGAGACCGCTTGG |
| | ATGTACATGCACCAGCAGGTACTTCAAGTTGTTCTTCTTCCATT |
| | TTTTGATTGCGTTTTGAAATCGGGCGTATGGGCTGTAAAGGAG |
| | GCTAGACAACTAACAGATTATATAGTACGTGAAGTGCTACTAA |
| | AATATACAGCCGATCCTGATCAACACAAATTTTTATTATTCAAG |
| | AAGCCCGTGATGGATCTAATTGCAAAGATTGTTACACATTATGC |
| | AGTTATTCATTCGGCAGCCGATAATGGAGGCGTGTGCCTTGCTT |
| | TCCCTAGAGATCCTCCCTTTATTGTAGAAAATGATACGTCTCTC |
| | AGATACTATACACTGACCGATACACCTCAGAGTATTTTGAATGG |
| | AGATAATGTAGCCGAAAACTTAAAGTCTGCCACTTCAGTCGCT |
| | TCTTCTCCATCATCCTCCTCCAGATATTCATCAGAAACCCCTATT |
| | AGGGTAGTTAATCTTCCTGTACCAACAGGACGGTTTTTAAAGA |
| | TGAATAAGGATCTGGAACTTTTCATTAATGTACCTCTAATATCCT |
| | CTAAGGAACAAAAACAACAACAGCAGCAAACAACTGCCACT |
| | GCTCCATTTTCTTCTGAAACCATTTCAAAATCGTTCTTAAATTAT |
| | GTACCACCTAAATCTCTAACAAGAAATGTGACATATGGTCAGA |
| | ATATCGCAGAAGATGGATTCCTTGGATTGAAAAATAAAGGTGA |
| | ATTGGTGTCGTACTTTAAAGTTGTAAAGAATACTGAACGCGAT |
| | GATGGTATAAAGGATATGGAAATTGGTGATATTAATAACCATCA |
| | AGACGACACTGGATCCCTTTCTTCTTCTTCTTCTTCATTCGTTG |
| | ATGGTGTACGGACAAGTTTTTCGGTTGATGGGAAAATTGAACA |
| | TGTTAGCGCATTCCTCCCTGGTACGACTTCTCAGCCCACAAATC |
| | TTCCTGTTCATGCATCTAAACAAGTCAAATATTCGGTAAAGGA |
| | ATTGGGCATGGAAACTGTATTCTTTGAACCCCTTCTCTCGTCTG |
| | CTGTGCTTTATGAAGCCTCAAAAACTAAATCGACACAACATTT |
| | AAGCCCAATGAGGATCTATAAGGAATGTGTTAGTCCTCTATCTA |
| | CAGGGAGGATCGATATTTTCCCCAGTAAAGTGGGCACAGTGGC |
| | AGGAACTGGATTCGAATTTATTTGGAAAGTTCTTCAGTATGATA |
| | CTGGATTACCCACAACTCTAGAAAGGTTGTCACCCAAGATTCC |
| | TTCTGTACCAATTTCAGGCGAAGACTCAAAAATGGAAGTAATT |
| | GCAGAATCTGGTAAAGGTGTACAAAACATCATTGCTATTGCAG |
| | CCGACCAACTCAGAGGCAGTAATAATATAGTAGGTGGTGGAAC |
| | AAGAAGAGCGATCCAACAACAACAACAGCAGCAACAACAAG |
| | AAACACAAGCGGTTGTTCCAGTTAATGTGCCTGCCCGTTTCGA |
| | GCCTACGTTCACAGAGATCGAACTATTTCTTCAGAACAAATTT |
| | AGGAATGTTATTGCTACTATTATATCTAGAATGATGATGCTTGTC |
| | AGCAACGAAGAGATGAAAATTATTAAAGAAGTGTGCGAGCAC |
| | GTGTCTCATATTATGGTCGATGGATTATATGTTGCACTAGATCCT |
| | AGAAAGGCCATTGAAGAAATACTGGAAAGGATAACAGCTGAA |
| | CAAAATGGGATTACTATTGATACTGGAAATGAAGGGTATGGATC |
| | TTTACGCTATGCTTCTTCGGGGAGACTTTTTATAAATGATGAAG |
| | CTAGTGAAGAAGCTGCAGCGGCCATCGGCGGTGGAGGTGCTC |
| | TTGGAACTGGTAGAAGGGTGCCTGTAGAATTACGTTCAATATT |
| | GGACAAATTAAATACCATTGGAAGTACTACTCAACAACAACAA |
| | CAGCAGCAGCGTCAACAGAGGCAGGCTAATAATAACAATACTG |
| | TTCCCGAGGATATAAAGGTTCACAATGAACAAATGCAAAAAAT |
| | TAGAGACTCTTCTCTATTCACATCTAAACTTTTGAATTACATTAG |
| | GGATGATGGAAGAAAGGACCGTATAAAGACAAACATTTCAGA |
| | AACACTAAAGAAATACAGTAGAATCCCATCATACTTTATTGCTT |
| | CAAAGGCACAGAAACCTATCCCATGGAAACACACCAAGGACA |
| | ATATCAACCTCAACAAGATTCCAGAAGACTTGAACTTTTCTCC |
| | TGCACAGAATCTGTTTGTTCCAGTTAATCCGCGCCATATCCTTA |
| | CTGACATGCAATGGCTCAACTGTATTTCCATCATCGAAACTGCA |
| | ACAAGAGATTCGGCTATCGTCATGCAAAGTTTCCAAGAGCAAG |
| | CGGACAAGACTACAACTCAATTGGAGGAACTTTTATCTCAATG |
| | GAATAATATTGTATCTCAAGTAACTGATGAAAAATCCCCAGCTT |
| | ATGTTTCAAGTGTAAAATTGGAATGGTTAAATAATGAAGCTAGC |
| | CGTATTGCTGCTATACGAGAGAATTCAGAAAAATCAAAAATTG |
| | TAATGGGTGTTCAGGGGAAGATTGTTAACATTGATGAGCTAGG |
| | TATAGTGGCTGTTGCCAGGTCAATAGTAGACGTTGATTTTTATAT |
| | AAAAATGCCCAATGTGTGGGCTTCTAGGGATTGGAAGAACCTG |
| | ATTTATTATGCTGTTAATATAGCAGCAACTCCCCTTATTAATAATA |
| | TATCGAGGGGTATCATGGCTGCTTCACAGACATCTGTTTTATAT |
| | GACTCTTCTCTTGCTCTTATTGCTGCCGAGCAAGCTACAAGAA |
| | ATATAACCATGTGA |
| VP190 (amino acid | MSTTQTQTIERPLPGKNNEDNSRLACLLAEGLQQQQQQQDGDSE |
| sequence; | ISLPLVNAGTFACYDSTLANLTEGRLGSETENAKIRVKIHPSVFIIE |
| AF332093.3: | TNKEMTIEEISTKSLNALVEKRAREARRFSSLTEQKFPRGGGGCY |
| c174441-169744) | SRKNERFIEGEINNIKLNMEETASSLERLAGLLPVVINIKDWTMH |
| | DEKEIRLDLKGNDGMEELVNISHLNQEEWEMERLSSSIVLKDAY |
| | GVFYAHHGILDIVLTTSRFTGKLLQHPVIFRLMDVKVWINTPLQI |
| | AFPDTSKNPNAKKILYQHPSLTRLRDLNDMASNSKSVSSIIIPELS |
| | KFNSTEFGMHYFTAQCFGKNTNSLKDLVTRYYQLSFKNKPQPKL |
| | YEPTATATAASSSSSTASLTTEQKEKIAQSILSSKGKSLGDVSSTLS |
| | KEYDENRKRTKRQKTSTDTNIVPSGAPTSISMKNPVTCFFGPQYT |
| | SIMDCISEKTDWIEMHLFLTSLNDAEHNKTLVVDRKSNVSEIHDS |
| | GRFLTFGQNNTTAFIPDVDIPTLKLILRDDSGESSAAIIASLIYYNN |
| | VNLEGREFSNVSDAVVGLFSGGSAITVGDIAREIASIYNIGRESNC |
| | DSILFPGEPILAGRRSYGRQYRWYDPINCVVGLYRSCLETMTRNI |
| | MRGQPVKVDETAWMYMHQQVLQVVLLPFFDCVLKSGVWAVKE |
| | ARQLTDYIVREVLLKYTADPDQHKFLLFKKPVMDLIAKIVTHYA |
| | VIHSAADNGGVCLAFPRD |
| | PPFIVENDTSLRYYTLTDTPQSILNGDNVAENLKSATSVASSPSSSS |
| | RYSSETPIRVVNLPVPTGRFLKMNKDLELFINVPLISSKEQKQQQQ |
| | QTTATAPFSSETISKSFLNYVPPKSLTRNVTYGQNIAEDGFLGLKN |
| | KGELVSYFKVVKNTERDDGIKDMEIGDINNHQDDTGSLSSSSSSF |
| | VDGVRTSFSVDGKIEHVSAFLPGTTSQPTNLPVHASKQVKYSVK |
| | ELGMETVFFEPLLSSAVLYEASKTKSTQHLSPMRIYKECVSPLSTG |
| | RIDIFPSKVGTVAGTGFEFIWKVLQYDTGLPTTLERLSPKIPSVPIS |
| | GEDSKMEVIAESGKGVQNIIAIAADQLRGSNNIVGGGTRRAIQQQ |
| | QQQQQQETQAVVPVNVPARFEPTFTEIELFLQNKFRNVIATIISRM |
| | MMLVSNEEMKIIKEVCEHVSHIMVDGLYVALDPRKAIEEILERITA |
| | EQNGITIDTGNEGYGSLRYASSGRLFINDEASEEAAAAIGGGGAL |
| | GTGRRVPVELRSILDKLNTIGSTTQQQQQQQRQQRQANNNNTVP |
| | EDIKVHNEQMQKIRDSSLFTSKLLNYIRDDGRKDRIKTNISETLK |
| | KYSRIPSYFIASKAQKPIPWKHTKDNINLNKIPEDLNFSPAQNLFV |
| | PVNPRHILTDMQWLNCISIIETATRDSAIVMQSFQEQADKTTTQLE |
| | ELLSQWNNIVSQVTDEKSPAYVSSVKLEWLNNEASRIAAIRENSE |
| | KSKIVMGVQGKIVNIDELGIVAVARSIVDVDFYIKMPNVWASRD |
| | WKNLIYYAVNIAATPLINNISRGIMAASQTSVLYDSSLALIAAEQA |
| | TRNITM |
| Putative protein kinase | ATGGAGGGTGGGGACCAACGGACAAAACTTACGCCAGCAACC |
| (wsv423)(nucleotide | GTGATGGGACTTTACCAATCGAAAACGCCAGGAGAAGGAGAA |
| sequence; | GGAGGAGAAGGAGGAGGGCAATTCAAGATACCTTCAGCCATA |
| AF332093.3: | GCTGTGAAATCTTGTTGCTCTAAAAACGCTACTCGCCGATCCC |
| c251771-249579) | CTCCCTCAGATTCTCCTTATTCTCTTAGGCCCATGAAGAGACTA |
| | AAGAAGAATAATGGAGAGGTGGGAGGAAAAGCACCGCCTCCT |
| | GTGACTTTGAGGCTCCGCGAGGACTACGAGAGCACACCTTAC |
| | AACTTTAATAGAAATAAGAAGAAGAGGCCTATTACTATTGATGA |
| | AAATCAATTTGCAACATTAAATCCAACGTATGCGACAGACATTA |
| | TCAAGAAGCAGCAATTGCCTTCTGTTAGTGCCGCGTCTGTGTT |
| | GAGGAAGCACCGCGCCAATGCCGACACCCAGTACAGAAAAA |
| | GATTCTCTCATCCAAATTGTGCAAAATTCTCTACTGTCAATTTG |
| | AAGGCTAGAGACTATACTCCACTGTCTGTCCTCCGTTCCCATGT |
| | CAAGGGGCCAAAACACTTGAAATCTTCTTGTGATACCGTGACT |
| | GAAACAAATGTAGTAAAGAGGAACTTTTCTTCCATTGACAAGT |
| | GGGTCAAGCTAGAAAAACCCCCGTGTTACTTTGCAGTGGCAG |
| | AGGCTGATACCAATATTGCAGCCGGTCTAGAATCTCCGTTCCAT |
| | TTGATTAGACAGGCCGCAAAATTAGGCCTCATTTCTGACGTGC |
| | AAGATGTGTCGTCCAACTACGAGACCATAAAACAGAGCTGTAT |
| | TGACGCAAAGGAAAAAGCGTCCAAGTTTTTGTGGTCTAACAA |
| | CCGTACTAAACAACCCCCTTCATCTTGGTGGCCTGTTGGGTTT |
| | GGTAGTAAAAACCTATCCGTTTTAGACACTAGCCCTCTCTTGA |
| | ACTGGAACAGGTTATGCAAGAATAATGGTAAAGGGTGGATAAA |
| | AACCATGAGCATCGATCACATGGCAAAGAATGTTTTTAAGCTT |
| | TCCCCTGGAGCATGTGAATCTATATTGGAGAAGAAAACTACAC |
| | TCTTGGGGGAGGTCACTGCCCAATGTAAGAAATGGGAAAGTT |
| | ACCGCAGAAATATTCCTGTACCAGCACACGTCCAACCAGAATA |
| | TGCTTCTCAAGTCGTAATGATTGGACCATCTGAATTATATCTCG |
| | AAGTTAAAGTCGGGGTATATTACATGCTTGAAACTGGAAAAGT |
| | TATCAAGTTTATGACGGACAAGGAAATGTACTGTGAATTTGTAT |
| | TTGAAACTGTTTTTAGTCACGCTCTTGAGGGAAGAATGAAAGG |
| | CGCAGTAGGTGTGAGAAAGATGTGTGTTGAAGGTTTTTGTGTC |
| | GAGATGGATTTTGCAGGCATTTCTGTGATTGATGTATTAAATGG |
| | AGACCTGAAATGTAAAATGGACGAGAATGTTGTACAGCAACCT |
| | AACCCCTCGACTACTTCCTCCAAGCCAGCCGCTGAGCTCATGC |
| | AAGATCATGGCAGCTTGTGTAGGATGAGGGATACTCTGTACGG |
| | TGTTAGGATGCTTCAAGCTACTGGCCGCCTGCCTGAAGGTCTA |
| | CAATCTAAATGCAAGAAACCCATTACGGATTCAATTTCAGCCAT |
| | AGCTATCGTTGGAAAAATGAGGGAGAGAATGTTAAACCAATTG |
| | CCCTTTGTTTTGGTAGAAATTGTAAATATTGTCACTCGGTTGTC |
| | TCAACAAGGATTAGTGAATCCGGACATAAAAAGTGACAATATA |
| | GTAATTGATGGAATAACTGGTCAACCTAAGATGATTGATTTTGG |
| | TTTAATTGTACCATGTAAAAAGTACTACAATTTTAAATGTTGGG |
| | GAACTGATGAGAGGTTCTTTAGTAACCATCCTCATACAGCTCCT |
| | GAATTTATTAACAGTGAGTTGTGTTCAGAAACTGCCATGACTTT |
| | TGGGTTGGCTTATTTGTTAATAGACATGTTGTCCATTTTGATTAA |
| | GAGAACTGCAGATTTGTCTGCCAATTCTATCTATACAAACATTC |
| | CATTTTTGTCTATTGTATCTAAAATGTATGACCAGGAAAAGACC |
| | AATAGGCCGAGAGCGTATGAAATTGCGCCTGTAATTGGTGCAT |
| | GTTTCCCGTTCAAGGATAATATTGCTAAACTTTTCCAGTCACCT |
| | AAACATTCATTGTATAGCAAGAAGGTTAAGTAG |
| Putative protein kinase | MEGGDQRTKLTPATVMGLYQSKTPGEGEGGEGGGQFKIPSAIAV |
| (wsv423)(amino acid | KSCCSKNATRRSPPSDSPYSLRPMKRLKKNNGEVGGKAPPPVTL |
| sequence; | RLREDYESTPYNFNRNKKKRPITIDENQFATLNPTYATDIIKKQQL |
| AF332093.3: | PSVSAASVLRKHRANADTQYRKRFSHPNCAKFSTVNLKARDYT |
| c251771-249579) | PLSVLRSHVKGPKHLKSSCDTVTETNVVKRNFSSIDKWVKLEKP |
| | PCYFAVAEADTNIAAGLESPFHLIRQAAKLGLISDVQDVSSNYETI |
| | KQSCIDAKEKASKFLWSNNRTKQPPSSWWPVGFGSKNLSVLDTS |
| | PLLNWNRLCKNNGKGWIKTMSIDHMAKNVFKLSPGACESILEK |
| | KTTLLGEVTAQCKKWESYRRNIPVPAHVQPEYASQVVMIGPSEL |
| | YLEVKVGVYYMLETGKVIKFMTDKEMYCEFVFETVFSHALEGR |
| | MKGAVGVRKMCVEGFCVEMDFAGISVIDVLNGDLKCKMDENV |
| | VQQPNPSTTSSKPAAELMQDHGSLCRMRDTLYGVRMLQATGRL |
| | PEGLQSKCKKPITDSISAIAIVGKMRERMLNQLPFVLVEIVNIVTR |
| | LSQQGLVNPDIKSDNIVIDGITGQPKMIDFGLIVPCKKYYNFKCW |
| | GTDERFFSNHPHTAPEFINSELCSETAMTFGLAYLLIDMLSILIKRT |
| | ADLSANSIYTNIPFLSIVSKMYDQEKTNRPRAYEIAPVIGACFPFK |
| | DNIAKLFQSPKHSLYSKKVK |
| VP15 (nucleotide | ATGGTTGCCCGAAGCTCCAAGACCAAATCCCGCCGTGGAAGC |
| sequence; | AAGAAGAGGTCCACCACTGCTGGACGCATCTCCAAGCGGAGG |
| AY374120.1) | AGCCCATCAATGAAGAAGCGTGCAGGAAAGAAGAGCTCCACT |
| | GTCCGTCGCCGTTCCTCAAAGAGCGGAAAGAAGTCTGGAGCC |
| | CGCAAGTCAAGGCGTTAA |
| VP15 (amino acid | MVARSSKTKSRRGSKKRSTTAGRISKRRSPSMKKRAGKKSSTVR |
| sequence; | RRSSKSGKKSGARKSRR |
| AY374120.1) | |
| WSSV447 (nucleotide | ATGTTTCTAAAGAATCTTATAACAGAAAATGTACCATCTGTTTT |
| sequence; | TTTCTGCACATATTTAACATCCGGGCAAGATTTTAACTTGGCAA |
| AF440570.1: | AATTTATTGGGGAAGGTTCTACCTTGAAATATGCGCTCTCAGTC |
| c264969-264733) | AAATCTTCCAATTCCATTTTTCTATGGAAAACTAAACAGTCGTG |
| | TTTTGCATTACATATTTTAGATGCCATACTAGCAGTAACTTTTTT |
| | ATCAAAGTATTTGTAG |
| ICP11 (nucleotide | TTGAAAGAAGACTTCTTGAAGAGGACCGATAAAAAAATGGCC |
| sequence; | ACCTTCCAGACTGACGCCGATTTCTTGCTGGTGGGGGATGATA |
| MH090824.1) | CTAGTAGATATGAAGAAGTGATGAAGACTTTTGATACTGTTGA |
| | GGCAGTCAGGAAGAGTGATCTAGATGACCGTGTTTACATGGTG |
| | TGCCTAAAGCAGGGATCTACTTTTGTCCTCAATGGAGGCATCG |
| | AAGAATTGCGTCTTTTGACTGGAGATTCAACGCTGGAGATTCA |
| | ACCCATGATTGTGCCAACAACAGAATAA |
| VP19 (nucleotide | ATGGCCACCACGACTAACACTCTTCCTTTCGGCAGGACCGGAG |
| sequence; | CCCAGGCCGCTGGCCCTTCTTACACCATGGAAGATCTTGAAGG |
| JX515788.1) | CTCCATGTCTATGGCTCGCATGGGTCTCTTTTTGATCGTTGCTAT |
| | CTCAATTGGTATCCTCGTCCTGGCCGTCATGAATGTATGGATGG |
| | GACCAAAGAAGGACAGCGATTCTGACACTGATAAGGACACCG |
| | ATGATGATGACGACACTGCCAACGATAACGATGATGAGGACAA |
| | ATATAAGAACAGGACCAGGGATATGATGCTTCTGGCTGGGTCC |
| | GCTCTTCTGTTCCTCGTTTCCGCCGCCACCGTTTTTATGTCTTA |
| | CCCCAAGAGGAGGCAGTAA |
| VP19 (nucleotide | ATGGCCACCACGACTAACACTCTTCCTTTCGGCAGGACCGGAG |
| sequence; | CCCAGGCCGCTGGCCCTTCTTACACCATGGAAGATCTTGAAGG |
| AF332093.3) | CTCCATGTCTATGGCTCGCATGGGTCTCTTTTTGATCGTTGCTAT |
| | CTCAATTGGTATCCTCGTCCTGGCCGTCATGAATGTATGGATGG |
| | GACCAAAGAAGGACAGCGATTCTGACACTGATAAGGACACCG |
| | ATGATGATGACGACACTGCCAACGATAACGATGATGAGGACAA |
| | ATATAAGAACAGGACCAGGGATATGATGCTTCTGGCTGGGTCC |
| | GCTCTTCTGTTCCTCGTTTCCGCCGCCACCGTTTTTATGTCTTA |
| | CCCCAAGAGGAGGCAGTAA |
| Collagen-like protein | ATGGCGTACATTGACCAAGGGGCGTTTGGAGCCAAATCTGTGA |
| (WSSV-CLP) | TGCAGCAACAATACCTTCTTCCACCCTCTAATAGGCTCTCCAA |
| (nucleotide sequence; | ACAACAACCTCCACTGGCATCATCTTCTCTTCAACCTTCCTCTT |
| KU216744.2) | CTAATAAACCTAGGAGTACATCAAGAGTAACAGACATATTTGTT |
| | GTGATAACTTGTGTTGTTTTAATTGCTGCTTTTATAAGCAACTC |
| | ATTTAGTGTTACAAAAAATGTGGTAAAACTTTCTAAAGAACAA |
| | ACCGAGAAAATACTAGAAAAAGATTTGCCTGATAAGGTGTACA |
| | AATTGTTTGAAAATTTAAAGGATGGAACATTTGGTATAGGGGA |
| | AGATGAGGAGGAAGAAAGGGAGGAGAGGGAGGAAGGGGAG |
| | GAAGAGCTTGAAACAAAAAACATAAGACTTAAAAGAAAGTGG |
| | AAAGAAATGACTGAACAAGGGGATCAAGGAATAAAAGTAAGG |
| | AAATTACATGGCCCGAGAGGAGAAAGAGGAGAAACTGGTCCA |
| | GCAGGAGCAGTTGGCCCTGCAGGCCCTCAAGGAGAAAGAGG |
| | AGCAATTGGACCGGCAGGAAAGGATGGAGCAGTTGGCCCTGC |
| | AGGCCCTCAAGGAGAAAGAGGAGCAATTGGACCGGCAGGAA |
| | AGGATGGAGCAGTTGGCCCTCAAGGCCCTCCAGGAGAAAGAG |
| | GAGAAAATGGACGCCCAGGAAGAGATGGAGCAGTTGGCCCTC |
| | AAGGAGAAAGAGGAGCAATTGGACCGGCAGGAAAGGATGGA |
| | GCAGTTGGCCCTCAAGGAGAAAGAGGAGCAATTGGACCGGCA |
| | GGAAAGGATGGAGCAGTTGGCCCTGCAGGCCCTCAAGGAGAA |
| | AGAGGAGAAAATGGACGCCCAGGAAGAGATGGAGCAGTTGG |
| | CCCTGCAGGCCCTCCAGGAGAAAGAGGAGCAATTGGACCGGC |
| | AGGAAGAGATGGAGCAGTTGGCCCTGCAGGCCCTCCAGGAGA |
| | AAGAGGAGCAACAGGTATACCAGGAAGGGATGGCGTGGACGG |
| | TTCTGTGGGCCCTCAAGGAGAAAGAGGAGAAATTGGACGCCC |
| | AGGAAGAGATGGAGCAGTTGGCCCTGCAGGCCCTCAAGGAAG |
| | AAGAGGAGCAACAGGACGCGCAGGAAAGGATGGTGCAGTTG |
| | GTCCTGCAGGCCCTCAAGGAGAAAAAGGAGAAGCTGGTAAG |
| | GACGGTTCTATAGGGCCTCAAGGAATACAAGGCCCAAGAGGA |
| | GAGACTGGACCACCGGGAAGGGACGGCACTGCAGCAGAAAG |
| | AGGAGAAAGAGGCTTCCCAGGACCACCAGGCGAAACTGGAC |
| | CACCAGGAAAGGATGGTGTGGATGGTTCTGAGGGCCCTCAAG |
| | GGAAAAGAGGAGAAACAGGACCCGTTGGACCTAGGGGTGAA |
| | CCAGGTCTAGCTGGCCTCCCAGGAAGAGATGGAGCAATTGGC |
| | CCTGCAGGCCCTCCAGGAGAAAGAGGAGCAACTGGTCTACCA |
| | GGAAGGAATGGTGTGGATGGTTCTATCGGCCCCCAAGGAAGA |
| | AGAGGAGCAACAGGCCGCGCAGGAAAGGATGGGGCAGTTGG |
| | CCCTGCAGGCCCTCCAGGAGAAAGAGGAGCAACAGGTATACC |
| | AGGAAGGGATGGTGTGGACGGTTCTGTGGGCCCTCCAGGAGA |
| | AAGAGGAGAAACTGGACCAGCAGGAAGGGACGGTTCAGTTG |
| | GCCCTGCTGGCCCTCAAGGAGAAAGAGGAGAAAATGGACGCC |
| | CAGGAAGAGATGGGGCAACTGGCCCTATAGGTCCTGCTGGTCC |
| | TCAAGGAGAAAAAGGAGAAAATGGACGCCCAGGAAGAGATG |
| | GAGCAACTGGCCCTATAGGCCCTAGAGGAGAAACTGGTGCAA |
| | TGGGAAAGAATGGCGTGGACGGTTCTATGGGTCCTCAAGGAA |
| | GAAGAGGAGCAACAGGCCGCGCAGGAAAGGATGGGGCAGTT |
| | GGCCCTGCTGGCCCTCCAGGAGAAAGAGGAGAAACTGGACC |
| | AGCAGGAAGGGACGGTTCAGTTGGCCCTGCTGGCCCTCAAGG |
| | AGAAACAGGATTAACTGGCAGCCCAGGAAGAGATGGAGCAAC |
| | TGGCCCTATAGGTCCTGCTGGCCCTCAAGGAGAAAAAGGAGA |
| | AAATGGACGCCCAGGAAGAGATGGAGCAACTGGCCCTATAGG |
| | TCCTGCTGGCCCTCAAGGAGAAAAAGGAGAAAATGGACGCCC |
| | AGGAAGAGATGGAGCAACTGGCCCTATAGGTCCTGCTGGCCCT |
| | CAAGGAGAAACAGGATTAACTGGACGCCCAGGAAGAGATGG |
| | AGCAACTGGCCCTATAGGTCCTAGAGGAGAAACTGGTGCAAT |
| | GGGAAAGAATGGTGTGGACGGTTCTACGGGTCCTCAAGGAAG |
| | AAGAGGAGCAACAGGCCGCGCAGGAAAGGATGGAGCAGTTG |
| | GCCCTGCTGGCCCTCCAGGAGAAAGAGGAGAAAATGGACGCC |
| | CAGGAAGAGATGGAGCAACTGGCCCTATAGGTCCTGCTGGCC |
| | CTCAAGGAGAAACAGGATTAGCTGGGCTGCCAGGAAGAGATG |
| | GAGCAATTGGTCCTCAAGGAGAAAAGGGAGAAAATGGACGCC |
| | CAGGAAAGGATGGGGCAACTGGCCCTATGGGTCCTCCAGGAG |
| | AAAGGGGAGAGACTGGTCCTATAGGTCCTGCTGGCCCTCAAG |
| | GAGCAACTGGTCTTCCAGGAAGGGATGGTGTGGATGGTTCTGT |
| | TGGCCCTCAAGGAAAAAGAGGATTAATAGGGCGCACAGGAAG |
| | GGATGGGGCAATTGGCCCTGTAGGTCCTGCAGGCCCTAAAGG |
| | AGAAACAGGATTAGCTGGCCTGCCAGGGATAGATGGAAAGGA |
| | CGGTTCCGTGGGTCCTCAAGGAGCAATTGGACCTATAGGCCCA |
| | CGAGGAGAAAGAGGAGAAACTGGACGACCAGGAAGGGACGG |
| | TGAGGATGGTTCCACAGGCCCTATGGGCCCCCAAGGACTAAG |
| | AGGAGCTACGGGAGCTCCAGGACCGCAAGGAGAAAGAGGAT |
| | TAAAGGGACGGCCAGGAAAAGATGGTGAAACAGGTCCTCCAG |
| | GGCGACAAGGAAGGGATGGAATAATGGGTCCTAGGGGTCTTC |
| | GAGGAGAAAAAGGAGCACCTGGTAATGATGGTCTAGAGGGAC |
| | CTGAAGGAAGAGATGGTGCACCTGGTCCCGCTGGCCCTATTGG |
| | ACCTCAAGGAATAAGAGGATTAAAAGGTATCCAGGGACGACC |
| | AGGAAGAGACGGAGAAATGGGACCAGCCGGCAAGGACGGAA |
| | TAGAAGGCCCTAGAGGTCAAGATGGAACAACTGGCGCTAAAG |
| | GACCTAGAGGATTAAGAGGTTTTCAAGGAAGAACAGGAGAAA |
| | CTGGTGCACAAGGATCTAGAGGAGAAAAAGGCGATAGAGGGC |
| | TAACAGGCCCTCAAGGAAGAGACGGTCCACCCGGTGAAGAA |
| | GGTCCTCAAGGTCTTAGAGGAGAAAGGGGAGCACCTGGCCCT |
| | AGAGGTCCTAGAGGTATTCGTGGCCGTTCAGGACCTCAAGGA |
| | AGTAACGGCGTGCAAGGACCTCGAGGTCCCCGAGGAACAAA |
| | AGGAAGAACAGGAATACAAGGCCTCACTGGCATAGAAGGTCC |
| | TCGAGGTCCTAGAGGTATACAAGGAAAGGAAGGAAGAATGGG |
| | GAAAATTGGACATCGAGGAGAAAAGGGTGATAAAGGAGACCG |
| | TGGAGAACAAGGCATCGCTGGAGCAGACGGGGAAAAAGGTC |
| | CAAGAGGTTTACGAGGAATTCGAGGCCCTATTGGTGCTCCTGG |
| | TAAGCCTGGCACGGAAGGGGTTAGAGGTCCTAGAGGGGTGAG |
| | AGGTGTTCCTGGCTATCCTGGCGCACAAGGGGAATTAGGTCCC |
| | CAAGGACCAACAGGTCCTCAAGGGCCAGCAGGTCCTCAAGGG |
| | CCGATGGGGCGTACAGGAGATACTGGTCCCATGGGCCCTCCTG |
| | GAGCAGTGGGACCAAGAGGAGAGAAAGGAGGTAGAGGAAGA |
| | AAGGGAAAAAATGGCCCTAAAGGAGCGGACGGAAAAGATGC |
| | CGTAAATATCATACAAAAATATTCAATCACCCATGCTCGTGCAG |
| | AGATAATGTGGGAAGGAAATGAAATCGGAGAAGCATACATTGG |
| | AAGATCTTATGGAACTGATACAATCCCTGTGATGATAGAAAATA |
| | GAATAGGGATGACAAATGAGGACAAAAAAAACGAATATTGTAT |
| | ACAAGTAATGACAATGCACTCAATAACAACTAGAGGAAGAAC |
| | ATCGGGTGTTTTTGTGGTAAGCAATAAGACAGATTATATCCTTT |
| | TAGTTACTTTACTGATGCCAGAAAGTGTTTCCTGTAGAACAGAT |
| | GTCAGTACAAATGCGAGGTCAGAGAGGGTGAATGCTGTTAGA |
| | GAAAGAGAAAGCAAATCGTACAGATTTATTAGGCCGTCTGACC |
| | AATCTATAGGTACTCATTCACGTTCAAAAATTGCCGTGGTAATG |
| | TATCCAGACGCAAGCATGAGTTACTCAGTTGATACATTAGACG |
| | CTGATGTGGCGCGAAGAGAAACAACGTCTGTGCTTTTATTAGC |
| | AGAAACCATACACGGGGAAAAAGATAGAGGTTTCTATGCTGAT |
| | AGAGGAACTGTAGGGAGGTTGATGGTACCTCCCACTGAAGAA |
| | GAGTTATTGGTATTGCAAGCGGATCCAGACATCACCCCAACAA |
| | TCGCAGACCCGATTACAGTAGCAGAAGTTGGAAACCCTGTCGT |
| | GAGGATAAAAACTCCTTCATGTGATGAGTTGAATAAATTCATG |
| | AGATTCTCTACTAATTTCTCATTTTTAAAATACGTCAAACGGTTT |
| | ATGGCGCAGAGAGCTTCATCTAATAGTAGGAATGATATGTCCGC |
| | TTTCTTATTAGATTTGATTTTTGAAAAATTGATAGAGTTTTTATT |
| | ATCAACAGGATGGAATAGAGAAGCTCTGGGAGGAACTACTGG |
| | GGCTATTCTTATGATCAGGACGGCAACTTGTCGTCTAGCGCAAT |
| | CTTATTACCCCCTCCCTGAAACCGTTGGTATGGACTTTTTGCAA |
| | ACACCTGGGTTTGACTACAATAAATCTTTCCCTAACAATGAAA |
| | GGTATATATATGAATTTCAAGCTACAATGTAA |
| DNA polymerase | ATGACATCTCCAGCTCCATCACCCTCTTCCACCCCCAAATCCAG |
| (DNApol) (nucleotide | TTGTACCACTATTGTAAACCGATGTGGTTTCCTCCTTGACAACA |
| sequence; | ACAAGGAAGTGGTCATCTACGACACCAATTCCAAATTCAAGTG |
| AF332093.3) | TGAACCCAAAAATCTGGAACTAATTGGTGTACTTTCTGGAGTC |
| | TCTGATAATGTTGTTACCCAGATATCCCCCGACCAGATATTTGT |
| | GGGAACATATATGGTCAAATATAACTGGTCTAAATCTGGTCATG |
| | AACGCTTCAGTGACATGAGTAACAACTGTCTGGACAATATTAC |
| | ACGCCCTTCAGAAGTGATTGAAAGTGTGATAAAGAAAACGTC |
| | CAGCGACTTTAAAATGAAGTACACACGTTCCTTGATGGACCAC |
| | ACCGAGAAATACTATTTTTCTGGTGACCAAAAATTGAGCAAAA |
| | TTAGTAGTTGGTGTACAACCCCTATAACGACAGTGGGTATGCA |
| | ACTCCGTCTAGAAAACTTTGTCAAGGAAGAACATGAAACTGT |
| | CGTGGTGCACAACCCTTCTGGAATGACTGGATTCAACATATTTA |
| | ATAGTTCCCCCGTGTATTTTGAAGTGCACAATGAGATGGACGC |
| | CCTAATTTTTATGGCGGCTTTCTTGAAGCACAATAGTTTATGGG |
| | GAGAAATTAACGCCAATATGGACTTGTACACGTTTGATTATGCG |
| | GGTGCTTTTCTGGACGAAAGATGGTGCCACCACGAGAAGAGT |
| | TTTTCTGTCGTCCGAGCACAACTTATCAACTCGTATTACAAGTG |
| | CAGGAGAAAAATCATGCAAGCCCTGGACAATAACTACAACAA |
| | CAAGAATAAGAAGAGGAAGAATGTTGGTGGAGCACCTGCGTT |
| | CACATTTATGAGCGGGGACGGAGAGGGAGGAAAGGAAGCCCT |
| | AGAAGCTAGITTCGATGTGATTGGGGGAACAAGAGGAGGAAG |
| | ATTTGGTGTTGATTCAACACCATGCCCCCATTCTTCAGCCATGC |
| | AACTAAAACTGGACAATGAAGGAAACTATGGATGTATTGCCTG |
| | CTTCGCATCAATGTTCTTTGTATTGGAGAACCCAGGTGATGAAT |
| | CTTCCTTCATATCAACGGATGCCTCTAAAATTGGACAAGCGCA |
| | AGCATGGATAGATGAACGACTACGAAACAATGAAAATGGAGG |
| | AGAAGAAAATAATGTCTTTAAAAAGACCTTCCATATGCTGGCT |
| | GATATTACCCAAAAGGCTCATGAAACTGCCTATTCCAATACCAT |
| | CCCACTTGGACCCAATGGCAGGCAGTGGAATTGGCCTACTCAC |
| | ACTGTGGAACCTATTGCCCATGAATTTGTTACCCATTCTCTAGT |
| | AAACACATTGAAAAATCTAGGGGATAGAAAACTTCCCCGATTC |
| | AATTTTGATATCTTGTACAACTTGCTTAATCCATTTGGAAAAAT |
| | GTTGCTAGTGTTTATTCAAAATTGTCACATTTTAACTGGACATA |
| | AAAACAATGAAAATGTGGTGCCTCGAGGTTCTGCTTCTGGGA |
| | AGTGGTGGACTATTAATTTTGTGGGTGTGAACATGTGGACTTTT |
| | CAAGTAACAAAATGTAAAGTTGAAAAGGATAGAAAAATATCCG |
| | ATTTGGCCTGTATGGAAACTCTCCCTCGTCTACCTAATCCAGGA |
| | AGCACTACCGTCGATGACAGAATAGTTTTTAAGGGATTCTGTA |
| | GAGGGGAAAATCTAGGGAGTGTAGGTGAAGTCGTATCCGACA |
| | TTACACAGAGTGTCAAGAATTTTTGTCTCATGGTTGAAAATAG |
| | GAAATTTAGTGTGGATAAAGAAACTGGTTTCATCTCTTCAGAA |
| | TCGATAGTGTCTGATCCCTTCTTTTCACTAGAAGTGACTGGCTG |
| | TAGATCTAATCGTGCCCAAGATACTATTAATAATGGCCGAGTTA |
| | GTGCTCGTGTAATGAGGATCCTAAAGTCACGTGAAGGTGCTCG |
| | TGTATGGTTGGCCAAGGATGAAAATGCCATCATCTTTGAAAAC |
| | GTTAACCACGATACGGCCATCTCTACGGACGCTATGGAGCGAG |
| | CTATAGGGCAGCACAAGATACTGTACTATGATATTGAAACAACA |
| | GATAAAGATTTCACCGACAAAAAATCAGTCATCACATCTATTG |
| | GGTTCTGTTTGTGTACGGGAGGCGATATGACACATGGAGGAGA |
| | GAGAGGAGTATTTGGACTGGTTGCACCTGGATCCGACGTGGA |
| | AAAGGTGAAAGAGACTATAATAAATTCGTACGATCCTGAAGAA |
| | AAGGAAGACATTATGAAACAGTGCCCTCAAGTGATTGAAATTT |
| | TCACCAACGAGTTTGAAATGTTGCTTGGTTTTGGAAAGTATATA |
| | GATAAAGTGAAGCCTCACGTGATTAGTGGGTGGAACAATGTAG |
| | CTTTTGACGACCCCTTTGTCTTTACTCGTATCGTCAAACATTTG |
| | AGTGATCACACCAAAGACATGTCTTATTGTGTAGCAGATGCAT |
| | CTACAGCAGAATCTGTCCTTCCTAGAGCAACAGAAGGAGGAG |
| | GAGGAGAAACTCCATATAGATTGAGCACCCCTCAAGAAAGAAT |
| | ACAACTAGCAAGCACTGGTATTTTCAATAAATTGGGAAAATTT |
| | GTAGACAAGAAAACTGGCATGTTGAAACCTGAAATGACTGCA |
| | GATTTATTGGCCGGGGCAGAAAGTCAGGCCAATACCAAGTTTA |
| | AGGAACGCAACAAGTTATCCTCCAGTAATAAAGGATCAGCAG |
| | GATGGTTCCAGAAAATTATTGGCGGTATGTGCAGTGCTATTCGG |
| | TTGGATCTCATGAAAGTGTGCGAAAAGGCCTATAAAGAATCCC |
| | TCTCTGAATTTAATTTGAACGCCGTGCTCGCCAAAGTGAGTAG |
| | TGTCGGCGACAAGGTTAAAAATGTAAAAGATGAAGTAGACCT |
| | ACACTTTCATCTATTGGGATTCTTGAAGCTGAAGAAGGCCCAG |
| | GATCAGGCAAAAGTACACGTCTATTGTTGCAAGGATGCCTACT |
| | TGACTGGTATAGTTTCTACCTCCATCAACAAGGAAGGGGAGAT |
| | TTTTAGGCTGTGTATGGACTCTGCTTTAACCGAGGCGGTCGTG |
| | ACAGCCAACCTGGCCACTCCTCTATGTATAGGAGAAGGAGCAA |
| | TCTGTAGAAATATGGGAGAAGAAAGGGCAGATAGAAGAGGTG |
| | TGGGAGTAAGAAGACACTCTATTGCCACAGACACAAAGGGAG |
| | GTATGGTGAGTCAACCTATCGTCAATCATGTTCCCTATCAAACG |
| | ATTGACATGACAAGTTTGTACCCGATGACCATGTGTCAGAATA |
| | ATCTGTGCACCACTACCTTTGTGACCCATCGACAAATTATGCAA |
| | CTGAGGGATAGATTGGTACTTGAAAAAATGAAAAACAAAACC |
| | ACCGACTCTTTATTGTTGTTGGACGTTATTGACGAGTGCAATCA |
| | GATTGTGTTGTCCGAGTACAGACCCATTGATATTGCAGTCGCAT |
| | CATGGAAGAATAGCAACTCTAATAGACAAACTCCAATTACTCG |
| | CATAGAGGAAAGTTTGGGTCTAAGATTCATAGAAAATTTGGAT |
| | GCCGAGAAGACAAATAATAAAACGTGGTGCACCAATACATCTC |
| | CCAATATGAATGTCACTGCCGCAGGTATGGATTACTTCCCCGAG |
| | ATTGTGTGTGACATTAATATGCAGTTTGCGGCCAAGGTGAATG |
| | ATGATATGCATATAGCCCCAGCAAGTTTAGAGTATATGCTTCAA |
| | GTATTGCCCATAATGTTAATCGACAGACCGTACATTGGCGCACA |
| | CATAACAGCTGGAAAATGTCGTACATTGGAGGATATTCTCTCA |
| | GAACTCGAAAAGGACTTTTCTGTTGAAAAAGATGAGGAAATT |
| | ATAAGAACCCATTGGACATTTAAGGGTCAAAAACAATACGATT |
| | TCTGTCATAGTCCTGTGACCCAAATGGCTCGTCACATTATTGAA |
| | TCTACGGGAAGAAATATCCGTGATTATGAAGGCAATGAAAAAT |
| | TCGAGAGGTTGGTTAGCTTATCAGACAGAATTTATCGCCGCGT |
| | TGGCGCATTTGATTCGGCCAATGATCCAGCTGTTAGACTGTGG |
| | TCTTCTCGTCTAATCAATGTTGGAATGTTGGTTAGGACATGGAA |
| | CGTAAAAACTGACATTCTTAAGGGAATCATCCCTCAAATGCAA |
| | GCCACTTACAGAGCCGATCGAGTTGTGATGCAGAACAAGGCC |
| | AAGGAGTTTGCCAAGATGGGAGACATGAAAAGAGCTGGTCTA |
| | AACAAAGTTGGACAAAATATTATGAAGCTCGGTATGAATTCCA |
| | TGTATGGTCATTTGGCCCTAAGAGCACGTTCGAGCCGTAAAGA |
| | GTTTGCGTCTGGATCTGCCAATACTGCCTCAAGTATTTCCAACA |
| | TGTCAGCCACCGGAGGAATTGGAGGAGGCACAAGGCACTCGG |
| | TGACGGCCAATCAGATTACAGAAAATGCTCGATGTGTATTTGG |
| | CAATATTGGTTGTGGATTACAGATGGCTCTTCCTGGTACTAAGC |
| | AGACGTACGGGGATACAGATTCTGTATTCTGCGTGCATAATATT |
| | GTAGGTGATGGAGGAATGATACCAGAATATGATGAACAAACTG |
| | GCAAATATTATTATGTGATGGATATTGCTCTAAAAAATAAAATGG |
| | CTGCAATTATTCCCATCCTAGTCAACTCGTTAACAAAGGGCATC |
| | CAGTTTGTAGAGCGCCGAGACGCTGGTGTGGGCATGATGAATA |
| | TCGCCCATGAACGTCTAGCTGTCGCTGGTCTTTTGTTTGCCAA |
| | GAAAACATACCATATGCTTCACTTTAATGAAAATAGTGCAGCGT |
| | TCAATGACATGATAAAATTGAAATCAACCGATAACAATAATAAG |
| | TTTGCATCCTTTATCAAGAGACCTAGCCACGCAGATGGGTATGT |
| | TGTTCCCCATAATCCTTCATTGATTCTTAGAGCGGCCGAAGGAC |
| | CCGCTGGTAAGAAATTGAAAAGCTTTTTGGAAGAGGAAGGAA |
| | TTCATGACGAGAAGAGTATGGAGGAATGGTTTACCTCTTCACC |
| | TACGTGGATGGCCATGGATGCTTCTGTTATCAACAACTTGTATG |
| | CCTCACAAATTGTAGGGGTGGAGAAGGGTAACTGGATTGACG |
| | CCATGACTTCCCGCCCCATAGAAGCGGGTACAGAAATGATGGA |
| | GGCGGTGACGCAAGCGAATGCAGCTTTCACCCCTTACAAAAA |
| | GGGAGCCTTTGTGAAGAAGGGAATTACACCCACCACCAAACT |
| | AAAGGGTCTCCAATCATTGATTGCAAGATTTTTACCAAAAATA |
| | GAGGAAAAGAAAAGCTGTTATTTGGATGTGATGAAGAATCATG |
| | TGGAGAATTTTGCATCCCATATAACAAATCCTGCTATGATGATTA |
| | CTAGTTCCCGAGTCAACAAGTTTGATACGTCAAAAGAACAGA |
| | GTAGACCTAATCCTCTAGCTCTAGCGATAAATAACCACCTGAAC |
| | CCTTCTTCAGAAATTTCATTGGGGCAGAAATTTAAGACGGTGA |
| | CATCAGTTTCTTCTTGGAGTCTTTCGGCAGAGGAAGGGGAAGT |
| | CCCTGCTGGTTATTTTAACGCTGGTAGCGTGCGTTGGGATGCC |
| | ACCAACATGAAGGGAAGTGTTCCTGCATTTTCAGTCAAGAATT |
| | TATCTGTTGTGCCCAACGCCATCACATCTGTATACAAGATGGTT |
| | GAGAGCGATAAGACGGCAATAAAATCCATGATTGCTAAAAATG |
| | TAGAAGTGTTGTGTTCTACATCTGCCAATACTGGATTTTCTCTG |
| | AGAAGAGGAGCATTGTCATTTAATACAGGCGTCATTGTTACAA |
| | AGGACGTGGCTATGGCTTGTATACGATCTCTAAATAATAAACAA |
| | ATGTTATTGTTTGTTGGAGGGGGAAAGGATTACGGTGAAGACG |
| | ACGACGACGACGACGAAGAAGCAGAAGAAGAGGACGAAGA |
| | AAATGGTGAAAACGAAGAGAACAAAGGTGACTGTGTCACGG |
| | AAAAAAAGATCCCTGGACGAAGCACTAACAAGGATGTTGGTG |
| | AAGAAACTAAAACAAGCGAGAAAACGGAGGGAGAAAGAAA |
| | GGGCTCTAAGACGGCAAAGGGAAAGACGGAGGAAATTGCTAG |
| | TTCGTTGAGTAAATGTGGGAAGAAAGATGCGAGAGATGTCATT |
| | CTTGACCGTTTACTAAAAGCAACACATTCTTCTTGCACCAACA |
| | ATGAAGAGAGAACCAGAGTCTTACAACAATATAGCAATTGTAC |
| | ATTATCTTCCTATATAACTTCAGTCATGAAATTGGACCAAAGAG |
| | TAGCAGACCAAATGGAAAATTTAATATCTCAATTGGATCAAATA |
| | CGTAATCTCTCCAACAAGAAGAGGCAAGAAAAGGGAGGGCCT |
| | TTTAAGTCTGAATTGGACGCCATGGTTGCTGCAGTTAAGGTTA |
| | AGTTTTTCCCAGTTTTAGATGCGTCTAGAAAATTGACTCAAGA |
| | CCATTGGAAAAAGTGCCCCGTGTCCATCCCAGAAACGCGTGA |
| | AGAAAAACCATTAATGGGTGTGCCTTTTGAAGTTGCACTCAAT |
| | TCTCTAATAGGAAAACACAAGTGCACAGATACATGCGACATGG |
| | CTTGTTGTCAATCATTGTATTTTGTCCTCTTGTACACGCTAGCTT |
| | TAAAATTTGAGAACGAAAGATTGGCCCGGCAAATTGGCCTAGA |
| | TGACTCTGTAGATTTGATGGCTGAGATGTTGTTCGGAGGGGAT |
| | AAACTATTGGCCCAGGAAGTGTTAAAAAGGGTAAAAGATGCT |
| | CAAGATAGAAAGTTGGTGAAATCTTTATTGCCTTTAAATTATAA |
| | CCATGACACAAATACAATTATATTTTTGTTTGAGTCTTTAAGGTT |
| | TGCTCAGAAACCTGTAGCTGGTATGAGTGTTAGTGAAATAAAA |
| | GACGCTGTTAGAGGTCTGGCCTTTTCTACCACTACAGGTACTG |
| | TGTGGAATTATACTGATGAAAGATTTTTTGGACCATTGTATAAC |
| | ATGGATGAACTTTGTAACGAACGTGTCAATGGAAATTGTAAAT |
| | TGTCCTTTATAACTGGTATTTATCATACGGCAGCAGTAGAATTG |
| | GCTGCTGCATGTCTATCTTGTGTTTTGTAA |
| Ribonucleotide | ATGGGTTCTAACCAGCAACAATCATTCATCTCAAAGAGGAATG |
| reductase subunit 1 | GCACTAAGCAAGAAATTAGTCTTGAAAAGATAATCAAGAGGAT |
| (RR1)(nucleotide | TGAAAATGCCTGTCTTCCAGTCAACCAGTATGTGCCCAAGCTT |
| sequence; | GACAAGAACGCAATTRACCCTCAAGAACTTGCATCTCACATCA |
| MH090824.1) | TGGACCGTCTTCCCGCTACCATCTCCTTCCAAGAAATGGACGA |
| | TTTTCTGGCCGATTATGCAAAGACAAAAATTGTTGACCACCCT |
| | GATTTTGGAAAACTGGCAGGAAGATTCATCTGTTCGAACATCC |
| | ACAAAAACACCAAAGAGTGGAATAGTTTTAGTGCAACAACTC |
| | AGAAATTGAGGCACGCAATCCACCCTGGAACTGGTAAACCAG |
| | CATCAGTAGTTAATGATACCTACTATGAAAATGTTATGGCCAAT |
| | GCTGAAATTCTCGATGCTGTTATCGATTACAAAATGGATTATCT |
| | CTTCACCTGTTTCGGACTGAGGACGCTAGAATACTCTTATTTGA |
| | TCAAGATTGGTTCCCCCACTGATAGAAAGAAGAGAATCTTGGT |
| | TGAGCGCCCTCAGGACATGATTATGCGTGTGGCTGTCGGCATT |
| | CACGGATCAGACATCAAATCTGTCATTGAAACATATGATCTCAT |
| | GTCGAGGCACTATTTTACCCATGCTTCCCCCACACTGTTTAATT |
| | GTGGAACAGTCACACCCCAACTTTCCTCGTGCTTCCTTCTGGG |
| | CCTTCAAGATGATAGCATTGAGGGTATTTATGATACTCTTAAGG |
| | AGGCGGCAATCATCTCCAAGACTGCTGGAGGACTCGGCATCC |
| | ACTTTCATGATTTGAGGGCAAAAGGAAGCCCCATTTCGTCATG |
| | GAGTGGTACCCACCCTGGTCTCATGGCGTTCCTCCAAATCTTTA |
| | ACGTCTCTGTGAAAAAGGTTAGCCAGGGAGGAGACAAGAGG |
| | AGAGGAGCTGCAGCCATCTATATTTCTGATTGGCATCTGGACGT |
| | GAAGGACTTTATTGACTGCAGAAAGAATGCCGGTAATGAAGAT |
| | TTGAGGACGAGGGATCTTTTCCCAGCTATCTGGGTATCTGATCT |
| | CTTCATGGAGAGAGTGAAGGCTGGGAAGAATTGGTCCCTGAT |
| | GTGCCCCCACGAGTGCCCTGGCCTTTCCGACGTCCATGGCGAA |
| | GAGTTTAAGGCGTTGTACGAGAAATATGAGGCTGAAGGCAAA |
| | GGTAAAGAGGTGGTGAAGGCACGTGCATTATTCGACCAAATTA |
| | ATTCTGCACGTATCGAAACTGGAACACCTTATGTGTGCTTTAAG |
| | GATACCATCAATAGAAAGTCTAACCAAGAAAATGTCGGCATCA |
| | TCAAGTCTTCAAATTTGTGCACTGAAATTGTCCAGTACAGTGA |
| | TTCGGAGGAAACTGCAGTGTGCAATTTGGCTTCTATCGCAGTC |
| | AACAAGTTTGTGAAGTATTCTCCCATCCCTTCCCTAAGGCCCTA |
| | TGTTGATTACCGGGAGATGAAGAGGGTTGTAAAAATCATGACC |
| | AGAAATCTCGACAAGGTGATTGATGTCAATTTCTATGCAGTTG |
| | ACAAGACCCGCATTTCCAATATGAAAACTAGGCCAATGGGATT |
| | GGGTGTGCAGGGACTAGCAGATTTGTTCTTCAAACTCAGAATC |
| | CCCTTCGAATCTGAAGAAGCGGCACTAATTAACAAGAGGATTT |
| | TTGAAACTATATACTATGGTGCCTTGGAAGCTTCATGTGAAATT |
| | GCCAAAGAAAAGGGAGAAACATATGAGCTGTTTGAGGGTAGT |
| | CCTTTGAGCAAAGGGATTTTCCAATTTGACATGGGGAAGGAAA |
| | ATATTAAGAATAGGGACATATATTTCAACTCTTTGCCAATTCAC |
| | GATTGGGAGCAATTGAGAAGGGACATTATGAAGTATGGTGTTC |
| | ACAATTCAATGTTTGTTGCTCCCATGCCTACTGCATCCACTGCA |
| | CAGATCCTGGGCAACTCTGAATCCTTTGAGCCTTTAACGTCCA |
| | ACATGTATAATCGTAATGTACTTTCAGGATCATTCCAAGTGGTG |
| | AACGAATATGTGATTAGAGAGCTCATAAAACTGGGAGAATGGA |
| | ATTCAGTAACTAAACAGAGGATTATGGCAAGTGGTGGATCTATT |
| | CAGACGCTTCCTAATATCCCTAAATCGACCAAGGAACTATTCAA |
| | AACTGTATGGGAAATTAATCCTCGTACTACTTTGGACATGGCTA |
| | TTCAGAGAGGTATGTTTGTTGACCAAGCTCAATCCCTCAACTT |
| | GTTTGTGGAAGAACCCGAACTCAGCAAGGTGCGGTCAATGAC |
| | TATGTATGCATGGGAGAAGGGGATCAAGACTCTCTATTATCTAC |
| | GCACAAAGGGCGCAGCTAGAGCTGTCCAGTTCACAGTCGACA |
| | AGAATGTACTCCAAGAAGTCAAGAAGGAAGCTCCCTCTCCTG |
| | TTGCAGCTTTTTCTGCTCCTGTCCGAGAAGAAGAAGAGGAGA |
| | AGAAGTCCTCTATTGTTGTTCCAGATCCTGCTGCTGCTCTTTTA |
| | TGTTCTATCAATAACCCTGGTGCTTGTGAAATGTGTTCTTCCTA |
| | G |
| VP28 (nucleotide | ATGGATCTTTCTTTCACTCTTTCGGTCGTGTCGGCCATCCTCGC |
| sequence; | CATCACTGCTGTGATTGCTGTATTTATTGTGATTTTTAGGTATCA |
| JX515788.1) | CAACACTGTGACCAAGACCATCGAAACCCACACAGACAATAT |
| | CGAGACAAACATGGATGAAAACCTCCGCATTCCTGTGACTGCT |
| | GAGGTTGGATCAGGCTACTTCAAGATGACTGATGTGTCCTTTG |
| | ACAGCGACACCTTGGGCAAAATCAAGATCCGCAATGGAAAGT |
| | CTGATGCACAGATGAAGGAAGAAGATGCGGATCTTGTCATCAC |
| | TCCCGTGGAGGGCCGAGCACTCGAAGTGACTGTGGGGCAGAA |
| | TCTCACCTTTGAGGGAACATTCAAGGTGTGGAACAACACATCA |
| | AGAAAGATCAACATCACTGGTATGCAGATGGTGCCAAAGATTA |
| | ACCCATCAAAGGCCTTTGTCGGTAGCTCCAACACCTCCTCCTT |
| | CACCCCCGTCTCTATTGATGAGGATGAAGTTGGCACCTTTGTG |
| | TGTGGTACCACCTTTGGCGCACCAATTGCAGCTACCGCCGGTG |
| | GAAATCTTTTCGACATGTACGTGCACGTCACCTACTCTGGCAC |
| | TGAGACCGAGTAA |

The present invention also features a method for inhibiting infection of or reducing replication of a virus belonging to the family Nimaviridae in an animal belonging to the family Penaeidae in need thereof. In some embodiments, the method comprises introducing to the animal, e.g., to a cell of said animal, a nuclease comprising one or more gene-binding moieties, wherein said one or more gene-binding moieties are configured to bind at least a portion of at least one gene of said virus.

The present invention also features a vector for introducing to an animal belonging to the family Penaeidae. In some embodiments, the vector comprises a sequence encoding at least one programmable nuclease configured to bind at least one viral gene of a virus from the family Nimaviridae. In other embodiments, the vector comprises a sequence encoding at least one programmable nuclease configured to bind at least one viral gene of a White spot syndrome virus (WSSV).

The present invention also features a feed composition for inhibiting infection of or reducing replication of a virus belonging to the family Nimaviridae in an animal belonging to the family Penaeidae in need thereof. In some embodiments, the composition comprises at least one programmable nuclease configured to bind at least one viral gene of said virus, wherein the feed composition is encapsulated and administered orally to said animal.

In some embodiments, the feed composition described herein is encapsulated with a polymer. Non-limiting examples of polymers may include but are not limited to poly (lactic-co-glycolic acid) (PLGA), chitosan, chitosan/alginate, polylactic acid, PLA/PLGA, or mannosylated chitosan. In some embodiments, the polymer may be formed into a nanoparticle or a microparticle.

In other embodiments, the feed composition described herein is encapsulated with a nanoparticle. In some embodiments, the nanoparticle is a chitosan nanoparticle. In other embodiments, the nanoparticle is a poly (γ-glutamic acid) (PGA) nanoparticle. In further embodiments, the nanoparticle is an alginate nanoparticle. In some embodiments, the feed composition described herein is encapsulated with a microparticle. In some embodiments, the microparticle is a chitosan microparticle, a poly (γ-glutamic acid) microparticle or an alginate microparticle. In some embodiments, the nanoparticle or the microparticle may further comprise a moiety comprising a chitosan binding peptide. In other embodiments, the nanoparticle or the microparticle may further comprise a moiety comprising a chitosan binding domain.

In some embodiments, the encapsulated feed composition described herein is attached to a feed particle. In some embodiments, the encapsulated feed composition described herein is incorporated into an oil and sprayed onto the feed. In other embodiments, the encapsulated feed composition described herein is top-coated on the feed. In other embodiments, the encapsulated feed composition described herein is incorporated into a feed particle. In further embodiments, the encapsulated feed composition described herein is cold extruded.

In some embodiments, the animal is a live animal, e.g., the animal is intact. In some embodiments, the method is for in vivo delivery of the nuclease. In some embodiments, the virus belongs to the genus Whispovirus. In some embodiments, the virus is White spot syndrome virus (WSSV). In some embodiments, the animal belongs to the genus Litopenaeus. In some embodiments, the animal is Litopenaeus vannamei.

In some embodiments, methods and compositions described herein are directed towards inhibiting infection of or reducing replication of a White spot syndrome virus (WSSV) species. Non-limiting examples of WSSV species genomes include but are not limited to GCA_003024735.1, GCA 003972705.1, GCA-00848085.2, GCA_003972345.1, GCA_003972365.1, GCA 003972405.1, GCA_003972425.1, GCA_003972445.1, GCA_003972465.1, GCA 003972485.1, GCA_003972505.1, GCA_003972525.1, GCA_00397545. It should be noted that other WSSV species genomes may be used in the methods and compositions described herein. In some embodiments, any gene (i.e., structural or nonstructural) within the WSSV genome may be targeted with the methods and compositions described herein. Non-limiting examples of WSSV genes may be targeted by the methods or compositions described herein include but are not limited to DNA polymerase, RR1, VP28, ICP11, VP19, VP26, collagen-like protein (WSSV-CLP), ie1, ie2, ie3, TK-TMK, RR2, VP19, VP24, VP35, VP39, VP12, VP190, or VP15 (see Table B for further examples). Furthermore, because WSSV is a double strand DNA virus, one skilled in the art would understand that either or both DNA strands may be targeted by the gene binding moieties or the programmable nucleases described herein.

In some embodiments, the one or more gene binding moieties are configured to bind a plurality of different portions of at least one gene of said virus. In other embodiments, the one or more gene binding moieties are configured to bind at least a portion of at least two genes of said virus. In some embodiments, the programmable nuclease is configured to bind a plurality of different portions of at least one gene of said virus. In other embodiments, the programmable nuclease is configured to bind at least a portion of at least two genes of said virus.

In some embodiments, the one or more gene binding moieties or the programmable nucleases described herein are configured to bind to virus genes that are structural, nonstructural, or a combination thereof. In other embodiments, the one or more gene binding moieties or the programmable nucleases described herein are configured to bind to any gene within the genome of the virus including but not limited to DNA polymerase, RR1, VP28, ICP11, VP19, VP26, collagen-like protein (WSSV-CLP) or a combination thereof.

In some embodiments, the portion of a gene of the virus (e.g., for targeting) comprises at least 15, at least 16, at least 17, at least 18, at least 19, at least 20, at least 21, at least 22, at least 23, at least 24, at least 25, at least 26, at least 27, at least 28, at least 29, or at least 30 consecutive nucleotides of said gene. In other embodiments, the portion of a gene of the virus may comprise about 15-30 consecutive nucleotides, about 20-30 consecutive nucleotides, about 25-30 consecutive nucleotides, about 20-30 consecutive nucleotides, about 20-25 consecutive nucleotides, or about 25-30 consecutive nucleotides.

In some embodiments, the one or more gene binding moieties comprises at least 80%, at least 90%, at least 95%, at least 99%, at least 100% homology to the portion of a gene of the virus (e.g., the wild type target sequence). In other embodiments, the programmable nuclease may comprise at least 80%, at least 90%, at least 95%, at least 99%, at least 100% homology to the portion of said gene of said virus that the programmable nuclease is configured to bind to. In some embodiments, the one or more gene binding moieties comprises at least 80%, at least 90%, at least 95%, at least 99%, at least 100% homology to the virus sequence being targeted by the one or more gene binding moieties. In some embodiments, the programmable nuclease comprises at least 80%, at least 90%, at least 95%, at least 99%, at least 100% homology to the virus sequence being targeted (i.e., the sequence the programmable nuclease is configured to bind to).

In some embodiments, the nuclease is a programmable nuclease comprising at least one of a CRISPR-associated (Cas) polypeptide, a zinc finger nuclease (ZFN), a transcription activator-like effector nuclease (TALEN), or a combination thereof. In other embodiments, the nuclease is a programmable nuclease comprises a CRISPR-associated (Cas) polypeptide, wherein said Cas polypeptide is a type I CRISPR-associated (Cas) polypeptide, a type II CRISPR-associated (Cas) polypeptide, a type III CRISPR-associated (Cas) polypeptide, a type IV CRISPR-associated (Cas) polypeptide, a type V CRISPR-associated (Cas) polypeptide, a type VI CRISPR-associated (Cas) polypeptide. In some embodiments, the gene-binding moiety of said nuclease comprises a heterologous RNA polynucleotide configured to hybridize to at least a portion of one or more genes of said virus. In other embodiments, the gene-binding moiety of said nuclease comprises a heterologous RNA polynucleotide configured to hybridize a plurality of different portions of at least one gene of said virus. In some embodiments, the nuclease comprises a ribonucleoprotein complex comprising a Cas polypeptide and at least one, at least two, or at least three heterologous RNA polynucleotides configured to hybridize to said one or more genes of said virus.

In some embodiments, introducing the nuclease comprising one or more gene-binding moieties to said cell of said animal comprises contacting said cell with said nuclease. In some embodiments, said nuclease comprises a ribonucleoprotein complex comprising a Cas polypeptide and at least one, at least two, or at least three heterologous RNA polynucleotides configured to hybridize to said one or more genes of said virus. In other embodiments, introducing the nuclease comprising one or more gene-binding moieties to said cell of said animal comprises contacting said cell with a capped mRNA comprising a sequence encoding said nuclease. In some embodiments, the nuclease comprises a Cas polypeptide, wherein introducing a nuclease comprising a gene-binding moiety to said cell of said animal further comprises contacting said cell with at least one, at least two, or at least three heterologous RNA polynucleotides configured to hybridize to said one or more genes of said virus. In some embodiments, capped mRNA and said heterologous RNA polynucleotide are separate RNAs. In further embodiments, introducing a nuclease comprising one or more gene-binding moieties to said cell of said animal comprises contacting said cell with a vector comprising a sequence encoding said nuclease. In some embodiments, the nuclease comprises a Cas polypeptide, wherein said vector further encodes at least one, at least two, or at least three heterologous RNA polynucleotides configured to hybridize to said one or more genes of said virus. In some embodiments, introducing occurs in vivo, ex vivo, or in vitro.

In some embodiments, the nuclease cleaves viral genomic DNA encoding said one or more genes of said virus within said cell of said animal. In other embodiments, the nuclease cleaves double stranded viral DNA encoding said one or more genes of said virus within said cell of said animal. In further embodiments, the nuclease cleaves double stranded viral DNA encoding said one or more genes therefore creating a double stranded break. In some embodiments, the viral genomic DNA encoding said one or more genes of said virus further comprises a protospacer adjacent motif (PAM) site. In other embodiments the viral genomic DNA encoding said one or more genes of said virus does not comprise a PAM site. In some embodiments, the PAM site is at least 2 nucleotides, at least 3 nucleotides, at least 5 nucleotides, at least 7 nucleotides, at least 8 nucleotides, or at least 10 nucleotides long.

In some embodiments, the vector is a plasmid, a minicircle, or a viral vector. In some embodiments, the vector is a viral vector, e.g., said viral vector is a baculoviral vector.

In some embodiments, methods and compositions described herein result in a delay of or reduced mortality of said animal upon infection with said virus belonging to the family Nimaviridae. In some embodiments, the delay of or reduced mortality of said animal is compared to an animal not treated with said methods or administered said compositions described herein. In other embodiments, methods and compositions described herein result in a delay of or reduced mortality of an animal belonging to the family Pendeidue upon infection with White spot syndrome virus (WSSV). In some embodiments, the delay of or reduced mortality of said animal belonging to the family Pendeidae is compared to an animal not treated with said methods or administered said compositions described herein. In further embodiments, methods and compositions described herein result in a delay of or reduced mortality of a Litopenaeus vannamei upon infection with White spot syndrome virus (WSSV). In some embodiments, the delay of or reduced mortality of the Litopenaeus vannamei is compared to a Litopenaeus vannamei not treated with said methods or administered said compositions described herein.

In some embodiments, the step of introducing to a cell of said animal said nuclease comprises injecting said animal with said nuclease or a vector encoding said nuclease. In other embodiments, the step of introducing to a cell of said animal said nuclease comprises administering orally to said animal said nuclease or a vector encoding said nuclease. In some embodiments, the step of introducing to a cell of an animal belonging to the family Pendeidae said nuclease comprises injecting the animal belonging to the family Pendeidae with said nuclease or a vector encoding said nuclease. In other embodiments, introducing to a cell of an animal belonging to the family Penaeidae said nuclease comprises administering orally to the animal belonging to the family Penaeidae said nuclease or a vector encoding said nuclease. In some embodiments, the step of introducing to a cell of a Litopenaeus vannamei said nuclease comprises injecting the Litopenaeus vannamei with said nuclease or a vector encoding said nuclease. In other embodiments, the step of introducing to a cell of a Litopenaeus vannamei said nuclease comprises administering orally to the Litopenaeus vannamei said nuclease or a vector encoding said nuclease.

The present invention also features a method for inhibiting infection of or reducing replication of a White spot syndrome virus (WSSV) in an animal belonging to the family Pengeidae in need thereof. In some embodiments, the method comprises introducing to a cell of said animal belonging to the family Pendeidae a nuclease comprising one or more gene-binding moieties, wherein said one or more gene-binding moieties are configured to bind at least a portion of at least one gene of said virus. In other embodiments, the present invention features a method for inhibiting infection of or reducing replication of a White spot syndrome virus (WSSV) in an animal belonging to the genus Litopenaeus in need thereof. In some embodiment, the method comprises introducing to a cell of said animal belonging to the genus Litopenaeus a nuclease comprising one or more gene-binding moieties, wherein said one or more gene-binding moieties are configured to bind at least a portion of at least one gene of said virus. In further embodiments, the present invention also features a method for inhibiting infection of or reducing replication of a White spot syndrome virus (WSSV) in a Litopenaeus vannamei in need thereof. In some embodiments, the method comprises introducing to a cell of the Litopenaeus vannamei nuclease comprising one or more gene-binding moieties, wherein said one or more gene-binding moieties are configured to bind at least a portion of at least one gene of said virus.

In some embodiments, the present invention also features a method for inhibiting infection of or reducing replication of a White spot syndrome virus (WSSV) in an animal belonging to the genus Litopenaeus in need thereof. In some embodiments, the method comprises introducing to a cell of said animal belonging to the genus Litopenaeus a CRISPR-associated (Cas) polypeptide nuclease comprising one or more gene-binding moieties, wherein said one or more gene-binding moieties are configured to bind at least a portion of at least one gene of said virus. In other embodiments, the present invention features a method for inhibiting infection of or reducing replication of a White spot syndrome virus (WSSV) in an animal belonging to the genus Litopenaeus in need thereof. In some embodiments, the method comprises introducing to a cell of said animal belonging to the genus Litopenaeus a transcription activator-like effector nuclease (TALEN) comprising one or more gene-binding moieties, wherein said one or more gene-binding moieties are configured to bind at least a portion of at least one gene of said virus. In further embodiments, the present invention features a method for inhibiting infection of or reducing replication of a White spot syndrome virus (WSSV) in an animal belonging to the genus Litopenaeus in need thereof. In some embodiments, the method comprises introducing to a cell of said animal belonging to the genus Litopenaeus a zinc finger nuclease (ZFN) comprising one or more gene-binding moieties, wherein said one or more gene-binding moieties are configured to bind at least a portion of at least one gene of said virus.

In some embodiments, the present invention features a feed composition for inhibiting infection of or reducing replication of a White spot syndrome virus (WSSV) in an animal belonging to the family Pendeidae in need thereof. In some embodiments, the composition comprises at least one programmable nuclease configured to bind at least one viral gene of said virus, wherein the feed composition is encapsulated and administered orally to said animal belonging to the family Penaeidae. In other embodiments, the present invention features a feed composition for inhibiting infection of or reducing replication of a White spot syndrome virus (WSSV) in an animal belonging to the genus Litopenaeus in need thereof. In some embodiments, the composition comprises at least one programmable nuclease configured to bind at least one viral gene of said virus, wherein the feed composition is encapsulated and administered orally to said animal belonging to the genus Litopenaeus. In further embodiments, the present invention features a feed composition for inhibiting infection of or reducing replication of a White spot syndrome virus (WSSV) in a Litopenaeus vannamei in need thereof. In some embodiments, the composition comprises at least one programmable nuclease configured to bind at least one viral gene of said virus, wherein the feed composition is encapsulated and administered orally to said Litopenaeus vannamei.

In some embodiments, the present invention features a feed composition for inhibiting infection of or reducing replication a White spot syndrome virus (WSSV) in an animal belonging to the genus Litopenaeus in need thereof, the composition comprising at least one programmable nuclease configured to bind at least one viral gene of said virus, wherein the feed composition is encapsulated in a chitosan nanoparticle wherein the chitosan nanoparticle moiety further comprises a chitosan binding domain, wherein the composition is administered orally to said animal.

In some embodiments, the present invention features a feed composition for inhibiting infection of or reducing replication of a White spot syndrome virus (WSSV) in a Litopenaeus vannamei in need thereof. In some embodiments, the composition comprises at least a CRISPR-associated (Cas) polypeptide nuclease configured to bind at least one viral gene of said virus. In other embodiments, the feed composition is encapsulated in a chitosan nanoparticle wherein the chitosan nanoparticle moiety further comprises a chitosan binding domain. In further embodiment, the composition is administered orally to said Litopenaeus vannamei. In other embodiments, the present invention features a feed composition for inhibiting infection of or reducing replication of a White spot syndrome virus (WSSV) in a Litopenaeus vannamei in need thereof. In some embodiments, the composition comprises at least a transcription activator-like effector nuclease (TALEN) configured to bind at least one viral gene of said virus. In other embodiments, wherein the feed composition is encapsulated in a chitosan nanoparticle wherein the chitosan nanoparticle moiety further comprises a chitosan binding domain. In further embodiments, the composition is administered orally to said Litopenaeus vannamei. In further embodiments, the present invention features a feed composition for inhibiting infection of or reducing replication of a White spot syndrome virus (WSSV) in a Litopenaeus vannamei in need thereof. In some embodiments, the composition comprises at least a zinc finger nuclease (ZFN) configured to bind at least one viral gene of said virus. In other embodiments, the feed composition is encapsulated in a chitosan nanoparticle wherein the chitosan nanoparticle moiety further comprises a chitosan binding domain. In further embodiments, the composition is administered orally to said Litopendeus vannamei.

In some embodiments, the present invention features a vector for introducing to an animal belonging to the genus Litopenaeus. In some embodiments, the vector comprises a sequence encoding at least one programmable nuclease configured to bind at least one viral gene of a White spot syndrome virus (WSSV). In other embodiments, the present invention features a vector for introducing to a Litopenaeus vannamei. In some embodiments, the vector comprises a sequence encoding at least one programmable nuclease configured to bind at least one viral gene of a White spot syndrome virus (WSSV).

### EXAMPLES

### Example 1: Expression of the Crispr/Cas Cassette and Delivery of in Vitro Transcribed mRNAs, Plasmid DNA or Recombinant Baculovirus

Different vehicles of delivery of CRISPR/Cas cassette are possible to treat WSSV-infected shrimp: 1) in vitro transcribed RNAs representing the sgRNA and Cas 9 nuclease, 2) plasmid DNA or 3) baculovirus carrying sgRNA and Cas 9 mRNA. Direct injection of these is used for maximum therapeutic impact and to test efficacy.

TABLE 1
Target sequences for DNApol, RR1, and VP28 used in vectors described herein

| | | | SEQ | | SEQ |
|---|---|---|---|---|---|
| | | Virus | ID | Targeting sequence (reverse | ID |
| # | Gene | Sequence Targeted | NO: | complement of virus sequence) | NO: |
| 1 | DNApol | GGTACAACTGGATTTGG | 1 | CCCCCCAAATCCAGTTGTACC | 10 |
| | | GGG | | | |
| 2 | DNApol | ACCACTATTGTAAACCG | 2 | CATCGGTTTACAATAGTGGT | 11 |
| | | ATG | | | |
| 3 | DNApol | CAAGTGTGAACCCAAA | 3 | GATTTTTGGGTTCACACTTG | 12 |
| | | AATC | | | |
| 4 | RR1 | GTTCCACAATTAAACAG | 4 | ACACTGTTTAATTGTGGAAC | 13 |
| | | TGT | | | |
| 5 | RR1 | TCTTGGAAGGAGATGGT | 5 | GCTACCATCTCCTTCCAAGA | 14 |
| | | AGC | | | |
| 6 | RR1 | TTGAGGCACGCAATCCA | 6 | GGGTGGATTGCGTGCCTCAA | 15 |
| | | CCC | | | |
| 7 | VP28 | GGATCTTTCTTTCACTCT | 7 | AAAGAGTGAAAGAAAGATCC | 16 |
| | | TT | | | |
| 8 | VP28 | CACAGCAGTGATGGCG | 8 | TCCTCGCCATCACTGCTGTG | 17 |
| | | AGGA | | | |
| 9 | VP28 | TGTGTGGGTTTCGATGG | 9 | AGACCATCGAAACCCACACA | 18 |
| | | TCT | | | |

Specific Pathogen Free (SPF) shrimp were infected with WSSV under laboratory conditions, then 6-12 hours post-infection the animals were injected with above described delivery vehicles to test efficacy.

Seven days post-injection, all animals had died and were dissected to analyze muscle tissues at the site of injection, hemolymph, hepatopancreas, and ovary samples to determine targeted insertion/deletion mutations (INDELS) in WSSV genes at the site of dsDNA break. DNA was isolated from dissected tissues, and the target loci were amplified using Phusion Hot Start II high-fidelity DNA polymerase (New England Biolabs) the target loci were sequence sequenced, and mutations flanking the site of dsDNA break were identified by comparing to the wild-type WSSV sequence for the corresponding gene.

### Example 2: Crispr/Cas-Based Editing of WSSV Genome Using Primary Cell Culture in Shrimp

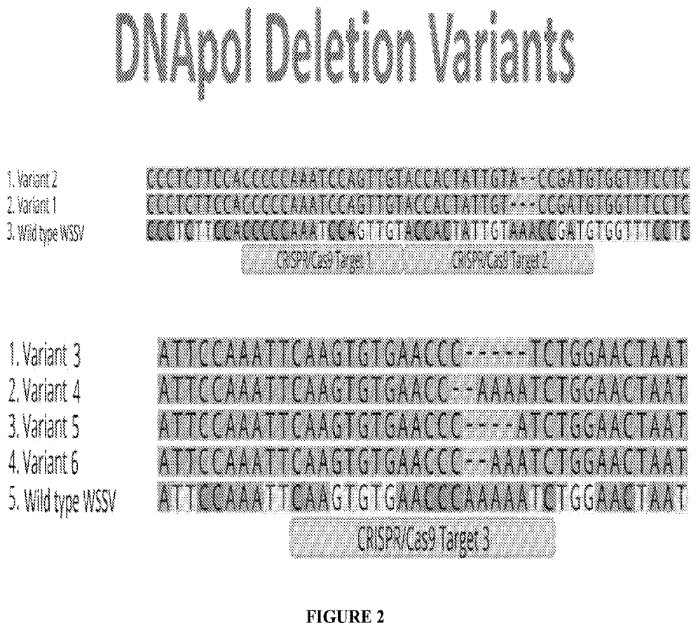

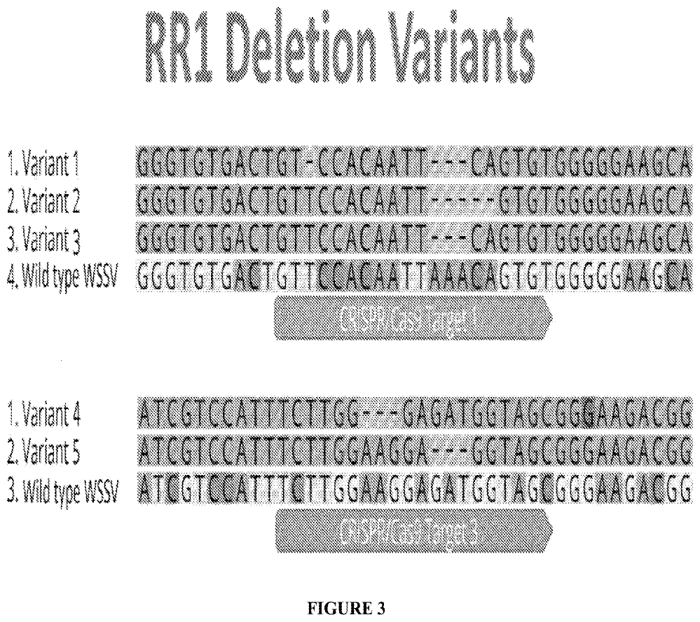

Shrimp primary cell culture was used to demonstrate CRISPR/Cas-based genome editing of the WSSV genome. Shrimp primary hemocyte culture was performed following a published protocol (see e.g., S. K. George et al. Multiplication of Taura syndrome virus in primary hemocyte culture of shrimp (Penaeus vannamei). J Virol Methods 172, 54-59). Primary culture were then transformed using either a liposome mediated delivery of the plasmid DNA or in vitro transcribed RNA as described in Example 1 or a recombinant baculovirus carrying sgRNA and Cas 9 mRNA cassette as described in Example 1. Upon transformation of the primary culture, confirmation of the INDELS in the target loci was carried out by isolating the genomic DNA and subsequently amplifying and sequencing the target loci by next-generation sequencing (NGS), as shown in FIG. 2 and FIG. 3.

### Example 3: Efficacy of Crispr/Cas-Based Antiviral Therapy Against White Spot Syndrome Disease in Shrimp

Recombinant bacterial or yeast biomass or homogenates of insect cells expressing different components of the CRISPR/Cas-system, as described in the Example 1, was mixed with shrimp diet. Specific-Pathogen-Free (SPF) shrimp were fed the diet for 7 days before challenging the animals with WSSV following OIE protocol (see e.g., Office international des epizooties. Mamial of diagnostic tests for aquatic animal diseases. (World Organization for Animal Health, Paris, France, ed. 6th, 2009). After WSSV challenge, the animals were fed CRISPR/Cas containing diets. SPF shrimp that were maintained on a regular diet (feed with no CRISPR/Cas component derived from bacteria, yeast, or insect cells) then challenged with WSSV were the positive control. SPF shrimp that were maintained on a regular diet (feed with no CRISPR/Cas component derived from bacteria/yeast/insect cells) and not challenged with WSSV are the negative control.

Another treatment includes shrimp infected with WSSV and are fed with a diet containing different components of the CRISPR/Cas system at 6 hours post-infection. Clinical signs and mortalities are recorded. Moribund animals are collected throughout the experiment. Similarly, all surviving animals are collected on termination. Moribund and surviving animals are preserved in Davidson Fixative for histopathology (see e.g., Office international des epizooties. Manual of diagnostic tests for aquatic animal diseases. (World Organization for Animal Health, Paris, France, ed. 6th, 2009 and Office international des epizooties. Animal Health Code. (World Organization for Animal Health, Paris, France, ed. 13th, 2010), pp. 301) Prior to fixing samples for histopathology, pleopod samples are collected for determining WSSV load by real-time PCR.

### Example 4: Production of Initial Crispr/Cas Constructs Targeting Essential WSSV Genes

Target Selection: Three WSSV target genes were selected: DNA polymerase, the major capsid protein VP28 and ribonucleotide reductase subunit 1 (abbreviated herein as DNApol, VP28, and RR1). These were selected due to their integral involvement in viral replication of the viral genome (e.g., DNApol and RR1) and in the overall structural integrity of the virus particle (i.e., VP28). DNApol and RR1 genes are early genes in the WSSV life cycle and VP28 is a late gene.

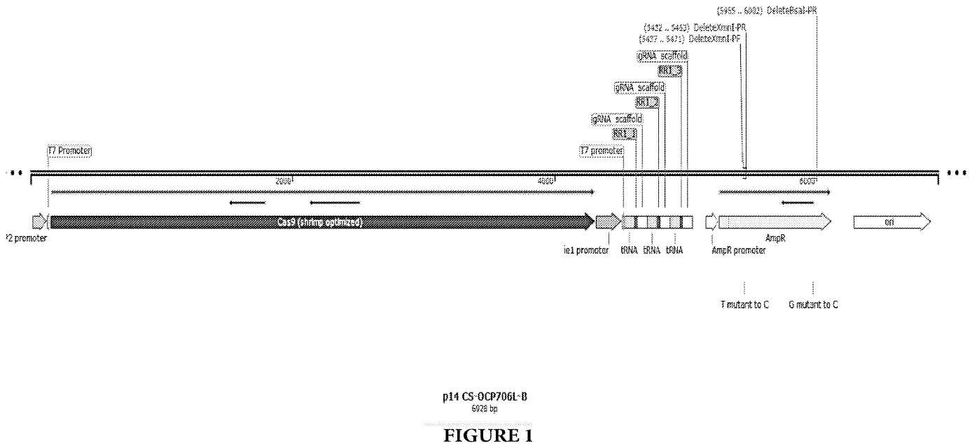

Vector Design: The WSSV sequence used was based on the China strain of WSSV (Accession number AF332093.3). Vectors can be designed containing a single guide RNA (sgRNA) targeting a site on the WSSV gene one at a time (e.g., would need 9 different vectors to properly address these three genes) However, use of multiplexed vectors would be expected to improve the probability of gene editing and inhibition through hitting three different targets on the gene. Three multiplexed vectors that each have three target sites (sgRNAs) were produced and each targets a single gene (e.g., VP28, DNApol or RR1, see Table 1 for targeting sequences). Targeting more than one site on the gene assures that the target gene function is eliminated despite variability of indel formation. The overall approach for all three vectors (p12/VP28, p13/DNApol, and p14/RR1) was the same and all were built on a backbone vector that had several unique features making this vector optimized for use in shrimp. The p14 plasmid is diagramed in FIG. 1 as an example of the approach used for multiplexed vector design. This vector contains the following elements: 1) a codon optimized Cas9 gene; the sequence was modified to contain codons used by Litopenaeus vannamei (Pacific white shrimp), 2) the P2 promoter of infectious hypodermal and hematopoietic necrosis virus (IHHNV) of shrimp was inserted to drive expression of this gene in shrimp, 3) a multiplex cassette containing sequence to produce three specific guide RNAs targeting RR1 (labeled RR1 1, RR1_2 and RR1-3) and the crRNA, 4) upstream of this cassette were the ie1 promoter of WSSV and the T7 promoter to drive production of these sgRNAs in shrimp or bacteria, respectively; alongside nonessential components facilitating replication such as 5) AmpR gene and promoter, a selective pressure cassette for amplification in bacteria, and 6) a bacterial origin of replication (ori).

TABLE 2
Component sequences that can be used in vectors described herein

| | | | SEQ |
|---|---|---|---|
| | Vector | | ID |
| # | Element | Sequence | NO: |
| 1 | Litopenaeus | ATGGACAAGAAGTACTCCATCGGCCTGGACATCGGCA | 19 |
| | vannamei | CCAACTCCGTGGGCTGGGCCGTGATCACCGACGAGTA | |
| | optimized | CAAGGTGCCCTCCAAGAAGTTCAAGGTGCTGGGCAAC | |
| | Cas9 | ACCGACCGCCACTCCATCAAGAAGAACCTGATCGGCG | |
| | | CCCTGCTGTTCGACTCCGGCGAGACCGCCGAGGCCAC | |
| | | CCGCCTGAAGCGCACCGCCCGCCGCCGCTACACCCGC | |
| | | CGCAAGAACCGCATCTGCTACCTGCAGGAGATCTTCTC | |
| | | CAACGAGATGGCCAAGGTGGACGACTCCTTCTTCCAC | |
| | | CGCCTGGAGGAGTCCTTCCTGGTGGAGGAGGACAAGA | |
| | | AGCACGAGCGCCACCCCATCTTCGGCAACATCGTGGA | |
| | | CGAGGTGGCCTACCACGAGAAGTACCCCACCATCTAC | |
| | | CACCTGCGCAAGAAGCTGGTGGACTCCACCGACAAGG | |
| | | CCGACCTGCGCCTGATCTACCTGGCCCTGGCCCACATG | |
| | | ATCAAGTTCCGCGGCCACTTCCTGATCGAGGGCGACCT | |
| | | GAACCCCGACAACTCCGACGTGGACAAGCTGTTCATC | |
| | | CAGCTGGTGCAGACCTACAACCAGCTGTTCGAGGAGA | |
| | | ACCCCATCAACGCCTCCGGCGTGGACGCCAAGGCCAT | |
| | | CCTGTCCGCCCGCCTGTCCAAGTCCCGCCGCCTGGAG | |
| | | AACCTGATCGCCCAGCTGCCCGGCGAGAAGAAGAACG | |
| | | GCCTGTTCGGCAACCTGATCGCCCTGTCCCTGGGCCTG | |
| | | ACCCCCAACTTCAAGTCCAACTTCGACCTGGCCGAGG | |
| | | ACGCCAAGCTGCAGCTGTCCAAGGACACCTACGACGA | |
| | | CGACCTGGACAACCTGCTGGCCCAGATCGGCGACCAG | |
| | | TACGCCGACCTGTTCCTGGCCGCCAAGAACCTGTCCG | |
| | | ACGCCATCCTGCTGTCCGACATCCTGCGCGTGAACACC | |
| | | GAGATCACCAAGGCCCCCCTGTCCGCCTCCATGATCAA | |
| | | GCGCTACGACGAGCACCACCAGGACCTGACCCTGCTG | |
| | | AAGGCCCTGGTGCGCCAGCAGCTGCCCGAGAAGTACA | |
| | | AGGAGATCTTCTTCGACCAGTCCAAGAACGGCTACGC | |
| | | CGGCTACATCGACGGCGGCGCCTCCCAGGAGGAGTTC | |
| | | TACAAGTTCATCAAGCCCATCCTGGAGAAGATGGACG | |
| | | GCACCGAGGAGCTGCTGGTGAAGCTGAACCGCGAGG | |
| | | ACCTGCTGCGCAAGCAGCGCACCTTCGACAACGGCTC | |
| | | CATCCCCCACCAGATCCACCTGGGCGAGCTGCACGCC | |
| | | ATCCTGCGCCGCCAGGAGGACTTCTACCCCTTCCTGAA | |
| | | GGACAACCGCGAGAAGATCGAGAAGATCCTGACCTTC | |
| | | CGCATCCCCTACTACGTGGGCCCCCTGGCCCGCGGCAA | |
| | | CTCCCGCTTCGCCTGGATGACCCGCAAGTCCGAGGAG | |
| | | ACCATCACCCCCTGGAACTTCGAGGAGGTGGTGGACA | |
| | | AGGGCGCCTCCGCCCAGTCCTTCATCGAGCGCATGAC | |
| | | CAACTTCGACAAGAACCTGCCCAACGAGAAGGTGCTG | |
| | | CCCAAGCACTCCCTGCTGTACGAGTACTTCACCGTGTA | |
| | | CAACGAGCTGACCAAGGTGAAGTACGTGACCGAGGG | |
| | | CATGCGCAAGCCCGCCTTCCTGTCCGGCGAGCAGAAG | |
| | | AAGGCCATCGTGGACCTGCTGTTCAAGACCAACCGCA | |
| | | AGGTGACCGTGAAGCAGCTGAAGGAGGACTACTTCAA | |
| | | GAAGATCGAGTGCTTCGACTCCGTGGAGATCTCCGGC | |
| | | GTGGAGGACCGCTTCAACGCCTCCCTGGGCACCTACC | |
| | | ACGACCTGCTGAAGATCATCAAGGACAAGGACTTCCT | |
| | | GGACAACGAGGAGAACGAGGACATCCTGGAGGACAT | |
| | | CGTGCTGACCCTGACCCTGTTCGAGGACCGCGAGATG | |
| | | ATCGAGGAGCGCCTGAAGACCTACGCCCACCTGTTCG | |
| | | ACGACAAGGTGATGAAGCAGCTGAAGCGCCGCCGCTA | |
| | | CACCGGCTGGGGCCGCCTGTCCCGCAAGCTGATCAAC | |
| | | GGCATCCGCGACAAGCAGTCCGGCAAGACCATCCTGG | |
| | | ACTTCCTGAAGTCCGACGGCTTCGCCAACCGCAACTT | |
| | | CATGCAGCTGATCCACGACGACTCCCTGACCTTCAAG | |
| | | GAGGACATCCAGAAGGCCCAGGTGTCCGGCCAGGGC | |
| | | GACTCCCTGCACGAGCACATCGCCAACCTGGCCGGCT | |
| | | CCCCCGCCATCAAGAAGGGCATCCTGCAGACCGTGAA | |
| | | GGTGGTGGACGAGCTGGTGAAGGTGATGGGCCGCCAC | |
| | | AAGCCCGAGAACATCGTGATCGAGATGGCCCGCGAGA | |
| | | ACCAGACCACCCAGAAGGGCCAGAAGAACTCCCGCG | |
| | | AGCGCATGAAGCGCATCGAGGAGGGCATCAAGGAGCT | |
| | | GGGCTCCCAGATCCTGAAGGAGCACCCCGTGGAGAAC | |
| | | ACCCAGCTGCAGAACGAGAAGCTGTACCTGTACTACC | |
| | | TGCAGAACGGCCGCGACATGTACGTGGACCAGGAGCT | |
| | | GGACATCAACCGCCTGTCCGACTACGACGTGGACCAC | |
| | | ATCGTGCCCCAGTCCTTCCTGAAGGACGACTCCATCGA | |
| | | CAACAAGGTGCTGACCCGCTCCGACAAGAACCGCGGC | |
| | | AAGTCCGACAACGTGCCCTCCGAGGAGGTGGTGAAG | |
| | | AAGATGAAGAACTACTGGCGCCAGCTGCTGAACGCCA | |
| | | AGCTGATCACCCAGCGCAAGTTCGACAACCTGACCAA | |
| | | GGCCGAGCGCGGCGGCCTGTCCGAGCTGGACAAGGC | |
| | | CGGCTTCATCAAGCGCCAGCTGGTGGAGACCCGCCAG | |
| | | ATCACCAAGCACGTGGCCCAGATCCTGGACTCCCGCAT | |
| | | GAACACCAAGTACGACGAGAACGACAAGCTGATCCGC | |
| | | GAGGTGAAGGTGATCACCCTGAAGTCCAAGCTGGTGT | |
| | | CCGACTTCCGCAAGGACTTCCAGTTCTACAAGGTGCG | |
| | | CGAGATCAACAACTACCACCACGCCCACGACGCCTAC | |
| | | CTGAACGCCGTGGTGGGCACCGCCCTGATCAAGAAGT | |
| | | ACCCCAAGCTGGAGTCCGAGTTCGTGTACGGCGACTA | |
| | | CAAGGTGTACGACGTGCGCAAGATGATCGCCAAGTCC | |
| | | GAGCAGGAGATCGGCAAGGCCACCGCCAAGTACTTCT | |
| | | TCTACTCCAACATCATGAACTTCTTCAAGACCGAGATC | |
| | | ACCCTGGCCAACGGCGAGATCCGCAAGCGCCCCCTGA | |
| | | TCGAGACCAACGGCGAGACCGGCGAGATCGTGTGGG | |
| | | ACAAGGGCCGCGACTTCGCCACCGTGCGCAAGGTGCT | |
| | | GTCCATGCCCCAGGTGAACATCGTGAAGAAGACCGAG | |
| | | GTGCAGACCGGCGGCTTCTCCAAGGAGTCCATCCTGC | |
| | | CCAAGCGCAACTCCGACAAGCTGATCGCCCGCAAGAA | |
| | | GGACTGGGACCCCAAGAAGTACGGCGGCTTCGACTCC | |
| | | CCCACCGTGGCCTACTCCGTGCTGGTGGTGGCCAAGG | |
| | | TGGAGAAGGGCAAGTCCAAGAAGCTGAAGTCCGTGA | |
| | | AGGAGCTGCTGGGCATCACCATCATGGAGCGCTCCTCC | |
| | | TTCGAGAAGAACCCCATCGACTTCCTGGAGGCCAAGG | |
| | | GCTACAAGGAGGTGAAGAAGGACCTGATCATCAAGCT | |
| | | GCCCAAGTACTCCCTGTTCGAGCTGGAGAACGGCCGC | |
| | | AAGCGCATGCTGGCCTCCGCCGGCGAGCTGCAGAAGG | |
| | | GCAACGAGCTGGCCCTGCCCTCCAAGTACGTGAACTT | |
| | | CCTGTACCTGGCCTCCCACTACGAGAAGCTGAAGGGC | |
| | | TCCCCCGAGGACAACGAGCAGAAGCAGCTGTTCGTGG | |
| | | AGCAGCACAAGCACTACCTGGACGAGATCATCGAGCA | |
| | | GATCTCCGAGTTCTCCAAGCGCGTGATCCTGGCCGACG | |
| | | CCAACCTGGACAAGGTGCTGTCCGCCTACAACAAGCA | |
| | | CCGCGACAAGCCCATCCGCGAGCAGGCCGAGAACATC | |
| | | ATCCACCTGTTCACCCTGACCAACCTGGGCGCCCCCGC | |
| | | CGCCTTCAAGTACTTCGACACCACCATCGACCGCAAG | |
| | | CGCTACACCTCCACCAAGGAGGTGCTGGACGCCACCC | |
| | | TGATCCACCAGTCCATCACCGGCCTGTACGAGACCCGC | |
| | | ATCGACCTGTCCCAGCTGGGCGGCGACGGCTCCCCCA | |
| | | AGAAGAAGCGCAAGGTGTCCTCCGGCGGCGCCGCCG | |
| | | GCTGA | |
| 2 | P2 promoter | CTGCGAGCGCTTCGCAGAAACCGTTACAACCTATGAC | 20 |
| | of infectious | GTCATAGGTCCTATATAAGAGATGACGGACTCACCGGT | |
| | hypodermal | CTCCCAGTTTCTAACTGACGAGTGAAGAGGCTATTCC | |
| | and | | |
| | hematopoietic | | |
| | necrosis virus | | |
| | (IHHNV) of | | |
| | shrimp | | |
| 3 | ie1 | TAATGAATTTCCTTGTTACTCATTTATTCCTAGAAATGG | 21 |
| | promoter of | TGTAATCGCTGTTGTGGGCGGAGCATATTTGTGTATATA | |
| | WSSV | AGAGCCCGTGTTAGCTCCTCGATTCAGTCACAAGAGC | |
| | | GCACACACACGCTTATAACTAGCTCTCTCTCTCCACTC | |
| | | AAGATGGCCTTTAATTTTGAAGACTCTACAAATCTCTTT | |
| | | GCCA | |
| 4 | ProAV | AAATTGTATCTGTTATTTAGTTTTATTATTACTGAAGCCA | 150 |
| | | CTCTTTCGCTTAGACTATAGAATTTCACGATATTCTTTTC | |
| | | TAAACGTTTGTGAACTAAGAAACCTACCCGTAAATCCT | |
| | | GCTTCCTACCCTGGGGTTTACCGCTCCAGATAGAGTGC | |
| | | GTGTCCATCATTTAATATTTTCTCTTCTTCGATTATATAC | |
| | | GTGTGTGTGTGTGTGTGTGTGTGTGTGTGTGTGTGTGT | |
| | | GTGTGTGTGTGTGTGTGTGTGTGTGTGTGTGTGTGTGT | |
| | | GTGTGTGTGTGTGTGTATGTGTATGTGTATGTGTATGTG | |
| | | TATGTGTATGTGTATGTGTATATATATGAGTATATATACAT | |
| | | ATGTGTATATATGTGTGTATATATGTGTGTGTGTGTGTGT | |
| | | GTGTGTGTGTGTGTGTGTGTGTGTGTGTGTGTGTGTGT | |
| | | GTGTGTGTGTGTGTGTGTGTGTGTGTGTGTGTGTGTGT | |
| | | GTGTGTGTAGTCCCACACTCCATCAATTATCAAACTATT | |
| | | TCTTAAATTTCCTCTGCCAGCCCTTCGTGGGAAGACAG | |
| | | CAAGTGTTGTTGATTTTTCCGTAAACACTTCATGATTCC | |
| | | TCTGTTATAAAAGGAAATGGAGAATCGCCCTGCACTCT | |
| | | GCCCCGCCGATGCCACAATGCGTCATACAATCCTAGGT | |
| | | GAGTCTGGCCACTGAGTGGTTTCGATTCTTTTCATAAA | |
| | | TGTATTTTTATTATACATGTTCTAACTACACAGCCAGATA | |
| | | GATAGATGTGTAGATAAATGTAGACATAAATATATAAAT | |
| | | ATATAGACATAGATAGATAGATGGATAGATAAACATTTA | |
| | | CGTACAAGATATTAATATTCCTTTCAGTTTTCCTTTCCCT | |
| | | CGGTGTTGTTGGGTCGGCTGTGGCAACATCATACGAG | |
| | | AAAAGTGCCAATGATTCCAAGGCTGGTAATGTTTATAC | |
| | | TCTTTTGAATTTGATTTTATATAAACTGGTACATTTCCTT | |
| | | GTGCATACATTTAATGGTTAATGGTTAATGGTTAAAAGC | |
| | | AAGATAAATGTGCTAGACATCTAAGGTCATGTAGCACT | |
| | | ATAGTTAATGTTAGGGAAGGGTGGTTGAGTTAGTGATT | |
| | | AGTTGTCTAAGCTGGGCAAAGGAGTTGGTGAGTGAAA | |
| | | GGGTTAGGGTCGGGTATGGTAAGGAGTTAGATTAGATG | |
| | | AAGAATATGTATGCGTTTGAGGAAAGAGAATAGGTTGT | |
| | | TGAAGCAAAAAGTGTGGGATTCTGTAAGGATGTCTGA | |
| | | TAGGTTGGGAGGTCGGTGAAGGGGGGATAGGTGGGGG | |
| | | AAAGCAGAGGTACGAGTTGTTTCAAAACGTGGGCACG | |
| | | GCAGTAGGATGTGTGGGATTGGAGTGTCTGGTGGTGT | |
| | | GTATTGACTTTATGAGAGTTAATGAGTTACATAAGGGAT | |
| | | TCACGTGCGTAAGGAGGGAGTGGAAGGGATGGTGGG | |
| | | AGGGTTAATGTGGGCGGGGTTGGGGTCGGGGGGGATG | |
| | | GGCTGTGTTGGGAGGGGGTAGGAGGATAGGGTGTAGG | |
| | | GGGGATATCGTTAGGAGGAGGATGGATATCAGCAGTTA | |
| | | CTTGGAGTGTGGAAGGAGCAGGAGTTGTAAGATTTGG | |
| | | AGGTTGAGTGTCAGTTTTCATTAGGTAATCTTGAATATT | |
| | | TTCAATGGTTTCTTCTTCTGAGTTGNTTCGGGAGTTTT | |
| | | GGGGNANAAANGATTTCTTGTGAGGGGGGGGGGGGA | |
| | | AGAGAGGACTGGTGTCTCAAGCATAGGAGCAAAGGA | |
| | | AGGTGGAGGTGATTGTGTGGTAGGGGAAGAGGGGGT | |
| | | GGAACGTTTATTCTGTCTGTTACGGGTAGTGCGAGGAG | |
| | | GGAGAGGGGAAGGTGTTGGGATTGTAGTGGTGATTGA | |
| | | AGTATCTGTAGTAGAGATTGGAGTGTCTGGATTTAGGA | |
| | | TGGCAANAGAGTTTGACTGGGGAAGGTAGGAGGTAG | |
| | | AAGAGGGGGGGTTATATGTAGGAGGGGTGGGTTTAGG | |
| | | AGAATTGGTAGAAATATTACTGGAGTAAGGAGTAAGG | |
| | | GAGAAACCACGTCGACGTGCTTCTTGTCTGGCTTCGC | |
| | | GTAGTGTGAGTCCAAGTTTGAATCTGAGAGTTGCTACC | |
| | | TCAGACTCAAATTTATAGGTGGGACAACCCCTATAAAA | |
| | | TACATTATGGGGGCCGCCACAGTTGGCACATGTGCGTG | |
| | | ATTGTGCAGGGCAGTTAGATCGGGTATGGCCAGGTTGG | |
| | | GCACATAGTGGCTGTGGAACGGCAATGTTTGGCTGGG | |
| | | TGTCCTAGACACCAACAATTTTGGCACTGACGTGGAG | |
| | | GGGGTTGGTATGGACGAACAGGGAGGGATTCTCCTCC | |
| | | TATGTAGACGTTAAAGGGAAGGTCATGCCTACGGAAG | |
| | | GTAATTTTGGCTATGTTAGTGGGTTTCTTTCGATGACCT | |
| | | CTGGGGGGAATGGAGTAGCATTGTACTGATATTGCATC | |
| | | ATAGTCTGTGAGACAGGCAAGTAGATCTTCTCCACAAT | |
| | | CTGACCAATCTTTGTCATAGATTGGGCAATTTGCTGGG | |
| | | GAGATTGGAACTGTTCCGGTGCAAGTATTGAGGGTTG | |
| | | GATGGGGTTCTGCAAGGATGGGNTTACCATATAGGTCT | |
| | | GTTAGTTTTGTTAATGCTATAGCTTGGTTTTCGGATGTT | |
| | | ACTGTGACAAGACGGGAGCGGTCGGGTCGGCTACGG | |
| | | AAAGAGACTTTACCTACTTGTTTTTGGAGACATTGTTG | |
| | | GAAGAGAAGGGTGTTGTCAGAGTAGGGTGTTGTCAGA | |
| | | GTAGGGAGCTGTGGGAGGGATCACGAAAAATCGGTCC | |
| | | CATTTGGCTGTGCTGAAGAGGGTATTCAAAATTGTTGT | |
| | | AGAGATAGGGGTAGTCGAAGGGCGGGGGCGTGACAA | |
| | | AGATGGAGTAGTGTTAAGGGAGGTAGTGTTATGGGGA | |
| | | GGTGGGCAATAAGGCTGTANGGTATTAATAAGGGAGG | |
| | | ATGGGGTGGTTGATGTTGGAGGTTGTGGGGGTAGAGG | |
| | | TGTTGAAGTTGAGATTGCTGGGAGAGTGGAAATGTTC | |
| | | CTTAAGGGTTGATTGTTGGCTACTGTACTAGTAGGTATT | |
| | | GATGAGGGGGTAGTTATTGCAGTGTTCGGAGCCGTGGT | |
| | | CAAAGGAGAGCCTGGGGTCAGGGAATCTGGTGGGTTT | |
| | | ACATCGTTGGGGCTATTTGATAAAGGGGCAAGCCTCAT | |
| | | TGCCCCTAATAGTGGGGATAAGTTTTCATTACTGGCCAT | |
| | | GGTAAGCCTGGATTATGTTGGGGATAAAAACAGTCCAC | |
| | | CCCTCAGGGTCCCCTTGAGGGGTAAGGGCTAGATAAC | |
| | | TAAATCAGGGGAATACCGTGCCCATGGCTCCCTCAGGC | |
| | | CGTTCAGGACTGGCACAAAGTCAGCCTTTCATCCTTTC | |
| | | AGCACGGCTCTCACACCTTAGGAGGTGGATAGCAGAA | |
| | | GGGTTTGGTGAAGGGACAGAAACGAAAAGTGGGAAG | |
| | | GAAAAAAAAAGACCATGCAAAATTAGTTGAGACGAG | |
| | | GGCCGAGTCCCAAGGTTGGGGAGTTCCCCATCATTGG | |
| | | GTCCCAGTCTCCGCCTCCTAAGCCCCCCCACGACAAC | |
| | | AACGGGCAAGGGATTGGGGGGCATGCATTGATTAATG | |
| | | ACTGCGAACGTTTTTACTAAACTTTCTTACTTCATGAG | |
| | | GTTAGAAAATTCAACCTAAAATTTCCTTTCACTTCCCAT | |
| | | GATATACTTCAGTGCCCGAGCTAATTTAATACTGCTCGC | |
| | | TCTAGCGATAATTAGAATGGCACAGGTTCGAGTCCTTA | |
| | | TTATGGAGGGCCGTTGCCCTACTTGTTAAAGCTTTTTC | |
| | | CTGTCATTACCTGGTATTTTCCTTTCGTTTTTTTTTACTT | |
| | | CTTGGTTAATTTTATTTAATATTTCAGTCTGCTATAGCCC | |
| | | CTATACTGCCATTGCGGATCGCTGTTTGTTTGTCGATCA | |
| | | CCAGACAGATGGAAGCTGGTATGACATGCGAGAGTAC | |
| | | TGTAACCTTATAAATGGAGACTTTCTCAAGCTGGATGA | |
| | | CGCTAATCTCCTTACTGATATCGTTGAGTACATTACTTA | |
| | | CCAAGGTAAGCTTCTTCTTAGGTTTATAGGATTTTGTTT | |
| | | GAACCATGTAACTTTTATCCAATTTGATGTGAACATTTT | |
| | | GCATATATGATATTAGAACTAATCTCAGCATGAGCACAG | |
| | | TCTATTACAAATATTTTAAGAACACAAAAGTAAAATATA | |
| | | GCTAAATGTAGCAACTGCTTTGTGTAGTGGGTGTGAAC | |
| | | AGAGACTACTGGATCGGGGGGAGTGACGAGAACCAC | |
| | | GAGGGTCTTTGGCTGTGGACGGACGGAACTCTCATGC | |
| | | GGACAGGTGTTCCCTTGTGGTACCATTGCACCTCCATC | |
| | | TCTCAACAACCAGATGGTGGCTCCTCAGAGAACTGTG | |
| | | CCGTCATGCGCTGGGACTCATTTTACCATATCCATGATG | |
| | | TGTCTTGCTACACTTCTCGGTCTGTCATTTGTGAAAGC | |
| | | AGGACACATTAATCTAACAAGTTTTGGCCACAGAAAA | |
| | | GATATACAAATTGTTTTTTATTACAAATTTTCTTTTATAT | |
| | | CATATTGTGCCACAAGCCGAGATCATTGGCAAAGTACA | |
| | | CATTAACCAAATGATATCTATATACTATGTACATTCGTTT | |
| | | ATCACGCTGCCCGTGGCCACACTGCCTTATGCATTTAA | |
| | | CTTTTCTAATCTGTCCTGAAATCTTTTTTAGCTGTAACA | |
| | | TTGCAAGAATAAATATTCTAAAGG | |
| 5 | Shrimp | AAATGAGGCGGCGGCAATGATTTACGGGCATATATTCG | 151 |
| | b-actin | GTCGAGGAGGACGAAATATTCTGAAATGGGACGAAAG | |
| | promoter | GGGATGACGCGGCGCGGCTCTCGTCTTCCCGCCTCGC | |
| | | ATTCAACGCTCGGCTCGACCAATCAGCGGCCGAGTTTT | |
| | | GCGCTATGACCATATAAGGCGATACGTTTGTCCGGGTG | |
| | | GGGTGGGACGAGCCATTGCGGCTTATCGCGCGGGGGA | |
| | | GTACCCTCTCAAAATGCACTATGCACTGCCGTAACACT | |
| | | CTTTCGGAAAGAATATAATACATCAGTAGATACCTCTTG | |
| | | AAAATTAGGATCCGATGCATACCATAAATCCCCAAATTA | |
| | | GAGAGAATAAAAGGGGTTAATTCGATCGAGAGTAATG | |
| | | ACACTTGGAACGACCTCCCCTCTGGAGAAAGTCGACG | |
| | | ATCCGAGAGGTGGAGTAAGCGCCCTACTCACTCTCTC | |
| 6 | Human | GACATTGATTATTGACTAGTTATTAATAGTAATCAATTAC | 152 |
| | cytomegalo | GGGGTCATTAGTTCATAGCCCATATATGGAGTTCCGCGT | |
| | virus | TACATAACTTACGGTAAATGGCCCGCCTGGCTGACCGC | |
| | promoter/ | CCAACGACCCCCGCCCATTGACGTCAATAATGACGTAT | |
| | enhancer | GTTCCCATAGTAACGCCAATAGGGACTTTCCATTGACG | |
| | | TCAATGGGTGGAGTATTTACGGTAAACTGCCCACTTGG | |
| | | CAGTACATCAAGTGTATCATATGCCAAGTCCGCCCCCT | |
| | | ATTGACGTCAATGACGGTAAATGGCCCGCCTGGCATTA | |
| | | TGCCCAGTACATGACCTTACGGGACTTTCCTACTTGGC | |
| | | AGTACATCTACGTATTAGTCATCGCTATTACCATGGTGA | |
| | | TGCGGTTTTGGCAGTACACCAATGGGCGTGGATAGCG | |
| | | GTTTGACTCACGGGGATTTCCAAGTCTCCACCCCATTG | |
| | | ACGTCAATGGGAGTTTGTTTTGGCACCAAAATCAACG | |
| | | GGACTTTCCAAAATGTCGTAATAACCCCGCCCCGTTGA | |
| | | CGCAAATGGGCGGTAGGCGTGTACGGTGGGAGGTCTA | |
| | | TATAAGCAGAGCT | |
| 7 | Carp | AAGCTTTTAGACCTTCTTACTTTTGGGGATTATATAAGT | 153 |
| | b-actin | ATTTTCTCAATAAATATCTCATATCTTACTGTGGTTTAAC | |
| | promoter | TGCTGAATCTAAAATTTTAATACAAAAGTAGTTATATTT | |
| | | GTTGTACATTGTAAACTATAACTTAACTTCAGTTTCAGA | |
| | | GAAACTCATGTGCTCAAAATGTAAAAAAAGTTTCCTGT | |
| | | TAAATATTTTGTAAATGTATTGAAGACAAAATAAGAAA | |
| | | AAAAAAAATATAAGCCACTAAATCACACTGTCCTTGGT | |
| | | ATCAGCAAGAGATTCTGACATAATCAGCTGTTTTTGTT | |
| | | TATTACTGCCATTGAAGGCCATGTGCATTAGTCCCAAG | |
| | | TTACACATTAAAAAGTCACATGTAGCTTACCAACATCA | |
| | | GTGCTGTTCAAGCACAGCCTCATCTACTATTCAAACTG | |
| | | TGGCACCATCTAAAATATGCCAGAATTTTTTTATTTAAT | |
| | | GAATTTGACCCTGAAATATGTATTAATATCACTCCTGTG | |
| | | ATTTTTTTGTAATCAGCTTACAATTACAGGAATGCAAGC | |
| | | CTGATTCATTACAAGTTTCACTACACTTTCTCTGACAAC | |
| | | ATCACCTACTGAACTCAGACCAGCTAGTTGCTCCTTAA | |
| | | GTATACAATCATGTCAGTAATCCTCATTTCAATGAAAAA | |
| | | TACCCGTATTGTACTTGGTACTTGGTAGATAACCACAG | |
| | | AGCAGTATTATGCCATTATTGTGAATACAATAAGAGGTA | |
| | | AATGACCTACAGAGCTGCTGCTGCTGTTGTGTTAGATT | |
| | | GTAAACACAGCACAGGATCAAGGAGGTGTCCATCACT | |
| | | ATGACCAATACTAGCACTTTGCACAGGCTCTTTGAAAG | |
| | | GCTGAAAAGAGCCTTATTGGCGTTATCACAACAAAATA | |
| | | CGCAAATACGGAAAACAACGTATTGAACTTCGCAAAC | |
| | | AAAAAACAGCGATTTTGATGAAAATCGCTTAGGCCTTG | |
| | | CTCTTCAAACAATCCAGCTTCTCCTTCTTTCACTCTCA | |
| | | AGTTGCAAGAAGCAAGTGTAGCAATGTGCACGCGACA | |
| | | GCCGGGTGTGTGACGCTGGACCAATCAGAGCGCAGAG | |
| | | CTCCGAAAGTTTACCTTTTATGGCTAGAGCCGGCATCT | |
| | | GCCGTCATATAAAAGAGCGCGCCCAGCGTCTCAGCCTC | |
| | | ACTTTGAGCTCCTCCACACGCAGCTAGTGCGGAATATC | |
| | | ATCTGCCTGTAACCCATTCTCTAAAGTCGACAAACCCC | |
| | | CCCAAACCTAAG | |
| 8 | EF-1alpha, | AATTCGATTCCGAGCTCCCGTTATAAGTTGTAATGGAAT | 154 |
| | Marsupenaeus | GTATCTTCACTCAAATGTATACGCACCTAAAAATCTAAA | |
| | japonicus | CCAGAGTATTTAAATAATTGCTGCTATCATTGARACTGA | |
| | | TAGAAGAAAAGAAGAGARAGAGAAGAGAAGAGGGA | |
| | | GAGAGAGAGAGAGAGAGAGAGAGAGAGAGAAAATTA | |
| | | TATTCGTTCACTGGTAGAAAGACTGATGGGAAATTTGT | |
| | | GATCTTGATATAACTGCAATTACTGCAAATATGTGCATA | |
| | | TCAAACCCATCAACATGTCATCTCATACAGTGATGACC | |
| | | TTTAAGAAAGAGCTGTTAATCCAATTACTTGTTAACAT | |
| | | GATTTACATTACAGTTTAGTGACCATTTAARACACATAC | |
| | | CGATGTGGTAAATTACTACAAATACAATTGATGTCTCTA | |
| | | TATTCTTAGCACTTGAGCTTAGCTAAAGAACAACAAAC | |
| | | TGAAAATAAGTCAACAAAGCGATTGAATATACCTTCCA | |
| | | CCAATTCAATCACAAATTAAGAATGCCAGACAATCTTA | |
| | | AATCGAATTTCTCAATTGTACAAAAATTGCTCGTTTGC | |
| | | CGCTCGCTCGTGAACGAAATTCCCGCCAGAAATAGAG | |
| | | GCGCGAGCGGTGCGCGCGCCGCTTCCCCGCTGACCAA | |
| | | TCAGAACCGCCTGCATATAGTGACGTATAGGTCTCATA | |
| | | ACCAATGGGCGAGTCGTATTGATGTGACGTCACGTGCC | |
| | | GCCAGCCAATGAAAAAGGAGACCGCGGCTATATAGTA | |
| | | CTTTCGGCTTCATCCATCGTGTCCTTTCCGTCTTGGCCA | |
| | | GTCGACGTGGGCCTGTTGCCAAGGTAAGAATTTGTGA | |
| | | AAGTGACGATTTTCAGTGAGATCCCGAAGGGAAAAGT | |
| | | ACATGAAAAGGTGTAATCCTGGTGCAATAATGCAGGTG | |
| | | CTATCCGAGATCAGGAAGAAAGTCCAGAATTCAGGCC | |
| | | TAAATAACGCATCACAAGCAGTGAAGTGTTGGCCAGC | |
| | | TGCGGCTGTCAGGATCCCACATTGGTCTGGTCGCCACC | |
| | | CGGCACATGTTTCGTAGAAAACGAAGGCTTTGAGAGG | |
| | | CTGATTCGCAAGGCTGAGGCTCGGTGGATATGTGTTCA | |
| | | GTGCAGCGCCACGAGAGATTTCAATATTTTCCACCTAG | |
| | | ATAGTCCAAGTAGAATAAGTAAAATAAGTGCGTGGGA | |
| | | GAGCGAGGGATCCAGCCGGCTGAAGCGAGCATGAGGT | |
| | | TACGGTCGCCGCCATTACGACCCTGTGCGCTCGACGAC | |
| | | GCCATGTGATGGACAAAGATCCGTGGAATATTTTGTGC | |
| | | TCTCTTAGTAACTACCCCCTGAAGGGTTAGATAAGCTT | |
| | | CTTGAAAAGTTGTATTATTACCTAGATTTAGTTATTTTTA | |
| | | AAACTTTTTTTTTAATTAGCCGAATAGTTTATTGTACAT | |
| | | CCCTCCCTTTTCAAAGTATTTCATAATAAGTTTTTCTTAT | |
| | | GTAAAAGGGTAATTAAAGTGTTGTAATTCAACGCAAGT | |
| | | AATTAAATCTTAATCATTAACTAATAAATTACCCCTTAA | |
| | | CCGCTATTTTTCCTGTGTCCATAACTCATGATTCGCTTC | |
| | | TTGCAGATCTGTAAGCTCTCGGTCAAGTAACCAACCAT | |
| | | GGGCAAGGA | |
| 9 | Translation | GATATGCATATACTTACTCATTTACTGTAAAGAGATAGT | 155 |
| | ally | AAAGCAGAGATCATTAATTCGGTTGCTCATCCTCGTTT | |
| | controlled | CCTTCCAATGTACGAAGGAGTGGCAACAGCAAGGTAA | |
| | tumor | TTGAAGTTCGTTAGTAGAGGGAAAAGGAAGCGTGAAA | |
| | protein | CTTAACCGGCGATTGGTGCCTTCGTATCTAGGTCATATT | |
| | (TCTP) | AAAAGCCACTGTCTCTTCACGTGATTAATTTACTTCGC | |
| | | TTATGTTGGTTCACTGATTAACTTGTAACATTGTATTGA | |
| | | TATATTTATATTTCAATGCCAGTGAGGTGAATGATCGTC | |
| | | ACTGACATTGAAGAAGAATATATATAAATTCAAAAACG | |
| | | CTAAGTAAAGAGTTCATTACTCGTAAATTGACTCCCCT | |
| | | TATAGGAATGTACATCGCCATTACCTATGAGAATCTGAA | |
| | | AGAAATGACCAAACAATAAAAAAAGAAAGATTGATAA | |
| | | GAATATCTGAATGGAAAGTGGCTGCGCTTGGCGGCCG | |
| | | TCACTTGTTTGTTTACAAGAACGGGCGAGGCGAATCC | |
| | | GGGTCGGTCCTTCCGTGCTCTTCCACGGTGAACGAGA | |
| | | CCAGCTCCCACACCGGTTGAGAGTTTAGCGACGAT | |
| 10 | Ptctp | GATATGCATATACTTACTCATTTACTGTAAAGAGATAGT | 155 |
| | promoter | AAAGCAGAGATCATTAATTCGGTTGCTCATCCTCGTTT | |
| | | CCTTCCAATGTACGAAGGAGTGGCAACAGCAAGGTAA | |
| | | TTGAAGTTCGTTAGTAGAGGGAAAAGGAAGCGTGAAA | |
| | | CTTAACCGGCGATTGGTGCCTTCGTATCTAGGTCATATT | |
| | | AAAAGCCACTGTCTCTTCACGTGATTAATTTACTTCGC | |
| | | TTATGTTGGTTCACTGATTAACTTGTAACATTGTATTGA | |
| | | TATATTTATATTTCAATGCCAGTGAGGTGAATGATCGTC | |
| | | ACTGACATTGAAGAAGAATATATATAAATTCAAAAACG | |
| | | CTAAGTAAAGAGTTCATTACTCGTAAATTGACTCCCCT | |
| | | TATAGGAATGTACATCGCCATTACCTATGAGAATCTGAA | |
| | | AGAAATGACCAAACAATAAAAAAAGAAAGATTGATAA | |
| | | GAATATCTGAATGGAAAGTGGCTGCGCTTGGCGGCCG | |
| | | TCACTTGTTTGTTTACAAGAACGGGCGAGGCGAATCC | |
| | | GGGTCGGTCCTTCCGTGCTCTTCCACGGTGAACGAGA | |
| | | CCAGCTCCCACACCGGTTGAGAGTTTAGCGACGAT | |
| 11 | Heat shock | TAAAAACAGAAACAATTTGTAATAATATTGTCACTGCG | 156 |
| | cognate 70 | TCTTGTAAGCCTGAAATAACTTGAGGGTGTACTTGCAG | |
| | promoter | CTATTATATGAATTCAGTTAGTGTAACCGTGTTATTATAT | |
| | | TACATTAACATAATAACTTTAACGTTATTGTTATGACTA | |
| | | GTAGCTATAAACATTCTAGATAATACTAGTTATCACAAT | |
| | | CGTGCAATATATCAATAAATTATTTGATGTTCATCAGTGT | |
| | | TTCTAAAGAAGTTATTTATACCACGGGCTACGGTTGCAT | |
| | | AAAGCCGGGCCACGTGATATTCTCGCAGACGGTCTCC | |
| | | CGCGTCTGGACCTCGCTGGCTGGCGGCTTTGCCATGA | |
| | | GGTCGCTCCCCACGTGGCGAAGGAACCGCTGTCATGT | |
| | | TGGCACGCCTGGTACGCCGAGCTGTTCTGCAGTGACG | |
| | | CGCGTAAATTTCCCGCTAAATACCGTCCAATAGCATCG | |
| | | ACCATTCCGGCATTGTTGACCAATAAGAACGTTTCACT | |
| | | TACCACGTCACGGTTATGAAGCCCTACGATTGGAACCA | |
| | | GAAGTGACGTCATAAACTAGGAACTCTGTGAATGGCC | |
| | | AACGTTCCGAAGAAGGTTCTAGAAGGTTTACAAAGGG | |
| | | GTCGATGTCGCCGCTCTATGGATTTGCCGAAGTCATAC | |
| | | CA | |
| | Penaeidin | ACCCCCTTTG AGAACTCTCC | 157 |
| | type 536 | TGACAACGCTTACTAACAGC TTGCCTGTTG | |
| | promoter | TATAGTGTCAGATGGTCTCC ATTACGTGTG | |
| | | GTATATGTTTAAAAAAAAAG GGAGGTTTAA | |
| | | AAGTTAAAAT GATGATGAT GATGGTGAAT | |
| | | AATGATGAAA CTGAAAAATC TTATATTTTT | |
| | | TCCATGTTTT TTATCTGTCT GTCTGTTAAT | |
| | | CTAATAGTTA GCTATCTATC TATTTACCAT | |
| | | TCTGTTTAGT TTTGAGTCTC TTTTTCTATT | |
| | | TGTCTCTATG CCTATTTATA TTTCTTTCTT | |
| | | TCCCTCTTAC CCCCCTTCCC TCTCTCTCTC | |
| | | TCTCTCTCTC TCTCTCTCTC TCTCTCTCCT | |
| | | TTCTCTATCT ATCTCTACCT TCCTGTCTTT | |
| | | CTCTCCCCCC GTCTCTCTCT TTAAAACTGC | |
| | | TTGCACAACC GCGTGGCGTC TCTATAAAAG | |
| | | CACCACAGCC CCCGGTGCCA GTCGGTGCTT | |
| | | GGCTCTCACC TGACCCCCAC CTGTAGAGGC | |
| | | CGAGACTCCT TGCCCGGGTT CCTTCCTGTG TCCGCC | |
| | Penaedin | CATTTACATG AAATTGAAAA GAACTGGACA | 158 |
| | type 411 | TCGTTTGAAA ACCCACTAGA TTCTCCCTCT | |
| | promoter | TTTCTCGTTC TCTCTGTCTG CCTTCCTGTT | |
| | | TGTCTGTCTG TCTGTCTGTC TCTCTCTCTA | |
| | | TATATGCTAT ATATGTATAC ATATGTATAC | |
| | | TTCAAAGACC TTACACATCT CAATATATAT | |
| | | GCATATATAT GTGTGTGTGT GTGTGTGTGT | |
| | | GTGTGTGTGT GTGTGTGTGT GTGCATGTGT | |
| | | GTATAGATGT ATGTAAAGCA TGGAAAACGA | |
| | | CTCAAGGTGC TTGCACAACA TCGTGGCGTC | |
| | | TCTATATAAG CCAGCCACCT CAGCTTCCAG | |
| | | TACCAGTCGG TGCTTGGCTC TCACCTGACC | |
| | | CCCACCTGTA GAGGCCGAGA CTCCTTGCCC | |
| | | GGGTTCCTTC CTGTGTCCGC C | |
| | Shrimp | AGTCCCACACTCCATCAATTATCAAACTATTTCTTAAAT | 159 |
| | PmAV | TTCCTCTGCCAGCCCTTCGTGGGAAGACAGCAAGTGT | |
| | promoter | TGTTGATTTTTCCGTAAACACTTCATGATTCCTCTGTTA | |
| | (DQ641258.1: | TAAAAGGAAATGGAGAATCGCCCTGCACTCTGCCCCG | |
| | 477-639) | CCGATGCCACA | |
| | Shrimp | CCTGGGGTCAGGGTGGGCTCACCCGTAGTGAACAGCT | 160 |
| | Pm VRP15 | CCGGGTCTTTCACTACAAAAGAGGATGAAACCGATAG | |
| | promoter | ATCGCGCTCTATCTTTGGCAATAACCGTACGGGTCTGA | |
| | | AACACGTA | |
| | FcCTL | TCTCACGTTTGAAAAAAGAGCTCCCTATTAGAAA | 161 |
| | promoter | | |
| | from | | |
| | Fenneropendeus | | |
| | chinensis | | |
| | WSSV | CTCGCCCACCACCAGATATCTGGGAAGCCTTGGCGAG | 162 |
| | immediate- | TCATGTTTCTTGCACCATGTATTTCTAGGAATTTCGCTG | |
| | early gene | ACGTCAATAAACTGGTTTGTCATCCTGTTTTATCCAGTT | |
| | wsv249 | TGCTGACGTCAATAGACCATATTTGGGCATCTCGCCCA | |
| | promoter | CACAGAAACAGGATATGATATCATAGATTTCTGGGAAG | |
| | | AGGGTGTTTTTTGTGCCGATCTGGGTGTATAAAAGAGC | |
| | | GCTGCGGAGAGGCAGAAACATCAGACAGACTTGATCT | |
| | | GTACAGCTAGCAGCAGCAGTAGCAGCAGCCAAGAGA | |
| | | AGATCGGACGCAAACCATTCTCGCAGCC | |

This vector should, on transformation in shrimp cells, produce both a shrimp codon optimized endonuclease (Cas9) and crRNA/gRNA targeting three sites on RR1 to enable genome editing of invading WSSV. The other two vectors were designed similarly to p14 but p12 targeted VP28 and p13 targeted DNApol genes from the WSSV genome.

Purified vector DNA was obtained using PureSyn's (Malvern, PA) TransfectionReady™ Plasmid purification service. This DNA preparation was used directly for challenge experiments.

### Example 5: Inhibition of WSSV Replication in Shrimp by Crispr/Cas Constructs Via Injection

Two oral WSSV oral challenge studies addressed the hypothesis that injection of shrimp with genome editing vectors optimized for expression of the CRISPR/Cas components in shrimp that target WSSV would impact the progression of WSD. Injection is a more direct route to test the effect than oral delivery.

The constructs produced in Example 4 targeted three different genes, the WSSV major capsid gene (VP28), the WSSV DNA polymerase (DNApol), and subunit 1 of ribonucleotide reductase (RR1). As described, these vectors were designed as multiplexed CRISPR/Cas9 vectors. Each vector had three different target sites for one respective gene to better assure that the gene was edited and inactivated by genome editing. Our expectation when planning this research project was that we would see no changes in shrimp morbidity and mortality on oral WSSV challenge but would be able to capture whether genome editing occurred in the shrimp through next generation sequencing to identify amplicon variants at the predicted target sites on the three genes.

Shrimp were received as post-larvae (PLs) and raised to between 1-3 g. For some large outliers, the shrimp could reach 5 g during the second study. Shrimp were sorted into five 4-foot diameter fiberglass tanks filled to between 5 and 8 cm depth with synthetic ocean salts (24 ppt Instant Ocean). Seven of these tanks were arranged in a straight line along the wall of the challenge room. Each tank was fitted with a bubbler and foam filter to diffuse the air in the tank. Shrimp were moved from the maintenance facility into the challenge facility and sorted to between 45 and 50 shrimp per tank (50 in Trial 1 and 45-50 in Trial 2). Shrimp were equilibrated in this system for at least 2 days. After the shrimp were acclimatized, shrimp were injected with 20 μL of treatment or control in the second segment up from the tail fin as described in (Table 3) for Trails #1 and #2 (Table 4). The average size of the shrimp in Trial #1 was 1.38, which was smaller than that of Trial #2 (average=2.77 g) which was performed a few weeks later.

TABLE 3
WSSV oral challenge trial 1; treatments and tank layouts

| Tank | | | Animals |
|---|---|---|---|
| # | Treatment | Description | (N) |
| 1 | A | Positive Control - Mock injection/oral | 50 |
| | | challenge | |
| 2 | C | VP28 target - p12 injection/oral challenge | 50 |
| 3 | B | Negative Control - Mock injection/no | 50 |
| | | challenge | |
| 4 | E | RR1 Target - p14 injection/oral challenge | 48 |
| 5 | F | All 3 genes targeted - p12/p13/p14 | 50 |
| | | injection/oral challenge | |
| 6 | D | DNApol Target - p13 injection/oral | 49 |
| | | challenge | |

To assure that no positional bias occurred during the trial, the assignment of each treatment to a tank was performed using a random number generator. Tank 1 was nearest to the door of the challenge facility while tank 7 was at the back wall. The facility was temperature controlled to −27° C. (80-81° F.).

TABLE 4
WSSV oral challenge Trial 2; treatments and tank layouts

| Tank | | | Animals |
|---|---|---|---|
| # | Treatment | Description | (N) |
| 1 | E | RR1 Target - p14 injection/oral challenge | 46 |
| 2 | C | VP28 target - p12 injection/oral challenge | 47 |
| 3 | A | Positive Control - Mock injection/oral | 44 |
| | | challenge | |
| 4 | B | Negative Control - Mock injection/no | 47 |
| | | challenge | |
| 5 | D | DNApol Target - p13 injection/oral | 48 |
| | | challenge | |
| 6 | F | All 3 genes were targeted - p12/p13/p14 | 49* |
| | | injection/oral challenge | |

*one animal was not injected and fell into tank

The shrimp were injected with 20 μL of injection mix comprising: 0.9% NaCl, 20% glycerol, 10 mM sodium butyrate and 10 μg plasmid DNA for treatment injections. For Treatment F (where all genes were targeted simultaneously) 3.3 μg of each vector were used to deliver a total of 10 μg of DNA per 20 μL injection. Approximately 12 hours after the control/treatment injection, shrimp were orally challenged with shrimp tissues infected with WSSV (treatments and Positive Control) or virus free shrimp tissues (Negative Control). Tissues were macerated with two razor blades into a fine paste then fed to the shrimp at −5% of body weight (3 g of tissue/tank).

The viral load of the oral challenge tissues was determined using qPCR as described by Durand and Lightner (see e.g., Durand and Lightner. Quantitative real time PCR for the measurement of white spot syndrome virus in shrimp. J Fish Dis 25, 381-389 (2002)). Briefly, the Taq Polymerase system was used in a real-time PCR instrument (Applied Biosystems 7500) using a standard curve generated using a synthetic vector containing the target sequence of the primer set. Shrimp tissues used for the oral challenge contained 104,369 to 651,610 and 2,966,454 to 6,194,523 mean viral quantity in 1 μL of isolated tissue DNA in trials #1 and #2, respectively. While viral load of the challenge tissues was substantially lower in #1 than #2, the biological relevance of this difference is not known. The LD50 for L. vannamei of this size was about 253,000 mean viral quantity. The tissues were spread about the tanks and broken up with a spatula to try and ensure that equal access to the virus was provided to the shrimp. Shrimp were not fed in the prior 24 h to oral challenge and were observed to rapidly begin feeding on the introduced muscle tissue. No residual materials were observed 2 hours after feeding.

TABLE 5
Viral loads for shrimp used for the first feeding experiment.
Presented viral quantities are in 1 μl of total DNA sample.

| | Shrimp | | | |
|---|---|---|---|---|
| Shrimp | Weight | Mean Viral | Standard | Range of Viral |
| No. | (g) | Quantity | Deviation | Quantity |
| 1 | 24.0 | 596,765 | 47,857 | 563,482-651,610 |
| 2 | 31.5 | 128,411 | 22,925 | 104,369-150,029 |

TABLE 6
Viral loads for shrimp tested used for second feeding experiment.
Presented viral quantities are in 1 μl of total DNA sample.

| | Shrimp | | | |
|---|---|---|---|---|
| Shrimp | Weight | Mean Viral | Standard | Range of Viral |
| No. | (g) | Quantity | Deviation | Quantity |
| 3 | 45.0 | 3,011,978 | 56,419 | 2,966,454-3,075,099 |
| 4 | 40.2 | 5,775,216 | 488.841 | 5,238,298-6,194,523 |

Animals were checked periodically for the onset of morbidity and mortality. During the first 24 hours, shrimp were checked at least every 6 hours during the day and every 8 hours at night. Shrimp mortalities did not start until 37.5 and 35 hours post-challenge in runs #1 and #2, respectively. Once mortalities began, the tanks were monitored at least every 4 hours until the intensity of the disease progression tapered off (as determined by mortality rate).

Sampling was done in the following manner. Morbid (D) or moribund (M) animals were removed to a balance and weighed. Animals were then decapitated with a scalpel and the tail muscle stored in screw capped tubes at −20° C. until transferred to a −80° C. for long-term storage. The heads were put into Davidson's solution for histology for at least a week then washed in ethanol for future evaluation for clinical signs of disease.

When the disease progression was at its highest rate (−50 h and 56 h in runs #1 and #2, respectively) live animals not displaying symptoms were harvested (live-harvest) and prepared as described above for the mortalities and moribund animals. The logic behind these live-harvest sampling was that most if not all the animals were infected and in some stage of WSSV infection. Therefore, those animals not currently displaying symptoms might have the lowest number of native viruses and the highest proportion of genome-edited animals.

Tissues for nucleic acid isolation were stored at −80° C. prior to isolation and analysis.

### Oral Challenge Run 1 (Run #1)

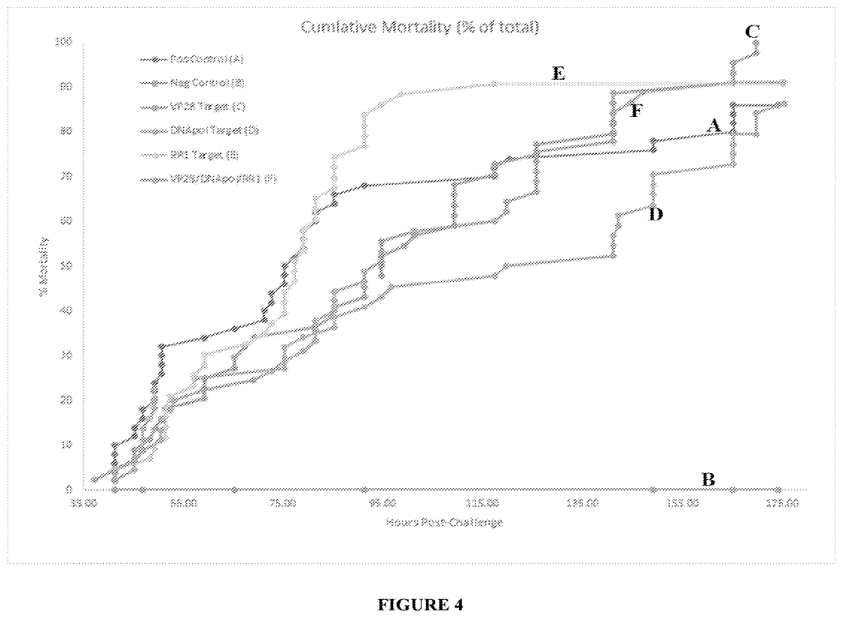

RUN #1 oral WSSV challenge cumulative mortalities were monitored over the life of the experiment (FIG. 4). Mortalities did not begin until about 50 h post oral challenge in both runs. At that point there was a rapid rate of mortalities in the positive control (A) and in the treatment targeting the large subunit of ribonucleotide reductase (RR1, Treatment E). All other treatments appeared to have a reduced rate of mortalities and a slowed progression of the infection (as determined by mortality rate). No mortalities were observed in the negative control. Four days post-challenge, both the RR1 and Positive Control mortalities flattened out while the other treatments slowly reached equivalent cumulative mortalities after about a week post-challenge.

### Oral Challenge Run 2 (Run #2)

This challenge run was done as described for Run #1 with the exception that the shrimp were several weeks older (and therefore larger). The amount of the treatment DNA injected was held constant and not adjusted for this change in weight and the oral challenge was still carried out at −5% of body weight. The number of animals available did not allow fifty animals per treatment, so the numbers ranged from 44 to 49 (Table 2).

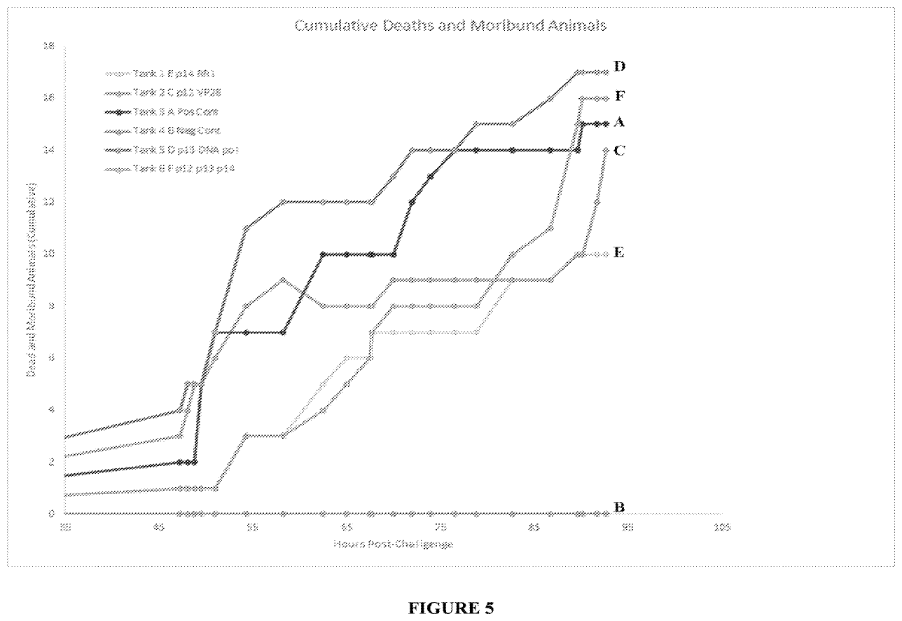

The progression of this challenge mirrored that of Run #1 with the Positive Control tank beginning to see mortalities at −50 h (FIG. 5). The treatments targeting DNA polymerase (D) and VP28 (Tank C) differed from the results of the first run. The combined vector treatment (F) and ribonucleotide reductase subunit 1 treatment (E) both seemed to delay the progression of the disease but slowly approached the same cumulative mortalities of the other treatments. No mortalities were observed in the negative controls.

The cumulative mortality data generated during the study showed a trend toward slowing the progression of the WSSV infection.

### Example 6: Production of 2″ Generation Crispr/Cas Constructs Targeting WSSV Genes

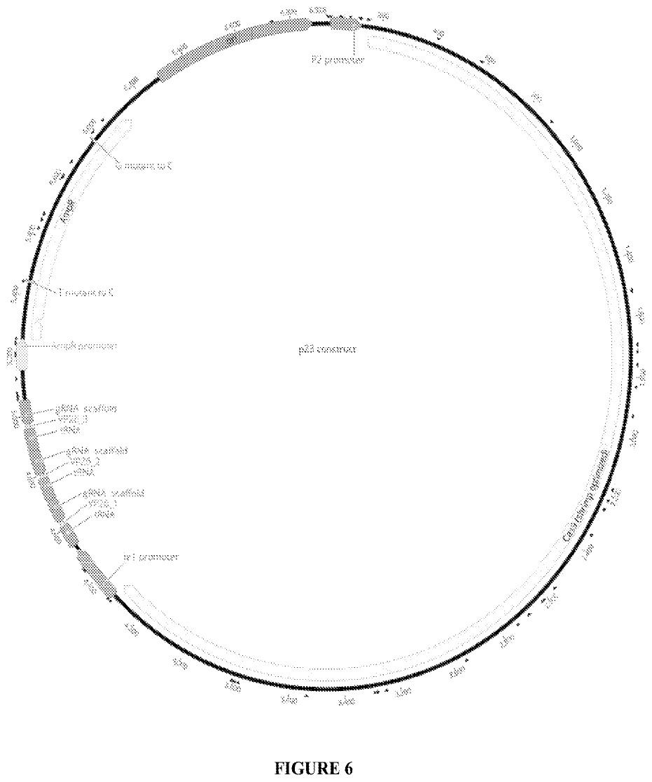

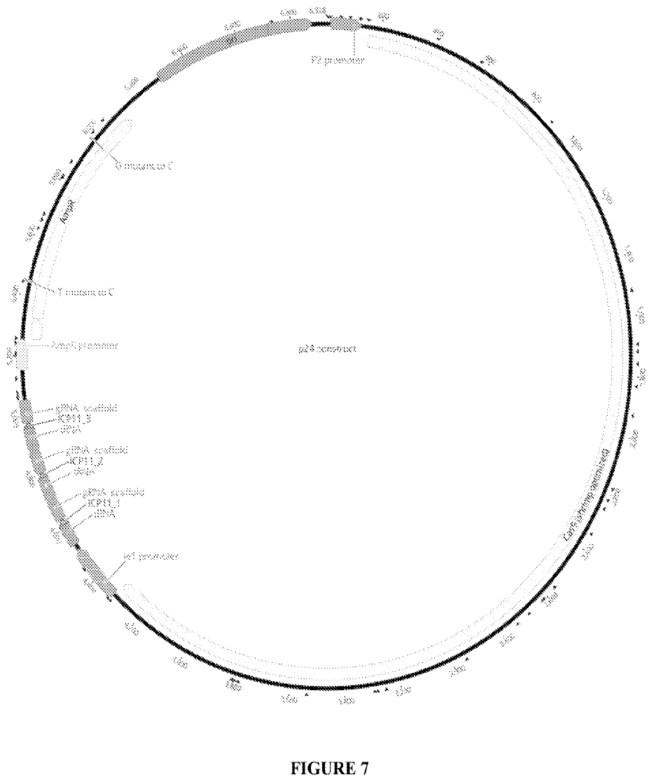

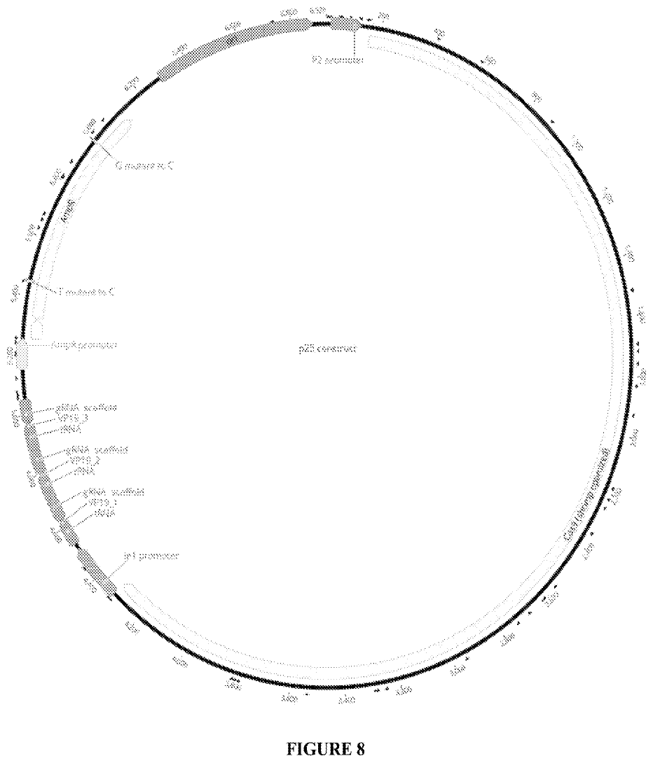

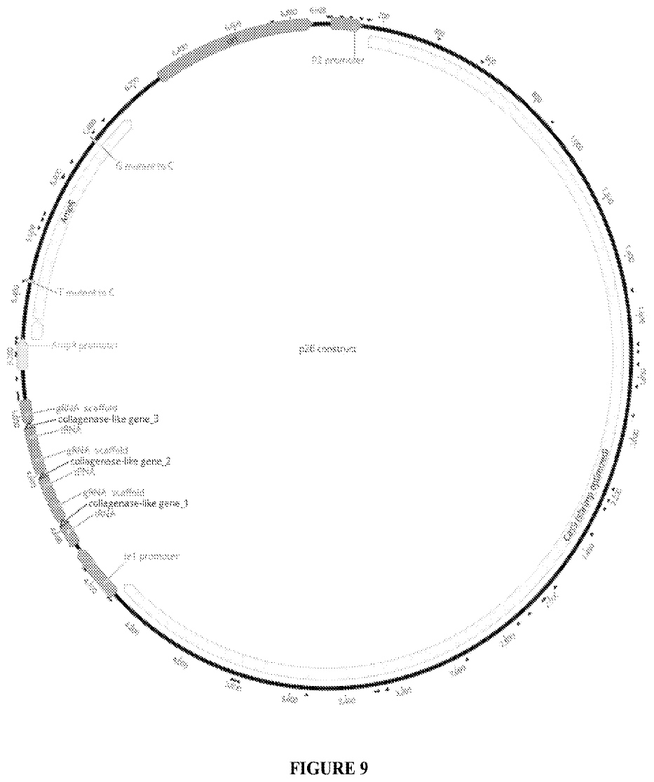

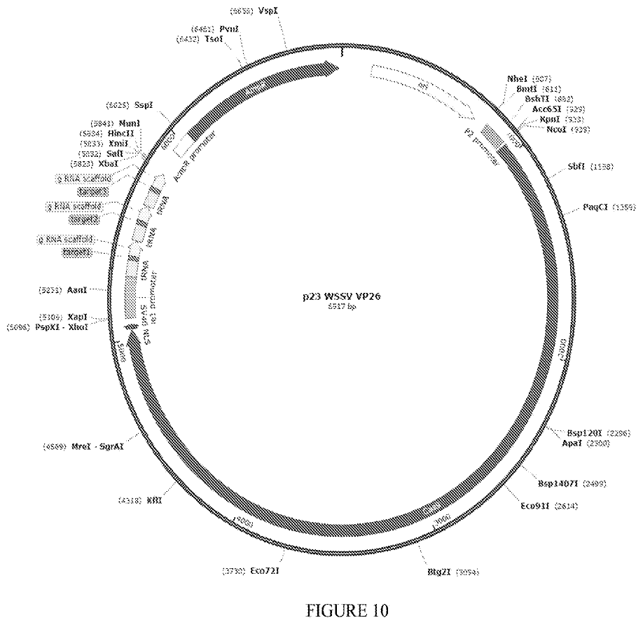

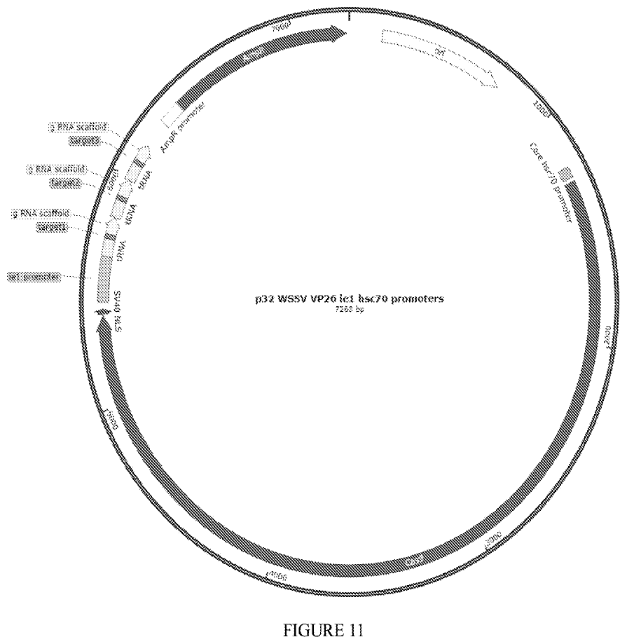

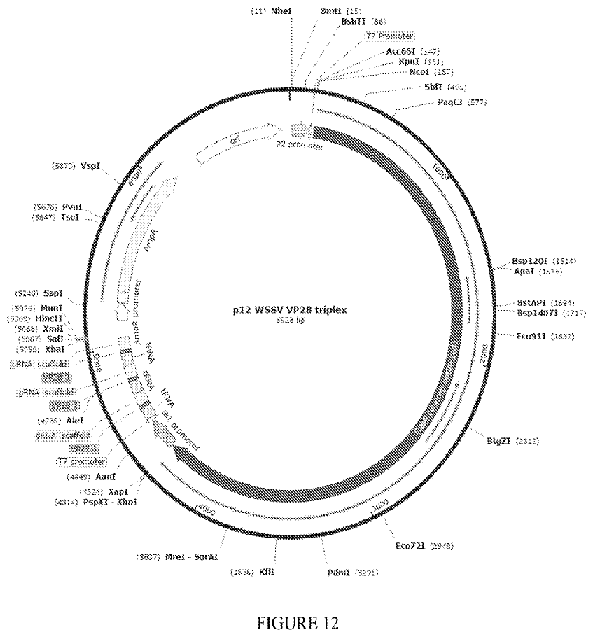

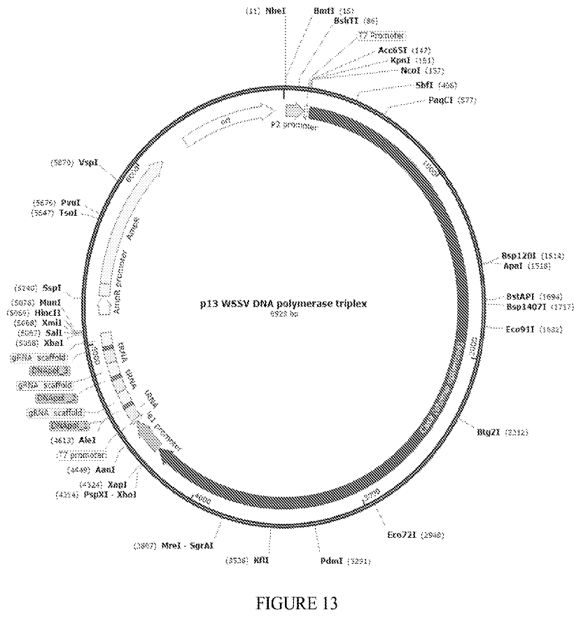

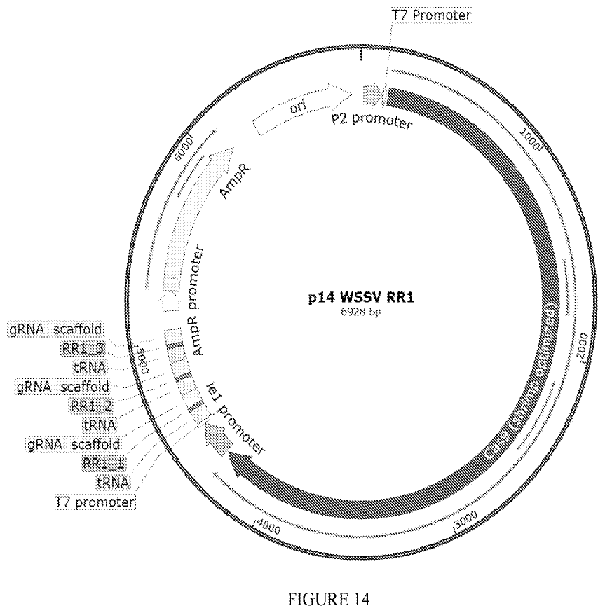

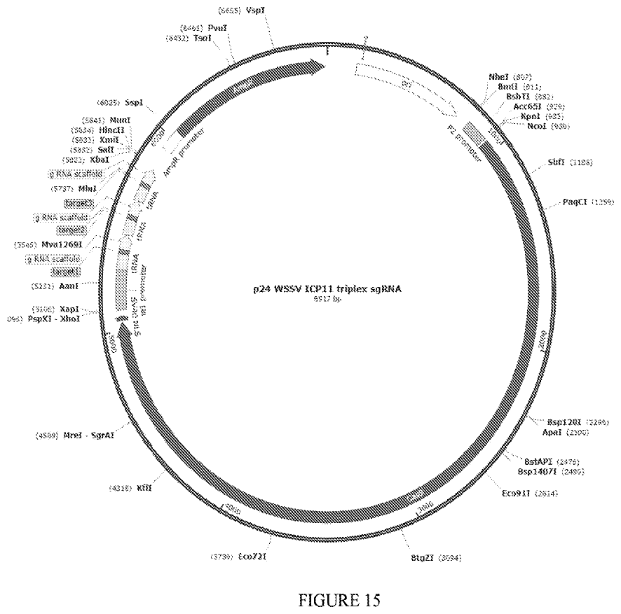

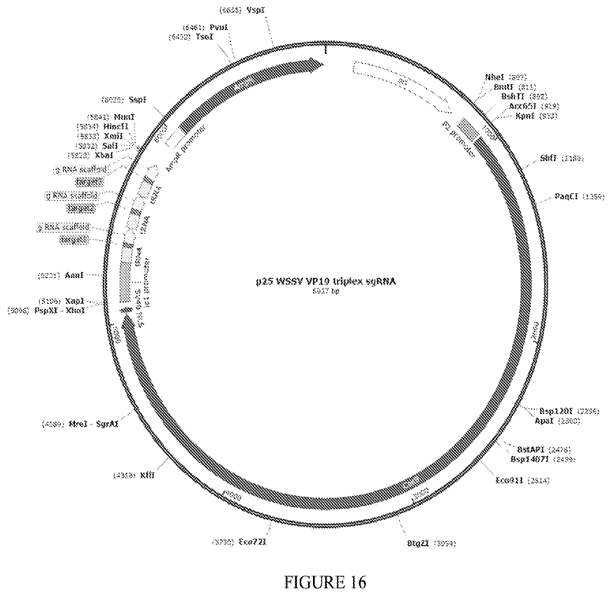

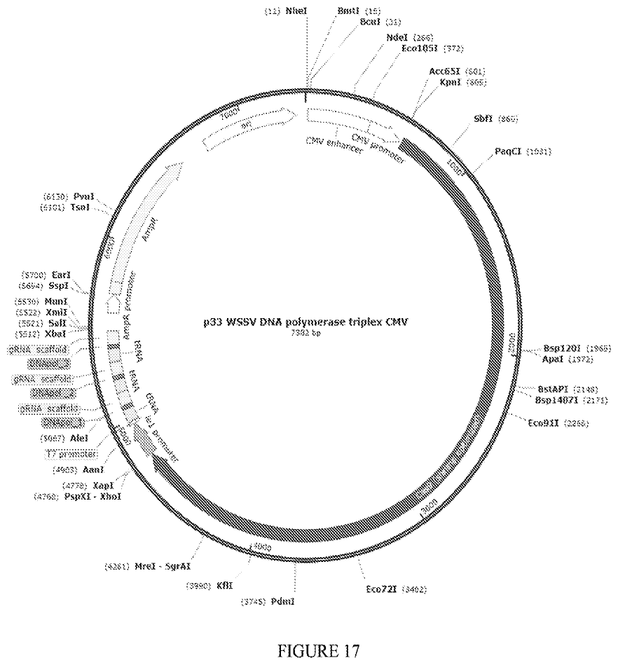

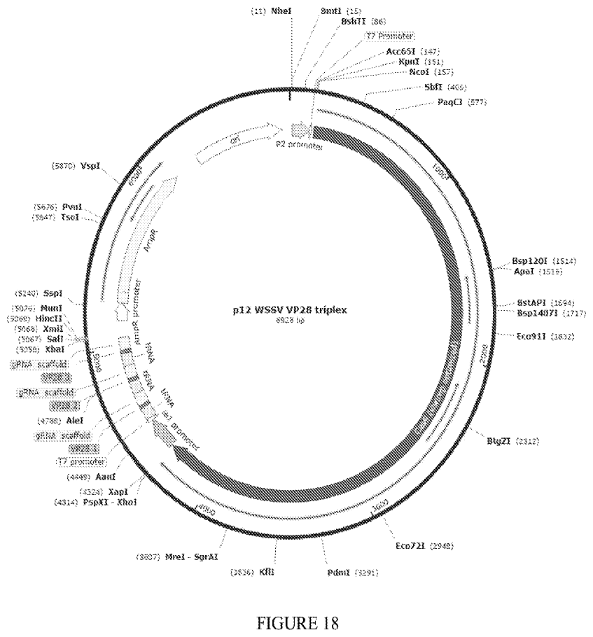

2^{nd} generation vectors (see e.g., p23, p24, p25, or p26 in FIG. 6, FIG. 7, FIG. 8, and FIG. 9) for delivery of Cas9: sgRNA were constructed based on the p14 vector described previously, but with the T7 promoter removed downstream from the shrimp functional promoters (P2 and ie1 promoters). These vectors were designed to target ICP11, VP19, VP26, and collagenase-like gene with three distinct sgRNAs directed against multiple target sites of each gene. Arrangement of elements from 5′-3′ comprised P2-Cas9 (shrimp optimized), followed by ie1 promoter−tRNA−sgRNA1 (target+scaffold)−tRNA−sgRNA2 (target+scaffold)−tRNA−sgRNA3 (target+scaffold), followed by UUUUUU, followed by ampicillin, followed by a bacterial origin of replication (ampicillin and bacterial origin being optional components assisting replication in bacteria). Example target sequences for sgRNAs for each target gene were designed for Cas9 compatibility and minimal off-target activity (see, Table 7 below).

The present invention is not limited to Cas9. Any appropriate programmable nuclease may be used, such as those described herein, those not listed specifically but are known to one of ordinary skill in the art, and those that may be discovered. The programmable nuclease may comprise a CRISPR-associated (Cas) nuclease, a zinc finger nuclease (ZFN), a transcription activator-like effector nuclease (TALEN), etc. Cas nucleases can include but are not limited to Class 1 CRISPR-associated (Cas) polypeptides, Class 2 Cas polypeptides, type I Cas polypeptides, type II Cas polypeptides, type III Cas polypeptides, type IV Cas polypeptides, type V Cas polypeptides, and type VI, CRISPR-associated RNA binding proteins, or functional fragments thereof. Cas polypeptides suitable for use with the present disclosure can include but are not limited to Cas9, Cas12, Cas13, Cpf1 (or Cas12a), C2C1, C2C2 (or Cas13a), Cas13b, Cas13c, Cas13d, C2C3, Cas1, Cas1B, Cas2, Cas3, Cas4, Cas5, Cas5e (CasD), Cas6, Case, Casof, Cas7, Cas8a, Cas8a1, Cas8a2, Cas8b, Cas8c, Csn1, Csx12, Cas10, Cas10d, Cas10, Cas10d, CasF, CasG, CasH, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, or Cul966. Cas13 can include Cas13a, Cas13b, Cas13c, and Cas 13d (e.g., CasRx). Cas can be DNA (e.g., Cpf1, Cas9) and/or RNA cleaving (e.g., Cas13).

TABLE 7
Non-limiting examples of target sequences, such as sequences for ICP11, VP19,
VP26, and collagenase-like gene used in vectors described herein. Note that
either strand of the virus DNA may be targeted.

| | | | SEQ | Targeting sequence | SEQ |
|---|---|---|---|---|---|
| | | | ID | (reverse complement | ID |
| # | Gene | Virus Sequence Targeted | NO: | of virus sequence)* | NO: |
| 1 | ICP11 | ATGATTGTGCCAACAACAGA | 22. | TCTGTTGTTGGCACAA | 23. |
| | | | | TCAT | |
| 2 | ICP11 | TGATTGTGCCAACAACAGAA | 24. | TTCTGTTGTTGGCACA | 25. |
| | | | | ATCA | |
| 3 | ICP11 | GCGTTGAATCTCCAGTCAAA | 26. | TTTGACTGGAGATTCA | 27. |
| | | | | ACGC | |
| 4 | ICP11 | GCGTTGAATCTCCAGTCAAA | 28. | TTTGACTGGAGATTCA | 29 |
| | | | | ACGC | |
| 5 | ICP11 | TCTGTTGTTGGCACAATCGT | 30. | TCTGTTGTTGGCACAA | 31. |
| | | ATGATTGTGCCAACAACAGA | | TCATACGATTGTGCCA | |
| | | | | ACAACAGA | |
| 6 | ICP11 | TTGAGGACAAAAGTAGATCC | 32. | GGATCTACTTTTGTCCT | 33. |
| | | | | CAA | |
| 7 | ICP11 | GTCAAAAGACGCAATTCTTC | 34. | GAAGAATTGCGTCTTT | 35. |
| | | | | TGAC | |
| 8 | ICP11 | TGTAAACACGGTCATCTAGA | 36. | TCTAGATGACCGTGTTT | 37. |
| | | | | ACA | |
| 9 | ICP11 | TGCTTTAGGCACACCATGTA | 38. | TACATGGTGTGCCTAA | 39 |
| | | | | AGCA | |
| 10 | ICP11 | CAACAGTATCAAAAGTCTTC | 40. | GAAGACTTTTGATACT | 41. |
| | | | | GTTG | |
| 11 | ICP11 | TTAGGCACACCATGTAAACA | 42. | TTAGGCACACCATGTA | 43. |
| | | TGTTTACATGGTGTGCCTAA | | AACATGTTTACATGGT | |
| | | | | GTGCCTAA | |
| 12 | ICP11 | CCATTGAGGACAAAAGTAG | 44. | TCTACTTTTGTCCTCAA | 45. |
| | | A | | TGG | |
| 13 | ICP11 | TTCTGTTGTTGGCACAATCAT | 46. | TTCTGTTGTTGGCACA | 47. |
| | | GATTGTGCCAACAACAGAA | | ATCATGATTGTGCCAAC | |
| | | | | AACAGAA | |
| 14 | ICP11 | GAAGGTGGCCATTTTTTTATA | 48. | GAAGGTGGCCATTTTT | 49. |
| | | TAAAAAAATGGCCACCTTC | | TTATATAAAAAAATGGC | |
| | | | | CACCTTC | |
| | ICP11 | ATTCTTCGATGCCTCCATTGC | 50. | ATTCTTCGATGCCTCCA | 51. |
| | | AATGGAGGCATCGAAGAAT | | TTGCAATGGAGGCATC | |
| | | | | GAAGAAT | |
| | ICP11 | ATCCCCCACCAGCAAGAAAT | 52. | ATCCCCCACCAGCAAG | 53. |
| | | ATTTCTTGCTGGTGGGGGAT | | AAATATTTCTTGCTGGT | |
| | | | | GGGGGAT | |
| | ICP11 | CTTCAAGAAGTCTTCTTTCA | 54. | TGAAAGAAGACTTCTT | 55. |
| | | | | GAAG | |
| 15 | VP19 | TCTTTGGTCCCATCCATACA | 56. | TGTATGGATGGGACCA | 57. |
| | | | | AAGA | |
| 16 | VP19 | CCTCTTGGGGTAAGACATAA | 58. | TTATGTCTTACCCCAAG | 59. |
| | | | | AGG | |
| 17 | VP19 | TCAAGATCTTCCATGGTGTA | 60. | TACACCATGGAAGATC | 61. |
| | | | | TTGA | |
| 18 | VP19 | GCCGTCATGAATGTATGGATA | 62. | GCCGTCATGAATGTATG | 63. |
| | | TCCATACATTCATGACGGC | | GATATCCATACATTCAT | |
| | | | | GACGGC | |
| 19 | VP19 | CTCAATTGGTATCCTCGTCCG | 64. | CTCAATTGGTATCCTCG | 65 |
| | | GACGAGGATACCAATTGAG | | TCC | |
| | | | | GGACGAGGATACCAAT | |
| | | | | TGAG | |
| 20 | VP19 | ACGATAACGATGATGAGGAC | 66. | GTCCTCATCATCGTTAT | 67. |
| | | | | CGT | |
| 21 | VP19 | TTGATCGTTGCTATCTCAATA | 68. | TTGATCGTTGCTATCTC | 69. |
| | | TTGAGATAGCAACGATCAA | | AAT | |
| | | | | ATTGAGATAGCAACGA | |
| | | | | TCAA | |
| 22 | VP19 | ACGACTAACACTCTTCCTTT | 70. | ACGACTAACACTCTTC | 71. |
| | | AAAGGAAGAGTGTTAGTCG | | CTTT | |
| | | T | | AAAGGAAGAGTGTTAG | |
| | | | | TCGT | |
| 23 | VP19 | TGGAAGATCTTGAAGGCTCC | 72. | GGAGCCTTCAAGATCT | 73. |
| | | | | TCCA | |
| 24 | VP19 | TGTATGGATGGGACCAAAGA | 74. | TGTATGGATGGGACCA | 75. |
| | | TCTTTGGTCCCATCCATACA | | AAGATCTTTGGTCCCAT | |
| | | | | CCATACA | |
| 25 | VP19 | TTATGTCTTACCCCAAGAGG | 76. | TTATGTCTTACCCCAAG | 77. |
| | | CCTCTTGGGGTAAGACATAA | | AGG | |
| | | | | CCTCTTGGGGTAAGAC | |
| | | | | ATAA | |
| 26 | VP19 | TACACCATGGAAGATCTTGA | 78. | TACACCATGGAAGATC | 79. |
| | | TCAAGATCTTCCATGGTGTA | | TTGATCAAGATCTTCCA | |
| | | | | TGGTGTA | |
| 27 | VP19 | ATGAGGACAAATATAAGAAC | 80. | ATGAGGACAAATATAA | 81. |
| | | GTTCTTATATTTGTCCTCAT | | GAACGTTCTTATATTTG | |
| | | | | TCCTCAT | |
| 28 | VP19 | TTTTTATGTCTTACCCCAAGC | 82. | TTTTTATGTCTTACCCC | 83. |
| | | TTGGGGTAAGACATAAAAA | | AAGCTTGGGGTAAGAC | |
| | | | | ATAAAAA | |
| 29 | VP19 | ACAAATATAAGAACAGGACC | 84. | ACAAATATAAGAACAG | 85. |
| | | GGTCCTGTTCTTATATTTGT | | GACC | |
| | | | | GGTCCTGTTCTTATATT | |
| | | | | TGT | |
| 30 | VP19 | CAAATATAAGAACAGGACCA | 86. | CAAATATAAGAACAGG | 87. |
| | | TGGTCCTGTTCTTATATTTG | | ACCATGGTCCTGTTCTT | |
| | | | | ATATTTG | |
| 31 | VP26 | TCATTGACGTGTGCAGCAAA | 88. | TTTGCTGCACACGTCA | 89. |
| | | | | ATGA | |
| 32 | VP26 | TCTTTGAATTGGGACTCGCA | 90. | TGCGAGTCCCAATTCA | 91. |
| | | | | AAGA | |
| 33 | VP26 | CCTTTCCAAGAGGAGTATTG | 92. | CAATACTCCTCTTGGA | 93. |
| | | | | AAGG | |
| 34 | VP26 | TCATTGACGTGTGCAGCAAA | 94. | TTTGCTGCACACGTCA | 95. |
| | | | | ATGA | |
| 35 | VP26 | GCAAAGGTAATGTCAATTCG | 96. | GCAAAGGTAATGTCAA | 97. |
| | | CGAATTGACATTACCTTTGC | | TTCGCGAATTGACATTA | |
| | | | | CCTTTGC | |
| 36 | VP26 | TGCGAGTCCCAATTCAAAGA | 98. | TGCGAGTCCCAATTCA | 99. |
| | | TCTTTGAATTGGGACTCGCA | | AAGATCTTTGAATTGG | |
| | | | | GACTCGCA | |
| 37 | VP26 | GAACACTTACTTCTCGAGCA | 100. | AGGTCCTACAATACTC | 101 |
| | | TGCTCGAGAAGTAAGTGTTC | | CTCTAGAGGAGTATTG | |
| | | | | TAGGACCT | |
| 38 | VP26 | GAACACTTACTTCTCGAGCA | 101. | GAACACTTACTTCTCG | 103. |
| | | TGCTCGAGAAGTAAGTGTTC | | AGCATGCTCGAGAAGT | |
| | | | | AAGTGTTC | |
| 39 | VP26 | ATTGTATTCAACACACGTGT | 104. | ATTGTATTCAACACAC | 105. |
| | | ACACGTGTGTTGAATACAAT | | GTGTACACGTGTGTTG | |
| | | | | AATACAAT | |
| 40 | VP26 | TGAAGAATGGTCTCTCCGAT | 106. | ATCGGAGAGACCATTC | 107. |
| | | | | TTCA | |
| 41 | VP26 | CATTGACGTGTGCAGCAAAT | 108. | ATTTGCTGCACACGTC | 109. |
| | | | | AATG | |
| 42 | VP26 | ACAATACTCCTCTTGGAAAG | 110. | CTTTCCAAGAGGAGTA | 111. |
| | | | | TTGT | |
| 43 | VP26 | TGATCAACGACATTACTGTT | 112. | TGATCAACGACATTAC | 113. |
| | | AACAGTAATGTCGTTGATCA | | TGTTAACAGTAATGTC | |
| | | | | GTTGATCA | |
| 44 | VP26 | TTGAAGATCCTCAACAACAC | 114. | TTGAAGATCCTCAACA | 115. |
| | | GTGTTGTTGAGGATCTTCAA | | ACACGTGTTGTTGAGG | |
| | | | | ATCTTCAA | |
| 45 | VP26 | CTTGGAAAGGTGGCCATGAA | 116. | TTCATGGCCACCTTTCC | 117. |
| | | | | AAG | |
| 46 | VP26 | TTCGAAGCTACAATGAACAT | 118. | TTCGAAGCTACAATGA | 119. |
| | | ATGTTCATTGTAGCTTCGAA | | ACATATGTTCATTGTAG | |
| | | | | CTTCGAA | |
| 47 | VP26 | TAATGTCAATTCGTGGAGAG | 120. | TAATGTCAATTCGTGG | 121. |
| | | CTCTCCACGAATTGACATTA | | AGAGCTCTCCACGAAT | |
| | | | | TGACATTA | |
| 48 | VP26 | CTACAATACTCCTCTTGGAAT | 122. | CTACAATACTCCTCTTG | 123. |
| | | TCCAAGAGGAGTATTGTAG | | GAATTCCAAGAGGAGT | |
| | | | | ATTGTAG | |
| 49 | VP26 | TTGCAATCATTGCTCTAATC | 124. | GATTAGAGCAATGATT | 125. |
| | | | | GCAA | |
| 50 | VP26 | ACTGTTATTGCAGGAAACAT | 126. | ATGTTTCCTGCAATAAC | 127. |
| | | | | AGT | |
| 51 | VP26 | CAAGAATGTGATCGATATCAT | 128. | CAAGAATGTGATCGAT | 129. |
| | | GATATCGATCACATTCTTG | | ATCATGATATCGATCAC | |
| | | | | ATTCTTG | |
| 52 | VP26 | CCCAATTCAAAGAAGGGCA | 130. | CCCAATTCAAAGAAGG | 131. |
| | | ATTGCCCTTCTTTGAATTGGG | | GCAATTGCCCTTCTTTG | |
| | | | | AATTGGG | |
| 53 | VP26 | AGAACTCTAACATGACTTTG | 132. | CAAAGTCATGTTAGAG | 133. |
| | | | | TTCT | |
| 54 | VP26 | CAATACTCCTCTTGGAAAGG | 134. | CAATACTCCTCTTGGA | 135. |
| | | CCTTTCCAAGAGGAGTATTG | | AAGGCCTTTCCAAGAG | |
| | | | | GAGTATTG | |
| 55 | VP26 | ATTCAAAGAAGGGCAAAGG | 136. | ACCTTTGCCCTTCTTTG | 137. |
| | | T | | AAT | |
| 56 | Collagen- | CTAATAGGCTCTCCAAACAA | 138. | TTGTTTGGAGAGCCTA | 139. |
| | like gene | | | TTAG | |
| 57 | Collagen- | TCTAATAGGCTCTCCAAACA | 140. | TGTTTGGAGAGCCTAT | 141. |
| | like gene | | | TAGA | |
| 58 | Collagen- | AACGCCCCTTGGTCAATGTA | 142. | TACATTGACCAAGGGG | 143. |
| | like gene | | | CGTT | |

As previously discussed, the present invention is not limited to Cas9 nuclease. Further, the present invention is not limited to the aforementioned target genes described in Table 7. Still further, the present invention is not limited to the specific target sequences listed in Table 7. Methods for target sequence selection are well known to one of ordinary skill in the art. For example, in some embodiments, a user first identifies a protospacer adjacent motif (PAM) in the viral DNA sequence. In some embodiments, a PAM site refers to a short DNA sequence that follows the DNA region targeted for cleavage. the user may then select the target sequence, wherein the target sequence is a sequence (or is based on the sequence) upstream of the PAM (not including the PAM). In some embodiments, the target sequence is the sequence (or is based on the sequence) that spans from the nucleotide 25 nucleotides upstream of the PAM to the PAM (not including the PAM). In some embodiments, the target sequence is the sequence (or is based on the sequence) that spans from the nucleotide 24 nucleotides upstream of the PAM to the PAM (not including the PAM). In some embodiments, the target sequence is the sequence (or is based on the sequence) that spans from the nucleotide 23 nucleotides upstream of the PAM to the PAM (not including the PAM). In some embodiments, the target sequence is the sequence (or is based on the sequence) that spans from the nucleotide 22 nucleotides upstream of the PAM to the PAM (not including the PAM). In some embodiments, the target sequence is the sequence (or is based on the sequence) that spans from the nucleotide 21 nucleotides upstream of the PAM to the PAM (not including the PAM). In some embodiments, the target sequence is the sequence (or is based on the sequence) that spans from the nucleotide 20 nucleotides upstream of the PAM to the PAM (not including the PAM). In some embodiments, the target sequence is the sequence (or is based on the sequence) that spans from the nucleotide 19 nucleotides upstream of the PAM to the PAM (not including the PAM). In some embodiments, the target sequence is the sequence (or is based on the sequence) that spans from the nucleotide 18 nucleotides upstream of the PAM to the PAM (not including the PAM). In some embodiments, the target sequence is the sequence (or is based on the sequence) that spans from the nucleotide 17 nucleotides upstream of the PAM to the PAM (not including the PAM).

While most CRISPR nucleases require a PAM, the recognized PAM sequences are not shared by all Cas nucleases and instead vary widely, with different sequences, lengths, complexities, orientations, and distances from the target.

The present invention is not limited to nucleases that require a PAM. In some embodiments, the target sequence is not determined based on a PAM. As previously discussed, the PAMs may differ based on the particular nuclease.

Non-limiting examples of potential PAM sites may include but are not limited to NGG (for Streptococcus pyogenes Cas9), NGRRT or NGRRN (for Staphylococcus aureus Cas9), NNNNGATT (for Neisseria meningitidis Cas9), NNNNRYAC (for Campylobacter jejuni Cas9), NNAGAAW (for Streptococcus thermophilus Cas9), TTTV (for Lachnospiraceae bacterium for Cas12a (LbCpf1)), TTTV (for Acidaminococcus sp. Cas12a (AsCpf1)), TTN (for Alicyclobacillus acidiphilus AacCas12b), or ATTN, TTTN or GTTN (for Bacillus hisashii) (where “N” can be any nucleotide base, “R” is a purine and “Y” is a pyrimidine). In some embodiments, nucleases described herein may be engineered to recognize other PAM sites. In other embodiments, nucleases described herein may require no PAM sits.

As previously discussed, the present invention is not limited to the use of Cas9, nor the targeted viral proteins disclosed in Table 7, nor the specific target sequences in Table 7. As a non-limiting example, target sequences that may be selected for Cas12b (AacCas12b) based on VP19 may include GGCCACCACGACTAACACTC (SEQ ID NO: 160), CAGGACCAGGGATATGATGC (SEQ ID NO: 161), or GGACCAAAGAAGGACAGCGA (SEQ ID NO: 162). Non-limiting examples of target sequences that may be selected for Bacillus hisashii based on VP19 may include GGCTGGGTCCGCTCTTCT (SEQ ID NO: 162), CATGGGTCTCTTTTTGATC (SEQ ID NO: 163), or TCCTCGTTTCCGCCGCCACC (SEQ ID NO: 164). One of ordinary skill in the art would be capable of determining additional target sequences for any of the programmable nucleases disclosed herein (or those not listed, or those discovered in the future).

In some embodiments, the target sequence is 15 nucleotides in length. In some embodiments, the target sequence is 16 nucleotides in length. In some embodiments, the target sequence is 17 nucleotides in length. In some embodiments, the target sequence is 18 nucleotides in length. In some embodiments, the target sequence is 19 nucleotides in length. In some embodiments, the target sequence is 20 nucleotides in length. In some embodiments, the target sequence is 21 nucleotides in length. In some embodiments, the target sequence is 22 nucleotides in length. In some embodiments, the target sequence is 23 nucleotides in length. In some embodiments, the target sequence is 24 nucleotides in length. In some embodiments, the target sequence is 25 nucleotides in length. In some embodiments, the target sequence is 26 nucleotides in length. In some embodiments, the target sequence is 27 nucleotides in length. In some embodiments, the target sequence is 28 nucleotides in length. In some embodiments, the target sequence is 29 nucleotides in length. In some embodiments, the target sequence is 30 nucleotides in length. In some embodiments, the target sequence is 31 nucleotides in length. In some embodiments, the target sequence is 32 nucleotides in length. In some embodiments, the target sequence is 33 nucleotides in length. In some embodiments, the target sequence is 34 nucleotides in length. In some embodiments, the target sequence is 35 nucleotides in length. In some embodiments, the target sequence is 36 nucleotides in length. In some embodiments, the target sequence is 37 nucleotides in length. In some embodiments, the target sequence is 38 nucleotides in length. In some embodiments, the target sequence is 39 nucleotides in length. In some embodiments, the target sequence is 40 nucleotides in length. In some embodiments, the target sequence is more than 40 nucleotides in length.

### Example 7: Inhibition of WSSV Infection in Shrimp Using 2Nd Generation Crispr/Cas Constructs Targeting WSSV Genes

2^{nd} generation vectors (see e.g., p23, p24, p25, or p26 in FIG. 6, FIG. 7, FIG. 8, and FIG. 9) are administered to shrimp by (a) feeding to shrimp by mixing homogenates of bacterial cells expressing the vectors with standard shrimp feed, (b) feeding to shrimp by mixing pure plasmid preparation as part of standard shrimp feed, or (c) direct injection into shrimp in saline vehicle. After a period, post-feeding, shrimp display resistance to WSSV infection as measured by either viral load over time, or decreased time to mortality.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Example 8: Prophylactic Use of Methods and Compositions Described Herein

The following is a non-limiting example of the present invention. It is to be understood that said example is not intended to limit the present invention in any way. Equivalents or substitutes are within the scope of the present invention.

A shrimp farmer hears that a nearby pond is experiencing the start of a White Spot Syndrome Virus (WSSV) outbreak. To prevent WSSV from spreading into his ponds the farmer immediately begins to feed his shrimp with feed coated with a composition that reduces the replication of the WSSV. The famer continues to feed the shrimp this feed until the WSSV outbreak at the other farmer has cleared.

### Example 9: Prophylactic Use of Methods and Compositions Described Herein

The following is a non-limiting example of the present invention. It is to be understood that said example is not intended to limit the present invention in any way. Equivalents or substitutes are within the scope of the present invention.

A new shrimp farmer has just gotten a broodstock of shrimp to begin shrimp farming. He has previously heard the WSSV can wipe out entire shrimp ponds quickly. Therefore, to prevent WSSV from spreading through his ponds the farmer makes the choice to only feed his broodstock shrimp with a feed that has a composition that reduces the replication of the WSSV incorporated into it. The famer continues to feed the brookstock shrimp this feed for the entire time he has these shrimp. No WSSV outbreak ever occurs in the shrimp.

### Example 10: Use of Methods and Compositions Described Herein

The following is a non-limiting example of the present invention. It is to be understood that said example is not intended to limit the present invention in any way. Equivalents or substitutes are within the scope of the present invention.

A shrimp farmer notices that a few shrimp have noticeable white spots, unfortunately, indicating that those shrimps have been infected with the White Spot Syndrome Virus (WSSV). The farmer immediately removes these shrimps from the pond. Then to make sure that the WSSV does not continue to spread to other shrimp in the pond the farmer immediately begins to feed his shrimp with feed coated with a composition that reduces the replication of the WSSV. The farmers continue to feed the shrimp with the coated feed for several weeks. After that time, no other shrimps have contracted WSSV. To be safe the farmer continues to feed his shrimp the coated feed for several more weeks.

### EMBODIMENTS

The following embodiments are intended to be illustrative only and not to be limiting in any way.

Embodiment 1. A method for inhibiting infection of or reducing replication of a virus in an animal in need thereof, comprising introducing to a cell of said animal a nuclease comprising a gene-binding moiety, wherein said gene binding moiety is configured to bind at least one gene of said virus.

Embodiment 2. The method of embodiment 1, wherein said virus is a DNA virus.

Embodiment 3. The method of embodiment 1 or embodiment 2, wherein the virus belongs to the genus Whispovirus.

Embodiment 4. The method of embodiment 3, wherein the virus belongs to the family Nimaviridae.

Embodiment 5. The method of embodiment 3, wherein said virus is white spot syndrome virus (WSSV).

Embodiment 6. The method of any one of embodiments 1-4, wherein said at least one gene of said virus comprises at least one gene described in Table B, or any combination thereof.

Embodiment 7. The method of any one of embodiments 1-5, wherein said one or more genes of said virus encode ICP11 or a fragment thereof, VP19 or a fragment thereof, VP26 or a fragment thereof, collagen-like protein (WSSV-CLP) or a fragment thereof, or any combination thereof.

Embodiment 8. The method of any one of embodiments 1-6, wherein said animal is a crustacean.

Embodiment 9. The method of embodiment 7, wherein said crustacean is a shrimp, a prawn, a crab, or a crayfish.

Embodiment 10. The method of embodiment 8, comprising inhibiting infection in a shrimp, a prawn, wherein said shrimp is Litopenaeus vannamei.

Embodiment 11. The method of any one of embodiments 1-10, wherein said gene-binding moiety is configured to bind a plurality of different portions of said one or more genes of said virus.

Embodiment 12. The method of any one of embodiments 1-11, wherein said gene-binding moiety is configured to bind a combination of at least two, at least three, or all four of ICP11, VP19, VP26, collagen-like protein (WSSV-CLP), or any combination thereof.

Embodiment 13 The method of any one of embodiments 1-12, wherein said gene binding moiety is configured to further bind at least additional gene of said virus comprising DNA polymerase, RR1, VP28, or any combination thereof.

Embodiment 14. The method of any one of embodiments 1-13, wherein said nuclease is a programmable nuclease comprising at least one of a CRISPR-associated (Cas) polypeptide, a zinc finger nuclease (ZFN), a transcription activator-like effector nuclease (TALEN), or a combination thereof.

Embodiment 15. The method of any one of embodiments 1-14, wherein said nuclease is configured to bind at least 5, or at least 18-24 consecutive nucleotides at least one sequence selected from SEQ ID NOs: 22-82 or a variant having at least 80%, 90%, 95%, or 99% identity thereto.

Embodiment 16. The method of any one of embodiments 1-15, wherein said nuclease is further configured to bind at least 5, or at least 18-24 consecutive nucleotides of at least one, at least two, or at least three sequences selected from SEQ ID NOs: 1-9 or a variant having at least 80%, 90%, 95%, or 99% identity thereto.

Embodiment 17. The method of any one of embodiments 14-16, wherein said nuclease is a programmable nuclease comprising a CRISPR-associated (Cas) polypeptide, wherein said Cas polypeptide is a type I CRISPR-associated (Cas) polypeptide, a type II CRISPR-associated (Cas) polypeptide, a type III CRISPR-associated (Cas) polypeptide, a type IV CRISPR-associated (Cas) polypeptide, a type V CRISPR-associated (Cas) polypeptide, a type VI CRISPR-associated (Cas) polypeptide.

Embodiment 18. The method of any one of embodiments 14-17, wherein said gene-binding moiety of said nuclease comprises a heterologous RNA polynucleotide configured to hybridize to said one or more genes of said virus.

Embodiment 19 The method of embodiment 18, wherein said heterologous RNA polynucleotide comprises at least one, at least two, or at least three targeting sequences, wherein said targeting sequence comprises at least 17 consecutive nucleotides of at least one sequence selected from SEQ ID NOs: 83-143 or a variant having at least 80%, 90%, 95%, or 99% identity thereto.

Embodiment 20. The method of embodiment 18 or 19, wherein said heterologous RNA polynucleotide further comprises at least one, at least two, or at least three targeting sequences, wherein said targeting sequence comprises at least 17 consecutive nucleotides of at least one sequence selected from SEQ ID NOs: 10-18 or a variant having at least 80%, 90%, 95%, or 99% identity thereto.

Embodiment 21. The method of any one of embodiments 1-20, wherein introducing a nuclease comprising a gene-binding moiety to said cell of said animal comprises contacting said cell with said nuclease.

Embodiment 22. The method of embodiment 21, wherein said nuclease comprises a ribonucleoprotein complex comprising a Cas polypeptide and at least one, at least two, or at least three heterologous RNA polynucleotides configured to hybridize to said one or more genes of said virus.

Embodiment 23. The method of any one of embodiments 1-22, wherein introducing a nuclease comprising a gene-binding moiety to said cell of said animal comprises contacting said cell with a capped mRNA comprising a sequence encoding said nuclease.

Embodiment 24. The method of embodiment 22, wherein said nuclease comprises a Cas polypeptide, wherein introducing a nuclease comprising a gene-binding moiety to said cell of said animal further comprises contacting said cell with at least one, at least two, or at least three heterologous RNA polynucleotides configured to hybridize to said one or more genes of said virus.

Embodiment 25. The method of embodiment 23, wherein said capped mRNA and said heterologous RNA polynucleotide are separate RNAs.

Embodiment 26. The method of any one of embodiments 1-25, wherein introducing a nuclease comprising a gene-binding moiety to said cell of said animal comprises contacting said cell with a vector comprising a sequence encoding said nuclease.

Embodiment 27. The method of embodiment 26, wherein said nuclease comprises a Cas polypeptide, wherein said vector further encodes at least one, at least two, or at least three heterologous RNA polynucleotides configured to hybridize to said one or more genes of said virus.

Embodiment 28 The method of embodiment 26 or 27, wherein said vector is a plasmid, a minicircle, or a viral vector.

Embodiment 29. The method of embodiment 28, wherein said vector is a viral vector, wherein said viral vector is a baculoviral vector.

Embodiment 30. The method of any one of embodiments 26-29, wherein said sequence encoding said nuclease is codon-optimized for expression in said crustacean.

Embodiment 31. The method of any one of embodiments 1-30, wherein said introducing occurs in vivo, ex vivo, or in vitro.

Embodiment 32 The method of any one of embodiments 1-31, wherein said nuclease cleaves viral genomic DNA encoding said one or more genes of said virus within said cell of said animal.

Embodiment 33. The method of any one of embodiments 1-32, wherein said method results in delay of mortality of said animal upon infection with said virus belonging to the family Nimaviridae.

Embodiment 34. The method of any one of embodiments 1-32, wherein said method results in reduced mortality of said animal upon infection with said virus belonging to the family

### Nimaviridae.

Embodiment 35. The method of any one of embodiments 1-34, wherein introducing to a cell of said animal said nuclease comprises injecting said animal with said nuclease or a vector encoding said nuclease.

Embodiment 36. The method of any one of embodiments 1-35, wherein introducing to a cell of said animal said nuclease comprises administering orally to said animal said nuclease or a vector encoding said nuclease.

Embodiment 37. A vector comprising a sequence encoding at least one programmable nuclease configured to bind at least one viral gene of a virus from the family Nimaviridae

Embodiment 38. The vector of embodiment 37, wherein said at least one viral gene comprises any of the genes in Table B or a fragment thereof, or any combination thereof.

Embodiment 39. The vector of embodiment 37, wherein said at least one viral gene comprises ICP11 or a fragment thereof, VP19 or a fragment thereof, VP26 or a fragment thereof, collagen like protein (WSSV-CLP) or a fragment thereof, or any combination thereof.

Embodiment 40. The vector of any one of embodiments 37-39, wherein said vector is a plasmid, a minicircle, or a viral vector.

Embodiment 41. The vector of embodiment 40, wherein said viral vector is a baculoviral vector.

Embodiment 42. The vector of any one of embodiments 37-41, wherein said nuclease is a programmable nuclease comprising at least one of a CRISPR-associated (Cas) polypeptide, a zinc finger nuclease (ZFN), a transcription activator-like effector nuclease (TALEN), or a combination thereof.

Embodiment 43. The vector of any one of embodiments 37-42, wherein said programmable nuclease is configured to bind a plurality of different portions of said one or more genes of said virus.

Embodiment 44. The vector of any one of embodiments 37-43, wherein said programmable nuclease is configured to bind a combination of at least two, at least three, or all four of ICP11, VP19, VP26, or collagen-like protein.

Embodiment 45. The vector of any one of embodiments 37-44, wherein said programmable nuclease is configured to further bind at least additional gene of said virus comprising DNA polymerase, RR1, VP28, or any combination thereof.

Embodiment 46. The vector of any one of embodiments 37-45, wherein said nuclease is configured to bind at least 5, or at least 18-24 consecutive nucleotides at least one sequence selected from SEQ ID NOs: 22-70 or a variant having at least 80%, 90%, 95%, or 99% identity thereto.

Embodiment 47. The vector of any one of embodiments 37-46, wherein said nuclease is further configured to bind at least 5, or at least 18-24 consecutive nucleotides of at least one, at least two, or at least three sequences selected from SEQ ID NOs: 1-9 or a variant having at least 80%, 90%, 95%, or 99% identity thereto.

Embodiment 48. The vector of any one of embodiments 37-47, wherein said programmable nuclease comprises a CRISPR-associated (Cas) polypeptide, wherein said Cas polypeptide is a type I CRISPR-associated (Cas) polypeptide, a type II CRISPR-associated (Cas) polypeptide, a type III CRISPR-associated (Cas) polypeptide, a type IV CRISPR-associated (Cas) polypeptide, a type V CRISPR-associated (Cas) polypeptide, a type VI CRISPR-associated (Cas) polypeptide.

Embodiment 49. The vector of embodiment 48, wherein said vector further comprises a second sequence encoding at least one, at least two, or at least three heterologous RNA polynucleotides configured to hybridize to said one or more genes of said virus.

Embodiment 50. The vector of embodiment 49, wherein said heterologous RNA polynucleotide comprises at least one, at least two, or at least three targeting sequences, wherein said targeting sequence comprises at least 17 consecutive nucleotides of at least one sequence selected from SEQ ID NOs: 83-143, a variant having at least 80%, 90%, 95%, or 99% identity thereto, or a variant substantially identical thereto.

Embodiment 51. The vector of any one of embodiments 48-50, wherein said heterologous RNA polynucleotide comprises at least one, at least two, or at least three targeting sequences, wherein said targeting sequence comprises at least 17 consecutive nucleotides of at least one sequence selected from SEQ ID NOs: 10-18.

Embodiment 52. The vector of any one of embodiments 49-51, wherein said sequence encoding said heterologous RNA polynucleotide is operably linked to a sequence comprising an ie1 promoter from said virus from the family Nimaviridae.

Embodiment 53. The vector of any one of embodiments 49-51, wherein said sequence encoding said heterologous RNA polynucleotide is operably linked to a sequence comprising an ie1 promoter from white spot syndrome virus (WSSV).

Embodiment 54. The vector of any one of embodiments 49-51, wherein said sequence encoding said heterologous RNA polynucleotide is operably linked to a sequence comprising at least 100 consecutive nucleotides of SEQ ID NO:21, a variant having at least 80%, at least 90%, at least 95%, at least 99% identity thereto, or a variant substantially identical thereto.

Embodiment 55. The vector of any one of embodiments 37-54, wherein said programmable nuclease is operably linked to a sequence comprising a P2 promoter from infectious hypodermal and hematopoietic necrosis virus (IHHNV) of shrimp.

Embodiment 56. The vector of any one of embodiments 37-54, wherein said programmable nuclease is operably linked to a sequence comprising at least 100 consecutive nucleotides of any one of SEQ ID NOs: 20-162, a variant having at least 80%, at least 90%, at least 95%, at least 99% identity thereto, or a variant substantially identical thereto.

Embodiment 57. The vector of any one of embodiments 37-56, wherein said sequence encoding said programmable nuclease is codon-optimized for expression in a crustacean species.

Embodiment 58. The vector of embodiment 57, wherein said crustacean species is a penaeid, a crab, or a crayfish.

Embodiment 59. The vector of embodiment 58, wherein said shrimp is Litopenaeus vannamei.

Embodiment 60. The vector of any one of claims 37-59, wherein said at least one programmable nuclease is configured to bind at least four different sites of at least one gene of said virus, wherein said at least one gene of said virus comprises DNA polymerase, RR1, VP28, or any combination thereof.

Embodiment 61. The method of any one of claims 1-36, wherein said gene binding moiety is configured to bind at least four different sites of at least one gene of said virus, wherein said at least one gene of said virus encodes DNA polymerase, RR1, VP28, or any combination thereof.

Embodiment 62. A vector comprising the sequence of any one of SEQ ID NOs: 145, 146, 147, 148, 149, 150, or 151.

Embodiment 63. A pharmaceutically-acceptable composition, comprising the vector of any one of embodiments 37-60 or 62 and a pharmaceutically-acceptable excipient.

1. A method for inhibiting infection of or reducing replication of a virus in an animal in need thereof, comprising introducing to a cell of said animal a nuclease comprising a gene-binding moiety, wherein said gene binding moiety is configured to bind at least one or more genes of said virus, wherein said one or more genes of said virus encode ICP11 or a fragment thereof, VP19 or a fragment thereof, VP26 or a fragment thereof, collagen-like protein (WSSV-CLP) or a fragment thereof, or any combination thereof, wherein said virus belongs to the family Nimaviridae.

2. The method of claim 1, wherein said animal is a crustacean, wherein said crustacean is a shrimp, a prawn, a crab, or a crayfish, wherein said shrimp is Litopenaeus vannamei; and wherein said virus belongs to the genus Whispovirus, wherein said virus is White spot syndrome virus (WSSV).

3-6. (canceled)

7. The method of claim 1, wherein said gene-binding moiety is configured to bind a plurality of different portions of said one or more genes of said virus, wherein said gene binding moiety is configured to bind at least four different portions of said one or more genes of said virus.

8. The method of claim 1, wherein said gene-binding moiety is configured to bind a combination of at least two, at least three, or [all] at least four of ICP11, VP19, VP26, collagen-like protein (WSSV-CLP), DNA polymerase, RR1, VP28, or any combination thereof.

9. (canceled)

10. The method of claim 1, wherein said nuclease is a programmable nuclease comprising at least one of a CRISPR-associated (Cas) polypeptide, a zinc finger nuclease (ZFN), a transcription activator-like effector nuclease (TALEN), or a combination thereof, wherein said nuclease is configured to bind at least 5 consecutive nucleotides of at least one sequence selected from SEQ ID NOs: 1-9, SEQ ID NOS: 22-82 or a variant having at least 80%, 90%, 95%, or 99% identity thereto.

11-13. (canceled)

14. The method of claim 10, wherein said gene-binding moiety of said nuclease comprises a heterologous RNA polynucleotide configured to hybridize to said one or more genes of said virus, wherein said heterologous RNA polynucleotide comprises at least one, at least two, or at least three targeting sequences, wherein said targeting sequence comprises at least 17 consecutive nucleotides of at least one sequence selected from SEQ ID NOs: 10-18, SEQ ID NOs: 83-143 or a variant having at least 80%, 90%, 95%, or 99% identity thereto.

15-16. (canceled)

17. The method of claim 1, wherein introducing a nuclease comprising a gene-binding moiety to said cell of said animal comprises (i) contacting said cell with said nuclease, (ii) contacting said cell with a capped mRNA comprising a sequence encoding said nuclease, or (iii) contacting said cell with a vector comprising a sequence encoding said nuclease, wherein said sequence encoding said nuclease is codon-optimized for expression in a crustacean.

18-19. (canceled)

20. The method of claim 17, wherein said nuclease comprises a Cas polypeptide, wherein introducing a nuclease comprising a gene-binding moiety to said cell of said animal further comprises contacting said cell with at least one, at least two, or at least three heterologous RNA polynucleotides configured to hybridize to said one or more genes of said virus.

21-22. (canceled)

23. The method of claim 17, wherein said nuclease comprises a Cas polypeptide, wherein said vector further encodes at least one, at least two, or at least three heterologous RNA polynucleotides configured to hybridize to said one or more genes of said virus, wherein said nuclease cleaves viral genomic DNA encoding said one or more genes of said virus within said cell of said animal.

24-30. (canceled)

31. The method of claim 1, wherein introducing to a cell of said animal said nuclease comprises (i)_injecting said animal with said nuclease or a vector encoding said nuclease or (ii) administering orally to said animal said nuclease or a vector encoding said nuclease.

32. (canceled)

33. A vector comprising a sequence encoding at least one programmable nuclease configured to bind at least one or more [viral] genes of a virus from the family Nimaviridae, wherein said at least one or more genes of said virus encode comprises-ICP11 or a fragment thereof, VP19 or a fragment thereof, VP26 or a fragment thereof, collagen like protein (WSSV-CLP) or a fragment thereof, or any combination thereof.

34. The vector of claim 33, wherein said vector is a plasmid, a minicircle, or a viral vector.

35. (canceled)

36. The vector of claim 33, wherein said nuclease is a programmable nuclease comprising at least one of a CRISPR-associated (Cas) polypeptide, a zinc finger nuclease (ZFN), a transcription activator-like effector nuclease (TALEN), or a combination thereof.

37. The vector of claim 33, wherein said programmable nuclease is configured to bind a plurality of different portions of said one or more genes of said virus; wherein said programmable nuclease is configured to bind a combination of at least two, at least three, or at least four of ICP11, VP19, VP26, or collagen-like protein, DNA polymerase, RR1, VP28, or any combination thereof.

38-39. (canceled)

40. The vector of claim 33, wherein said programmable nuclease is configured to bind at least 5 consecutive nucleotides at least one sequence selected from SEQ ID NOs: 1-9, SEQ ID NOs: 22-70 or a variant having at least 80%, 90%, 95%, or 99% identity thereto.

41. (canceled)

42. The vector of claim 33, wherein said programmable nuclease comprises a CRISPR-associated (Cas) polypeptide, wherein said Cas polypeptide is a type I CRISPR-associated (Cas) polypeptide, a type II CRISPR-associated (Cas) polypeptide, a type III CRISPR-associated (Cas) polypeptide, a type IV CRISPR-associated (Cas) polypeptide, a type V CRISPR-associated (Cas) polypeptide, a type VI CRISPR-associated (Cas) polypeptide.

43. The vector of claim 42, wherein said vector further comprises a second sequence encoding at least one, at least two, or at least three heterologous RNA polynucleotides configured to hybridize to said one or more genes of said virus, wherein said heterologous RNA polynucleotide comprises at least one, at least two, or at least three targeting sequences, wherein said targeting sequence comprises at least 17 consecutive nucleotides of at least one sequence selected from SEQ ID NOs: 10-18, SEQ ID NOs: 83-143, a variant having at least 80%, 90%, 95%, or 99% identity thereto, or a variant substantially identical thereto.

44-45. (canceled)

46. The vector of claim 43, wherein said sequence encoding said heterologous RNA polynucleotide is operably linked to a sequence comprising an ie1 promoter from said virus from the family Nimaviridae, or from White spot syndrome virus (WSSV).

47-48. (canceled)

49. The vector of claim 33, wherein said programmable nuclease is operably linked to a sequence comprising a P2 promoter from infectious hypodermal and hematopoietic necrosis virus (IHHNV) of shrimp.

50. (canceled)

51. The vector of claim 33, wherein said sequence encoding said programmable nuclease is codon-optimized for expression in a crustacean species, wherein said crustacean species is a shrimp, prawn, a crab, or a crayfish, wherein said shrimp is Litopenaeus vannamei.

52-170. (canceled)
